SEQUENCE LISTING (4 sequences)

SEQ ID NO: 1 - RNA, 20 nt, synthetic construct
  source 1..20: mol_type = other RNA
tttctttgggtctcacctcc

SEQ ID NO: 2 - DNA, 187 bp, synthetic construct
  source 1..187: mol_type = other DNA
agactctgtgtcctcacagtgtacaaagtcagcatggttccagctttctttgggtctcaccttgtcatcgtcatccttgtaatcgatgtcatgatctttataatcaccgtcatggtctttgtagtcctccaggtttgcttctgggcctcttggactagaaatgagaaaaccacagagaatgaaaaga

SEQ ID NO: 3 - DNA, 25 bp, synthetic construct
  source 1..25: mol_type = other DNA
aggtcagctcccgagttcacaggcc

SEQ ID NO: 4 - DNA, 25 bp, synthetic construct
  source 1..25: mol_type = other DNA
cagtgggtcagtggtggcacgtggg

(19) United States
(12) Patent Application Publication
(10) Pub. No.: US 2026/0116867 A1
(43) Pub. Date: Apr. 30, 2026

(54) PROTEOLYSIS TARGETING CHIMERAS FOR HUMAN PREGNANE X RECEPTOR AND FOR DEGRADATION OF GSPT1

(71) Applicant: ST. JUDE CHILDREN'S RESEARCH HOSPITAL, Memphis, TN (US)

(72) Inventors: Taosheng Chen, Germantown, TN (US); Yongtao LI, Changsha (CN); Andrew Huber, Collierville, TN (US); Wenwei Lin, Memphis, TN (US)

(21) Appl. No.: 18/858,684

(22) PCT Filed: Apr. 24, 2023
(86) PCT No.: PCT/US2023/066121

§ 371 Date: Jun. 23, 2025

Related U.S. Application Data
(60) Provisional application No. 63/333,925, filed on Apr. 22, 2022.

(51) Int. Cl. C07D 401/14; A61K 31/454; C07D 401/04

(52) U.S. Cl. CPC C07D 401/14; A61K 31/454; C07D 401/04

(57) ABSTRACT

The present disclosure relates to 1,4,5-substituted 1,2,3-triazoles useful as modulators of pregnane X receptor (PXR) and in the treatment of disorders associated with PXR dysfunction (e.g., disorders of uncontrolled cellular proliferation, bowel disorders). The invention further relates to uses of the disclosed compounds in decreasing adverse drug reactions such as, for example, adverse reactions associated with administration of an anticancer agent, an antibacterial agent, a non-steroidal anti-inflammatory agent, and an anticonvulsant agent. This abstract is intended as a scanning tool for purposes of searching in the particular art and is not intended to be limiting of the present invention.

### CROSS-REFERENCE TO RELATED APPLICATIONS

This application is a national phase application under 35 U.S.C. § 371 of International Application No. PCT/US2023/066121, filed on Apr. 24, 2023, which claims the benefit of U.S. Provisional Application No. 63/333,925, filed on Apr. 22, 2022, the contents of which are incorporated herein by reference in their entireties entirety.

### STATEMENT REGARDING FEDERALLY SPONSORED RESEARCH

This invention was made with U.S. Government support under grant number GM118041, awarded by the National Institutes of Health. The U.S. government has certain rights in the invention.

### REFERENCE TO SEQUENCE LISTING

The Sequence Listing submitted Jun. 23, 2025 as a xml file named “19116.0050U2_SL.xml”, created on Jun. 22, 2025, and having a size of 8,192 bytes is hereby incorporated by reference pursuant to 37 C.F.R. § 1.52(e)(5).

### BACKGROUND

Over the last decade, targeted protein degradation has emerged as a useful tool to expand the scope of druggable proteins encoded by the human genome. In 2010, the CUL4-RBX1-DDB1-CRBN (CRL4CRB^{N}) E3 ubiquitin ligase complex was identified as the molecular target of the immunomodulatory drug thalidomide, and it was subsequently found that thalidomide and its derivatives act as “molecular glues” that bind to the E3 substrate receptor CRBN, thereby altering the substrate proteins recognized by CRL4^{CRBN}. Shortly thereafter, linkage of thalidomide to various target protein ligands resulted in proteolysis targeting chimeras (PROTACs) that specifically induced CRL4^{CRBN}-mediated degradation of a range of proteins, such as the epigenetic readers BRD2, BRD3, and BRD4. The demonstration that thalidomide-linked molecules could induce degradation of predetermined target proteins resulted in an explosion of the targeted protein degradation field. Importantly, this finding also led to small molecules that induce protein degradation by two distinct modes. PROTACs are bivalent molecules with an E3 ubiquitin ligase ligand linked to a ligand for the protein of interest; molecular glue-type degraders, on the other hand, are monovalent and facilitate specific protein-protein interactions.

Thalidomide was widely used in the 1950s and 1960s as a sedative and morning sickness treatment; however, it was discontinued due to teratogenic effects. Because of extensive biological characterization, thalidomide and derivatives pomalidomide and lenalidomide have now been reclassified as immunomodulatory drugs and approved for use as multiple myeloma treatments. Antimyeloma activity is attributed to CRL4^{CRBN}-mediated degradation of the transcription factors IKZF1 and IKZF3, which play critical roles in B cell development and are overexpressed in B cell malignancies. Further derivatization of thalidomide resulted in CC-885 and CC-90009, potent cytotoxic agents that induce degradation of the translation termination factor GSPT1. GSPT1 is upregulated in many cancers, particularly hematopoietic malignancies, and acute leukemia cells have been shown to be highly sensitive to GSPT1 degradation. GSPT1 is therefore a potential drug target for future chemotherapies.

Because CRL4^{CRBN}-directed PROTACs contain a phthalimide moiety that binds CRBN, these molecules have the potential to either (1) specifically induce degradation of the target protein of interest or (2) act as molecular glues that induce degradation of unintended targets. A class of phthalimide-conjugated molecules designed to degrade kinases was previously shown to degrade GSPT1 through a molecular glue mechanism rather than the intended kinases through the PROTAC mechanism. Furthermore, a simple structural modification to the MDM2-degrading PROTAC MD-222 resulted in loss of MDM2 degradation with a corresponding gain of GSPT1 degradation. These findings indicate the importance of off-target evaluation in PROTAC development. However, conversion of PROTACs to molecular glues can also be a means of deriving new chemical matter that reprograms CRL4^{CRBN} substrate specificity.

The discovery of a potent and selective dual inverse agonist and antagonist (SPA70) of the human nuclear receptor pregnane X receptor (PXR) was previously reported. Derivatization of SPA70 yielded only antagonists and agonists, suggesting that PXR inverse agonism is difficult to achieve. Furthermore, a single PXR mutation (W299A) converts SPA70 from an inverse agonist to an agonist. Thus, an alternative approach to developing PXR-specific inverse agonists is desirable.

Accordingly, there remains a need for compounds that induce protein degradation such as, for example, degradation of PXR or GSPT1, and methods of making and using same.

### SUMMARY

In accordance with the purpose(s) of the invention, as embodied and broadly described herein, the invention, in one aspect, relates to 1,4,5-substituted 1,2,3-triazoles that are useful as modulators of pregnane X receptor (PXR) and in the treatment of disorders associated with PXR dysfunction (e.g., cancer, bowel disorders). The invention further relates to the use of the disclosed compounds in decreasing adverse drug reactions such as, for example, adverse drug reactions associated with administration of an anticancer agent, an antibacterial agent, a non-steroidal anti-inflammatory agent, or an anticonvulsant agent.

Disclosed are compounds having a structure represented by a formula:

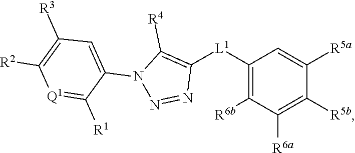

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷; wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein each of R^{6a} and R^{6b} is independently selected from hydrogen, C1-C8 alkyl, and R⁷; wherein R⁷ is a structure represented by a formula selected from:

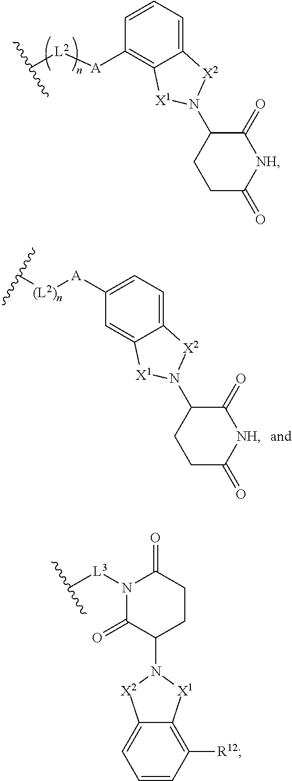

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

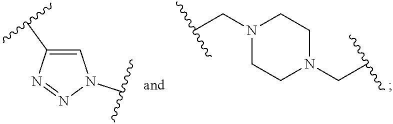

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, provided that one and only one of R², R^{6a}, and R^{6b} is R⁷, or a pharmaceutically acceptable salt thereof.

Also disclosed are pharmaceutical compositions comprising an effective amount of a disclosed compound or a pharmaceutically acceptable salt thereof, and a pharmaceutically acceptable carrier.

Also disclosed are methods for modulating pregnane X receptor (PXR) activity in a subject in need thereof, the method comprising administering to the subject an effective amount of a disclosed compound or a pharmaceutically acceptable salt thereof.

Also disclosed are methods for modulating pregnane X receptor (PXR) activity in a cell, the method comprising contacting the cell with an effective amount of a disclosed compound or a pharmaceutically acceptable salt thereof.

Also disclosed are methods for treating a disorder of uncontrolled cellular proliferation in a subject in need thereof, the method comprising administering to the subject an effective amount of a disclosed compound or a pharmaceutically acceptable salt thereof.

Also disclosed are kits comprising a disclosed compound or a pharmaceutically acceptable salt thereof, and one or more of (a) an agent known to increase pregnane X receptor (PXR) activity; (b) an agent known for the treatment of a disorder associated with pregnane X receptor (PXR) dysfunction; (c) administering the compound in connection with a disorder associated with pregnane X receptor (PXR) dysfunction; (d) instructions for decreasing an adverse drug reaction; and € instructions for treating a disorder associated with pregnane X receptor (PXR) dysfunction.

Also disclosed are methods for decreasing an adverse drug reaction in a subject in need thereof, the method comprising administering to the subject an effective amount of a compound having a structure represented by a formula:

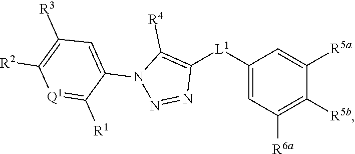

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{5a}and R^{5b} is independently selected from hydrogen and C1-C8 alkyl; wherein R^{6a} is R⁷; wherein R⁷ is a structure represented by a formula selected from:

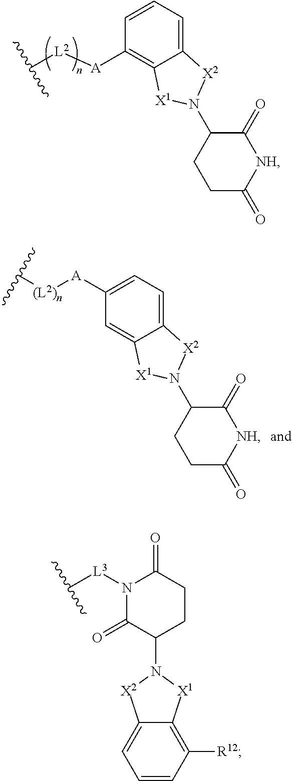

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

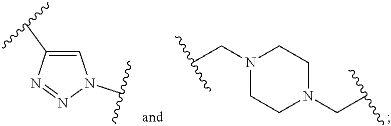

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof.

Also disclosed are methods for treating a bowel disorder in a subject in need thereof, the method comprising administering to the subject an effective amount of a compound having a structure represented by a formula:

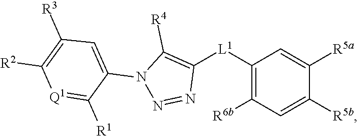

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein R^{6b} is R⁷; wherein R⁷ is a structure represented by a formula selected from:

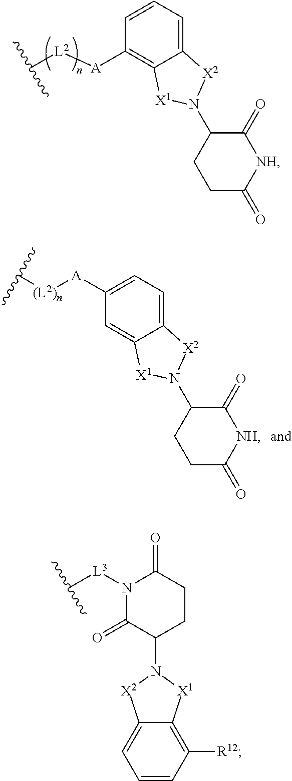

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

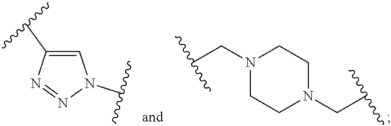

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof.

Also disclosed are kits comprising a compound having a structure represented by a formula:

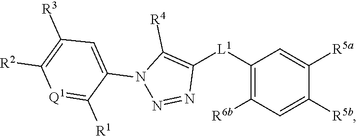

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein R^{6b} is a structure represented by a formula selected from:

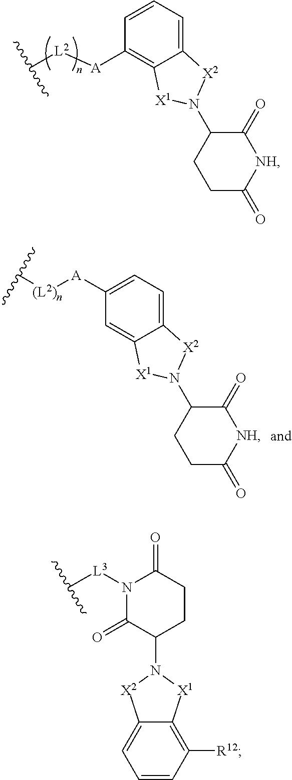

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

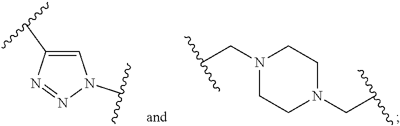

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof, and one or more of: (a) an agent known for the treatment of a disorder associated with pregnane X receptor (PXR) dysfunction; (b) instructions for administering the compound in connection with a disorder associated with pregnane X receptor (PXR) dysfunction; and (c) instructions for treating a disorder associated with pregnane X receptor (PXR) dysfunction.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative phthalimide-based molecular glues and proteolysis targeting chimeras (PROTACS) as further described herein.

FIG. 2A-D shows representative data illustrating the development of a high-throughput assay for degradation of overexpressed protein. Specifically, FIG. 2A shows a representative diagram illustrating the degradation tag (dTAG) system used to identify PXR degraders. FIG. 2B shows a representative structure of the PROTAC molecule dTAG-13.

FIG. 2C shows representative data illustrating that dTAG-13 results in only a slight reduction in HiBiT signal. FIG. 2D shows a representative Western blot image of HiBiT-FKBP12^{F36V}-PXR degradation using an HA tag for detection.

FIG. 3 shows representative synthesized compounds that link PXR ligands to CRBN ligands as further described herein.

FIG. 4A-D show representative data illustrating the identification of PXR degrader SJPYT-195. Specifically, FIG. 4A shows representative data illustrating that SJPYT-195 reduced HiBiT signal. FIG. 4B shows representative data illustrating that SJPYT-195 potently and efficaciously reduced endogenous PXR protein in the model system. FIG. 4C and FIG. 4D show representative data illustrating that the loss of PXR protein was dependent on the proteasome.

FIG. 5A-C show representative data illustrating that SJPYT-195 impacts PXR in manners inconsistent with direct degradation. Specifically, FIG. 5A and FIG. 5B show representative data illustrating that the kinetics of PXR protein decrease in response to SJYPT-195 were quite slow. FIG. 5C shows representative data illustrating that SJPYT-195 drastically reduced PXR RNA level.

FIG. 6A-F show representative data illustrating a whole-cell assessment of SJYPT-195 protein degrading activity. Specifically, FIG. 6A and FIG. 6B show representative data illustrating at 12 h, 5 μM SJPYT-195 reduced PXR protein ˜50% with negligible reduction ofPXR RNA. FIG. 6C shows representative data illustrating that SJPYT-195 is remarkably selective, only down-regulating five proteins (GSPT1, GSPT2, ZFP91, CYP1A1, and BRIP1) at the relatively high treatment concentration (5 μM) and duration (12 h). FIG. 6D and FIG. 6E show representative data illustrating that SJPYT-195 depletes GSPT1 at concentrations much lower than those required to reduce PXR. FIG. 6F shows representative data illustrating that GSPT1 loss was proteasome-dependent.

FIG. 7A-D show representative data illustrating that SJPYT-195 is a GSPT1 degrader. Specifically, FIG. 7A shows representative data illustrating that SJPYT-195 exhibited nanomolar toxicity in the SNU—C4 3xFLAG-PXR KI cell line that was proportional to GSPT1 degradation with respect to CC-885. FIG. 7B and FIG. 7C show representative data illustrating cell viability as a function of CC-885 (FIG. 7B) or SJPYT-195 (FIG. 7C) concentration. FIG. 7D shows a representative western blot image illustrating that the plasmid-derived GSPT1 was expressed at a significantly lower level than the endogenous GSPT1.

FIG. 8 shows representative analogs of SJPYT-195.

FIG. 9A-D shows representative data illustrating the SJPYT-195 structure-activity relationship. Specifically, FIG. 9A shows a representative western blot image illustrating that SJPYT-195 analogs have substantially reduced activity for both PXR and GSPT1. FIG. 9B and FIG. 9C show representative data illustrating that SJPYT-195 analogs have substantially reduced activity for both PXR (FIG. 9C) and GSPT1 (FIG. 9B). FIG. 9D shows representative data illustrating that SJPYT-195 analogs have similar binding affinity to CRBN.

FIG. 10A-C shows representative data illustrating the characterization of compound M1. Specifically, FIG. 10A shows a representative high resolution mass spectrum collected using electrospray ionization in positive mode. FIG. 10B shows a representative HPLC chromatogram using evaporative light scattering detection. FIG. 10C shows a representative HPLC chromatogram using diode array detection.

FIG. 11A-C shows representative data illustrating the characterization of compound M9. Specifically, FIG. 11A shows a representative high resolution mass spectrum collected using electrospray ionization in positive mode. FIG. 11B shows a representative HPLC chromatogram using evaporative light scattering detection. FIG. 11C shows a representative HPLC chromatogram using diode array detection.

FIG. 12A-C shows representative data illustrating the characterization of compound M2. Specifically, FIG. 12A shows a representative high resolution mass spectrum collected using electrospray ionization in positive mode. FIG. 12B shows a representative HPLC chromatogram using evaporative light scattering detection. FIG. 12C shows a representative HPLC chromatogram using diode array detection.

FIG. 13A-C shows representative data illustrating the characterization of compound M20. Specifically, FIG. 13A shows a representative high resolution mass spectrum collected using electrospray ionization in positive mode. FIG. 13B shows a representative HPLC chromatogram using evaporative light scattering detection. FIG. 13C shows a representative HPLC chromatogram using diode array detection.

FIG. 14A-C shows representative data illustrating the characterization of compound M16. Specifically, FIG. 14A shows a representative high resolution mass spectrum collected using electrospray ionization in positive mode. FIG. 14B shows a representative HPLC chromatogram using evaporative light scattering detection. FIG. 14C shows a representative HPLC chromatogram using diode array detection.

FIG. 15A-C shows representative data illustrating the characterization of compound M18. Specifically, FIG. 15A shows a representative high resolution mass spectrum collected using electrospray ionization in positive mode. FIG. 15B shows a representative HPLC chromatogram using evaporative light scattering detection. FIG. 15C shows a representative HPLC chromatogram using diode array detection.

FIG. 16A-C shows representative data illustrating the characterization of compound M13. Specifically, FIG. 16A shows a representative high resolution mass spectrum collected using electrospray ionization in positive mode. FIG. 16B shows a representative HPLC chromatogram using evaporative light scattering detection. FIG. 16C shows a representative HPLC chromatogram using diode array detection.

FIG. 17A-C shows representative data illustrating the characterization of compound M14. Specifically, FIG. 17A shows a representative high resolution mass spectrum collected using electrospray ionization in positive mode. FIG. 17B shows a representative HPLC chromatogram using evaporative light scattering detection. FIG. 17C shows a representative HPLC chromatogram using diode array detection.

FIG. 18A-C shows representative data illustrating the characterization of compound M4. Specifically, FIG. 18A shows a representative high resolution mass spectrum collected using electrospray ionization in positive mode. FIG. 18B shows a representative HPLC chromatogram using evaporative light scattering detection. FIG. 18C shows a representative HPLC chromatogram using diode array detection.

FIG. 19A-D shows representative data illustrating the characterization of compound SJPYT-216. Specifically, FIG. 19A shows a representative high resolution mass spectrum collected using electrospray ionization in positive mode. FIG. 19B shows a representative HPLC chromatogram of the molecular ion peak. FIG. 19C shows a representative HPLC chromatogram using evaporative light scattering detection. FIG. 19D shows a representative HPLC chromatogram using diode array detection.

FIG. 20A-D shows representative data illustrating the characterization of compound SJPYT-217. Specifically, FIG. 20A shows a representative high resolution mass spectrum collected using electrospray ionization in positive mode. FIG. 20B shows a representative HPLC chromatogram of the molecular ion peak. FIG. 20C shows a representative HPLC chromatogram using evaporative light scattering detection. FIG. 20D shows a representative HPLC chromatogram using diode array detection.

FIG. 21A-D shows representative data illustrating the characterization of compound M8. Specifically, FIG. 21A shows a representative high resolution mass spectrum collected using electrospray ionization in positive mode. FIG. 21B shows a representative HPLC chromatogram of the molecular ion peak. FIG. 21C shows a representative HPLC chromatogram using evaporative light scattering detection. FIG. 21D shows a representative HPLC chromatogram using diode array detection.

FIG. 22A-D shows representative data illustrating the characterization of compound M7. Specifically, FIG. 22A shows a representative high resolution mass spectrum collected using electrospray ionization in positive mode. FIG. 22B shows a representative HPLC chromatogram of the molecular ion peak. FIG. 22C shows a representative HPLC chromatogram using evaporative light scattering detection. FIG. 22D shows a representative HPLC chromatogram using diode array detection.

FIG. 23A-D shows representative data illustrating the characterization of compound M6. Specifically, FIG. 23A shows a representative high resolution mass spectrum collected using electrospray ionization in positive mode. FIG. 23B shows a representative HPLC chromatogram of the molecular ion peak. FIG. 23C shows a representative HPLC chromatogram using evaporative light scattering detection. FIG. 23D shows a representative HPLC chromatogram using diode array detection.

FIG. 24A-D shows representative data illustrating the characterization of compound M9. Specifically, FIG. 24A shows a representative high resolution mass spectrum collected using electrospray ionization in positive mode. FIG. 24B shows a representative HPLC chromatogram of the molecular ion peak. FIG. 24C shows a representative HPLC chromatogram using evaporative light scattering detection. FIG. 24D shows a representative HPLC chromatogram using diode array detection.

FIG. 25A-D shows representative data illustrating the characterization of compound M10. Specifically, FIG. 25A shows a representative high resolution mass spectrum collected using electrospray ionization in positive mode. FIG. 25B shows a representative HPLC chromatogram of the molecular ion peak. FIG. 25C shows a representative HPLC chromatogram using evaporative light scattering detection. FIG. 25D shows a representative HPLC chromatogram using diode array detection.

FIG. 26A-D shows representative data illustrating the characterization of compound M11. Specifically, FIG. 26A shows a representative high resolution mass spectrum collected using electrospray ionization in positive mode. FIG. 26B shows a representative HPLC chromatogram of the molecular ion peak. FIG. 26C shows a representative HPLC chromatogram using evaporative light scattering detection. FIG. 26D shows a representative HPLC chromatogram using diode array detection.

FIG. 27A-D shows representative data illustrating the characterization of compound SJPYT-231. Specifically, FIG. 27A shows a representative high resolution mass spectrum collected using electrospray ionization in positive mode. FIG. 27B shows a representative HPLC chromatogram of the molecular ion peak. FIG. 27C shows a representative HPLC chromatogram using evaporative light scattering detection. FIG. 27D shows a representative HPLC chromatogram using diode array detection.

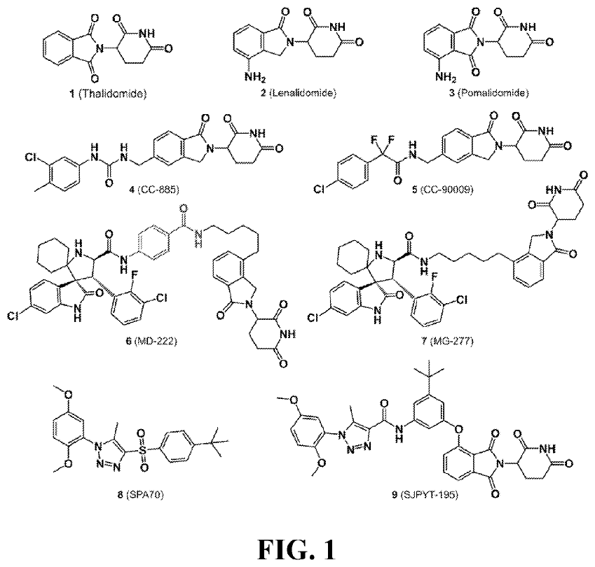

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### a. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the terms “PXR” and “pregnane X receptor” can be used interchangeably and refer to a nuclear receptor protein encoded by the NR1I2 gene, which is a transcriptional regulator of cytochrome P450 genes such as CYP3A4 and CYP3A5. PXR has a human gene map locus given as 3q12-q13.3, 3q13.3, and 3q12-q13.3 by Entrez Gene, Ensembl, and HGNC, respectively. The corresponding rat and mouse genes are given the gene symbol Nr1i2, and the respective gene map loci are 11q21 and 16 B3. The gene and protein have variously been referred to in the scientific literature as ONRI, BXR, SXR, PAR2, Orphan nuclear receptor PAR1, pregnane-activated receptor, steroid and xenobiotic receptor, MGC108643, pregnane X receptor (nuclear receptor sub family 1, group I, member 2), nuclear receptor subfamily 1 group I member 2, NR1I2, orphan nuclear receptor PXR, PXR.1, PXR.2, mPXR, and nuclear receptor subfamily 1, group 1, member 2. It can be appreciated that these terms can also be used to refer to PXR. The term PXR is understood to be inclusive of related homologous proteins in other species. The human form can be specifically designated by the term “hPXR.” The PXR protein is characterized by a DNA binding domain and a ligand binding domain (also referred to by the term “LBD”). The PXR protein forms a heterodimer with the 9-cis retinoic acid receptor RXR, and the formation of the heterodimer is required for transcriptional activation of target genes, and the heterodimer binds to the response element of the CYP3A4 or CYP3A5 promoter. The heterodimer is also believed to bind to the response elements of the ABCB1 MDR1 gene.

The major human, rat, and mouse PXR protein isoforms encoded by the PXR gene (NR1I2) are, respectively, 434, 431, and 431 amino acids. However, several major splice variants have been described at least for human encoding different isoforms, some of which have been described as using non-AUG translation initiation codons. For example, a significant human isoform is the “long isoform”, that is 473 amino acids comprising 39 additional amino acids added to the N-terminus of the major human isoform which is 434 amino acids. The LBD of the major human isoform is from amino acids 141-434, whereas the LBD of the long isoform is from amino acids 180-473.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, the subject is a mammal. A patient refers to a subject afflicted with a disease or disorder. The term “patient” includes human and veterinary subjects.

In some aspects of the disclosed methods, the subject has been diagnosed with a need for treatment of an infectious disease prior to the administering step. In some aspects of the disclosed methods, the subject has been diagnosed with a need for modulating PXR activity prior to the administering step. In some aspects of the disclosed methods, the subject has been diagnosed with having a gram positive or gram negative infection prior to the administering step. In some aspects of the disclosed methods, the subject has been identified with an infectious disease that is treatable by antagonizing the activity of PXR prior to the administering step. In some aspects of the disclosed methods, the subject has been identified with a gram positive bacterial infection prior to the administering step. In various aspects of the disclosed methods, the subject has been identified with a gram negative bacterial infection prior to the administering step. In an aspect, a subject can be treated prophylactically with a compound or composition disclosed herein, as discussed herein elsewhere.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a need for modulating PXR activity” means having been subjected to a physical examination by a person of skill, for example, a physician, and found to have a condition that can be diagnosed or treated by a compound or composition that can modulate PXR activity. As a further example, “diagnosed with a need for treatment of a disorder of uncontrolled cellular proliferation” refers to having been subjected to a physical examination by a person of skill, for example, a physician, and found to have a condition characterized by disorder of uncontrolled cellular proliferation, such as cancer.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, a target protein (e.g. the PXR protein), or other biological entity together in such a manner that the compound can affect the activity of the target, either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “EC₅₀,” is intended to refer to the concentration of a substance (e.g., a compound or a drug) that is required for 50% activation or enhancement of a biological process, or component of a process. For example, EC₅₀ can refer to the concentration of a compound that provokes a response halfway between the baseline and maximum response in an appropriate assay of the target activity. For example, an EC₅₀ for the PXR can be determined in an in vitro assay system. Such in vitro assay systems include assay such as the assays as described herein.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In an aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance. For example, an EC₅₀ for the PXR can be determined in an in vitro assay system. Such in vitro assay systems include assay such as the assays as described herein.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the 11 clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “R¹¹,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR—, SC(S)SR°; —(CH₂)₀₋₄SC(O)R°; —(CH₂)₀₋₄C(O)NR°₂; —C(S)NR°₂; —C(S)SR°; —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂₀R°; —(CH₂)₀₋₄OS(O)₂R°; —S(O)₂NR°₂; —(CH₂)₀₋₄S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°₂; —OP(O)(OR°)₂; SiR°₃; —(C₁₋₄ straight or branched alkylene)O—N(R°)₂; or —(C₁₋₄ straight or branched alkylene)C(O)O—N(R°)₂, wherein each R° may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR′, —(CH₂)₀₋₂SH, —(CH₂)₀₋₂NH₂, —(CH₂)₀₋₂NHR^{●}, —(CH₂)₀₋₂NR′2, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR*2, ═NNHC(O)R*, ═NNHC(O)OR*, =NNHS(O)₂R*, =NR*, =NOR*, —O(C(R*2))₂₋₃O—, or —S(C(R*₂))₂₋₃S—, wherein each independent occurrence of R*is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)₂₋₃O—, wherein each independent occurrence of R*is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

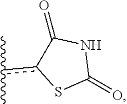

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

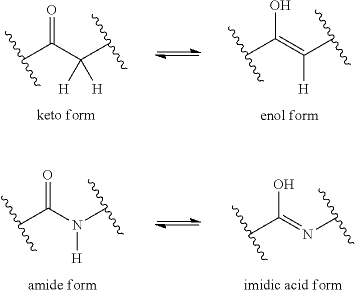 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

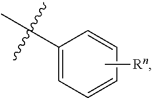

which is understood to be equivalent to a formula:

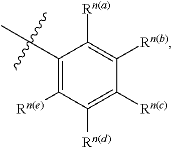

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(hu b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental Volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In an aspect, disclosed are compounds useful as modulators of the pregnane X receptor (PXR). In a further aspect, the disclosed compounds are useful for treatment of a disorder of uncontrolled cellular proliferation, such as a cancer. In a still further aspect, the disclosed compounds are useful for decreasing an adverse drug reaction in a mammal such as, for example, an adverse drug reaction associated with administration of an anticancer agent, an antibacterial agent, a non-steroidal anti-inflammatory agent, or an anticonvulsant agent.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

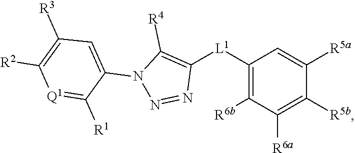

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷; wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl; wherein R⁴ is selected from hydrogen and C1-C4 alkyl; wherein each of R^{1a} and R^{1b} is independently selected from hydrogen and C1-C8 alkyl; wherein each of R^{6a} and R^{6b} is independently selected from hydrogen, C1-C8 alkyl, and R⁷; wherein R⁷ is a structure represented by a formula selected from:

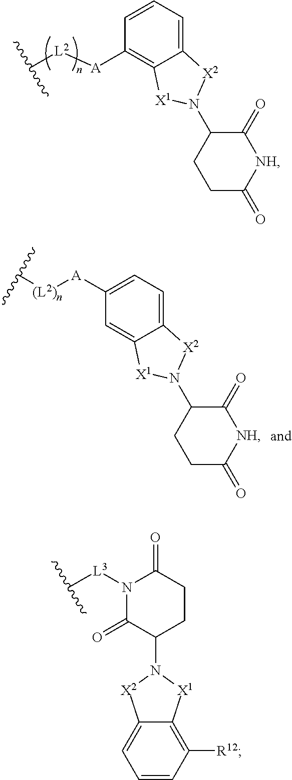

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

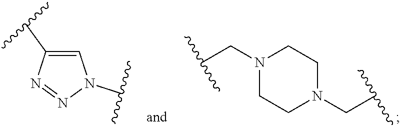

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, provided that one and only one of R², R^{6a}, and R^{6b} is R⁷, or a pharmaceutically acceptable salt thereof.

In one aspect, the compound has a structure represented by a formula:

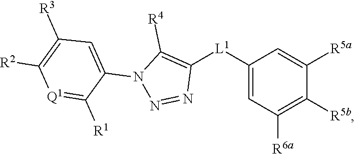

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein R^{6a} is R⁷; wherein R⁷ is a structure represented by a formula selected from:

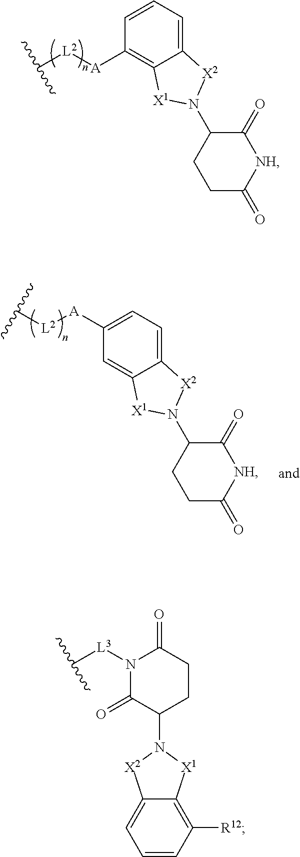

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

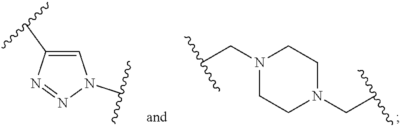

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof.

In one aspect, disclosed are compounds having a structure represented by a formula:

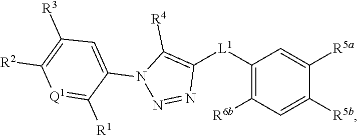

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, C1-C6 haloalkoxy, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{1a} and R^{5b} is independently selected from hydrogen and C1-C8 alkyl; wherein R^{6b} is R⁷; wherein R⁷ is a structure represented by a formula selected from:

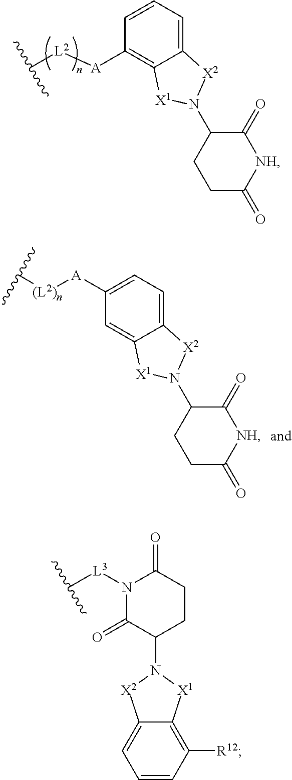

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

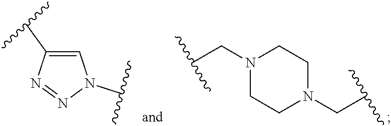

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof.

In various aspects, the compound has a structure represented by a formula selected from:

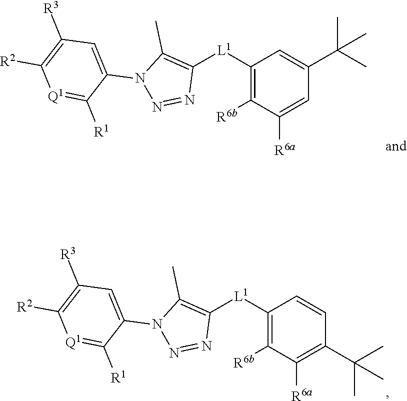

or a pharmaceutically acceptable salt thereof.

In various aspects, the compound has a structure represented by a formula:

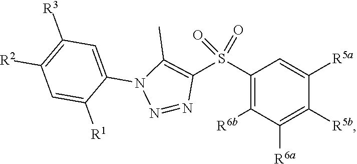

or a pharmaceutically acceptable salt thereof.

In various aspects, the compound has a structure represented by a formula:

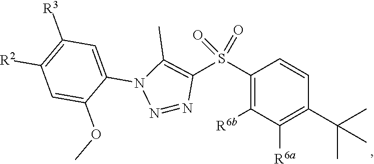

or a pharmaceutically acceptable salt thereof.

In various aspects, the compound has a structure represented by a formula:

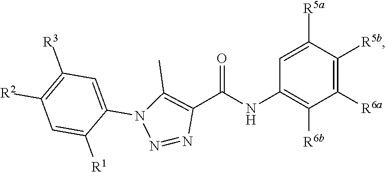

or a pharmaceutically acceptable salt thereof.

In various aspects, the compound has a structure represented by a formula:

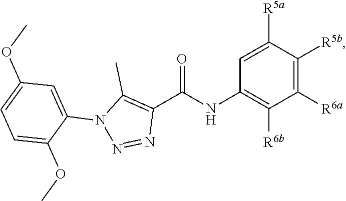

or a pharmaceutically acceptable salt thereof.

In various aspects, the compound has a structure represented by a formula:

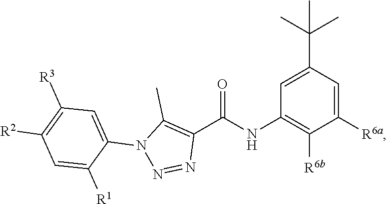

or a pharmaceutically acceptable salt thereof.

In various aspects, the compound has a structure represented by a formula:

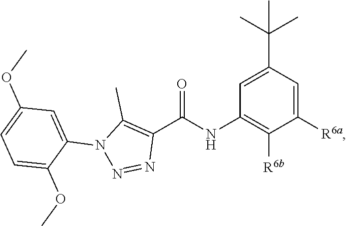

or a pharmaceutically acceptable salt thereof.

In various aspects, the compound has a structure represented by a formula:

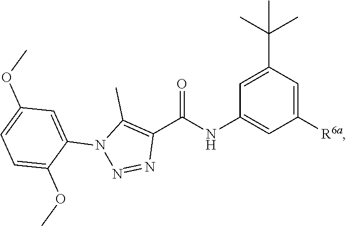

or a pharmaceutically acceptable salt thereof.

In various aspects, the compound is:

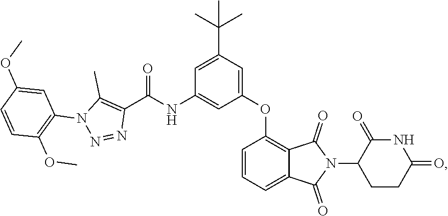

or a pharmaceutically acceptable salt thereof.

In various aspects, the compound is selected from:

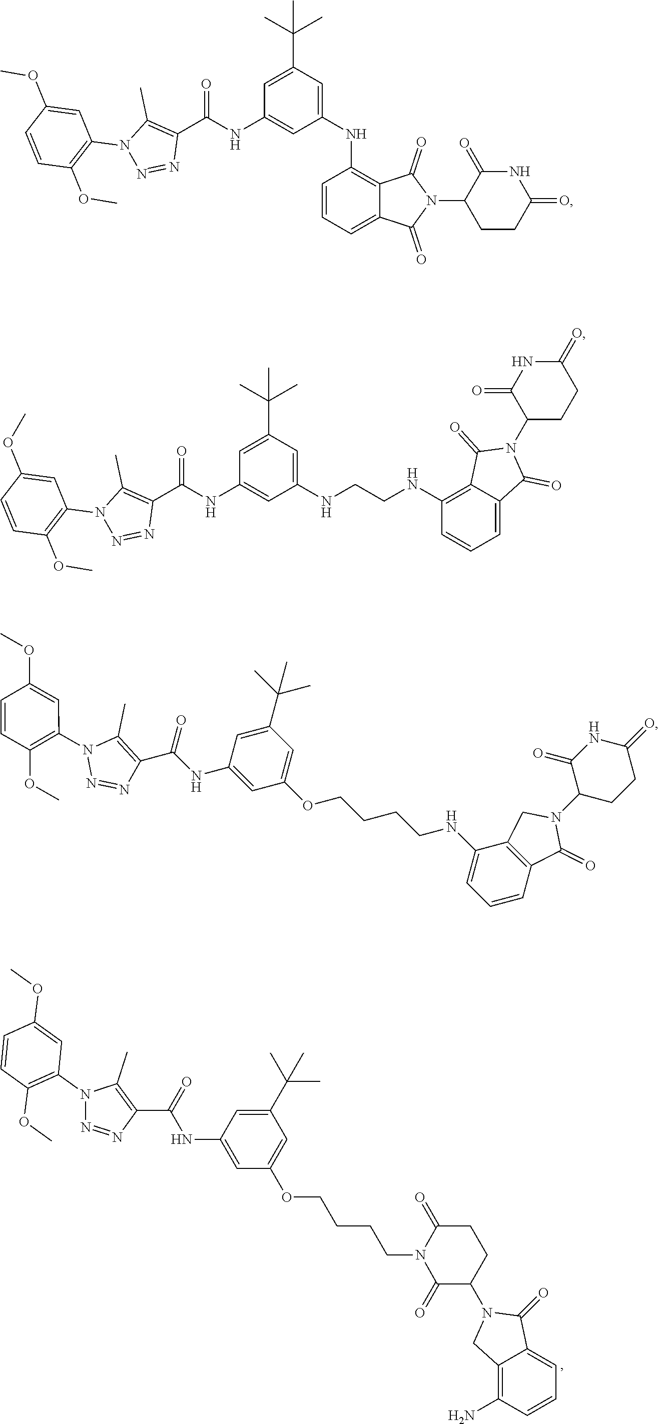 [SMILES: COc1ccc(OC)c(-n2nnc(C(=O)Nc3cc(Nc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(NCCNc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(OCCCCNc4cccc5c4CN(C4CCC(=O)NC4=O)C5=O)cc(C(C)(C)C)c3)c2C)c1 ; COc1ccc(OC)c(-n2nnc(C(=O)Nc3cc(OCCCCN4C(=O)CCC(N5Cc6c(N)cccc6C5=O)C4=O)cc(C(C)(C)C)c3)c2C)c1]

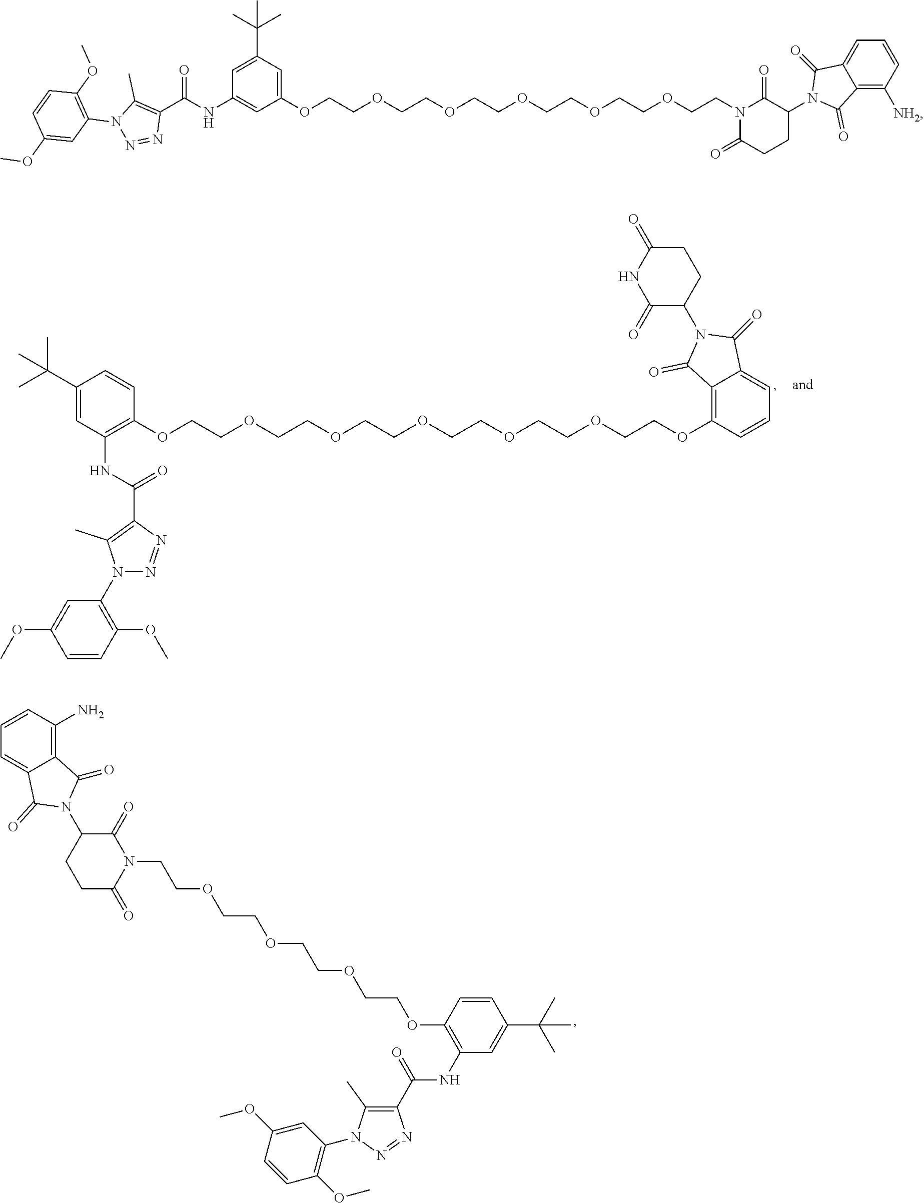 [SMILES: COc1ccc(OC)c(-n2nnc(C(=O)Nc3cc(OCCOCCOCCOCCOCCOCCN4C(=O)CCC(N5C(=O)c6cccc(N)c6C5=O)C4=O)cc(C(C)(C)C)c3)c2C)c1 ; COc1ccc(OC)c(-n2nnc(C(=O)Nc3cc(C(C)(C)C)ccc3OCCOCCOCCOCCOCCOCCOc3cccc4c3C(=O)N(C3CCC(=O)NC3=O)C4=O)c2C)c1 ; COc1ccc(OC)c(-n2nnc(C(=O)Nc3cc(C(C)(C)C)ccc3OCCOCCOCCOCCN3C(=O)CCC(N4C(=O)c5cccc(N)c5C4=O)C3=O)c2C)c1]

or a pharmaceutically acceptable salt thereof.

In various aspects, the compound has a structure represented by a formula:

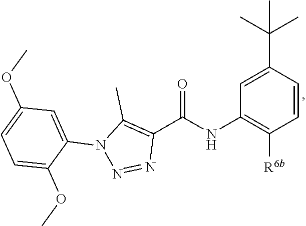

or a pharmaceutically acceptable salt thereof.

In various aspects, the compound is:

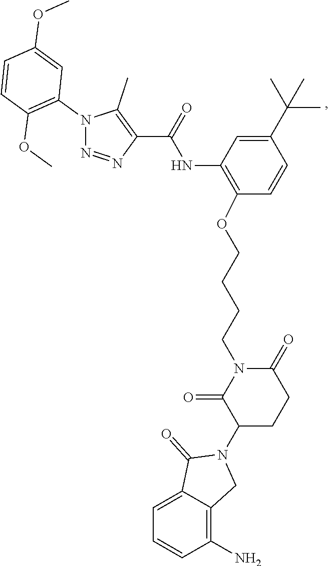 [SMILES: COc1ccc(OC)c(-n2nnc(C(=O)Nc3cc(C(C)(C)C)ccc3OCCCCN3C(=O)CCC(N4Cc5c(N)cccc5C4=O)C3=O)c2C)c1]

or a pharmaceutically acceptable salt thereof.

In various aspects, the compound has a structure represented by a formula:

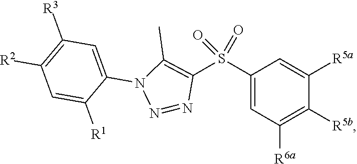

or a pharmaceutically acceptable salt thereof.

In various aspects, the compound has a structure represented by a formula:

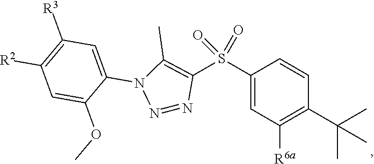

or a pharmaceutically acceptable salt thereof.

In various aspects, the compound has a structure represented by a formula:

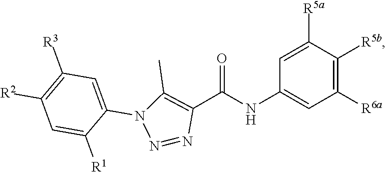

or a pharmaceutically acceptable salt thereof.

In various aspects, the compound has a structure represented by a formula:

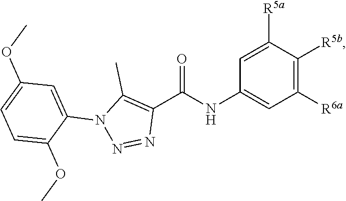

or a pharmaceutically acceptable salt thereof.

In various aspects, the compound has a structure represented by a formula:

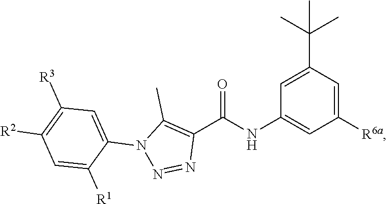

or a pharmaceutically acceptable salt thereof.

In various aspects, the compound has a structure represented by a formula:

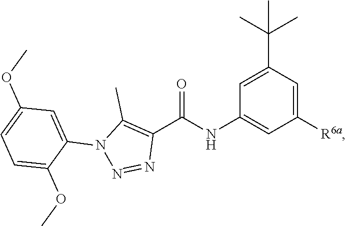

or a pharmaceutically acceptable salt thereof.

In various aspects, the compound is:

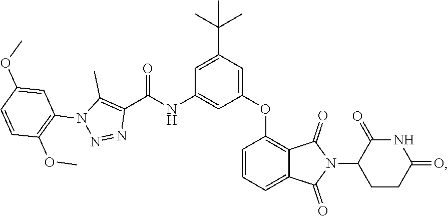

or a pharmaceutically acceptable salt thereof.

In various aspects, the compound is selected from:

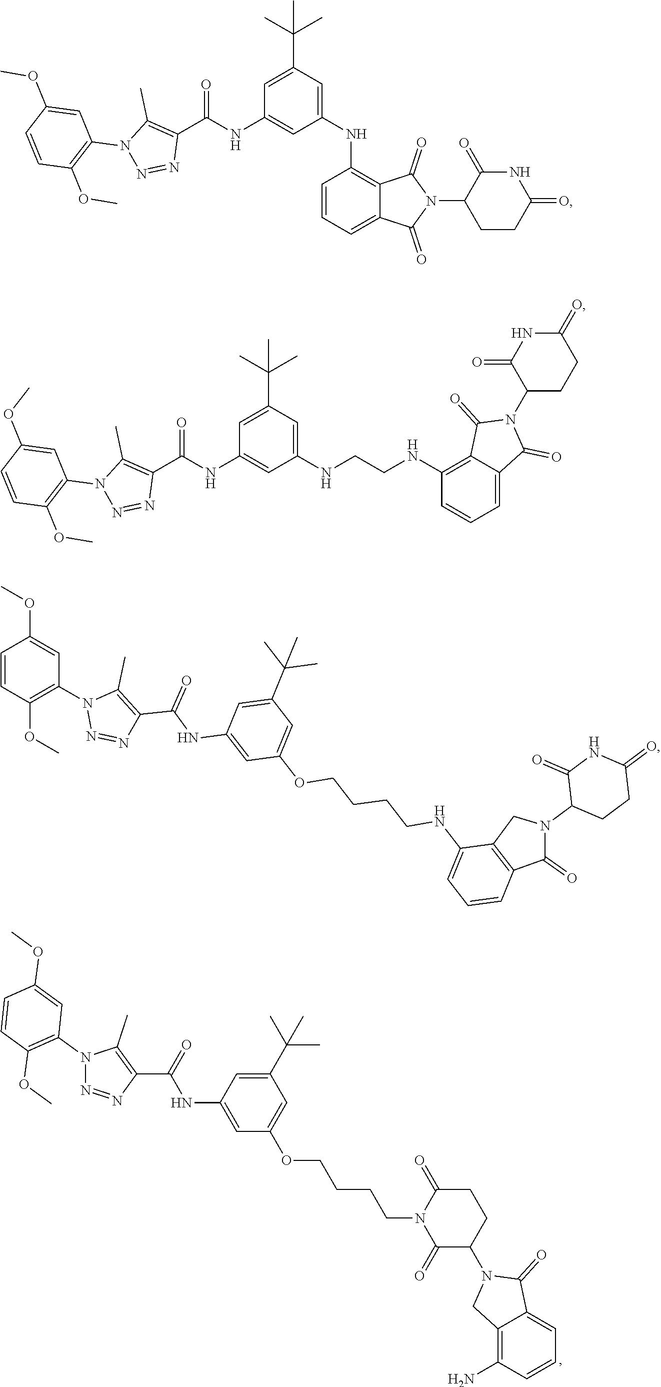 [SMILES: COc1ccc(OC)c(-n2nnc(C(=O)Nc3cc(Nc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(NCCNc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(OCCCCNc4cccc5c4CN(C4CCC(=O)NC4=O)C5=O)cc(C(C)(C)C)c3)c2C)c1 ; COc1ccc(OC)c(-n2nnc(C(=O)Nc3cc(OCCCCN4C(=O)CCC(N5Cc6c(N)cccc6C5=O)C4=O)cc(C(C)(C)C)c3)c2C)c1]

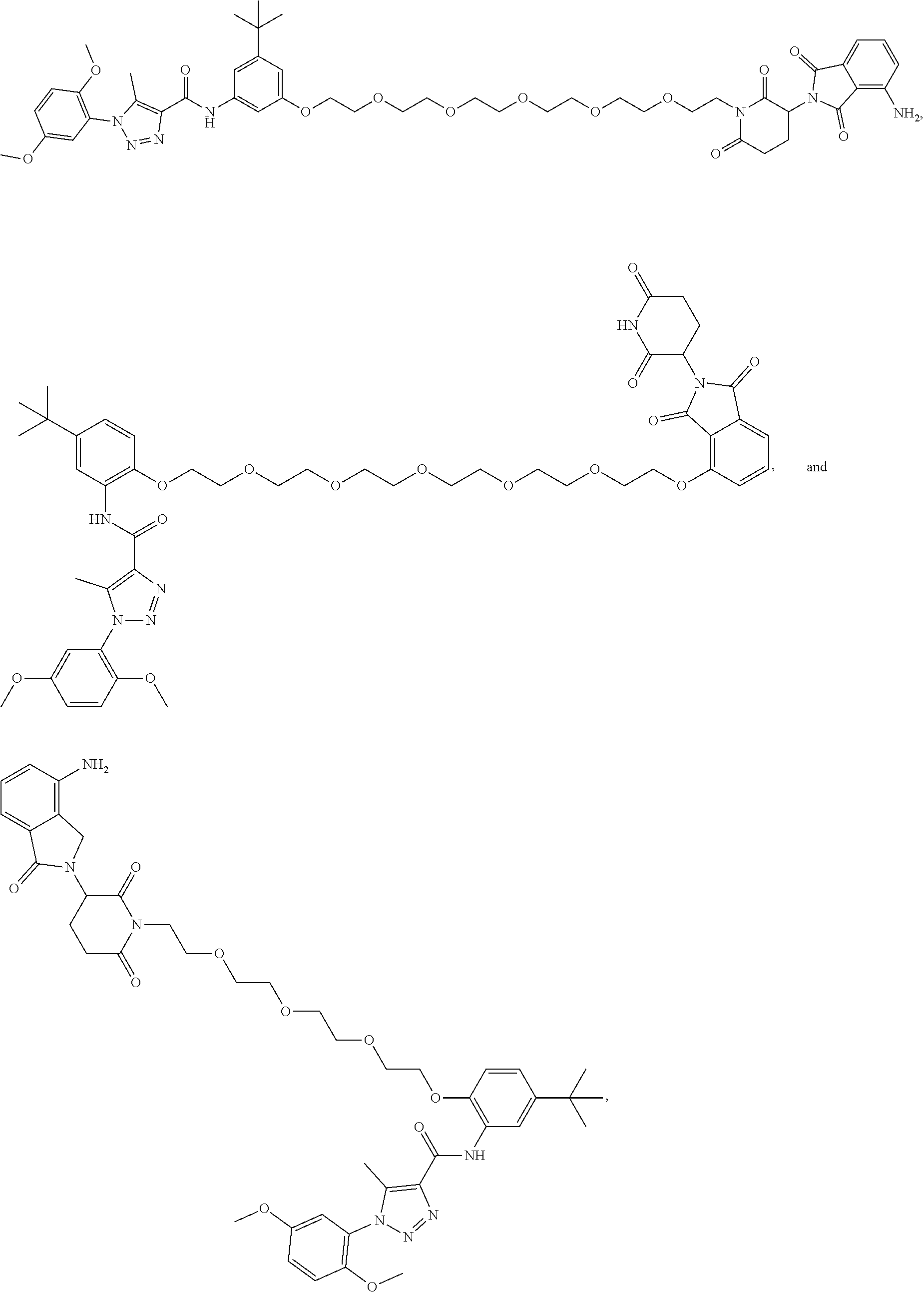 [SMILES: COc1ccc(OC)c(-n2nnc(C(=O)Nc3cc(OCCOCCOCCOCCOCCOCCN4C(=O)CCC(N5C(=O)c6cccc(N)c6C5=O)C4=O)cc(C(C)(C)C)c3)c2C)c1 ; COc1ccc(OC)c(-n2nnc(C(=O)Nc3cc(C(C)(C)C)ccc3OCCOCCOCCOCCOCCOCCOc3cccc4c3C(=O)N(C3CCC(=O)NC3=O)C4=O)c2C)c1 ; COc1ccc(OC)c(-n2nnc(C(=O)Nc3cc(C(C)(C)C)ccc3OCCOCCOCCOCCN3C(=O)CCC(N4Cc5c(N)cccc5C4=O)C3=O)c2C)c1]

or a pharmaceutically acceptable salt thereof.

In various aspects, the compound has a structure represented by a formula:

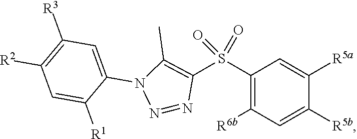

or a pharmaceutically acceptable salt thereof.

In various aspects, the compound has a structure represented by a formula:

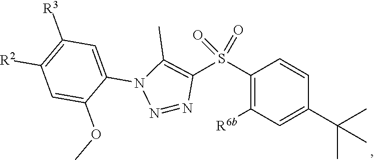

or a pharmaceutically acceptable salt thereof.

In various aspects, the compound has a structure represented by a formula:

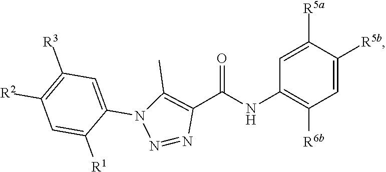

or a pharmaceutically acceptable salt thereof.

In various aspects, the compound has a structure represented by a formula:

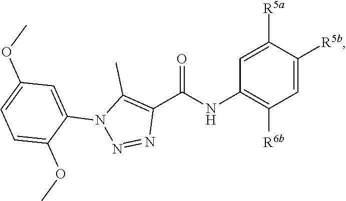

or a pharmaceutically acceptable salt thereof.

In various aspects, the compound has a structure represented by a formula:

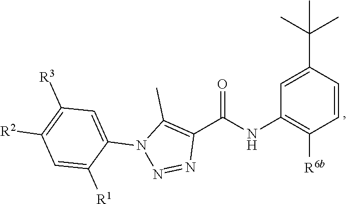

or a pharmaceutically acceptable salt thereof.

In various aspects, the compound has a structure represented by a formula:

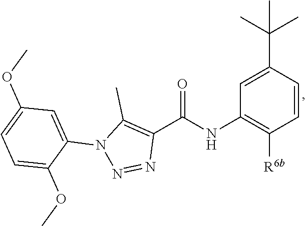

or a pharmaceutically acceptable salt thereof.

In various aspects, n is selected from 0 and 1. In a further aspect, n is 0. In a still further aspect, n is 1.

In various aspects, q, when present, is selected from 1, 2, 3, 4, 5, 6, 7, and 8. In a further aspect, q, when present, is selected from 1, 2, 3, 4, 5, 6, and 7. In a still further aspect, q, when present, is selected from 1, 2, 3, 4, 5, and 6. In yet a further aspect, q, when present, is selected from 1, 2, 3, 4, and 5. In an even further aspect, q, when present, is selected from 1, 2, 3, and 4. In a still further aspect, q, when present, is selected from 1, 2, and 3. In yet a further aspect, q, when present, is selected from 1 and 2. In an even further aspect, q, when present, is selected from 2, 3, 4, 5, 6, 7, and 8. In a still further aspect, q, when present, is selected from 3, 4, 5, 6, 7, and 8. In yet a further aspect, q, when present, is selected from 5, 6, 7, and 8. In an even further aspect, q, when present, is selected from 6, 7, and 8. In a still further aspect, q, when present, is selected from 7 and 8. In yet a further aspect, q, when present, is 1. In an even further aspect, q, when present, is 2. In a still further aspect, q, when present, is 3. In yet a further aspect, q, when present, is 4. In an even further aspect, q, when present, is 5. In a still further aspect, q, when present, is 6. In yet a further aspect, q, when present, is 7. In an even further aspect, q, when present, is 8.

In various aspects, r, when present, is selected from 1, 2, 3, 4, 5, 6, 7, and 8. In a further aspect, r, when present, is selected from 1, 2, 3, 4, 5, 6, and 7. In a still further aspect, r, when present, is selected from 1, 2, 3, 4, 5, and 6. In yet a further aspect, r, when present, is selected from 1, 2, 3, 4, and 5. In an even further aspect, r, when present, is selected from 1, 2, 3, and 4. In a still further aspect, r, when present, is selected from 1, 2, and 3. In yet a further aspect, r, when present, is selected from 1 and 2. In an even further aspect, r, when present, is selected from 2, 3, 4, 5, 6, 7, and 8. In a still further aspect, r, when present, is selected from 3, 4, 5, 6, 7, and 8. In yet a further aspect, r, when present, is selected from 5, 6, 7, and 8. In an even further aspect, r, when present, is selected from 6, 7, and 8. In a still further aspect, r, when present, is selected from 7 and 8. In yet a further aspect, r, when present, is 1. In an even further aspect, r, when present, is 2. In a still further aspect, r, when present, is 3. In yet a further aspect, r, when present, is 4. In an even further aspect, r, when present, is 5. In a still further aspect, r, when present, is 6. In yet a further aspect, r, when present, is 7. In an even further aspect, r, when present, is 8.

### a. A Groups

In one aspect, A is selected from —O—, —NH—, —CH₂—, and —C≡C—. In a further aspect, A is selected from —O—, —NH—, and —CH₂—. In a still further aspect, A is selected from —O— and —NH—. In yet a further aspect, A is selected from —NH—, —CH₂—, and —C≡C—. In a still further aspect, A is selected from —CH₂— and —C≡C—. In yet a further aspect, A is selected from —O—, —CH₂—, and —C≡C—. In an even further aspect, A is selected from —O— and —CH₂—. In a still further aspect, A is selected from —O— and —C≡C—. In yet a further aspect, A is selected from —O—, —NH—, and —C≡C—.

In various aspects, A is —O—. In a further aspect, A is —NH—. In a still further aspect, A is —CH₂—. In yet a further aspect, A is —C≡C—.

### b. L¹ Groups

In one aspect, L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—. In a further aspect, L₁ is selected from —SO₂—, —CO₂—, —OC(O)—, —C(O)NR¹⁰—, —NR¹⁰C(O)—, —SO₂NR¹⁰—, and —NR¹⁰SO₂—. In a still further aspect, L¹ is selected from —SO₂—, —CO₂—, —OC(O)—, —C(O)NR¹⁰—, —NR¹⁰C(O)—, and —SO₂NR¹⁰—. In yet a further aspect, L¹ is selected from —SO₂—, —CO₂—, —OC(O)—, —C(O)NR¹⁰—, and —NR¹⁰C(O)—. In an even further aspect, L¹ is selected from —SO₂—, —CO₂—, —OC(O)—, and —C(O)NR¹⁰—. In a still further aspect, L¹ is selected from —SO₂—, —CO₂—, and —OC(O)—. In yet a further aspect, L¹ is selected from —SO₂— and —CO₂—. In an even further aspect, L¹ is selected from —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—. In a still further aspect, L¹ is selected from —OC(O)—, —C(O)NR¹⁰—, —NR¹⁰C(O)—, —SO₂NR¹⁰—, —NR¹⁰SO₂—, and —N(R¹⁰)C(O)NR¹¹—. In yet a further aspect, L¹ is selected from —C(O)NR¹⁰—, —NR¹⁰C(O)—, —SO₂NR¹⁰—, —NR¹⁰SO₂—, and —N(R¹⁰)C(O)NR¹¹—. In an even further aspect, L¹ is selected from —NR¹⁰C(O)—, —SO₂NR¹⁰—, —NR¹⁰SO₂—, and —N(R¹⁰)C(O)NR¹¹—. In a still further aspect, L¹ is selected from —SO₂NR¹⁰—, —NR¹⁰SO₂—, and —N(R¹⁰)C(O)NR¹¹—. In yet a further aspect, L¹ is selected from —NR¹⁰SO₂— and —N(R¹⁰)C(O)NR¹¹—.

In various aspects, L¹ is selected from —C(O)NR¹⁰—, —NR¹⁰C(O)—, and —N(R¹⁰)C(O)NR¹¹—. In a further aspect, L¹ is selected from —C(O)NR¹⁰— and —NR¹⁰C(O)—. In a still further aspect, L¹ is selected from —C(O)NR¹⁰— and —N(R¹⁰)C(O)NR¹¹—. In yet a further aspect, L¹ is selected from —NR¹⁰C(O)— and —N(R¹⁰)C(O)NR¹¹—. In an even further aspect, L¹ is —C(O)NR¹⁰—. In a still further aspect, L¹ is —NR¹⁰C(O)—. In yet a further aspect, L¹ is —NR¹⁰C(O)—.

In various aspects, L¹ is selected from —SO₂—, —SO₂NR¹⁰—, and —NR¹⁰SO₂—. In a further aspect, L¹ is selected from —SO₂— and —NR¹⁰SO₂—. In a still further aspect, L¹ is selected from —SO₂— and —SO₂NR¹⁰—. In yet a further aspect, L¹ is selected from —SO₂NR¹⁰— and —NR¹⁰SO₂—. In an even further aspect, L¹ is —SO₂—. In a still further aspect, L¹ is —SO₂NR¹⁰—. In yet a further aspect, L¹ is —NR¹⁰SO₂—.

In various aspects, L¹ is selected from —C(O)NR¹⁰— and —SO₂NR¹⁰—.

In various aspects, L¹ is selected from —CO₂— and —OC(O)—. In a further aspect, L¹ is —CO₂—. In a still further aspect, L¹ is —OC(O)—.

### c. L² Groups

In one aspect, L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-. In a further aspect, L² is selected from —O(C1-C4 alkyl)-, —NH(C1-C4 alkyl)-, —OC(O)—, —NHC(O)—, —OC(O)(C1-C4 alkyl)-, —NHC(O)(C1-C4 alkyl)-, —O(C1-C4 alkyl)C(O)—, —NH(C1-C4 alkyl)C(O)—, —NHC(O)(C1-C4 alkyl)NHC(O)(C1-C4 alkyl)-, —NHC(O)(C1-C4 alkyl)(OCH₂CH₂)_{q}—, —(OCH₂CH₂)_{q}—, and —O(C1-C4 alkyl)Ar¹(C1-C4 alkyl)NHC(O)(C1-C4 alkyl)-. In a further aspect, L² is selected from —OCH₂—, —OCH₂CH₂—, —OCH(CH₃)CH₂—, —OCH₂CH₂CH₂—, —NHCH₂—, —NHCH₂CH₂—, —NHCH(CH₃)CH₂—, —NHCH₂CH₂CH₂—, —OC(O)—, —NHC(O)—, —OC(O)CH₂—, —OC(O)CH₂CH₂—, —OC(O)CH(CH₃)CH₂—, —OC(O)CH₂CH₂CH₂—, —NHC(O)CH₂—, —NHC(O)CH₂CH₂—, —NHC(O)CH(CH₃)CH₂—, —NHC(O)CH₂CH₂CH₂—, —OCH₂C(O)—, —OCH₂CH₂C(O)—, —OCH(CH₃)CH₂C(O)—, —OCH₂CH₂CH₂C(O)—, —NHCH₂C(O)—, —NHCH₂CH₂C(O)—, —NHCH(CH₃)CH₂C(O)—, —NHCH₂CH₂CH₂C(O)—, —NHCH₂NHC(O)CH₂—, —NHCH₂CH₂NHC(O)CH₂CH₂—, —NHCH(CH₃)CH₂NHC(O)CH₂—, —NHCH₂CH₂CH₂NHC(O)CH₂CH₂—, —NHCH₂OCH₂CH₂—, —NHCH₂CH₂OCH₂CH₂OCH₂CH₂—, —NHCH(CH₃)CH₂OCH₂CH₂—, —NHCH₂CH₂CH₂(OCH₂CH₂)₃—, —OCH₂CH₂—, —OCH₂CH₂OCH₂CH₂—, —(OCH₂CH₂)₃—, —OCH₂Ar¹CH₂NHC(O)CH₂—, —OCH₂CH₂Ar¹CH₂CH₂NHC(O)CH₂CH₂—, —OCH(CH₃)CH₂Ar¹CH₂NHC(O)CH₂—, and —OCH₂CH₂CH₂Ar¹CH₂CH₂NHC(O)CH₂CH₂—. In a still further aspect, L² is selected from —OCH₂—, —OCH₂CH₂—, —NHCH₂—, —NHCH₂CH₂—, —OC(O)—, —NHC(O)—, —OC(O)CH₂—, —OC(O)CH₂CH₂—, —NHC(O)CH₂—, —NHC(O)CH₂CH₂—, —OCH₂C(O)—, —OCH₂CH₂C(O)—, —NHCH₂C(O)—, —NHCH₂CH₂C(O)—, —NHCH₂NHC(O)CH₂—, —NHCH₂CH₂NHC(O)CH₂CH₂—, —NHCH₂OCH₂CH₂—, —NHCH₂CH₂OCH₂CH₂OCH₂CH₂—, —OCH₂CH₂—, —OCH₂CH₂OCH₂CH₂—, —OCH₂Ar¹CH₂NHC(O)CH₂—, and —OCH₂CH₂Ar¹CH₂CH₂NHC(O)CH₂CH₂—. In yet a further aspect, L² is selected from —OCH₂—, —NHCH₂—, —OC(O)—, —NHC(O)—, —OC(O)CH₂—, —NHC(O)CH₂—, —OCH₂C(O)—, —NHCH₂C(O)—, —NHCH₂NHC(O)CH₂—, —NHCH₂OCH₂CH₂—, —OCH₂CH₂—, and —OCH₂Ar¹CH₂NHC(O)CH₂—.

In various aspects, L² is selected from —O(C1-C8 alkyl)-, —OC(O)—, —OC(O)(C1-C8 alkyl)-, —O(C1-C8 alkyl)C(O)—, —(OCH₂CH₂)_{q}—, and —O(C1-C8 alkyl)Ar¹(C1-C8 alkyl)NHC(O)(C1-C4 alkyl)-. In a further aspect, L² is selected from —O(C1-C4 alkyl)-, —OC(O)—, —OC(O)(C1-C4 alkyl)-, —O(C1-C4 alkyl)C(O)—, —(OCH₂CH₂)_{q}—, and —O(C1-C4 alkyl)Ar¹(C1-C4 alkyl)NHC(O)(C1-C4 alkyl)-. In a further aspect, L² is selected from —OCH₂—, —OCH₂CH₂—, —OCH(CH₃)CH₂—, —OCH₂CH₂CH₂—, —OC(O)—, —OC(O)CH₂—, —OC(O)CH₂CH₂—, —OC(O)CH(CH₃)CH₂—, —OC(O)CH₂CH₂CH₂—, —OCH₂C(O)—, —OCH₂CH₂C(O)—, —OCH(CH₃)CH₂C(O)—, —OCH₂CH₂CH₂C(O)—, —OCH₂CH₂—, —OCH₂CH₂OCH₂CH₂—, —(OCH₂CH₂)₃—, —OCH₂Ar¹CH₂NHC(O)CH₂—, —OCH₂CH₂Ar¹CH₂CH₂NHC(O)CH₂CH₂—, —OCH(CH₃)CH₂Ar¹CH₂NHC(O)CH₂—, and —OCH₂CH₂CH₂Ar¹CH₂CH₂NHC(O)CH₂CH₂—. In a still further aspect, L² is selected from —OCH₂—, —OCH₂CH₂—, —OC(O)—, —OC(O)CH₂—, —OC(O)CH₂CH₂—, —OCH₂C(O)—, —OCH₂CH₂C(O)—, —OCH₂CH₂—, —OCH₂CH₂OCH₂CH₂—, —OCH₂Ar¹CH₂NHC(O)CH₂—, and —OCH₂CH₂Ar¹CH₂CH₂NHC(O)CH₂CH₂—. In yet a further aspect, L² is selected from —OCH₂—, —OC(O)—, —OC(O)CH₂—, —OCH₂C(O)—, —OCH₂CH₂—, and —OCH₂Ar¹CH₂NHC(O)CH₂—.

In various aspects, L² is selected from —NH(C1-C8 alkyl)-, —NHC(O)—, —NHC(O)(C1-C8 alkyl)-, —NH(C1-C8 alkyl)C(O)—, —NHC(O)(C1-C8 alkyl)NHC(O)(C1-C8 alkyl)-, and —NHC(O)(C1-C4 alkyl)(OCH₂CH₂)_{q}—. In a further aspect, L² is selected from —NH(C1-C4 alkyl)-, —NHC(O)—, —NHC(O)(C1-C4 alkyl)-, —NH(C1-C4 alkyl)C(O)—, —NHC(O)(C1-C4 alkyl)NHC(O)(C1-C4 alkyl)-, and —NHC(O)(C1-C4 alkyl)(OCH₂CH₂)_{q}—. In a further aspect, L² is selected from —NHCH₂—, —NHCH₂CH₂—, —NHCH(CH₃)CH₂—, —NHCH₂CH₂CH₂—, —NHC(O)—, —NHC(O)CH₂—, —NHC(O)CH₂CH₂—, —NHC(O)CH(CH₃)CH₂—, —NHC(O)CH₂CH₂CH₂—, —NHCH₂C(O)—, —NHCH₂CH₂C(O)—, —NHCH(CH₃)CH₂C(O)—, —NHCH₂CH₂CH₂C(O)—, —NHCH₂NHC(O)CH₂—, —NHCH₂CH₂NHC(O)CH₂CH₂—, —NHCH(CH₃)CH₂NHC(O)CH₂—, —NHCH₂CH₂CH₂NHC(O)CH₂CH₂—, —NHCH₂OCH₂CH₂—, —NHCH₂CH₂OCH₂CH₂OCH₂CH₂—, —NHCH(CH₃)CH₂OCH₂CH₂—, and —NHCH₂CH₂CH₂(OCH₂CH₂)₃—. In a still further aspect, L² is selected from —NHCH₂—, —NHCH₂CH₂—, —NHC(O)—, —NHC(O)CH₂—, —NHC(O)CH₂CH₂—, —NHCH₂C(O)—, —NHCH₂CH₂C(O)—, —NHCH₂NHC(O)CH₂—, —NHCH₂CH₂NHC(O)CH₂CH₂—, —NHCH₂OCH₂CH₂—, and —NHCH₂CH₂OCH₂CH₂OCH₂CH₂—. In yet a further aspect, L² is selected from —NHCH₂—, —NHC(O)—, —NHC(O)CH₂—, —NHCH₂C(O)—, —NHCH₂NHC(O)CH₂—, and —NHCH₂OCH₂CH₂—.

### d. L³ Groups

In one aspect, L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-. In a further aspect, L³ is selected from —O(C1-C4 alkyl)-, —NH(C1-C4 alkyl)-, —OC(O)—, —NHC(O)—, —NHC(O)(C1-C4 alkyl)-, —O(C1-C4 alkyl)C(O)—, —NH(C1-C4 alkyl)C(O)—, —NHC(O)(C1-C4 alkyl)NHC(O)(C1-C4 alkyl)-, —NHC(O)(C1-C4 alkyl)(OCH₂CH₂)ᵣ—, —(OCH₂CH₂)ᵣ—, and —O(C1-C4 alkyl)Ar¹(C1-C4 alkyl)NHC(O)(C1-C4 alkyl)-. In a further aspect, L³ is selected from —OCH₂—, —OCH₂CH₂—, —OCH(CH₃)CH₂—, —OCH₂CH₂CH₂—, —NHCH₂—, —NHCH₂CH₂—, —NHCH(CH₃)CH₂—, —NHCH₂CH₂CH₂—, —OC(O)—, —NHC(O)—, —NHC(O)CH₂—, —NHC(O)CH₂CH₂—, —NHC(O)CH(CH₃)CH₂—, —NHC(O)CH₂CH₂CH₂—, —OCH₂C(O)—, —OCH₂CH₂C(O)—, —OCH(CH₃)CH₂C(O)—, —OCH₂CH₂CH₂C(O)—, —NHCH₂C(O)—, —NHCH₂CH₂C(O)—, —NHCH(CH₃)CH₂C(O)—, —NHCH₂CH₂CH₂C(O)—, —NHCH₂NHC(O)CH₂—, —NHCH₂CH₂NHC(O)CH₂CH₂—, —NHCH(CH₃)CH₂NHC(O)CH₂—, —NHCH₂CH₂CH₂NHC(O)CH₂CH₂—, —NHCH₂OCH₂CH₂—, —NHCH₂CH₂OCH₂CH₂OCH₂CH₂—, —NHCH(CH₃)CH₂OCH₂CH₂—, —NHCH₂CH₂CH₂(OCH₂CH₂)₃—, —OCH₂CH₂—, —OCH₂CH₂OCH₂CH₂—, —(OCH₂CH₂)₃—, —OCH₂Ar¹CH₂NHC(O)CH₂—, —OCH₂CH₂Ar¹CH₂CH₂NHC(O)CH₂CH₂—, —OCH(CH₃)CH₂Ar¹CH₂NHC(O)CH₂—, and —OCH₂CH₂CH₂Ar¹CH₂CH₂NHC(O)CH₂CH₂—. In a still further aspect, L³ is selected from —OCH₂—, —OCH₂CH₂—, —NHCH₂—, —NHCH₂CH₂—, —OC(O)—, —NHC(O)—, —NHC(O)CH₂—, —NHC(O)CH₂CH₂—, —OCH₂C(O)—, —OCH₂CH₂C(O)—, —NHCH₂C(O)—, —NHCH₂CH₂C(O)—, —NHCH₂NHC(O)CH₂—, —NHCH₂CH₂NHC(O)CH₂CH₂—, —NHCH₂OCH₂CH₂—, —NHCH₂CH₂OCH₂CH₂OCH₂CH₂—, —OCH₂CH₂—, —OCH₂CH₂OCH₂CH₂—, —OCH₂Ar¹CH₂NHC(O)CH₂—, and —OCH₂CH₂Ar¹CH₂CH₂NHC(O)CH₂CH₂—. In yet a further aspect, L³ is selected from —OCH₂—, —NHCH₂—, —OC(O)—, —NHC(O)—, —NHC(O)CH₂—, —OCH₂C(O)—, —NHCH₂C(O)—, —NHCH₂NHC(O)CH₂—, —NHCH₂OCH₂CH₂—, —OCH₂CH₂—, and —OCH₂Ar¹CH₂NHC(O)CH₂—.

In various aspects, L³ is selected from —O(C1-C8 alkyl)-, —OC(O)—, —O(C1-C8 alkyl)C(O)—, —(OCH₂CH₂)ᵣ—, and —O(C1-C8 alkyl)Ar¹(C1-C8 alkyl)NHC(O)(C1-C4 alkyl)-. In a further aspect, L³ is selected from —O(C1-C4 alkyl)-, —OC(O)—, —O(C1-C4 alkyl)C(O)—, —(OCH₂CH₂)—, and —O(C1-C4 alkyl)Ar¹(C1-C4 alkyl)NHC(O)(C1-C4 alkyl)-. In a further aspect, L³ is selected from —OCH₂—, —OCH₂CH₂—, —OCH(CH₃)CH₂—, —OCH₂CH₂CH₂—, —OC(O)—, —OCH₂C(O)—, —OCH₂CH₂C(O)—, —OCH(CH₃)CH₂C(O)—, —OCH₂CH₂CH₂C(O)—, —OCH₂CH₂—, —OCH₂CH₂OCH₂CH₂—, —(OCH₂CH₂)₃—, —OCH₂Ar¹CH₂NHC(O)CH₂—, —OCH₂CH₂Ar¹CH₂CH₂NHC(O)CH₂CH₂—, —OCH(CH₃)CH₂Ar¹CH₂NHC(O)CH₂—, and —OCH₂CH₂CH₂Ar¹CH₂CH₂NHC(O)CH₂CH₂—. In a still further aspect, L³ is selected from —OCH₂—, —OCH₂CH₂—, —OC(O)—, —OCH₂C(O)—, —OCH₂CH₂C(O)—, —OCH₂CH₂—, —OCH₂CH₂OCH₂CH₂—, —OCH₂Ar¹CH₂NHC(O)CH₂—, and —OCH₂CH₂Ar¹CH₂CH₂NHC(O)CH₂CH₂—. In yet a further aspect, L³ is selected from —OCH₂—, —OC(O)—, —OCH₂C(O)—, —OCH₂CH₂—, and —OCH₂Ar¹CH₂NHC(O)CH₂—.

In various aspects, L³ is selected from —NH(C1-C8 alkyl)-, —NHC(O)—, —NHC(O)(C1-C8 alkyl)-, —NH(C1-C8 alkyl)C(O)—, —NHC(O)(C1-C8 alkyl)NHC(O)(C1-C8 alkyl)-, and —NHC(O)(C1-C4 alkyl)(OCH₂CH₂)—. In a further aspect, L³ is selected from —NH(C1-C4 alkyl)-, —NHC(O)—, —NHC(O)(C1-C4 alkyl)-, —NH(C1-C4 alkyl)C(O)—, —NHC(O)(C1-C4 alkyl)NHC(O)(C1-C4 alkyl)-, and —NHC(O)(C1-C4 alkyl)(OCH₂CH₂)—. In a further aspect, L³ is selected from —NHCH₂—, —NHCH₂CH₂—, —NHCH(CH₃)CH₂—, —NHCH₂CH₂CH₂—, —NHC(O)—, —NHC(O)CH₂—, —NHC(O)CH₂CH₂—, —NHC(O)CH(CH₃)CH₂—, —NHC(O)CH₂CH₂CH₂—, —NHCH₂C(O)—, —NHCH₂CH₂C(O)—, —NHCH(CH₃)CH₂C(O)—, —NHCH₂CH₂CH₂C(O)—, —NHCH₂NHC(O)CH₂—, —NHCH₂CH₂NHC(O)CH₂CH₂—, —NHCH(CH₃)CH₂NHC(O)CH₂—, —NHCH₂CH₂CH₂NHC(O)CH₂CH₂—, —NH CH₂OCH₂CH₂—, —NHCH₂CH₂OCH₂CH₂OCH₂CH₂—, —NHCH(CH₃)CH₂OCH₂CH₂—, and —NHCH₂CH₂CH₂(OCH₂CH₂)₃—. In a still further aspect, L³ is selected from —NHCH₂—, —NHCH₂CH₂—, —NHC(O)—, —NHC(O)CH₂—, —NHC(O)CH₂CH₂—, —NHCH₂C(O)—, —NHCH₂CH₂C(O)—, —NHCH₂NHC(O)CH₂—, —NHCH₂CH₂NHC(O)CH₂CH₂—, —NHCH₂OCH₂CH₂—, and —NHCH₂CH₂OCH₂CH₂OCH₂CH₂—. In yet a further aspect, L³ is selected from —NHCH₂—, —NHC(O)—, —NHC(O)CH₂—, —NHCH₂C(O)—, —NHCH₂NHC(O)CH₂—, and —NHCH₂OCH₂CH₂—.

### e. Q¹ Groups

In one aspect, Q¹ is selected from N and CH. In a further aspect, Q¹ is N. In a still further aspect, Q¹ is CH.

### f. X¹ and X² Groups

In one aspect, each of X¹ and X² is independently selected from —CH₂— and —C(O)—, provided that at least one of X¹ and X² is —C(O)—. In a further aspect, one of X¹ and X² is —C(O)— and the other is —CH₂—. In a still further aspect, X¹ is —CO— and X² is —CH₂—. In yet a further aspect, X¹ is —CH₂— and X² is —CO—. In an even further aspect, each of X¹ and X² is —C(O)—.

### g. R¹ Groups

In one aspect, R¹ is selected from hydrogen, halogen, haloalkoxy, —OH, C1-C6 alkyl, C1-C6 alkoxy, C1-C6 haloalkyl, C1-C6 hydroxyalkyl, and C1-C6 haloalkoxy. In a further aspect, R¹ is selected from hydrogen, halogen, —OH, C1-C4 alkyl, C1-C4 alkoxy, C1-C4 haloalkyl, C1-C4 hydroxyalkyl, and C1-C4 haloalkoxy. In a still further aspect, R¹ is selected from hydrogen, —F, —Cl, —OH, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —CH₂OH, —CH₂CH₂OH, —CH(CH₃)CH₂OH, —CH₂CH₂CH₂OH, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CHClCH₃. In yet a further aspect, R¹ is selected from hydrogen, —F, —Cl, —OH, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —CH₂OH, —CH₂CH₂OH, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCHClCH₃. In an even further aspect, R¹ is selected from hydrogen, —F, —Cl, —OH, methyl, —OCH₃, —CF₃, —CHF₂, —CH₂F, —CCl₃, —CHCl₂, —CH₂Cl, —CH₂OH, —OCF₃, —OCHF₂, —OCH₂F, —OCCl₃, —OCHCl₂, and —OCH₂Cl.

In various aspects, R¹ is selected from hydrogen, halogen, haloalkoxy, —OH, C1-C6 alkyl, C1-C6 alkoxy, C1-C6 haloalkyl, C1-C6 hydroxyalkyl, and C1-C6 haloalkoxy. In a further aspect, R¹ is selected from hydrogen, halogen, —OH, C1-C4 alkyl, C1-C4 alkoxy, C1-C4 haloalkyl, and C1-C4 hydroxyalkyl. In a still further aspect, R¹ is selected from hydrogen, —F, —Cl, —OH, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —CH₂OH, —CH₂CH₂OH, —CH(CH₃)CH₂OH, and —CH₂CH₂CH₂OH. In yet a further aspect, R¹ is selected from hydrogen, —F, —Cl, —OH, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —CH₂OH, and —CH₂CH₂OH. In an even further aspect, R¹ is selected from hydrogen, —F, —Cl, —OH, methyl, —OCH₃, —CF₃, —CHF₂, —CH₂F, —CCl₃, —CHCl₂, —CH₂Cl, and —CH₂OH.

In yet a further aspect, R¹ is selected from hydrogen, —F, —Cl, —OH, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —CH₂OH, —CH₂CH₂OH, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCHClCH₃. In a still further aspect, R¹ is selected from hydrogen, —F, —Cl, —OH, methyl, —OCH₃, —CF₃, —CHF₂, —CH₂F, —CCl₃, —CHCl₂, —CH₂Cl, —CH₂OH, —OCF₃, —OCHF₂, —OCH₂F, —OCCl₃, —OCHCl₂, and —OCH₂Cl.

In various aspects, R¹ is selected from hydrogen, —OH, C1-C6 alkoxy, and C1-C6 hydroxyalkyl. In a further aspect, R¹ is selected from hydrogen, —OH, C1-C4 alkoxy, and C1-C4 hydroxyalkyl. In a still further aspect, R¹ is selected from hydrogen, —OH, —OCH₃, —OCH₂CH₃, —OCH(CH₃)₂, —OCH₂CH₂CH₃, —CH₂OH, —CH₂CH₂OH, —CH(CH₃)CH₂OH, and —CH₂CH₂CH₂OH. In yet a further aspect, R¹ is selected from hydrogen, —OH, —OCH₃, —OCH₂CH₃, —CH₂OH, and —CH₂CH₂OH. In a still further aspect, R¹ is selected from hydrogen, —OH, —OCH₃, and —CH₂OH.

In various aspects, R¹ is C1-C6 alkoxy. In a further aspect, R¹ is C1-C4 alkoxy. In a still further aspect, R¹ is selected from —OCH₃, —OCH₂CH₃, —OCH(CH₃)₂, and —OCH₂CH₂CH₃. In yet a further aspect, R¹ is selected from —OCH₃ and —OCH₂CH₃. In a still further aspect, R¹ is —OCH₃.

In various aspects, R¹ is selected from hydrogen, halogen, C1-C6 haloalkyl, and C1-C6 haloalkoxy. In a further aspect, R¹ is selected from hydrogen, halogen, C1-C4 haloalkyl, and C1-C4 haloalkoxy. In a still further aspect, R¹ is selected from hydrogen, —F, —Cl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CHClCH₃. In yet a further aspect, R¹ is selected from hydrogen, —F, —Cl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCHClCH₃. In a still further aspect, R¹ is selected from hydrogen, —F, —Cl, —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl.

In various aspects, R¹ is selected from hydrogen and C1-C6 alkyl. In a further aspect, R¹ is selected from hydrogen and C1-C4 alkyl. In a still further aspect, R¹ is selected from hydrogen, methyl, ethyl, n-propyl, and isopropyl. In yet a further aspect, R¹ is selected from hydrogen, methyl, and ethyl. In a still further aspect, R¹ is selected from hydrogen and methyl.

In various aspects, R¹ is C1-C6 alkyl. In a further aspect, R¹ is C1-C4 alkyl. In a still further aspect, R¹ is selected from methyl, ethyl, n-propyl, and isopropyl. In yet a further aspect, R¹ is selected from methyl and ethyl. In a still further aspect, R¹ is methyl.

In various aspects, R¹ is selected from hydrogen and halogen. In a further aspect, R¹ is selected from hydrogen, —F, —Cl, and —Br. In a still further aspect, R¹ is selected from hydrogen, —F and —Cl. In yet a further aspect, R¹ is selected from hydrogen and —Cl. In an even further aspect, R¹ is selected from hydrogen and —F.

In various aspects, R¹ is halogen. In a further aspect, R¹ is selected from —F, —Cl, and —Br. In a still further aspect, R¹ is selected from —F and —Cl. In yet a further aspect, R¹ is —Cl. In an even further aspect, R¹ is —F.

In various aspects, R¹ is hydrogen.

### h. R² Groups

In one aspect, R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷. In a further aspect, R² is selected from hydrogen, halogen, —OH, —CN, C1-C4 alkyl, C1-C4 alkoxy, C1-C4 haloalkyl, C1-C4 haloalkoxy, —CO₂H, —(C═O)H, —C(O)(C1-C4 alkyl), —CO₂(C1-C4 alkyl), and R⁷. In a still further aspect, R² is selected from hydrogen, —F, —Cl, —OH, —CN, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CHClCH₃, —CO₂H, —(C═O)H, —C(O)CH₃, —C(O)CH₂CH₃, —C(O)CH(CH₃)₂, —C(O)CH₂CH₂CH₃, —CO₂CH₃, —CO₂CH₂CH₃, —CO₂CH(CH₃)₂, —CO₂CH₂CH₂CH₃, and R⁷. In yet a further aspect, R² is selected from hydrogen, —F, —Cl, —OH, —CN, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCCl₃, —OCHCl₂, —OCH₂Cl, —OCH₂CCl₃, —OCH₂CHCl₂, —OCH₂CH₂Cl, —OCHClCH₃, —CO₂H, —(C═O)H, —C(O)CH₃, —C(O)CH₂CH₃, —CO₂CH₃, —CO₂CH₂CH₃, and R⁷. In an even further aspect, R² is selected from hydrogen, —F, —Cl, —OH, —CN, methyl, —OCH₃, —CF₃, —CHF₂, —CH₂F, —CCl₃, —CHCl₂, —CH₂Cl, —OCF₃, —OCHF₂, —OCH₂F, —OCCl₃, —OCHCl₂, —OCH₂Cl, —CO₂H, —(C═O)H, —C(O)CH₃, —CO₂CH₃, and R⁷.

In one aspect, R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl). In a further aspect, R² is selected from hydrogen, halogen, —OH, —CN, C1-C4 alkyl, C1-C4 alkoxy, C1-C4 haloalkyl, C1-C4 haloalkoxy, —CO₂H, —(C═O)H, —C(O)(C1-C4 alkyl), and —CO₂(C1-C4 alkyl). In a still further aspect, R² is selected from hydrogen, —F, —Cl, —OH, —CN, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CHClCH₃, —CO₂H, —(C═O)H, —C(O)CH₃, —C(O)CH₂CH₃, —C(O)CH(CH₃)₂, —C(O)CH₂CH₂CH₃, —CO₂CH₃, —CO₂CH₂CH₃, —CO₂CH(CH₃)₂, and —CO₂CH₂CH₂CH₃. In yet a further aspect, R² is selected from hydrogen, —F, —Cl, —OH, —CN, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCCl₃, —OCHCl₂, —OCH₂Cl, —OCH₂CCl₃, —OCH₂CHCl₂, —OCH₂CH₂Cl, —OCHClCH₃, —CO₂H, —(C═O)H, —C(O)CH₃, —C(O)CH₂CH₃, —CO₂CH₃, and —CO₂CH₂CH₃. In an even further aspect, R² is selected from hydrogen, —F, —C1, —OH, —CN, methyl, —OCH₃, —CF₃, —CHF₂, —CH₂F, —CCl₃, —CHCl₂, —CH₂Cl, —OCF₃, —OCHF₂, —OCH₂F, —OCCl₃, —OCHCl₂, —OCH₂Cl, —CO₂H, —(C═O)H, —C(O)CH₃, and —CO₂CH₃.

In various aspects, R² is selected from hydrogen, —CO₂H, —(C═O)H, —C(O)(C1-C6 alkyl), —CO₂(C1-C6 alkyl), and R⁷. In a further aspect, R² is selected from hydrogen, halogen, —CO₂H, —(C═O)H, —C(O)(C1-C4 alkyl), —CO₂(C1-C4 alkyl), and R⁷. In a still further aspect, R² is selected from hydrogen, —CO₂H, —(C═O)H, —C(O)CH₃, —C(O)CH₂CH₃, —C(O)CH(CH₃)₂, —C(O)CH₂CH₂CH₃, —CO₂CH₃, —CO₂CH₂CH₃, —CO₂CH(CH₃)₂, —CO₂CH₂CH₂CH₃, and R⁷. In yet a further aspect, R² is selected from hydrogen, —CO₂H, —(C═O)H, —C(O)CH₃, —C(O)CH₂CH₃, —CO₂CH₃, —CO₂CH₂CH₃, and R⁷. In an even further aspect, R² is selected from hydrogen, —CO₂H, —(C═O)H, —C(O)CH₃, —CO₂CH₃, and R⁷.

In various aspects, R² is selected from hydrogen, —CO₂H, —(C═O)H, —C(O)(C1-C6 alkyl), and —CO₂(C1-C6 alkyl). In a further aspect, R² is selected from hydrogen, halogen, —CO₂H, —(C═O)H, —C(O)(C1-C4 alkyl), and —CO₂(C1-C4 alkyl). In a still further aspect, R² is selected from hydrogen, —CO₂H, —(C═O)H, —C(O)CH₃, —C(O)CH₂CH₃, —C(O)CH(CH₃)₂, —C(O)CH₂CH₂CH₃, —CO₂CH₃, —CO₂CH₂CH₃, —CO₂CH(CH₃)₂, and —CO₂CH₂CH₂CH₃. In yet a further aspect, R² is selected from hydrogen, —CO₂H, —(C═O)H, —C(O)CH₃, —C(O)CH₂CH₃, —CO₂CH₃, and —CO₂CH₂CH₃. In an even further aspect, R² is selected from hydrogen, —CO₂H, —(C═O)H, —C(O)CH₃, and —CO₂CH₃.

In various aspects, R² is selected from hydrogen, halogen, —OH, —CN, C1-C6 alkyl, C1-C6 alkoxy, C1-C6 haloalkyl, C1-C4 haloalkoxy, and R⁷. In a further aspect, R² is selected from hydrogen, halogen, —OH, —CN, C1-C4 alkyl, C1-C4 alkoxy, C1-C4 haloalkyl, C1-C4 haloalkoxy, and R⁷. In a still further aspect, R² is selected from hydrogen, —F, —Cl, —OH, —CN, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CHClCH₃, and R⁷. In yet a further aspect, R² is selected from hydrogen, —F, —Cl, —OH, —CN, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCCl₃, —OCHCl₂, —OCH₂Cl, —OCH₂CCl₃, —OCH₂CHCl₂, —OCH₂CH₂Cl, —OCHClCH₃, and R⁷. In an even further aspect, R² is selected from hydrogen, —F, —Cl, —OH, —CN, methyl, —OCH₃, —CF₃, —CHF₂, —CH₂F, —CCl₃, —CHCl₂, —CH₂Cl, —OCF₃, —OCHF₂, —OCH₂F, —OCCl₃, —OCHCl₂, —OCH₂Cl, and R⁷.

In various aspects, R² is selected from hydrogen, halogen, —OH, —CN, C1-C6 alkyl, C1-C6 alkoxy, C1-C6 haloalkyl, C1-C4 haloalkoxy, and R⁷. In a further aspect, R² is selected from hydrogen, halogen, —OH, —CN, C1-C4 alkyl, C1-C4 alkoxy, C1-C4 haloalkyl, and C1-C4 haloalkoxy. In a still further aspect, R² is selected from hydrogen, —F, —Cl, —OH, —CN, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CHClCH₃. In yet a further aspect, R² is selected from hydrogen, —F, —Cl, —OH, —CN, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCCl₃, —OCHCl₂, —OCH₂Cl, —OCH₂CCl₃, —OCH₂CHCl₂, —OCH₂CH₂Cl, and —OCHClCH₃. In an even further aspect, R² is selected from hydrogen, —F, —Cl, —OH, —CN, methyl, —OCH₃, —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl.

In various aspects, R² is selected from hydrogen, halogen, C1-C6 haloalkyl, C1-C4 haloalkoxy, and R⁷. In a further aspect, R² is selected from hydrogen, halogen, C1-C4 haloalkyl, C1-C4 haloalkoxy, and R⁷. In a still further aspect, R² is selected from hydrogen, —F, —Cl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CHClCH₃, and R⁷. In yet a further aspect, R² is selected from hydrogen, —F, —Cl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCCl₃, —OCHCl₂, —OCH₂Cl, —OCH₂CCl₃, —OCH₂CHCl₂, —OCH₂CH₂Cl, —OCHClCH₃, and R⁷. In an even further aspect, R² is selected from hydrogen, —F, —Cl, —CF₃, —CHF₂, —CH₂F, —CCl₃, —CHCl₂, —CH₂Cl, —OCF₃, —OCHF₂, —OCH₂F, —OCCl₃, —OCHCl₂, —OCH₂Cl, and R⁷.

In various aspects, R² is selected from hydrogen, halogen, C1-C6 haloalkyl, and C1-C4 haloalkoxy. In a further aspect, R² is selected from hydrogen, halogen, C1-C4 haloalkyl, and C1-C4 haloalkoxy. In a still further aspect, R² is selected from hydrogen, —F, —Cl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CHClCH₃. In yet a further aspect, R² is selected from hydrogen, —F, —Cl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCCl₃, —OCHCl₂, —OCH₂Cl, —OCH₂CCl₃, —OCH₂CHCl₂, —OCH₂CH₂Cl, and —OCHClCH₃. In an even further aspect, R² is selected from hydrogen, —F, —Cl, —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl.

In various aspects, R² is selected from hydrogen, —OH, —CN, C1-C6 alkyl, C1-C6 alkoxy, and R⁷. In a further aspect, R² is selected from hydrogen, —OH, —CN, C1-C4 alkyl, C1-C4 alkoxy, and R⁷. In a still further aspect, R² is selected from hydrogen, —OH, —CN, methyl, ethyl, n-propyl, isopropyl, —OCH₃, —OCH₂CH₃, —OCH(CH₃)₂, —OCH₂CH₂CH₃, and R⁷. In yet a further aspect, R² is selected from hydrogen, —OH, —CN, methyl, ethyl, —OCH₃, —OCH₂CH₃, and R⁷. In an even further aspect, R² is selected from hydrogen, —OH, —CN, methyl, —OCH₃, and R⁷.

In various aspects, R² is selected from hydrogen, —OH, —CN, C1-C6 alkyl, and C1-C6 alkoxy. In a further aspect, R² is selected from hydrogen, —OH, —CN, C1-C4 alkyl, and C1-C4 alkoxy. In a still further aspect, R² is selected from hydrogen, —OH, —CN, methyl, ethyl, n-propyl, isopropyl, —OCH₃, —OCH₂CH₃, —OCH(CH₃)₂, and —OCH₂CH₂CH₃. In yet a further aspect, R² is selected from hydrogen, —OH, —CN, methyl, ethyl, —OCH₃, and —OCH₂CH₃. In an even further aspect, R² is selected from hydrogen, —OH, —CN, methyl, and —OCH₃.

In various aspects, R² is selected from hydrogen, C1-C6 alkyl, and R⁷. In a further aspect, R² is selected from hydrogen, C1-C4 alkyl, and R⁷. In a still further aspect, R² is selected from hydrogen, methyl, ethyl, n-propyl, isopropyl, and R⁷. In yet a further aspect, R² is selected from hydrogen, methyl, ethyl, and R⁷. In an even further aspect, R² is selected from hydrogen, methyl, and R⁷.

In various aspects, R² is selected from hydrogen and C1-C6 alkyl. In a further aspect, R² is selected from hydrogen and C1-C4 alkyl. In a still further aspect, R² is selected from hydrogen, methyl, ethyl, n-propyl, and isopropyl. In yet a further aspect, R² is selected from hydrogen, methyl, and ethyl. In an even further aspect, R² is selected from hydrogen and methyl.

In various aspects, R² is C1-C6 alkyl. In a further aspect, R² is C1-C4 alkyl. In a still further aspect, R² is selected from methyl, ethyl, n-propyl, and isopropyl. In yet a further aspect, R² is selected from methyl and ethyl. In an even further aspect, R² is methyl.

In various aspects, R² is selected from hydrogen and halogen. In a further aspect, R² is selected from hydrogen, —F, —Cl, and —Br. In a still further aspect, R² is selected from hydrogen, —F, and —Cl. In yet a further aspect, R² is selected from hydrogen and —Cl. In an even further aspect, R² is selected from hydrogen and —F.

In various aspects, R² is halogen. In a further aspect, R² is selected from —F, —Cl, and —Br. In a still further aspect, R² is selected from —F and —Cl. In yet a further aspect, R² is —Cl. In an even further aspect, R² is —F.

In various aspects, R² is R⁷.

In various aspects, R² is hydrogen.

### i. R³ Groups

In one aspect, R³ is selected from hydrogen, halogen, —OH, C1-C6 alkyl, C1-C6 alkoxy, C1-C6 haloalkyl, C1-C6 haloalkoxy, and C1-C6 hydroxyalkyl. In a further aspect, R³ is selected from hydrogen, halogen, —OH, C1-C4 alkyl, C1-C4 alkoxy, C1-C4 haloalkyl, C1-C4 haloalkoxy, and C1-C4 hydroxyalkyl. In a still further aspect, R³ is selected from hydrogen, —F, —Cl, —OH, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CHClCH₃, —CH₂OH, —CH₂CH₂OH, —CH(CH₃)CH₂OH, and —CH₂CH₂CH₂OH. In yet a further aspect, R³ is selected from hydrogen, —F, —Cl, —OH, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, —OCHClCH₃, —CH₂OH, and —CH₂CH₂OH. In a still further aspect, R³ is selected from hydrogen, —F, —Cl, —OH, methyl, —OCH₃, —CF₃, —CHF₂, —CH₂F, —CCl₃, —CHCl₂, —CH₂Cl, —OCF₃, —OCHF₂, —OCH₂F, —OCCl₃, —OCHCl₂, —OCH₂Cl, and —CH₂OH.

In one aspect, R³ is selected from hydrogen, halogen, —OH, C1-C6 alkyl, C1-C6 alkoxy, and C1-C6 hydroxyalkyl. In a still further aspect, R³ is selected from hydrogen, —F, —C1, —OH, methyl, ethyl, n-propyl, isopropyl, —OCH₃, —OCH₂CH₃, —OCH(CH₃)₂, —OCH₂CH₂CH₃, —CH₂OH, —CH₂CH₂OH, —CH(CH₃)CH₂OH, and —CH₂CH₂CH₂OH. In yet a further aspect, R³ is selected from hydrogen, —F, —Cl, —OH, methyl, ethyl, —OCH₃, —OCH₂CH₃, —CH₂OH, and —CH₂CH₂OH. In a still further aspect, R³ is selected from hydrogen, —F, —Cl, —OH, methyl, —OCH₃, and —CH₂OH.

In various aspects, R³ is selected from hydrogen, —OH, C1-C6 alkoxy, and C1-C6 hydroxyalkyl. In a further aspect, R³ is selected from hydrogen, —OH, C1-C4 alkoxy, and C1-C4 hydroxyalkyl. In a still further aspect, R³ is selected from hydrogen, —OH, —OCH₃, —OCH₂CH₃, —OCH(CH₃)₂, —OCH₂CH₂CH₃, —CH₂OH, —CH₂CH₂OH, —CH(CH₃)CH₂OH, and —CH₂CH₂CH₂OH. In yet a further aspect, R³ is selected from hydrogen, —OH, —OCH₃, —OCH₂CH₃, —CH₂OH, and —CH₂CH₂OH. In a still further aspect, R³ is selected from hydrogen, —OH, —OCH₃, and —CH₂OH.

In various aspects, R³ is C1-C6 alkoxy. In a further aspect, R¹ is C1-C4 alkoxy. In a still further aspect, R³ is selected from —OCH₃, —OCH₂CH₃, —OCH(CH₃)₂, and —OCH₂CH₂CH₃. In yet a further aspect, R³ is selected from —OCH₃ and —OCH₂CH₃. In a still further aspect, R³ is —OCH₃.

In various aspects, R³ is selected from hydrogen, halogen, C1-C6 haloalkyl, and C1-C6 haloalkoxy. In a further aspect, R³ is selected from hydrogen, halogen, C1-C4 haloalkyl, and C1-C4 haloalkoxy. In a still further aspect, R³ is selected from hydrogen, —F, —Cl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CHClCH₃. In yet a further aspect, R³ is selected from hydrogen, —F, —Cl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCHClCH₃. In a still further aspect, R³ is selected from hydrogen, —F, —Cl, —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl.

In various aspects, R³ is selected from C1-C6 haloalkyl and C1-C6 haloalkoxy. In a further aspect, R³ is selected from C1-C4 haloalkyl and C1-C4 haloalkoxy. In a still further aspect, R³ is selected from —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CHClCH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CHClCH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCHClCH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CHClCH₃. In yet a further aspect, R³ is selected from —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CHClCH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCHClCH₃. In a still further aspect, R³ is selected from —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl. In yet a further aspect, R³ is selected from —CF₃ and —OCF₃.

In various aspects, R³ is selected from hydrogen and C1-C6 alkyl. In a further aspect, R³ is selected from hydrogen and C1-C4 alkyl. In a still further aspect, R³ is selected from hydrogen, methyl, ethyl, n-propyl, and isopropyl. In yet a further aspect, R³ is selected from hydrogen, methyl, and ethyl. In a still further aspect, R³ is selected from hydrogen and methyl.

In various aspects, R³ is C1-C6 alkyl. In a further aspect, R³ is C1-C4 alkyl. In a still further aspect, R³ is selected from methyl, ethyl, n-propyl, and isopropyl. In yet a further aspect, R³ is selected from methyl and ethyl. In a still further aspect, R³ is methyl.

In various aspects, R³ is selected from hydrogen and halogen. In a further aspect, R³ is selected from hydrogen, —F, —Cl, and —Br. In a still further aspect, R³ is selected from hydrogen, —F and —Cl. In yet a further aspect, R³ is selected from hydrogen and —Cl. In an even further aspect, R³ is selected from hydrogen and —F.

In various aspects, R³ is halogen. In a further aspect, R³ is selected from —F, —Cl, and —Br. In a still further aspect, R³ is selected from —F and —Cl. In yet a further aspect, R³ is —Cl. In an even further aspect, R³ is —F.

In various aspects, R³ is hydrogen.

### j. R⁴ Groups

In one aspect, R⁴ is selected from hydrogen and C1-C4 alkyl. In a further aspect, R⁴ is selected from hydrogen, methyl, ethyl, n-propyl, and isopropyl. In a still further aspect, R⁴ is selected from hydrogen, methyl, and ethyl. In yet a further aspect, R⁴ is selected from hydrogen and ethyl. In an even further aspect, R⁴ is selected from hydrogen and methyl.

In various aspects, R⁴ is C1-C4 alkyl. In a further aspect, R⁴ is selected from methyl, ethyl, n-propyl, and isopropyl. In a still further aspect, R⁴ is selected from methyl and ethyl. In yet a further aspect, R⁴ is methyl.

In various aspects, R⁴ is hydrogen.

### k. R^{5A} and R^{5B} Groups

In one aspect, each of R^{5a} and R^{5b} is independently selected from hydrogen and C1-C8 alkyl. In a further aspect, each of R^{5a} and R^{5b} is independently selected from hydrogen and C1-C4 alkyl. In a still further aspect, each of R^{5a} and R^{5b} is independently selected from hydrogen, methyl, ethyl, n-propyl, and isopropyl. In yet a further aspect, each of R^{5a} and R^{5b} is independently selected from hydrogen, methyl, and ethyl. In an even further aspect, each of R^{5a} and R^{5b} is independently selected from hydrogen and ethyl. In a still further aspect, each of R^{5a} and R^{5b} is independently selected from hydrogen and methyl.

In various aspects, each of R^{5a} and R^{5b} is independently C1-C8 alkyl. In a further aspect, each of R^{5a} and R^{5b} is independently C1-C4 alkyl. In a still further aspect, each of R^{5a} and R^{5b} is independently selected from methyl, ethyl, n-propyl, and isopropyl. In yet a further aspect, each of R^{5a} and R^{5b} is independently selected from methyl and ethyl. In an even further aspect, each of R^{5a} and R^{5b} is ethyl. In a still further aspect, each of R^{5a} and R^{5b} is methyl.

In various aspects, R^{5a} is C1-C8 alkyl. In a further aspect, R^{5a} is C1-C4 alkyl. In a still further aspect, R^{5a} is selected from methyl, ethyl, n-propyl, and isopropyl. In yet a further aspect, R^{5a} is selected from methyl and ethyl. In an even further aspect, R^{5a} is ethyl. In a still further aspect, R^{5a} is methyl. In various aspects, R^{5a} is tert-butyl.

In various aspects, R^{5b} is C1-C8 alkyl. In a further aspect, R^{5b} is C1-C4 alkyl. In a still further aspect, R^{5b} is selected from methyl, ethyl, n-propyl, and isopropyl. In yet a further aspect, R^{5b} is selected from methyl and ethyl. In an even further aspect, R^{5b} is ethyl. In a still further aspect, R^{5b} is methyl. In various aspects, R^{5b} is tert-butyl.

In various aspects, R^{5a} is hydrogen and R^{5b} is selected from hydrogen and C1-C8 alkyl. In a further aspect, R^{5a} is hydrogen and R^{5b} is selected from hydrogen and C1-C4 alkyl. In a still further aspect, R^{5a} is hydrogen and R^{5b} is selected from hydrogen, methyl, ethyl, n-propyl, and isopropyl. In yet a further aspect, R^{5a} is hydrogen and R^{5b} is selected from hydrogen, methyl, and ethyl. In an even further aspect, R^{5a} is hydrogen and R^{5b} is selected from hydrogen and ethyl. In a still further aspect, R^{5a} is hydrogen and R^{5b} is selected from hydrogen and methyl.

In various aspects, R^{5b} is hydrogen and R^{5a} is selected from hydrogen and C1-C8 alkyl. In a further aspect, R^{5b} is hydrogen and R^{5a} is selected from hydrogen and C1-C4 alkyl. In a still further aspect, R^{5b} is hydrogen and R^{5a} is selected from hydrogen, methyl, ethyl, n-propyl, and isopropyl. In yet a further aspect, R^{5b} is hydrogen and R^{5a} is selected from hydrogen, methyl, and ethyl. In an even further aspect, R^{5b} is hydrogen and R^{5a} is selected from hydrogen and ethyl. In a still further aspect, R^{5b} is hydrogen and R^{5a} is selected from hydrogen and methyl.

In various aspects, each of R^{5a} and R^{5b} is hydrogen.

### l. R^{6A} and R^{6B} Groups

In one aspect, each of R^{6a} and R^{6b} is independently selected from hydrogen, C1-C8 alkyl, and R⁷. In a further aspect, each of R^{6a} and R^{6b} is independently selected from hydrogen, C1-C4 alkyl, and R⁷. In a still further aspect, each of R^{6a} and R^{6b} is independently selected from hydrogen, methyl, ethyl, n-propyl, isopropyl, and R⁷. In yet a further aspect, each of R^{6a} and R^{6b} is independently selected from hydrogen, methyl, ethyl, and R⁷. In an even further aspect, each of R^{6a} and R^{6b} is independently selected from hydrogen, methyl, and R⁷.

In one aspect, R^{6a} is R⁷.

In one aspect, R^{6b} is R⁷.

In various aspects, each of R^{6a} and R^{6b} is independently selected from hydrogen and C1-C8 alkyl. In a further aspect, each of R^{6a} and R^{6b} is independently selected from hydrogen and C1-C4 alkyl. In a still further aspect, each of R^{6a} and R^{6b} is independently selected from hydrogen, methyl, ethyl, n-propyl, and isopropyl. In yet a further aspect, each of R^{6a} and R^{6b} is independently selected from hydrogen, methyl, and ethyl. In an even further aspect, each of R^{6a} and R^{6b} is independently selected from hydrogen and methyl.

In various aspects, each of R^{6a} and R^{6b} is independently C1-C8 alkyl. In a further aspect, each of R^{6a} and R^{6b} is independently C1-C4 alkyl. In a still further aspect, each of R^{6a} and R^{6b} is independently selected from methyl, ethyl, n-propyl, and isopropyl. In yet a further aspect, each of R^{6a} and R^{6b} is independently selected from methyl and ethyl. In an even further aspect, each of R^{6a} and R^{6b} is methyl.

In various aspects, each of R^{6a} and R^{6b} is independently selected from hydrogen and R⁷.

In various aspects, each of R^{6a} and R^{6b} is hydrogen. In a further aspect, R^{6a} is hydrogen. In a still further aspect, R^{6b} is hydrogen.

In various aspects, one of R^{6a} and R^{6b} is a structure represented by a formula:

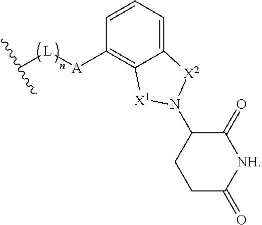

In a further aspect, n is 0. In a still further aspect, n is 1.

In various aspects, one of R^{6a} and R^{6b} is a structure selected from:

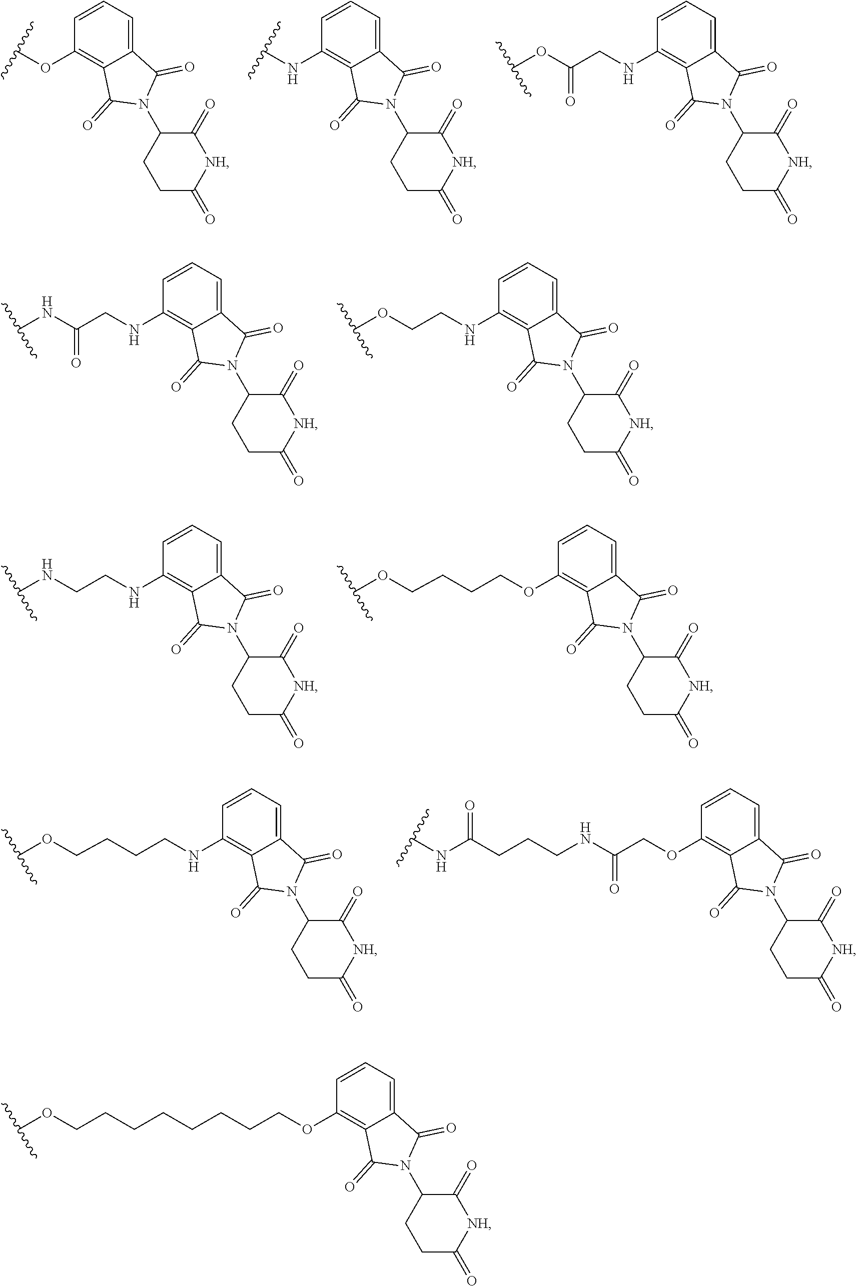

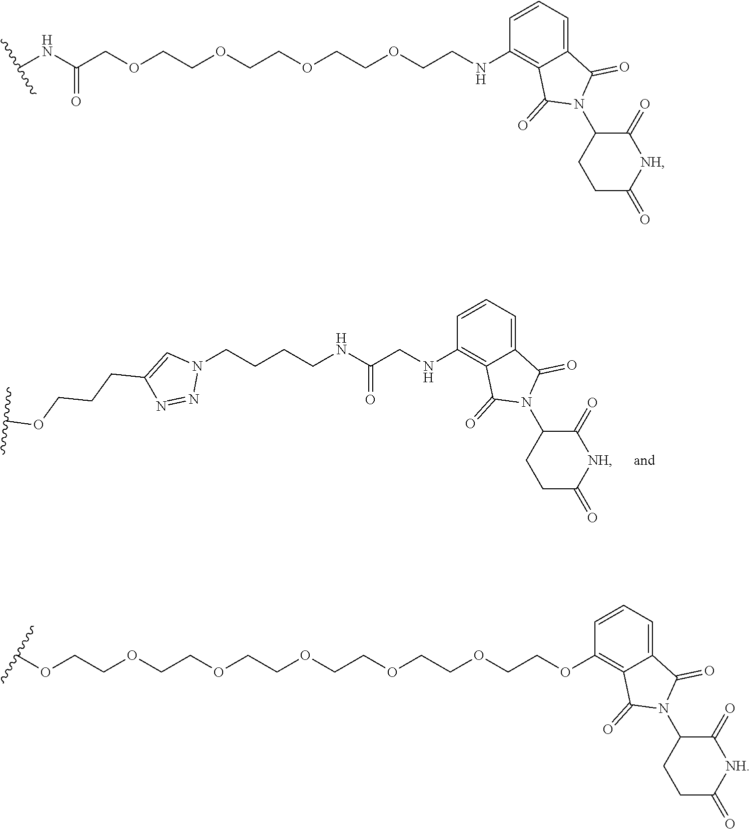 [SMILES: *NC(=O)COCCOCCOCCOCCNc1cccc2c1C(=O)N(C1CCC(=O)NC1=O)C2=O ; *OCCCc1cn(CCCCNC(=O)CNc2cccc3c2C(=O)N(C2CCC(=O)NC2=O)C3=O)nn1 ; *OCCOCCOCCOCCOCCOCCOc1cccc2c1C(=O)N(C1CCC(=O)NC1=O)C2=O]

In various aspects, one of R^{6a} and R^{6b} is a structure represented by a formula:

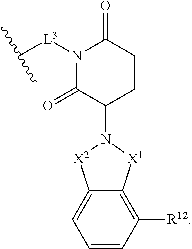

In a further aspect, each of X¹ and X² is —C(O)—. In a still further aspect, R¹² is —NH₂.

In various aspects, one of R^{6a} and R^{6b} is a structure selected from:

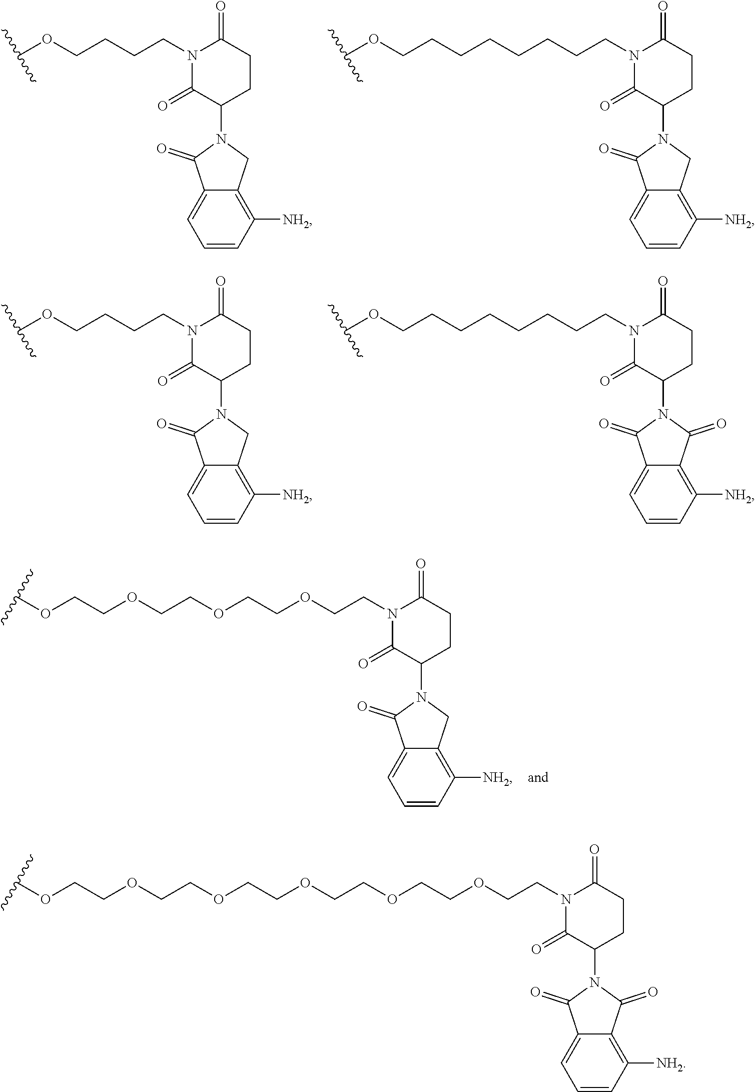

### m. R⁷ Groups

In one aspect, R⁷ is a structure represented by a formula selected from:

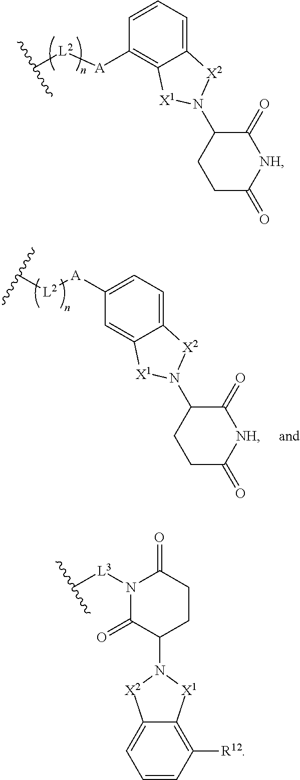

In various aspects, R⁷ is a structure represented by a formula:

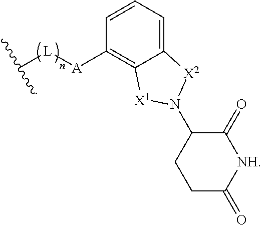

In various aspects, R⁷ is a structure represented by a formula:

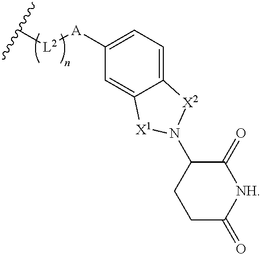

In various aspects, R⁷ is a structure represented by a formula:

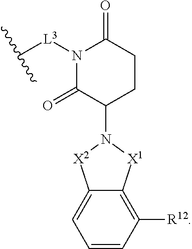

n. R¹⁰ Groups

In one aspect, R¹⁰ is selected from hydrogen and C1-C4 alkyl. In a further aspect, R¹⁰ is selected from hydrogen, methyl, ethyl, n-propyl, and isopropyl. In a still further aspect, R¹⁰ is selected from hydrogen, methyl, and ethyl. In yet a further aspect, R¹⁰ is selected from hydrogen and ethyl. In an even further aspect, R¹⁰ is selected from hydrogen and methyl.

In various aspects, R¹⁰ is C1-C4 alkyl. In a further aspect, R¹⁰ is selected from methyl, ethyl, n-propyl, and isopropyl. In a still further aspect, R¹⁰ is selected from methyl and ethyl. In yet a further aspect, R¹⁰ is methyl.

In various aspects, R¹⁰ is hydrogen.

### o. R¹¹ Groups

In one aspect, R¹¹, when present, is selected from hydrogen and C1-C4 alkyl. In a further aspect, R¹¹, when present, is selected from hydrogen, methyl, ethyl, n-propyl, and isopropyl. In a still further aspect, R¹¹, when present, is selected from hydrogen, methyl, and ethyl. In yet a further aspect, R¹¹, when present, is selected from hydrogen and ethyl. In an even further aspect, R¹¹, when present, is selected from hydrogen and methyl.

In various aspects, R¹¹, when present, is C1-C4 alkyl. In a further aspect, R¹¹, when present, is selected from methyl, ethyl, n-propyl, and isopropyl. In a still further aspect, R¹¹, when present, is selected from methyl and ethyl. In yet a further aspect, R¹¹, when present, is methyl.

In various aspects, R¹¹, when present, is hydrogen.

### p. R¹² Groups

In one aspect, R¹² is selected from hydrogen, —OH, and —NH₂. In a further aspect, R¹² is selected from hydrogen and —OH. In a still further aspect, R¹² is selected from hydrogen and —NH₂. In yet a further aspect, R¹² is selected from —OH and —NH₂. In an even further aspect, R¹² is —OH. In a still further aspect, R¹² is —NH₂. In yet a further aspect, R¹² is hydrogen.

### q. Ar¹ Groups

In one aspect, Ar¹ is a structure selected from:

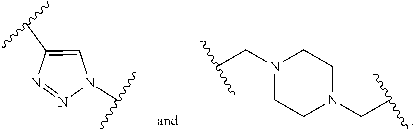 [SMILES: *c1cn(*)nn1 ; *CN1CCN(C*)CC1]

In a further aspect, Ar¹ is a structure:

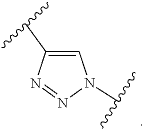 [SMILES: *c1cn(*)nn1]

In a further aspect, Ar¹ is a structure:

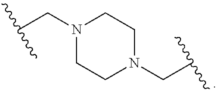 [SMILES: *CN1CCN(C*)CC1]

### 2. Example Compounds

In one aspect, a compound can be present as the following structure:

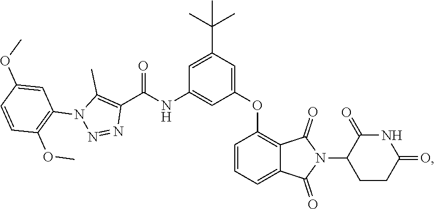

or a pharmaceutically acceptable salt thereof.

In one aspect, a compound can be present as one or more of the following structures:

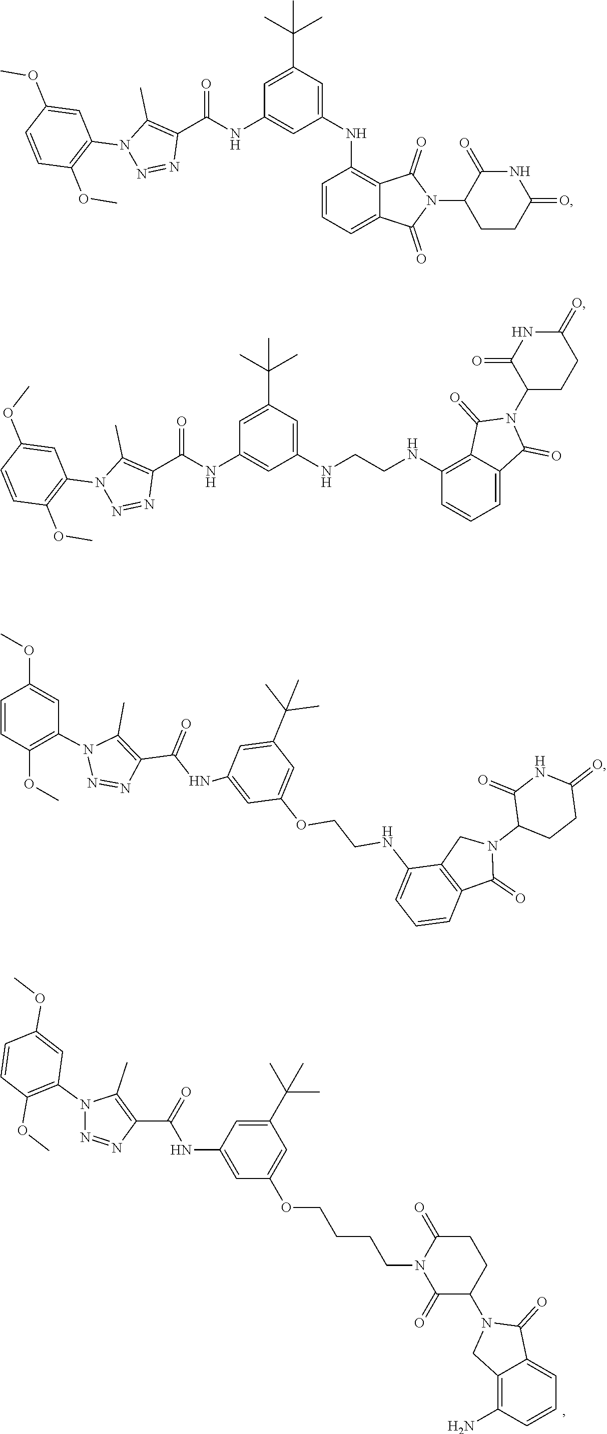 [SMILES: COc1ccc(OC)c(-n2nnc(C(=O)Nc3cc(Nc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(NCCNc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(OCCNc4cccc5c4CN(C4CCC(=O)NC4=O)C5=O)cc(C(C)(C)C)c3)c2C)c1 ; COc1ccc(OC)c(-n2nnc(C(=O)Nc3cc(OCCCCN4C(=O)CCC(N5Cc6c(N)cccc6C5=O)C4=O)cc(C(C)(C)C)c3)c2C)c1]

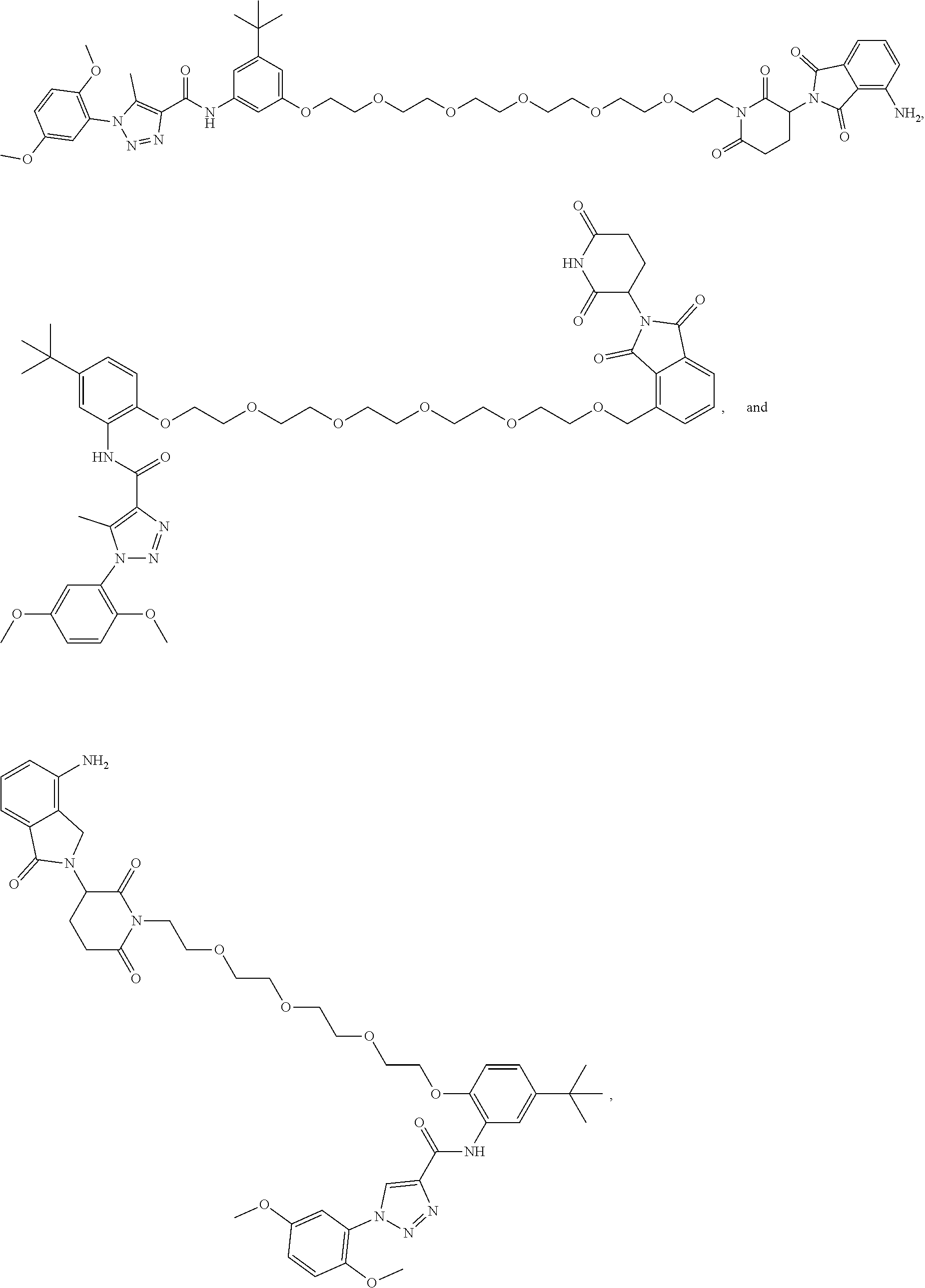 [SMILES: COc1ccc(OC)c(-n2nnc(C(=O)Nc3cc(OCCOCCOCCOCCOCCOCCN4C(=O)CCC(N5C(=O)c6cccc(N)c6C5=O)C4=O)cc(C(C)(C)C)c3)c2C)c1 ; COc1ccc(OC)c(-n2nnc(C(=O)Nc3cc(C(C)(C)C)ccc3OCCOCCOCCOCCOCCOCc3cccc4c3C(=O)N(C3CCC(=O)NC3=O)C4=O)c2C)c1 ; COc1ccc(OC)c(-n2cc(C(=O)Nc3cc(C(C)(C)C)ccc3OCCOCCOCCOCCN3C(=O)CCC(N4Cc5c(N)cccc5C4=O)C3=O)nn2)c1]

or a pharmaceutically acceptable salt thereof.

In one aspect, a compound can be present as the following structure:

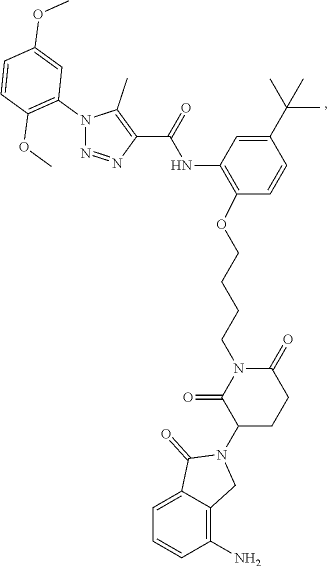 [SMILES: COc1ccc(OC)c(-n2nnc(C(=O)Nc3cc(C(C)(C)C)ccc3OCCCCN3C(=O)CCC(N4Cc5c(N)cccc5C4=O)C3=O)c2C)c1]

or a pharmaceutically acceptable salt thereof.

### C. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-X, as described and exemplified below. The following examples are provided so that the invention might be more fully understood, are illustrative only, and should not be construed as limiting.

### 1. Route I

In one aspect, 1,4,5-substituted 1,2,3-triazole compounds can be prepared as shown below.

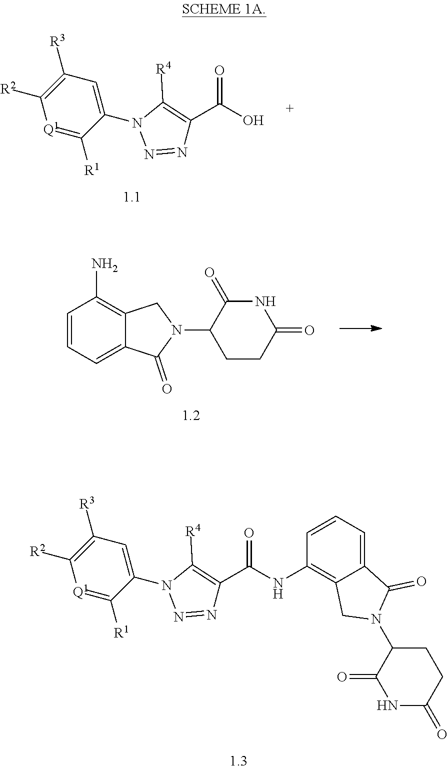

Compounds are represented in generic form, with substituents as noted in compound descriptions elsewhere herein. A more specific example is set forth below.

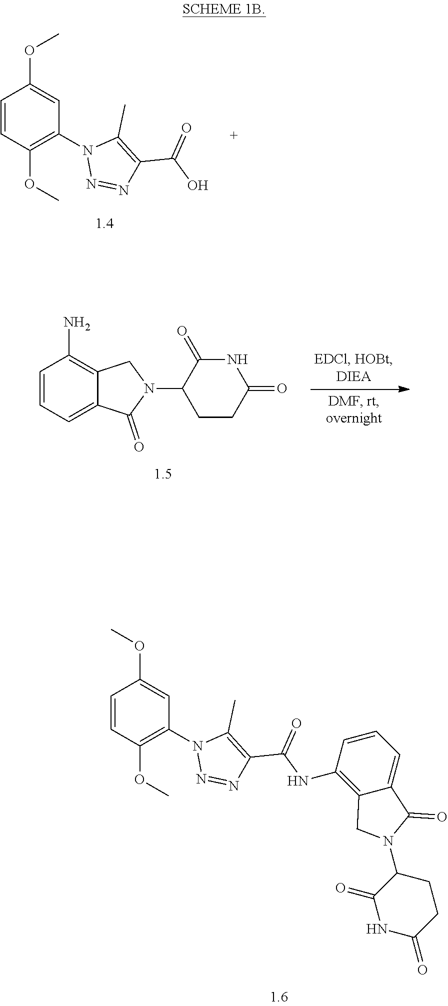 [SMILES: COc1ccc(OC)c(-n2nnc(C(=O)O)c2C)c1 ; Nc1cccc2c1CN(C1CCC(=O)NC1=O)C2=O ; COc1ccc(OC)c(-n2nnc(C(=O)Nc3cccc4c3CN(C3CCC(=O)NC3=O)C4=O)c2C)c1]

In one aspect, compounds of type 1.6, and similar compounds, can be prepared according to reaction Scheme 1B above. Thus, compounds of type 1.6 can be prepared by a coupling reaction of an appropriate carboxylic acid, e.g., 1.4 as shown above, and an appropriate aniline derivative, e.g., 1.5 as shown above. Appropriate carboxylic acids and appropriate aniline derivatives are commercially available or prepared by methods known to one skilled in the art. The coupling reaction is carried out in the presence of an appropriate coupling agent, e.g., 1-ethyl-3-(3-dimethylaminopropyl)carbodiimide (EDCI), an appropriate activating agent, e.g., hydroxybenzotriazole (HOBt), and an appropriate base, e.g., N, N-diisopropylethylamine (DIPEA), in an appropriate solvent, e.g., dimethylformamide (DMF). As can be appreciated by one skilled in the art, the above reaction provides an example of a generalized approach wherein compounds similar in structure to the specific reactants above (compounds similar to compounds of type 1.1 and 1.2), can be substituted in the reaction to provide 1,4,5-substituted 1,2,3-triazole compounds similar to Formula 1.3.

### 2. Route II

In one aspect, 1,4,5-substituted 1,2,3-triazole compounds can be prepared as shown below.

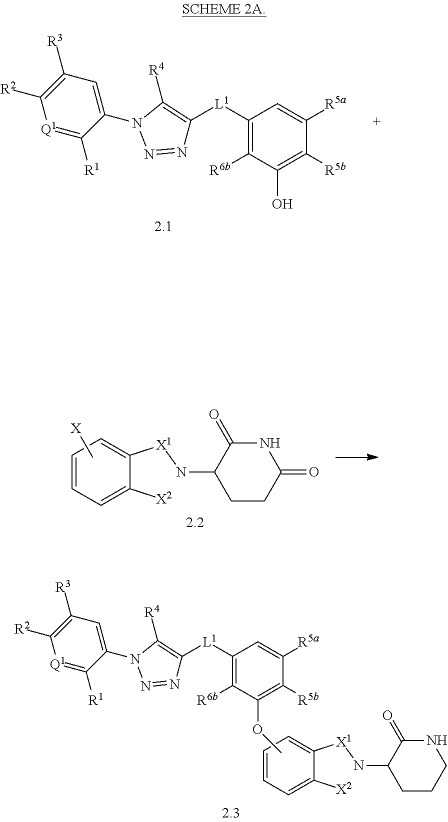

Compounds are represented in generic form, wherein X is a halogen, and with other substituents as noted in compound descriptions elsewhere herein. As would be understood by one of ordinary skill in the art, a similar synthetic method can be utilized for compounds in which R^{6b} is —OH and R^{6a} is as defined elsewhere herein. A more specific example is set forth below.

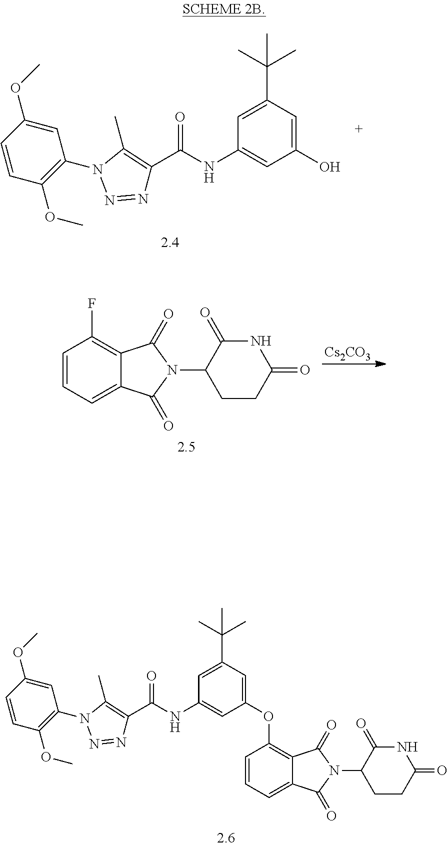

In one aspect, compounds of type 2.6, and similar compounds, can be prepared according to reaction Scheme 2B above. Thus, compounds of type 2.6 can be prepared by a nucleophilic aromatic substitution reaction between an appropriate phenol, e.g., 2.4 as shown above, and an appropriate aryl halide, e.g., 2.5 as shown above. Appropriate phenols and appropriate aryl halides are commercially available or prepared by methods known to one skilled in the art. The nucleophilic aromatic substitution reaction is carried out in the presence of an appropriate base, e.g., cesium carbonate. As can be appreciated by one skilled in the art, the above reaction provides an example of a generalized approach wherein compounds similar in structure to the specific reactants above (compounds similar to compounds of type 2.1 and 2.2), can be substituted in the reaction to provide 1,4,5-substituted 1,2,3-triazole compounds similar to Formula 2.3.

### 3. Route III

In one aspect, 1,4,5-substituted 1,2,3-triazole compounds can be prepared as shown below.

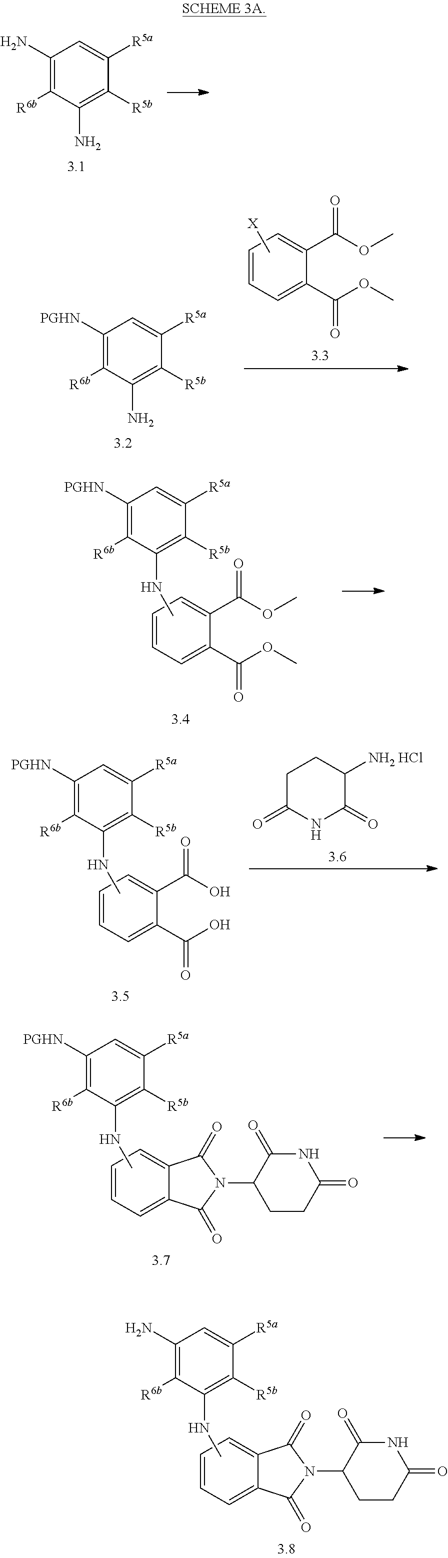

Compounds are represented in generic form, wherein X is a halogen, PG is an amine protecting group, and with other substituents as noted in compound descriptions elsewhere herein. As would be understood by one of ordinary skill in the art, a similar synthetic method can be utilized for compounds in which R^{6b} is —NH₂ and R^{6a} is as defined elsewhere herein. A more specific example is set forth below.

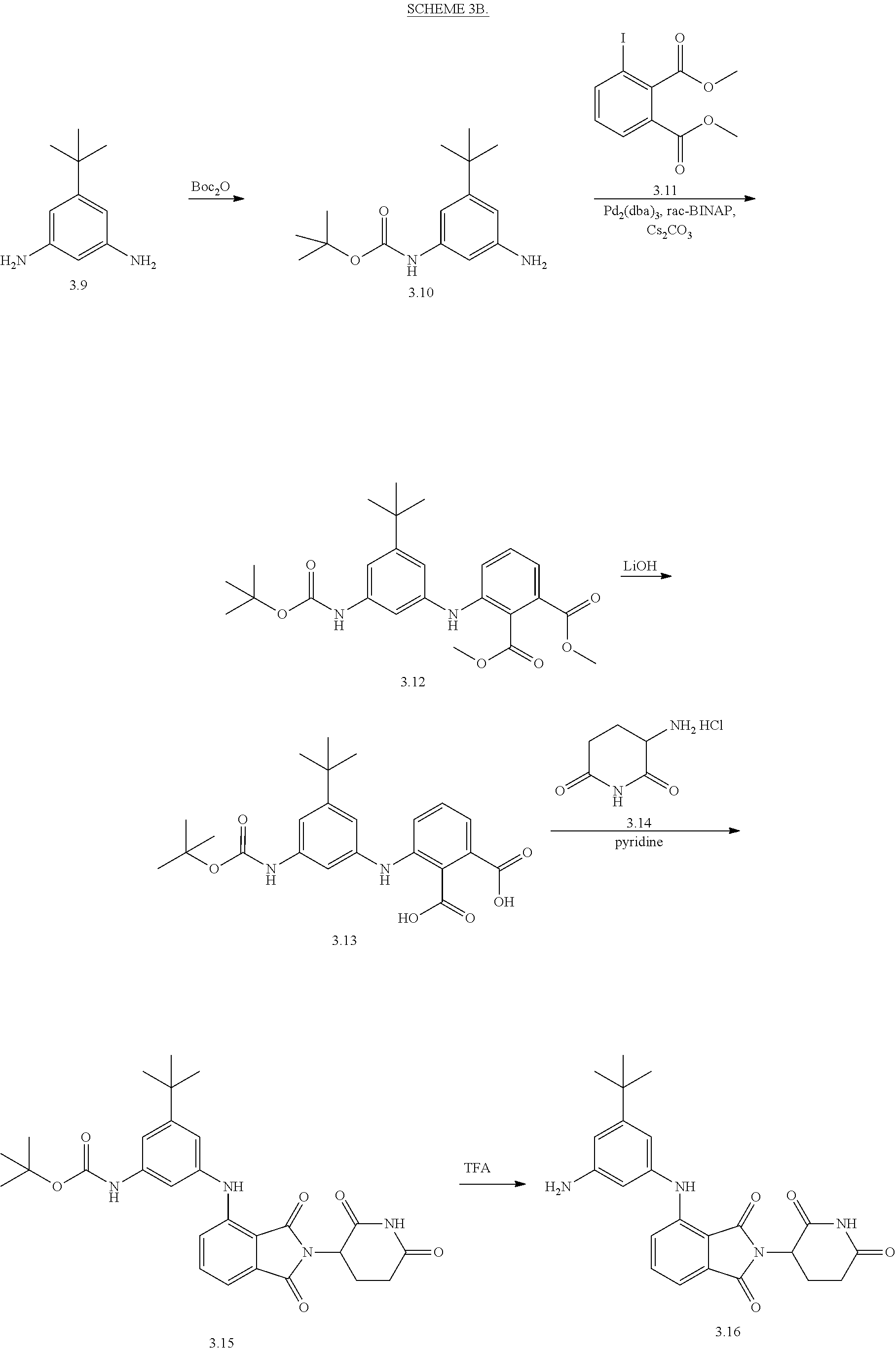

In one aspect, compounds of type 3.17, and similar compounds, can be prepared according to reaction Scheme 3B above. Thus, compounds of type 3.10 can be prepared by protection of an appropriate aniline, e.g., 3.9 as shown above, using an appropriate protecting group agent, e.g., di-tert-butyl dicarbonate. Appropriate anilines are commercially available or prepared by methods known to one skilled in the art. Compounds of type 3.12 can be prepared by coupling an appropriate aniline, e.g., 3.10 as shown above, and an appropriate aryl halide, e.g., 3.11 as shown above. Appropriate aryl halides are commercially available or prepared by methods known to one skilled in the art. The coupling reaction is carried out in the presence of an appropriate catalyst, e.g., tris(dibenzylideneacetone)dipalladium (0), an appropriate ligand, e.g., (±)-2,2′-bis(diphenylphosphino)-1,1′-binaphthalene (rac-BINAP), and an appropriate base, e.g., cesium carbonate. Compounds of type 3.13 can be prepared by saponification of an appropriate diester, e.g., 3.12 as shown above. The saponification is carried out in the presence of an appropriate base, e.g., lithium hydroxide. Compounds of type 3.15 can be prepared by cyclization of an appropriate dicarboxylic acid, e.g., 3.13 as shown above. The cyclization is carried out in the presence of an appropriate dione, e.g., 3.14 as shown above, and an appropriate base, e.g., pyridine. Appropriate diones are commercially available or prepared by methods known to one skilled in the art. Compounds of type 3.16 can be prepared by deprotection of an appropriate phenylcarbamate, e.g. 3.15 as shown above. The deprotection is carried out in the presence of an appropriate acid, e.g., trifluoroacetic acid. As can be appreciated by one skilled in the art, the above reaction provides an example of a generalized approach wherein compounds similar in structure to the specific reactants above (compounds similar to compounds of type 3.1, 3.2, 3.3, 3.4, 3.5, 3.6, and 3.7), can be substituted in the reaction to provide 1,4,5-substituted 1,2,3-triazole compounds similar to Formula 3.8.

### 4. Route IV

In one aspect, 1,4,5-substituted 1,2,3-triazole compounds can be prepared as shown below.

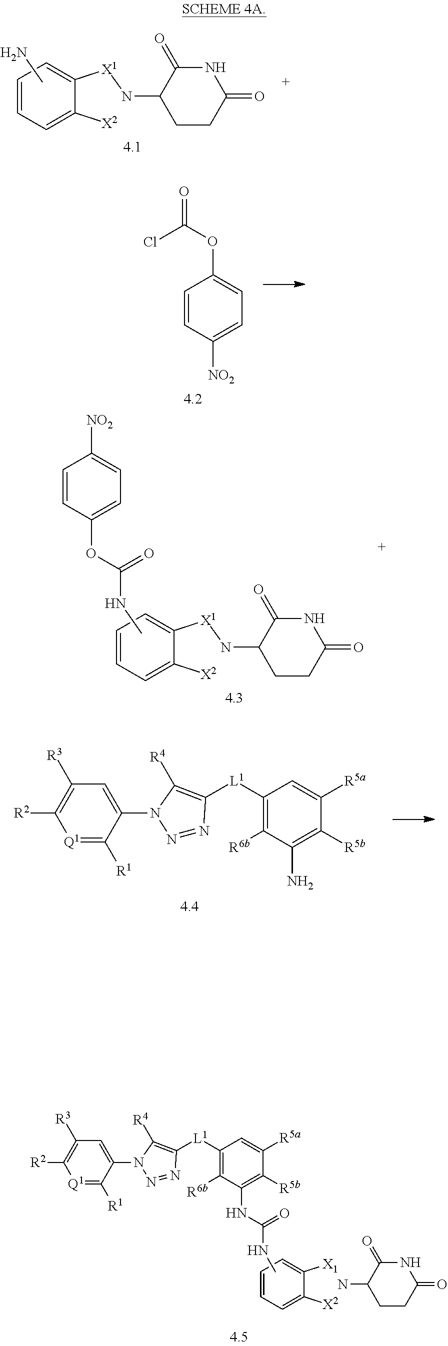

Compounds are represented in generic form, with substituents as noted in compound descriptions elsewhere herein. As would be understood by one of ordinary skill in the art, a similar synthetic method can be utilized for compounds in which R^{6b} is —NH₂ and R^{6a} is as defined elsewhere herein. A more specific example is set forth below.

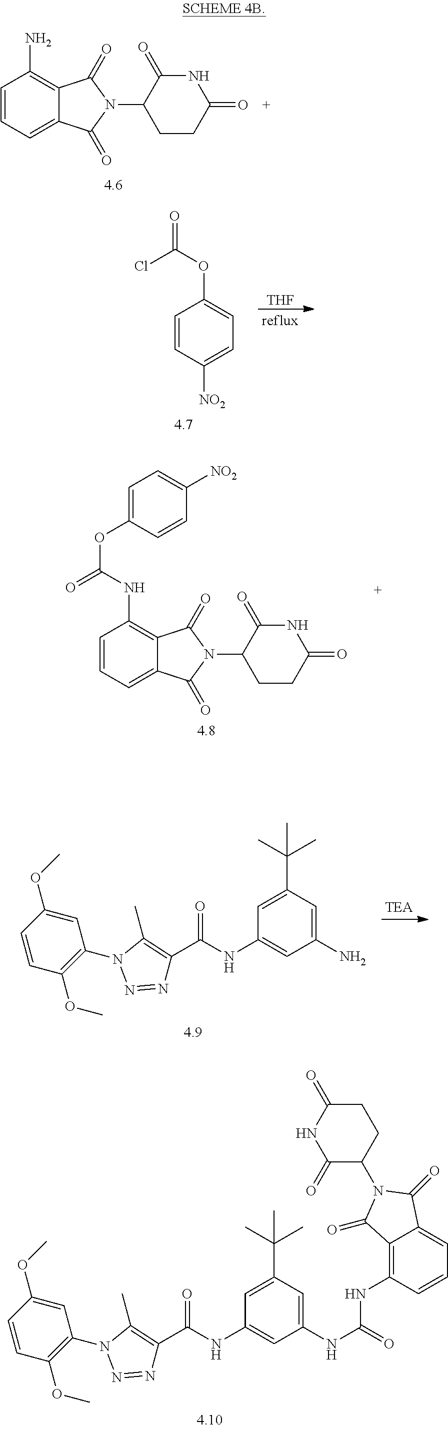 [SMILES: Nc1cccc2c1C(=O)N(C1CCC(=O)NC1=O)C2=O ; O=C(Cl)Oc1ccc([N+](=O)[O-])cc1 ; O=C1CCC(N2C(=O)c3cccc(NC(=O)Oc4ccc([N+](=O)[O-])cc4)c3C2=O)C(=O)N1 ; "THF" ; COc1ccc(OC)c(-n2nnc(C(=O)Nc3cc(N)cc(C(C)(C)C)c3)c2C)c1 ; COc1ccc(OC)c(-n2nnc(C(=O)Nc3cc(NC(=O)Nc4cccc5c4C(=O)N(C4CCC(=O)NC4=O)C5=O)cc(C(C)(C)C)c3)c2C)c1]

In one aspect, compounds of type 4.10, and similar compounds, can be prepared according to reaction Scheme 4B above. Thus, compounds of type 4.8 can be prepared by a coupling reaction between an appropriate aniline derivative, e.g., 4.6 as shown above, and an appropriate carbonohalidate, e.g., 4.7 as shown above. Appropriate aniline derivatives and appropriate carbonohalidates are commercially available or prepared by methods known to one skilled in the art. The coupling reaction is carried out in an appropriate solvent, e.g., tetrahydrofuran (THF), at an appropriate temperature, e.g., reflux conditions. Compounds of type 4.10 can be prepared by displacement of an appropriate leaving group, see, e.g., 4.8 as shown above, with an appropriate aniline derivative, e.g., 4.9 as shown above. Appropriate aniline derivatives are commercially available or prepared by methods known to one skilled in the art. The displacement reaction is carried out in the presence of an appropriate base, e.g., triethylamine (TEA). As can be appreciated by one skilled in the art, the above reaction provides an example of a generalized approach wherein compounds similar in structure to the specific reactants above (compounds similar to compounds of type 4.1, 4.2, 4.3, and 4.4), can be substituted in the reaction to provide 1,4,5-substituted 1,2,3-triazole compounds similar to Formula 4.5.

### 5. Route V

In one aspect, 1,4,5-substituted 1,2,3-triazole compounds can be prepared as shown below.

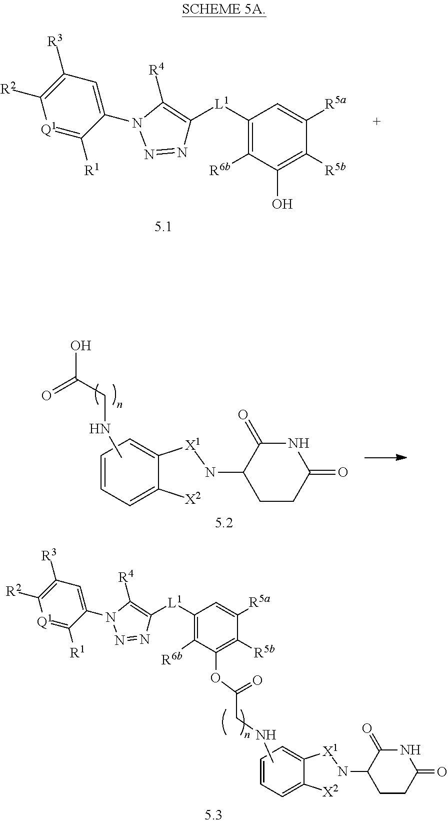

Compounds are represented in generic form, wherein n is 1, 2, 3, 4, 5, 6, 7, or 8, and with other substituents as noted in compound descriptions elsewhere herein. As would be understood by one of ordinary skill in the art, a similar synthetic method can be utilized for compounds in which R^{6b} is —OH and R^{6a} is as defined elsewhere herein. A more specific example is set forth below.

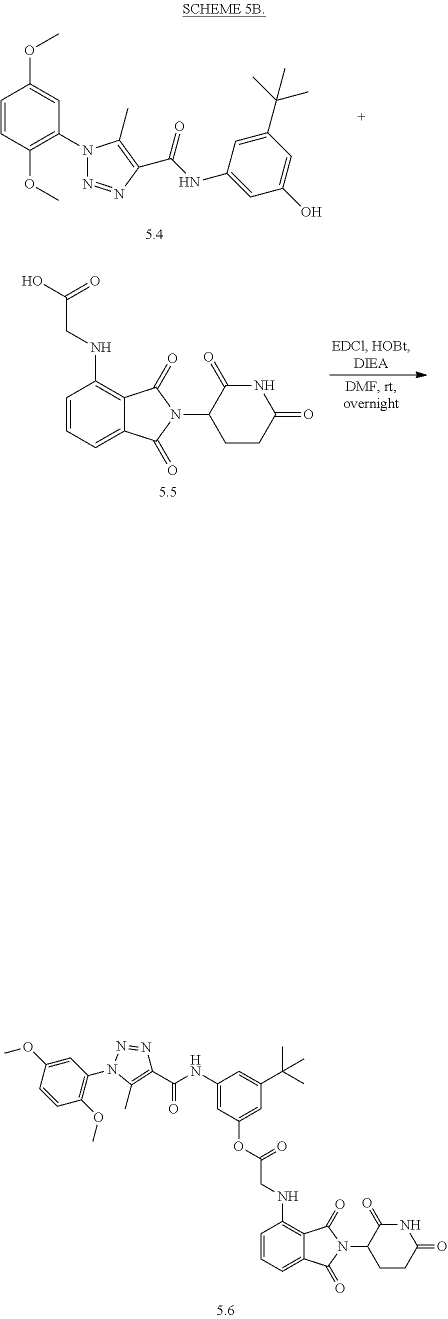

In one aspect, compounds of type 5.6, and similar compounds, can be prepared according to reaction Scheme 5B above. Thus, compounds of type 5.6 can be prepared by a, coupling reaction between an appropriate phenol derivative, e.g., 5.4 as shown above, and an appropriate carboxylic acid, e.g., 5.5 as shown above. Appropriate phenol derivatives and appropriate carboxylic acids are commercially available or prepared by methods known to one skilled in the art. The coupling reaction is carried out in the presence of an appropriate coupling agent, e.g., 1-ethyl-3-(3-dimethylaminopropyl)carbodiimide (EDCI), an appropriate activating agent, e.g., hydroxybenzotriazole (HOBt), and an appropriate base, e.g., N, N-diisopropylethylamine (DIPEA), in an appropriate solvent, e.g., dimethylformamide (DMF). As can be appreciated by one skilled in the art, the above reaction provides an example of a generalized approach wherein compounds similar in structure to the specific reactants above (compounds similar to compounds of type 5.1 and 5.2), can be substituted in the reaction to provide 1,4,5-substituted 1,2,3-triazole compounds similar to Formula 5.3.

### 6. Route VI

In one aspect, 1,4,5-substituted 1,2,3-triazole compounds can be prepared as shown below.

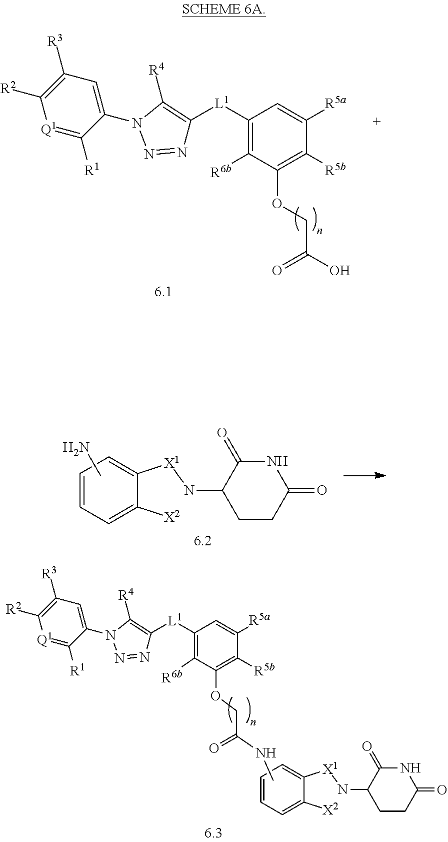

Compounds are represented in generic form, wherein n is 1, 2, 3, 4, 5, 6, 7, or 8, and with other substituents as noted in compound descriptions elsewhere herein. As would be understood by one of ordinary skill in the art, a similar synthetic method can be utilized for compounds in which R^{6b} is —OH and R^{6a} is as defined elsewhere herein. A more specific example is set forth below.

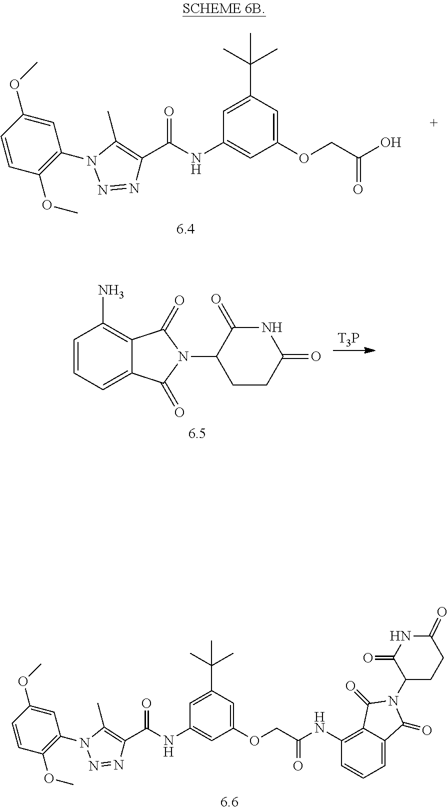 [SMILES: COc1ccc(OC)c(-n2nnc(C(=O)Nc3cc(OCC(=O)O)cc(C(C)(C)C)c3)c2C)c1 ; Nc1cccc2c1C(=O)N(C1CCC(=O)NC1=O)C2=O ; "T3P" ; COc1ccc(OC)c(-n2nnc(C(=O)Nc3cc(OCC(=O)Nc4cccc5c4C(=O)N(C4CCC(=O)NC4=O)C5=O)cc(C(C)(C)C)c3)c2C)c1]

In one aspect, compounds of type 6.6, and similar compounds, can be prepared according to reaction Scheme 6B above. Thus, compounds of type 6.6 can be prepared by a coupling reaction between an appropriate carboxylic acid, e.g., 6.4 as shown above, and an appropriate aniline derivative, e.g., 6.5 as shown above. Appropriate carboxylic acids and appropriate aniline derivatives are commercially available or prepared by methods known to one skilled in the art. The coupling reaction is carried out in the presence of an appropriate coupling agent, e.g., propylphosphonic anhydride. As can be appreciated by one skilled in the art, the above reaction provides an example of a generalized approach wherein compounds similar in structure to the specific reactants above (compounds similar to compounds of type 6.1 and 6.2), can be substituted in the reaction to provide 1,4,5-substituted 1,2,3-triazole compounds similar to Formula 1.4.

### 7. Route VII

In one aspect, 1,4,5-substituted 1,2,3-triazole compounds can be prepared as shown below.

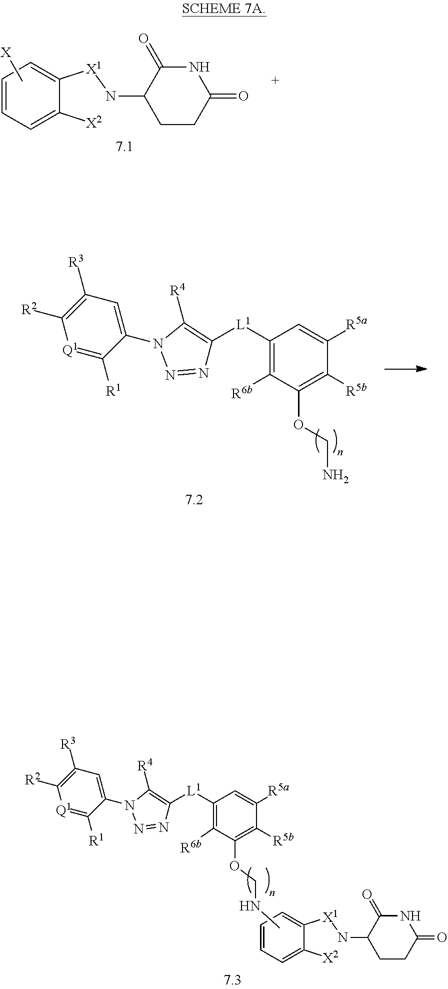

Compounds are represented in generic form, wherein X is halogen, n is 1, 2, 3, 4, 5, 6, 7, or 8, and with other substituents as noted in compound descriptions elsewhere herein. As would be understood by one of ordinary skill in the art, a similar synthetic method can be utilized for compounds in which R^{6b} is —O(CH₂)ₙNH₂ and R^{6a} is as defined elsewhere herein. A more specific example is set forth below.

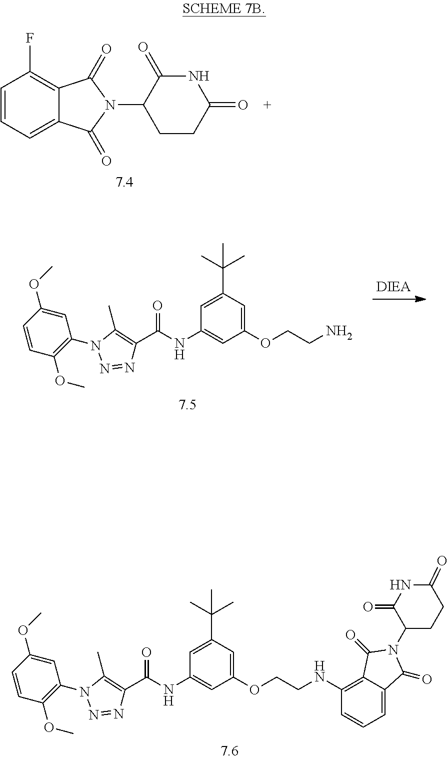 [SMILES: O=C1CCC(N2C(=O)c3cccc(F)c3C2=O)C(=O)N1 ; COc1ccc(OC)c(-n2nnc(C(=O)Nc3cc(OCCN)cc(C(C)(C)C)c3)c2C)c1 ; "DIEA" ; COc1ccc(OC)c(-n2nnc(C(=O)Nc3cc(OCCNc4cccc5c4C(=O)N(C4CCC(=O)NC4=O)C5=O)cc(C(C)(C)C)c3)c2C)c1]

In one aspect, compounds of type 7.6, and similar compounds, can be prepared according to reaction Scheme 7B above. Thus, compounds of type 7.6 can be prepared by a nucleophilic aromatic substitution reaction of an appropriate aryl halide, e.g., 7.4 as shown above, and an appropriate amine, e.g., 7.5 as shown above. Appropriate aryl halides and appropriate amines are commercially available or prepared by methods known to one skilled in the art. The nucleophilic aromatic substitution reaction is carried out in the presence of an appropriate base, e.g., N,N-diisopropylethylamine. As can be appreciated by one skilled in the art, the above reaction provides an example of a generalized approach wherein compounds similar in structure to the specific reactants above (compounds similar to compounds of type 7.1 and 7.2), can be substituted in the reaction to provide compounds similar to Formula 7.3.

### 8. Route VIII

In one aspect, 1,4,5-substituted 1,2,3-triazole compounds can be prepared as shown below.

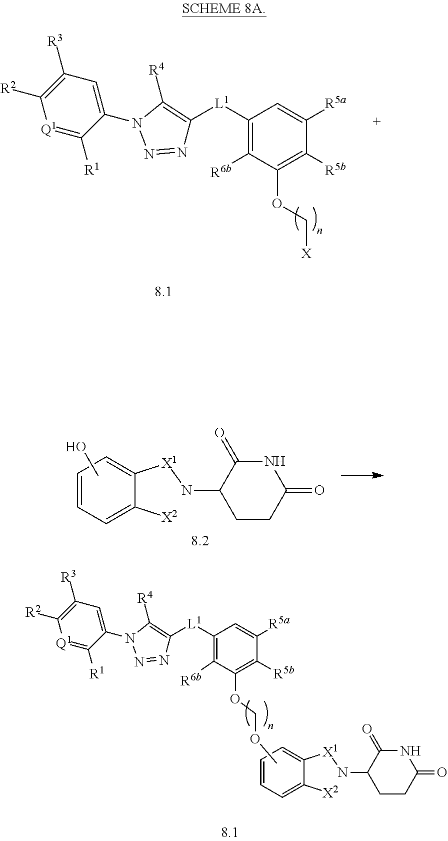

Compounds are represented in generic form, wherein X is halogen, n is 1, 2, 3, 4, 5, 6, 7, or 8, and with other substituents as noted in compound descriptions elsewhere herein. As would be understood by one of ordinary skill in the art, a similar synthetic method can be utilized for compounds in which R^{6b} is —O(CH₂)ₙX and R^{6a} is as defined elsewhere herein. A more specific example is set forth below.

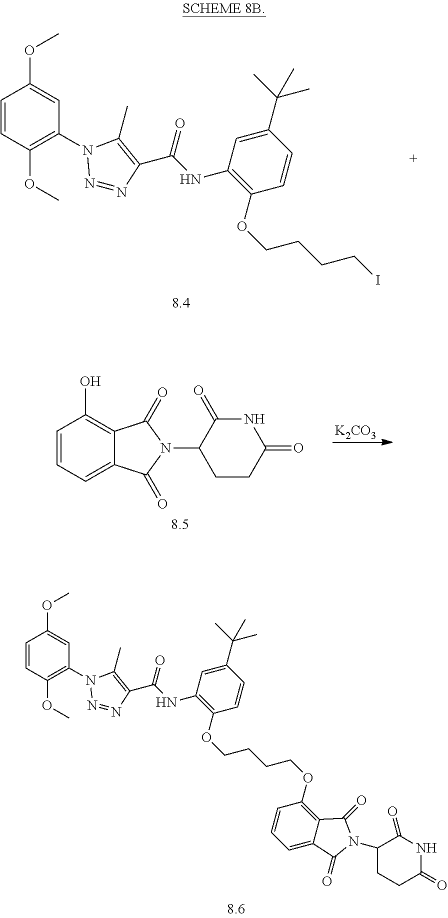

In one aspect, compounds of type 8.6, and similar compounds, can be prepared according to reaction Scheme 8B above. Thus, compounds of type 8.6 can be prepared by a nucleophilic substitution reaction of an appropriate alkyl halide, e.g., 8.4 as shown above, and an appropriate phenol derivative, e.g., 8.5 as shown above. Appropriate alkyl halides and appropriate phenol derivatives are commercially available or prepared by methods known to one skilled in the art. The nucleophilic substitution reaction is carried out in the presence of an appropriate base, e.g., potassium carbonate. As can be appreciated by one skilled in the art, the above reaction provides an example of a generalized approach wherein compounds similar in structure to the specific reactants above (compounds similar to compounds of type 8.1 and 8.2), can be substituted in the reaction to provide 1,4,5-substituted 1,2,3-triazole compounds similar to Formula 8.3.

### 9. Route IX

In one aspect, 1,4,5-substituted 1,2,3-triazole compounds can be prepared as shown below.

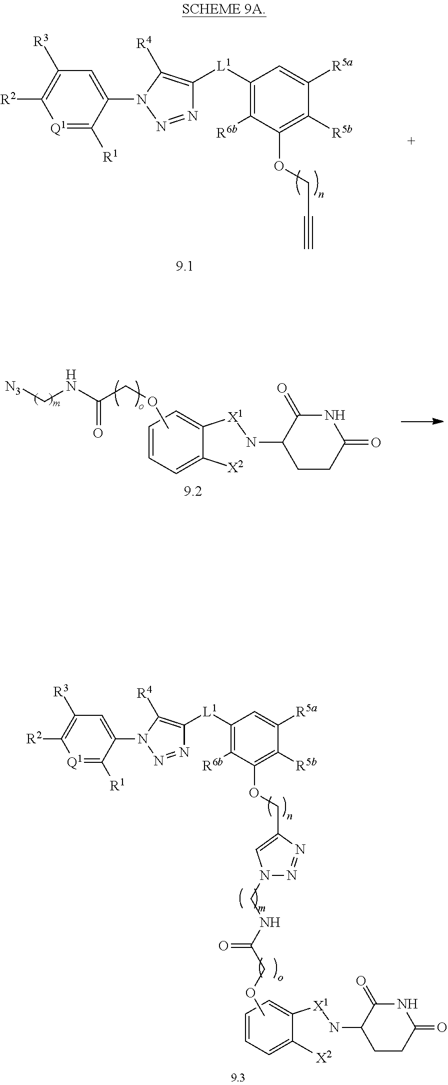

Compounds are represented in generic form, wherein each of m, n, and o is independently 1, 2, 3, 4, 5, 6, 7, or 8, and with other substituents as noted in compound descriptions elsewhere herein. As would be understood by one of ordinary skill in the art, a similar synthetic method can be utilized for compounds in which R^{6b} is —O(CH₂)ₙC≡CH and R^{6a} is as defined elsewhere herein. A more specific example is set forth below.

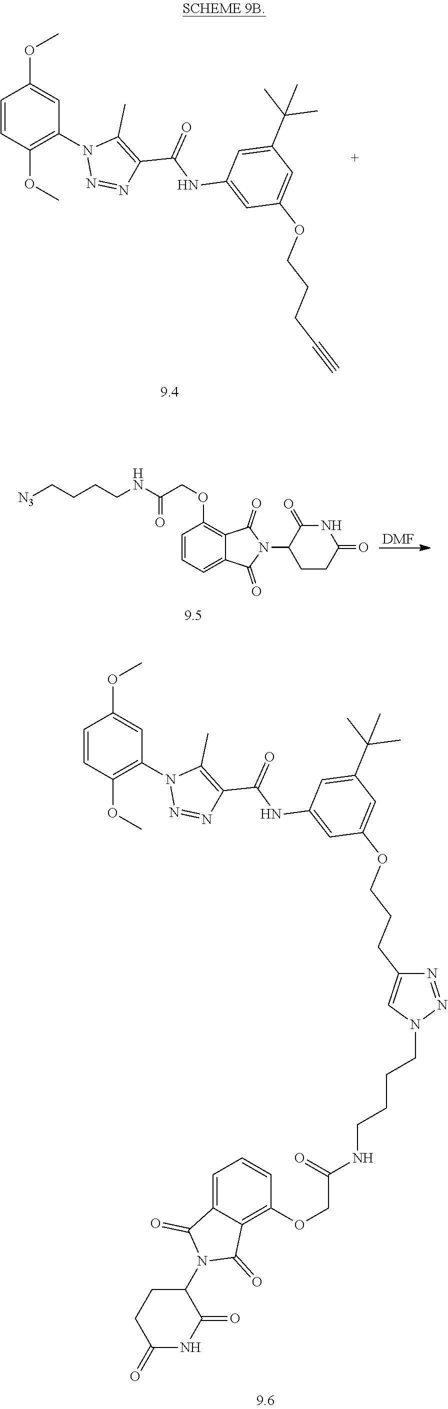

In one aspect, compounds of type 9.6, and similar compounds, can be prepared according to reaction Scheme 9B above. Thus, compounds of type 9.6 can be prepared by a cycloaddition of an appropriate alkyne, e.g., 9.4 as shown above, and an appropriate azide, e.g., 9.5 as shown above. Appropriate alkynes and appropriate azides are commercially available or prepared by methods known to one skilled in the art. The cycloaddition is carried out in the presence of an appropriate solvent, e.g., dimethylformamide (DMF). As can be appreciated by one skilled in the art, the above reaction provides an example of a generalized approach wherein compounds similar in structure to the specific reactants above (compounds similar to compounds of type 9.1 and 9.2), can be substituted in the reaction to provide 1,4,5-substituted 1,2,3-triazole compounds similar to Formula 9.3.

### 10. Route X

In one aspect, 1,4,5-substituted 1,2,3-triazole compounds can be prepared as shown below.

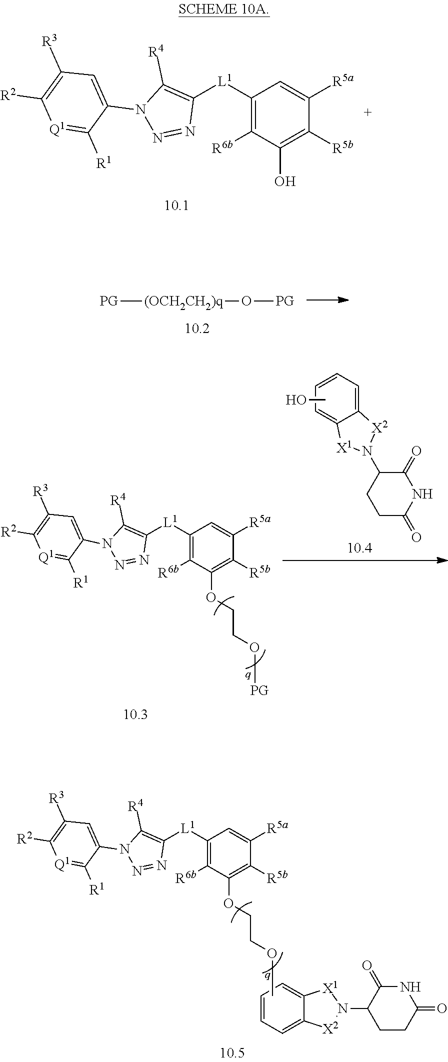

Compounds are represented in generic form, wherein each occurrence of PG is an alcohol protecting group and with other substituents as noted in compound descriptions elsewhere herein. As would be understood by one of ordinary skill in the art, a similar synthetic method can be utilized for compounds in which R^{6b} is —OH and R^{6a} is as defined elsewhere herein. A more specific example is set forth below.

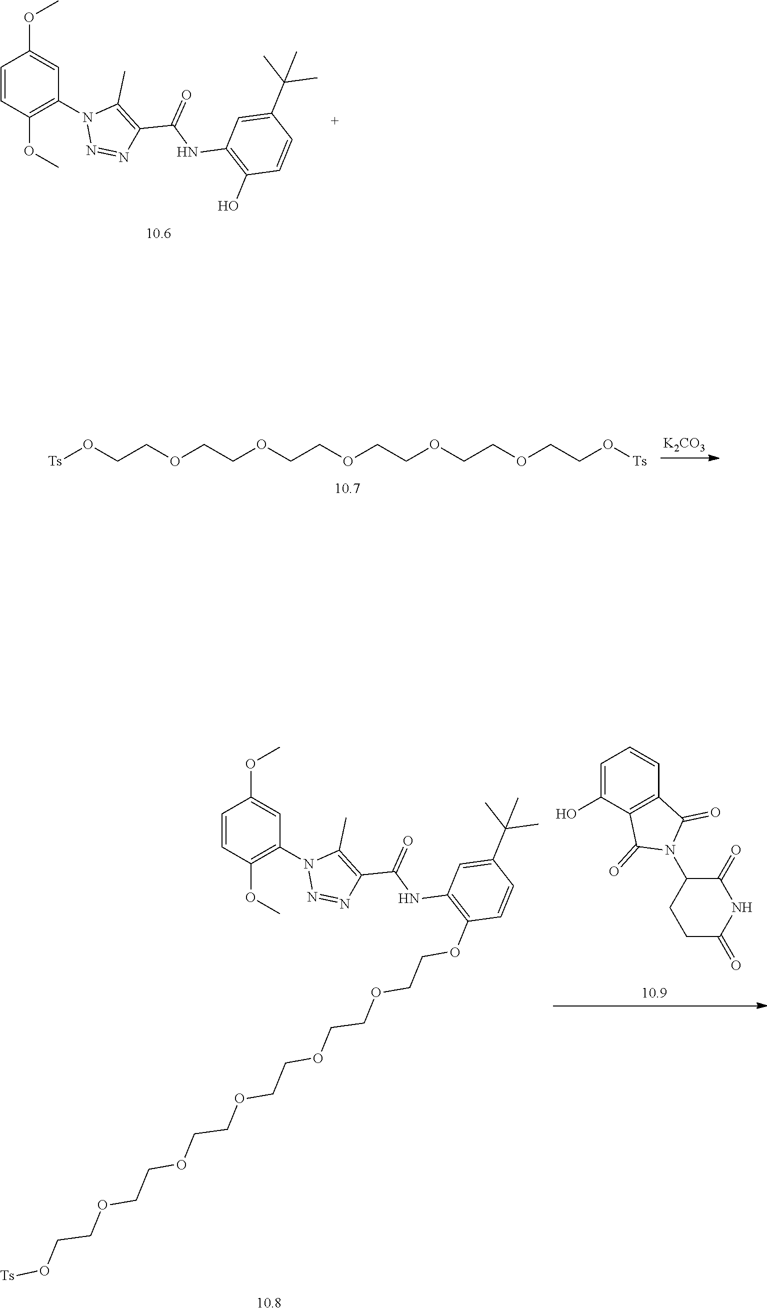

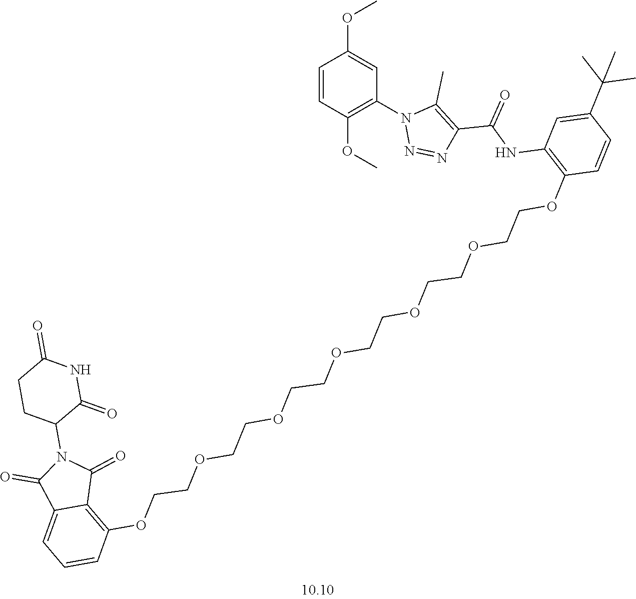 [SMILES: COc1ccc(OC)c(-n2nnc(C(=O)Nc3cc(C(C)(C)C)ccc3OCCOCCOCCOCCOCCOCCOc3cccc4c3C(=O)N(C3CCC(=O)NC3=O)C4=O)c2C)c1]

In one aspect, compounds of type 10.10, and similar compounds, can be prepared according to reaction Scheme 10B above. Thus, compounds of type 10.8 can be prepared by a nucleophilic substitution reaction of an appropriate phenol, e.g., 10.6 as shown above, and an appropriate di-tosylate, e.g., 10.7 as shown above. Appropriate phenols and appropriate tosylates are commercially available or prepared by methods known to one skilled in the art. The nucleophilic substitution reaction is carried out in the presence of an appropriate base, e.g., potassium carbonate. Compounds of type 10.10 can be prepared by a nucleophilic substitution reaction of an appropriate tosylate, e.g., 10.8 as shown above, and an appropriate phenol, e.g., 10.9 as shown above. Appropriate phenols are commercially available or prepared by methods known to one skilled in the art. As can be appreciated by one skilled in the art, the above reaction provides an example of a generalized approach wherein compounds similar in structure to the specific reactants above (compounds similar to compounds of type 10.1, 10.2, 10.3, and 10.4), can be substituted in the reaction to provide 1,4,5-substituted 1,2,3-triazole compounds similar to Formula 10.5.

### D. Pharmaceutical Compositions

In one aspect, the invention relates to pharmaceutical compositions comprising the disclosed compounds and products of disclosed methods. That is, a pharmaceutical composition can be provided comprising an effective amount of at least one disclosed compound, at least one product of a disclosed method, or a pharmaceutically acceptable salt, thereof, and a pharmaceutically acceptable carrier. In one aspect, the invention relates to pharmaceutical compositions comprising a pharmaceutically acceptable carrier and an effective amount of at least one disclosed compound; or a pharmaceutically acceptable salt, thereof.

Thus, in one aspect, disclosed are pharmaceutical compositions comprising an effective amount of a disclosed compound or a pharmaceutically acceptable salt thereof, and a pharmaceutically acceptable carrier. In a further aspect, the compound has a structure represented by a formula:

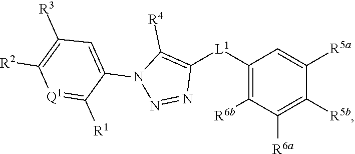

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷; wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl; wherein R⁴ is selected from hydrogen and C1-C4 alkyl; wherein each of R^{1a} and R^{5b} is independently selected from hydrogen and C1-C8 alkyl; wherein each of R^{6a} and R^{6b} is independently selected from hydrogen, C1-C8 alkyl, and R⁷; wherein R⁷ is a structure represented by a formula selected from:

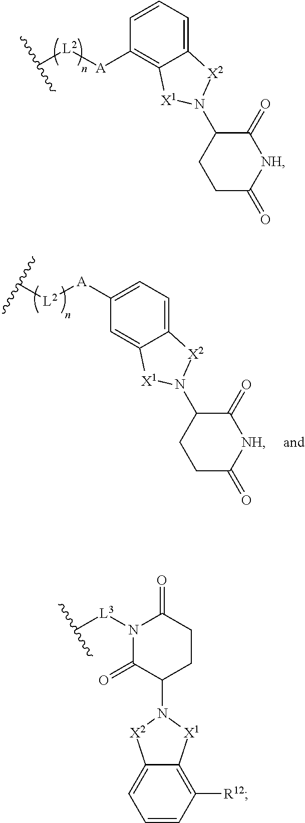

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

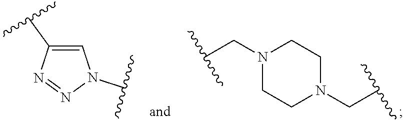

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, provided that one and only one of R², R^{6a}, and R^{6b} is R⁷, or a pharmaceutically acceptable salt thereof.

In a further aspect, the effective amount is a therapeutically effective amount. In a still further aspect, the effective amount is a prophylactically effective amount. In a still further aspect, the pharmaceutical composition comprises a compound that is a product of a disclosed method of making.

In a further aspect, the pharmaceutical composition comprises a disclosed compound. In a yet further aspect, the pharmaceutical composition comprises a product of a disclosed method of making.

In a further aspect, the mammal has been diagnosed with a need for treatment of a disorder of uncontrolled cellular proliferation such as, for example, a cancer. In a yet further aspect, the mammal has been diagnosed with a need for treatment of a disorder of uncontrolled cellular proliferation prior to the administering step. In an even further aspect, the mammal has been identified to be in need of treatment of a disorder of uncontrolled cellular proliferation. In a still further aspect, a therapeutic agent known to treat a disorder of uncontrolled cellular proliferation is co-administered with the pharmaceutical composition. In a yet further aspect, the therapeutic agent known to treat a disorder of uncontrolled cellular proliferation that is co-administered with the pharmaceutical composition is paclitaxel, irinotecan, leucovorin, dasatinib, or erlotinib.

In a further aspect, the pharmaceutical composition is used to treat a mammal. In a further aspect, the mammal is a human. In a still further aspect, the mammal has been diagnosed with a need for modulating an adverse drug reaction prior to the administering step. In an even further aspect, the mammal has been identified to be in need of treatment of an adverse drug reaction. In a still further aspect, the adverse drug reaction is enhanced toxicity, increased metabolism, and/or decreased efficacy. In a yet further aspect, the adverse drug reaction is associated with another therapeutic agent, and the pharmaceutical composition is administered to treat the adverse drug reaction associated with the other therapeutic agent. In an even further aspect, the adverse drug reaction is associated with another therapeutic agent, and the pharmaceutical composition is co-administered with the other therapeutic agent in order to treat the adverse drug reaction associated with the other therapeutic agent. In a further aspect, the pharmaceutical composition is used to decrease an adverse drug reaction.

In a further aspect, the mammal has been diagnosed with a need for treatment of a bowel disorder such as, for example, irritable bowel syndrome (IBS). In a yet further aspect, the mammal has been diagnosed with a need for treatment of a bowel disorder prior to the administering step. In an even further aspect, the mammal has been identified to be in need of treatment of a bowel disorder.

In various aspects, the pharmaceutical composition of the present invention comprises a pharmaceutically acceptable carrier; an effective amount of at least one disclosed compound; or a pharmaceutically acceptable salt thereof; and an anticancer agent. In a further aspect, the anticancer agent comprises a compound selected from paclitaxel, irinotecan, leucovorin, dasatinib, and erlotinib, or combinations thereof. In a still further aspect, the anticancer agent comprises a compound selected from paclitaxel, docetaxel, vinblastine, vincristine, vinorelbine, camptothecin, topotecan, irinotecan, belotecan, gimatecan, inidimitecan, indotecan, Genz-644282, daunorubicin, epirubicin, etoposide, teniposide, mitoxantrone, ellipticinium, vasaroxin, dexrazoxane, mebarone, 3-hydroxy-2-[(1R)-6-isopropenyl-3-methyl-cyclohex-2-en-1-yl]-5-pentyl-1,4-benzoquinone (HU-331), axitinib, crizotinib, dasatinib, erlotinib, gefitinib, imatinib, lapatinib, nilotinib, pazopanib, regorafenib, ruxolitinib, sorafenib, sunitinib, vandetanib, vemurafenib, doxorubicin, mitoxantrone, bleomycin, daunorubicin, dactinomycin, epirubicin, idarubicin, plicamycin, mitomycin, pentostatin, valrubicin, gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, methotrexate coadministered with leucovorin, thioguanine, carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin, etopside, vincristine, ixabepilone, vinorelbine, vinblastine, teniposide, everolimus, siroliumus, temsirolimus, or combinations thereof.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids,” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions that require modulation of PXR activity, an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for modulating PXR activity (e.g., modulating adverse reactions, treating a disease of uncontrolled cellular proliferation, or treating a bowel disorder) in mammals (e.g., humans) comprising combining one or more disclosed compounds, products, or compositions with a pharmaceutically acceptable carrier or diluent. Thus, in an aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### E. Methods of Modulating Pregnane X Receptor Activity in a Subject

In one aspect, disclosed are methods of modulating pregnane X receptor (PXR) activity in a subject in need thereof, the method comprising administering to the subject an effective amount of a disclosed compound or a pharmaceutically acceptable salt thereof.

Thus, in one aspect, disclosed are methods of modulating PXR activity in a subject in need thereof, the method comprising administering to the subject an effective amount of a compound having a structure represented by a formula:

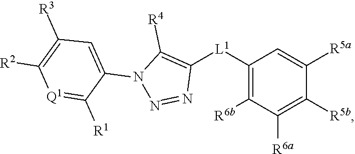

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷; wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein each of R^{6a} and R^{6b} is independently selected from hydrogen, C1-C8 alkyl, and R⁷; wherein R⁷ is a structure represented by a formula selected from:

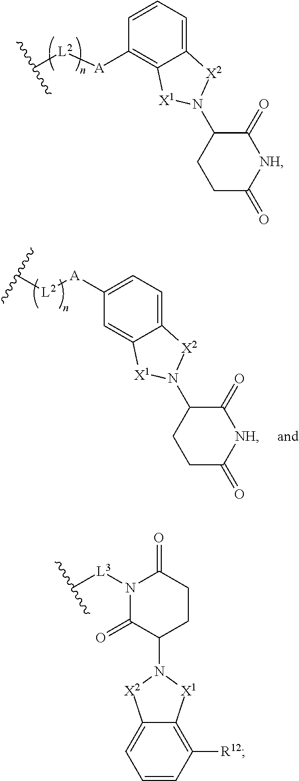

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar^{l}(C1-C8 alkyl)NHC(O)(C1-C4 alkyl)-; wherein q, when present, is selected from 1, 2, 3, 4, 5, 6, 7, and 8; wherein Ar¹ is a structure selected from:

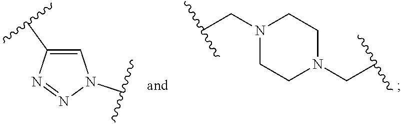

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, provided that one and only one of R², R^{6a}, and R^{6b} is R⁷, or a pharmaceutically acceptable salt thereof.

In a further aspect, the subject is a mammal. In a still further aspect, the subject is a human.

In a further aspect, modulating is increasing. In a still further aspect, modulating is activating. In yet a further aspect, modulating is activating and the compound has a structure represented by a formula:

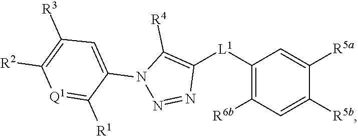

or a pharmaceutically acceptable salt thereof.

In a further aspect, modulating is decreasing. In a still further aspect, modulating is inhibiting. In yet a further aspect, modulating is inhibiting, and wherein the compound has a structure represented by a formula:

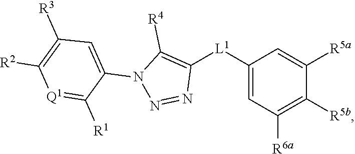

or a pharmaceutically acceptable salt thereof.

In a further aspect, the subject has been diagnosed with a need for modulating PXR activity prior to the administering step. For example, in various aspects, the subject has been diagnosed as having a disorder for which modulation of PXR activity is beneficial (e.g., cancer, a bowel disorder). In various further aspects, the subject has been diagnosed as having an adverse drug reaction for which modulation of PXR activity can be beneficial.

In a further aspect, the method further comprises identifying a subject in need for modulating PXR activity.

### F. Methods of Modulating Cellular Proliferation Activity in a Cell

In one aspect, disclosed are methods for modulating pregnane X receptor (PXR) activity in a cell, the method comprising contacting the cell with an effective amount of a disclosed compound or a pharmaceutically acceptable salt thereof.

Thus, in one aspect, disclosed are methods for modulating PXR activity in a cell, the method comprising contacting the cell with an effective amount of a compound having a structure represented by a formula:

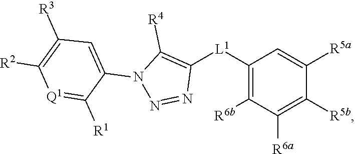

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷; wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl; wherein R⁴ is selected from hydrogen and C1-C4 alkyl; wherein each of R^{5a} and R^{1b} is independently selected from hydrogen and C1-C8 alkyl; wherein each of R^{6a} and R^{6b} is independently selected from hydrogen, C1-C8 alkyl, and R⁷; wherein R⁷ is a structure represented by a formula selected from:

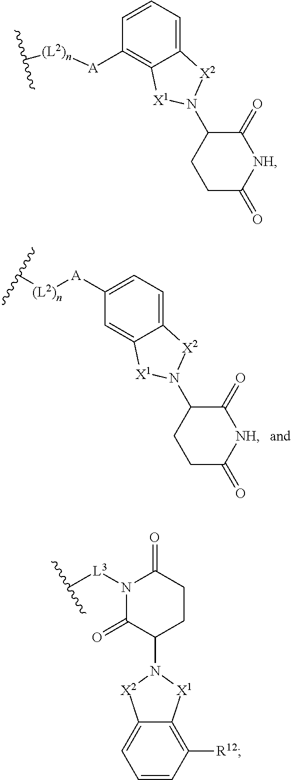

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1—C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

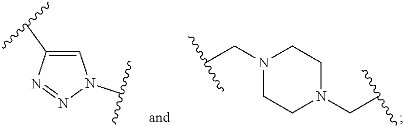

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, provided that one and only one of R², R^{6a}, and R^{6b} is R⁷, or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is ex vivo. In a still further aspect, contacting is in vitro.

In a further aspect, contacting is via administration to a mammal.

In a further aspect, the mammal has been diagnosed with a need for modulating PXR activity prior to the administering step. In a still further aspect, the mammal has been diagnosed with a need for treatment of a disorder related to PXR activity prior to the administering step.

### G. Methods of Treating a Disorder Associated with Uncontrolled Cellular Proliferation in a Subject

In various aspects, the compounds and compositions disclosed herein are useful for treating, preventing, ameliorating, controlling, or reducing the risk of a variety of disorders associated with uncontrolled cellular proliferation, such as, for example cancer. Thus, in one aspect, disclosed are methods of treating a disorder associated with uncontrolled cellular proliferation activity in a subject, the method comprising administering to the subject an effective amount of a disclosed compound or a pharmaceutically acceptable salt thereof.

In various aspects, disclosed are methods of treating a disorder associated with uncontrolled cellular proliferation activity in a subject, the method comprising administering to the subject an effective amount of a compound having a structure represented by a formula:

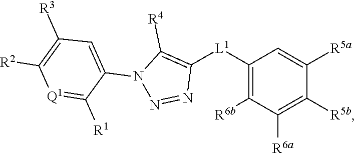

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷; wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein each of R^{6a} and R^{6b} is independently selected from hydrogen, C1-C8 alkyl, and R⁷; wherein R⁷ is a structure represented by a formula selected from:

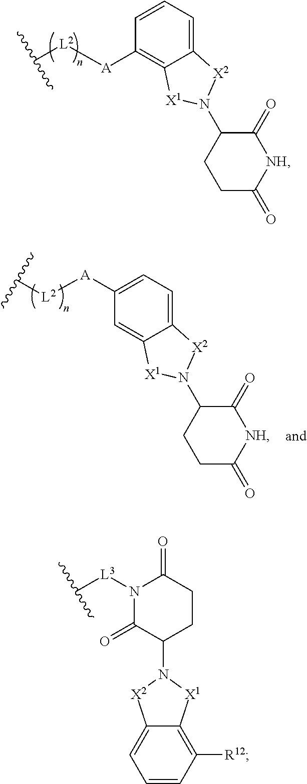

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

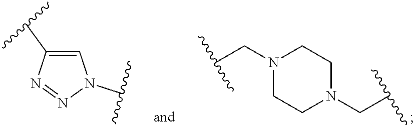

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, provided that one and only one of R², R^{6a}, and R^{6b} is R⁷, or a pharmaceutically acceptable salt thereof.

In various aspects, the disclosed compounds can be used in combination with one or more other drugs in the treatment, prevention, control, amelioration, or reduction of risk of disorders associated with uncontrolled cellular proliferation, such as for example cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia, activity for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly. Accordingly, the pharmaceutical compositions include those that contain one or more other active ingredients, in addition to a compound of the present invention.

In one aspect, the one or more other active ingredients is a chemotherapeutic agent. The chemotherapeutic agent can be selected from an alkylating agent, an antimetabolite agent, an antineoplastic antibiotic agent, a mitotic inhibitor agent, and a mTor inhibitor agent. In one aspect, the one or more other active ingredients is an antineoplastic antibiotic agent selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof. In a further aspect, the one or more other active ingredients is an antimetabolite agent selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof. In a further aspect, the one or more other active ingredients is an alkylating agent selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof. In a further aspect, the one or more other active ingredients is a mitotic inhibitor agent selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof. In a further aspect, the one or more other active ingredients is an mTor inhibitor agent selected from everolimus, siroliumus, and temsirolimus, or a pharmaceutically acceptable salt, hydrate, solvate, or polymorph thereof.

In a further aspect, the subject is a mammal. In a still further aspect, the mammal is human.

In a further aspect, the subject has been diagnosed with a need for treatment of the disorder prior to the administering step. In a still further aspect, the subject is at risk for developing the disorder prior to the administering step.

In a further aspect, the method further comprises identifying a subject at risk for developing the disorder prior to the administering step.

In a further aspect, the disorder associated with uncontrolled cellular proliferation is cancer. In a still further aspect, the cancer is selected from breast cancer, renal cancer, gastric cancer, and colorectal cancer. In yet a further aspect, the cancer is selected from lymphoma, cancers of the brain, genitourinary tract cancer, lymphatic system cancer, stomach cancer, larynx cancer, lung, pancreatic cancer, breast cancer, and malignant melanoma. In an even further aspect, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, plasma cell neoplasm (myeloma), and acute lymphoblastic leukemia, activity.

### H. Methods of Decreasing an Adverse Drug Reaction in a Subject

In various aspects, the compounds and compositions disclosed herein are useful for treating, preventing, ameliorating, controlling, reducing the risk of, or otherwise decreasing an adverse reaction associated with a drug such as, for example, an anticancer agent, an antibacterial agent, a non-steroidal anti-inflammatory agent, or an anticonvulsant agent. Thus, in one aspect, disclosed are methods for decreasing an adverse drug reaction in a subject in need thereof, the method comprising administering to the subject an effective amount of a compound having a structure represented by a formula:

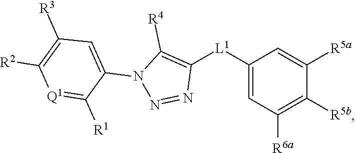

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{1a} and R^{5b} is independently selected from hydrogen and C1-C8 alkyl; wherein R^{6a} is R⁷; wherein R⁷ is a structure represented by a formula selected from:

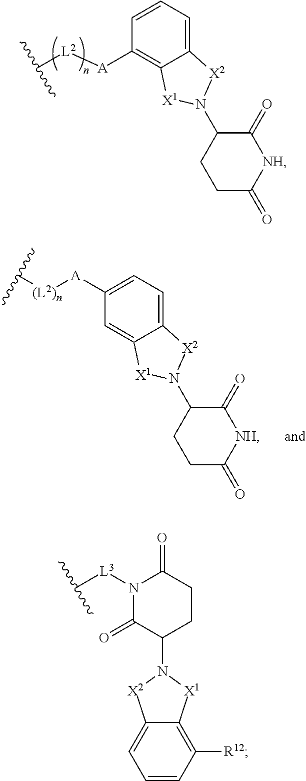

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

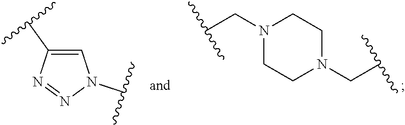

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof.

In various aspects, the compound has a structure represented by a formula:

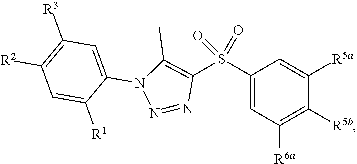

or a pharmaceutically acceptable salt thereof.

In various aspects, the compound has a structure represented by a formula:

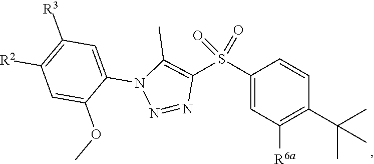

or a pharmaceutically acceptable salt thereof.

In various aspects, the compound has a structure represented by a formula:

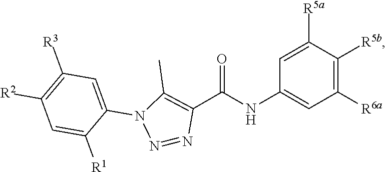

or a pharmaceutically acceptable salt thereof.

In various aspects, the compound has a structure represented by a formula:

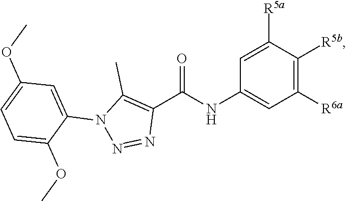

or a pharmaceutically acceptable salt thereof.

In various aspects, the compound has a structure represented by a formula:

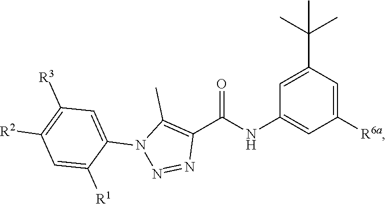

or a pharmaceutically acceptable salt thereof.

In various aspects, the compound has a structure represented by a formula:

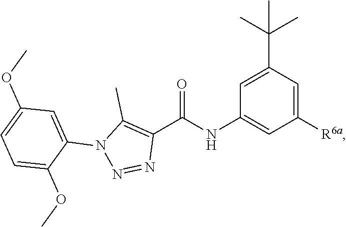

or a pharmaceutically acceptable salt thereof.

In various aspects, the compound is:

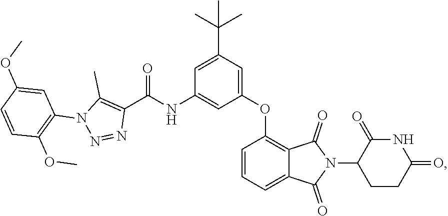

or a pharmaceutically acceptable salt thereof.

In various aspects, the compound is selected from:

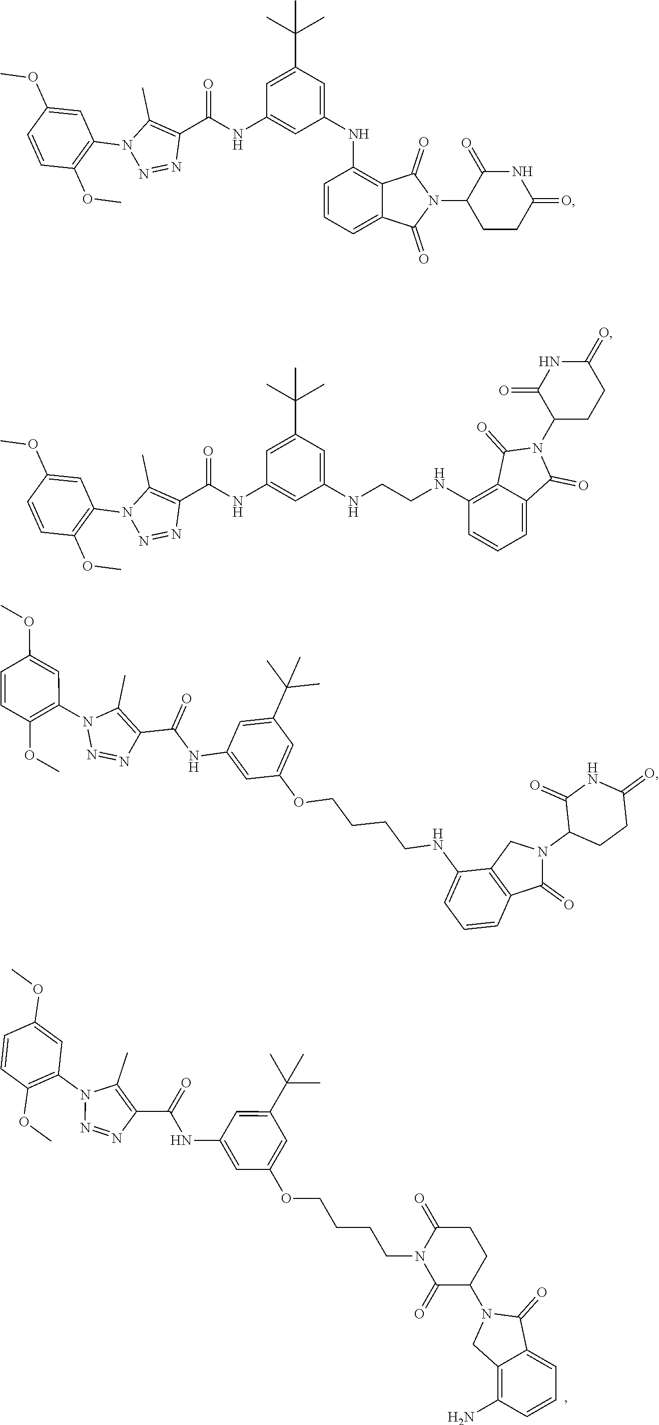 [SMILES: COc1ccc(OC)c(-n2nnc(C(=O)Nc3cc(Nc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(NCCNc4cccc5c4C(=O)N(C4CCC(=O)NC4=O)C5=O)cc(C(C)(C)C)c3)c2C)c1 ; COc1ccc(OC)c(-n2nnc(C(=O)Nc3cc(OCCCCNc4cccc5c4CN(C4CCC(=O)NC4=O)C5=O)cc(C(C)(C)C)c3)c2C)c1 ; COc1ccc(OC)c(-n2nnc(C(=O)Nc3cc(OCCCCN4C(=O)CCC(N5Cc6c(N)cccc6C5=O)C4=O)cc(C(C)(C)C)c3)c2C)c1]

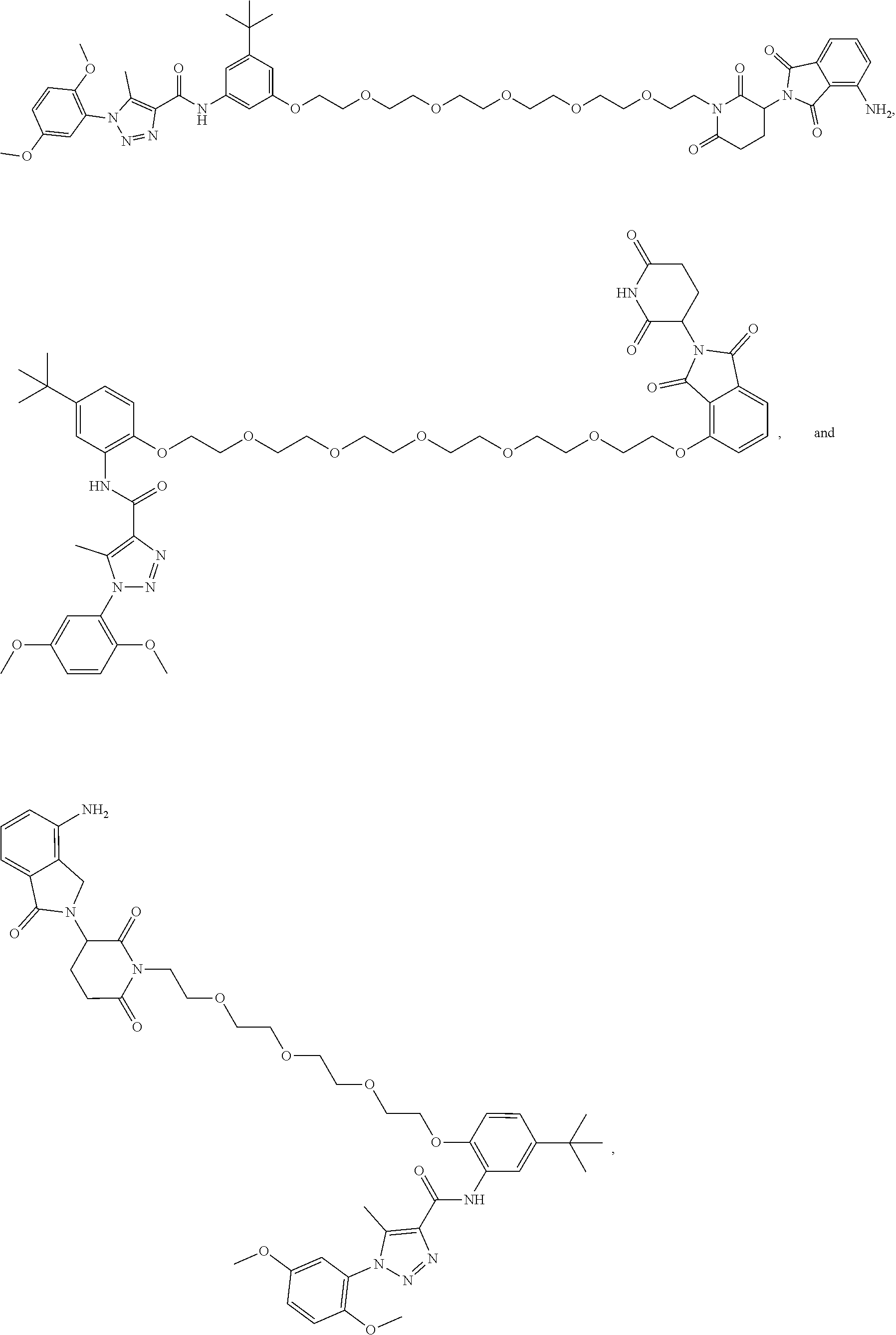 [SMILES: COc1ccc(OC)c(-n2nnc(C(=O)Nc3cc(OCCOCCOCCOCCOCCOCCN4C(=O)CCC(N5C(=O)c6cccc(N)c6C5=O)C4=O)cc(C(C)(C)C)c3)c2C)c1 ; COc1ccc(OC)c(-n2nnc(C(=O)Nc3cc(C(C)(C)C)ccc3OCCOCCOCCOCCOCCOCCOc3cccc4c3C(=O)N(C3CCC(=O)NC3=O)C4=O)c2C)c1 ; COc1ccc(OC)c(-n2nnc(C(=O)Nc3cc(C(C)(C)C)ccc3OCCOCCOCCOCCN3C(=O)CCC(N4Cc5c(N)cccc5C4=O)C3=O)c2C)c1]

or a pharmaceutically acceptable salt thereof.

In a further aspect, the subject is a mammal. In a still further aspect, the mammal is human.

In a further aspect, the subject has been diagnosed with a need for modulating an adverse drug reaction prior to the administering step. In a still further aspect, the subject is at risk for developing an adverse drug reaction prior to the administering step.

In a further aspect, the method further comprises identifying a subject in need of decreasing an adverse drug reaction.

In a further aspect, decreasing an adverse drug reaction is associated with the subject receiving treatment for a disorder of uncontrolled cellular proliferation.

In a further aspect, the method further comprises administering an anticancer agent. Examples of anticancer agents include, but are not limited to, paclitaxel, irinotecan, leucovorin, dasatinib, and erlotinib. In a still further aspect, the compound and the anticancer agent are administered simultaneously. In yet a further aspect, the compound and the anticancer agent are administered sequentially.

In a further aspect, the anticancer agent is a topoisomerase inhibitor.

Examples of topoisomerase inhibitors include, but are not limited to, camptothecin, topotecan, irinotecan, belotecan, gimatecan, inidimitecan, indotecan, Genz-644282, daunorubicin, epirubicin, etoposide, teniposide, mitoxantrone, ellipticinium, vasaroxin, dexrazoxane, mebarone, and 3-hydroxy-2-[(1R)-6-isopropenyl-3-methyl-cyclohex-2-en-1-yl]-5-pentyl-1,4-benzoquinone (HU-331). In a still further aspect, the topoisomerase inhibitor is irinotecan.

In a further aspect, the anticancer agent is a tyrosine kinase inhibitor.

Examples of tyrosine kinase inhibitors include, but are not limited to, axitinib, crizotinib, dasatinib, erlotinib, gefitinib, imatinib, lapatinib, nilotinib, pazopanib, regorafenib, ruxolitinib, sorafenib, sunitinib, vandetanib, and vemurafenib. In a still further aspect, the tyrosine kinase inhibitor is dasatinib or erlotinib.

In a further aspect, the anticancer agent is a mitotic inhibitor. Examples of mitotic inhibitors include, but are not limited to, paclitaxel, docetaxel, vinblastine, vincristine, and vinorelbine. In a still further aspect, the mitotic inhibitor is selected from paclitaxel and docetaxel. In yet a further aspect, the mitotic inhibitor is paclitaxel.

In a further aspect, the anticancer agent is a chemotherapeutic agent. In a still further aspect, the chemotherapeutic agent is selected from an alkylating or alkylating-like agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin, or a pharmaceutically acceptable salt thereof), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, or a pharmaceutically acceptable salt thereof), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin, or a pharmaceutically acceptable salt thereof), a mitotic inhibitor agent (e.g., etopside, vincristine, ixabepilone, vinorelbine, vinblastine, teniposide, or a pharmaceutically acceptable salt thereof), and an mTor inhibitor agent (e.g., everolimus, siroliumus, temsirolimus, or a pharmaceutically acceptable salt thereof).

In a further aspect, decreasing an adverse drug reaction is associated with the subject receiving treatment for a bacterial infection.

In a further aspect, the method further comprises administering an antibacterial agent. Examples of antibacterial agents include, but are not limited to, isoniazid, rifampicin, and flucloxacillin, or a combination thereof. In a still further aspect, the compound and the antibacterial agent are administered simultaneously. In yet a further aspect, the compound and the antibacterial agent are administered sequentially.

In a further aspect, decreasing an adverse drug reaction is associated with the subject receiving treatment for pain and/or inflammation.

In a further aspect, the method further comprises administering a non-steroidal anti-inflammatory agent. Examples of non-steroidal anti-inflammatory agents include, but are not limited to, acetaminophen. In a still further aspect, the compound and the non-steroidal anti-inflammatory agent are administered simultaneously. In yet a further aspect, the compound and the non-steroidal anti-inflammatory agent are administered sequentially.

In a further aspect, decreasing an adverse drug reaction is associated with the subject receiving treatment for seizures.

In a further aspect, the method further comprises administering an anticonvulsant agent. Examples of anticonvulsant agents include, but are not limited to, phenytoin. In a still further aspect, the compound and the anticonvulsant agent are administered simultaneously. In yet a further aspect, the compound and the anticonvulsant agent are administered sequentially.

### I. Methods of Treating a Bowel Disorder in a Subject

In various aspects, the compounds and compositions disclosed herein are useful for treating, preventing, ameliorating, controlling, or reducing the risk of a variety of bowel disorders, such as, for example, irritable bowel syndrome (IBS), Crohn's disease, celiac disease, and intestinal obstruction. Thus, in one aspect, disclosed are methods of treating a bowel disorder in a subject, the method comprising administering to the subject an effective amount of a disclosed compound or a pharmaceutically acceptable salt thereof.

In various aspects, disclosed are methods of treating a bowel disorder in a subject, the method comprising administering to the subject an effective amount of a compound having a structure represented by a formula:

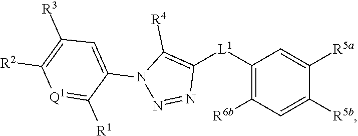

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein R^{6b} is R⁷; wherein R⁷ is a structure represented by a formula selected from:

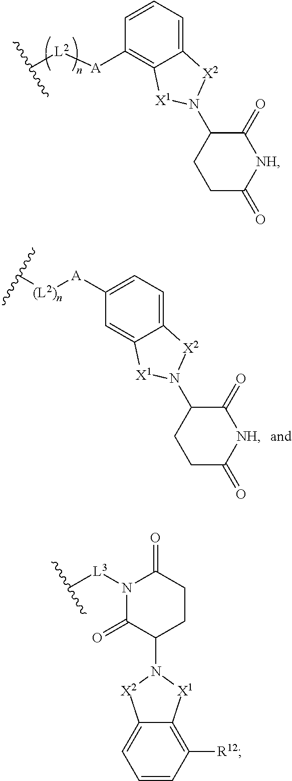

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

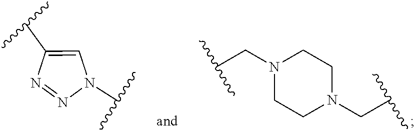

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof.

In various aspects, the compound has a structure represented by a formula:

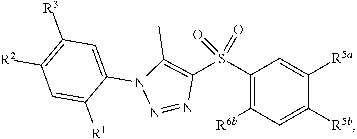

or a pharmaceutically acceptable salt thereof.

In various aspects, the compound has a structure represented by a formula:

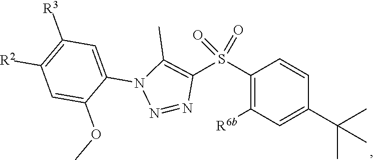

or a pharmaceutically acceptable salt thereof.

In various aspects, the compound has a structure represented by a formula:

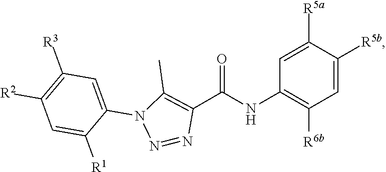

or a pharmaceutically acceptable salt thereof.

In various aspects, the compound has a structure represented by a formula:

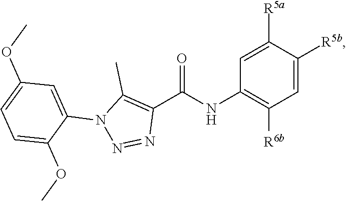

or a pharmaceutically acceptable salt thereof.

In various aspects, the compound has a structure represented by a formula:

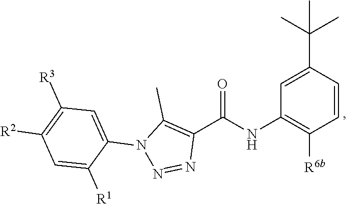

or a pharmaceutically acceptable salt thereof.

In various aspects, the compound has a structure represented by a formula:

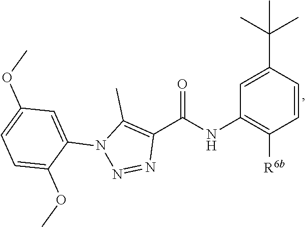

or a pharmaceutically acceptable salt thereof.

In a further aspect, the subject is a mammal. In a still further aspect, the mammal is human.

In a further aspect, the subject has been diagnosed with a need for treatment of the disorder prior to the administering step. In a still further aspect, the subject is at risk for developing the disorder prior to the administering step.

In a further aspect, the method further comprises identifying a subject at risk for developing the disorder prior to the administering step.

In a further aspect, the bowel disorder is selected from irritable bowel syndrome (IBS), Crohn's disease, celiac disease, and intestinal obstruction. In a still further aspect, the bowel disorder is irritable bowel syndrome (IBS).

### J. Additional Methods of Using the Compositions

Provided are methods of using of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders, and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein, which are usually applied in the treatment of the above-mentioned pathological conditions.

### 1. Manufacture of a Medicament

In one aspect, the invention relates to a method for the manufacture of a medicament for treating a disorder associated with dysfunctional PXR activity such as, for example, a disorder of uncontrolled cellular proliferation and a bowel disorder, in a mammal, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in modulating PXR activity as further disclosed herein. The dose administered to an animal, particularly a human, in the context of the present invention should be sufficient to affect a therapeutic response in the animal over a reasonable time-frame. One skilled in the art will recognize that dosage will depend upon a variety of factors including the condition of the animal, the body weight of the animal, as well as the severity and stage of the disorder.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 2. Use of Compounds and Compositions

Also provided are the uses of the disclosed compounds and compositions. Thus, in one aspect, the invention relates to the use of the disclosed compounds and compositions as modulators of PXR activity.

In a further aspect, the invention relates to the use of a disclosed compound or product of a disclosed method in the manufacture of a medicament for the treatment of a disorder associated with uncontrolled cellular proliferation, for example, cancer, including, but not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

In a further aspect, the invention relates to the use of the disclosed compounds and compositions in decreasing an adverse drug reaction.

In a further aspect, the invention relates to the use of the disclosed compounds and compositions in treating a bowel disorder such as, for example, irritable bowel syndrome.

In a further aspect, the use relates to a process for preparing a pharmaceutical composition comprising a therapeutically effective amount of a disclosed compound or a product of a disclosed method, and a pharmaceutically acceptable carrier, for use as a medicament.

In a further aspect, the use relates to a process for preparing a pharmaceutical composition comprising a therapeutically effective amount of a disclosed compound or a product of a disclosed method, wherein a pharmaceutically acceptable carrier is intimately mixed with a therapeutically effective amount of the disclosed compound or the product of a disclosed method.

In various aspects, the use relates to the treatment of uncontrolled cellular proliferation in a vertebrate animal. In a further aspect, the use relates to the treatment of uncontrolled cellular proliferation in a human subject.

In a further aspect, the use is the treatment of uncontrolled cellular proliferation, for example cancer, for example a cancer selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and, in another example, acute lymphoblastic leukemia.

In various aspects, the use relates to decreasing an adverse drug reaction in a vertebrate animal. In a further aspect, the use relates to decreasing an adverse drug reaction in a human subject.

In a further aspect, the use is decreasing an adverse drug reaction such as, for example an adverse drug reaction associated with administration of an anticancer agent, an antibacterial agent, a non-steroidal anti-inflammatory agent, or an anticonvulsant.

In various aspects, the use relates to the treatment of a bowel disorder in a vertebrate animal. In a further aspect, the use relates to the treatment of a bowel disorder in a human subject.

In a further aspect, the use is the treatment of a bowel disorder such as, for example, irritable bowel syndrome (IBS), Crohn's disease, celiac disease, and intestinal obstruction.

It is understood that the disclosed uses can be employed in connection with the disclosed compounds, methods, compositions, and kits. In a further aspect, the invention relates to the use of a disclosed compound or composition of a medicament for the treatment of a disorder or condition associated with dysfunctional PXR activity in a mammal.

In a further aspect, the invention relates to the use of a disclosed compound or composition in the manufacture of a medicament for the treatment of a disorder or condition associated with dysfunctional PXR activity such as, for example, cancer, a bowel disorder, and an adverse drug reaction.

### 3. Kits

In various aspects, the agents and pharmaceutical compositions described herein can be provided in a kit. The kit can also include combinations of the agents and pharmaceutical compositions described herein.

Thus, in one aspect, disclosed are kits comprising a disclosed compound and one or more selected from: (a) an agent known to increase pregnane X receptor (PXR) activity; (b) an agent known for the treatment of a disorder associated with pregnane X receptor (PXR) dysfunction; (c) administering the compound in connection with a disorder associated with pregnane X receptor (PXR) dysfunction; (d) instructions for decreasing an adverse drug reaction; and (e) instructions for treating a disorder associated with pregnane X receptor (PXR) dysfunction.

In a further aspect, the compound has a structure represented by a formula:

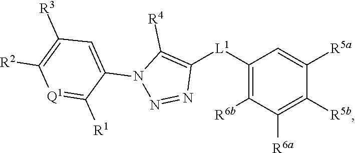

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷; wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl; wherein R⁴ is selected from hydrogen and C1-C4 alkyl; wherein each of R^{5a} and R^{5b} is independently selected from hydrogen and C1-C8 alkyl; wherein each of R^{6a} and R^{6b} is independently selected from hydrogen, C1-C8 alkyl, and R⁷; wherein R⁷ is a structure represented by a formula selected from:

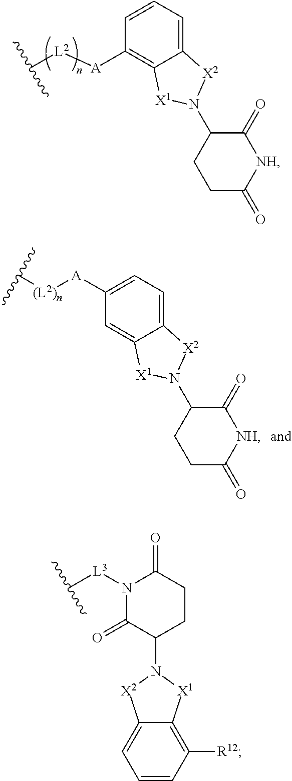

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

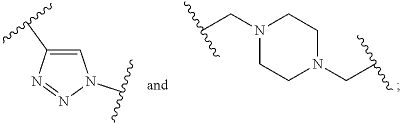

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, provided that one and only one of R², R^{6a}, and R^{6b} is R⁷, or a pharmaceutically acceptable salt thereof.

In a further aspect, the compound has a structure represented by a formula:

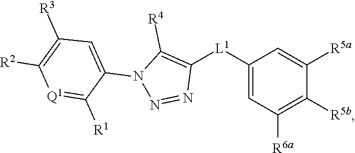

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)N¹¹—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{1a} and R^{5b} is independently selected from hydrogen and C1-C8 alkyl; wherein R^{6a} is R⁷; wherein R⁷ is a structure represented by a formula selected from:

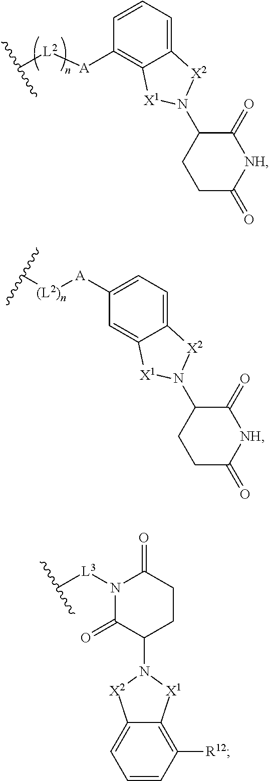

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

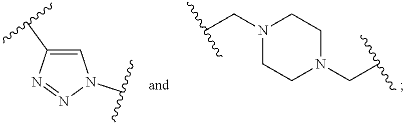

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof.

In a further aspect, the compound and the agent known to increase PXR activity are co-formulated. In a still further aspect, the compound and the agent known to increase PXR activity are co-packaged.

In a further aspect, the agent known to increase PXR activity is selected from an agent known to treat a disorder of uncontrolled cellular proliferation (e.g., paclitaxel, irinotecan, leucovorin, dasatinib, erlotinib), an agent known to treat an infectious disease (e.g., an antibacterial agent such as, for example, isoniazid, rifampicin, and flucloxacillin), a non-steroidal anti-inflammatory agent (e.g., acetaminophen), and an anticonvulsant agent (e.g., phenytoin).

In a further aspect, the kit further comprises a plurality of dosage forms, the plurality comprising one or more doses; wherein each dose comprises an effective amount of the compound and the agent known to increase PXR activity. In a still further aspect, the effective amount is a therapeutically effective amount. In yet a further aspect, the effective amount is a prophylactically effective amount. In an even further aspect, each dose of the compound and the agent known to increase PXR activity are co-packaged. In a still further aspect, each dose of the compound and the agent known to increase PXR activity are co-formulated.

In a further aspect, the compound and the agent known for the treatment of a disorder associated with pregnane X receptor (PXR) dysfunction are co-formulated. In a still further aspect, the compound and the agent known for the treatment of a disorder associated with pregnane X receptor (PXR) dysfunction are co-packaged.

In a further aspect, the agent known for the treatment of a disorder associated with pregnane X receptor (PXR) dysfunction is an agent known to treat a disorder of uncontrolled cellular proliferation.

In a further aspect, the kit further comprises a plurality of dosage forms, the plurality comprising one or more doses; wherein each dose comprises an effective amount of the compound and the agent known for the treatment of a disorder associated with pregnane X receptor (PXR) dysfunction. In a still further aspect, the effective amount is a therapeutically effective amount. In yet a further aspect, the effective amount is a prophylactically effective amount. In an even further aspect, each dose of the compound and the agent known for the treatment of a disorder associated with pregnane X receptor (PXR) dysfunction. In a still further aspect, each dose of the compound and the agent known for the treatment of a disorder associated with pregnane X receptor (PXR) dysfunction are co-formulated.

In one aspect, disclosed are kits comprising a compound having a structure represented by a formula:

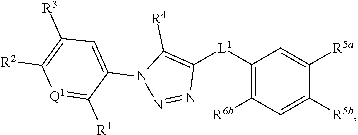

wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹⁰—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is selected from hydrogen, halogen, —OH, C1-C6 alkyl, C1-C6 alkoxy, C1-C6 hydroxyalkyl, and C1-C6 haloalkoxy; wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl); wherein R³ is selected from hydrogen, halogen, —OH, C1-C6 alkyl, C1-C6 alkoxy, and C1-C6 hydroxyalkyl; wherein R⁴ is selected from hydrogen and C1-C4 alkyl; wherein each of R^{1a} and R^{5b} is independently selected from hydrogen and C1-C8 alkyl; wherein R^{6b} is a structure represented by a formula selected from:

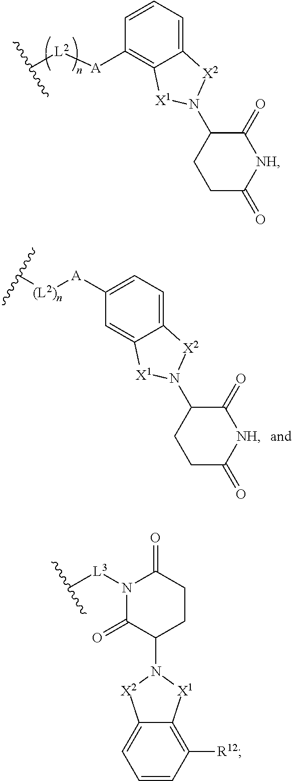

wherein n is selected from 0 and 1; wherein A is selected from —O—, —NH—, —CH₂—, and —C≡C—; wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-; wherein q, when present, is selected from 1, 2, 3, 4, 5, 6, 7, and 8; wherein Ar¹ is a structure selected from:

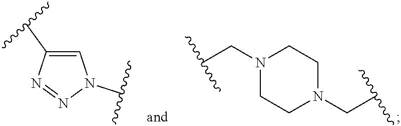

wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-; wherein r, when present, is selected from 1, 2, 3, 4, 5, 6, 7, and 8; wherein each of X¹ and X² is independently selected from —CH₂— and —C(O)—, provided that at least one of X¹ and X² is —C(O)—; and wherein R¹² is selected from hydrogen, —OH, and —NH₂, or a pharmaceutically acceptable salt thereof, and one or more selected from: (a) an agent known for the treatment of a disorder associated with pregnane X receptor (PXR) dysfunction; (b) instructions for administering the compound in connection with a disorder associated with pregnane X receptor (PXR) dysfunction; and (c) instructions for treating a disorder associated with pregnane X receptor (PXR) dysfunction.

In various aspects, the compound and the agent are co-formulated. In a further aspect, the compound and the agent are co-packaged.

In a further aspect, the disorder is a bowel disorder.

In a further aspect, the agent is selected from an anti-inflammatory agent, an immune system suppressor, a biologic, and antibiotic. In a still further aspect, the anti-inflammatory agent is selected from a corticosteroid and an aminosalicylate. In yet a further aspect, the anti-inflammatory agent is selected from mesalamine, balsalazie, and olsalazine. In an even further aspect, the immune system suppressor is selected from azathioprine, mercaptopurine, and methotrexate. In a still further aspect, the biologic is selected from infliximab, adalimumab, golimumab, certolizumab, vedolizumab, and ustekinumab. In yet a further aspect, the antibiotic is selected from ciprofloxacin and metronidazole.

In various aspects, the informational material can be descriptive, instructional, marketing or other material that relates to the methods described herein and/or to the use of the agents for the methods described herein. For example, the informational material may relate to the use of the agents herein to treat a subject who has, or who is at risk for developing, a disorder or condition associated with dysfunctional PXR activity such as, for example, cancer, a bowel disorder, and an adverse drug reaction. The kits can also include paraphernalia for administering the agents of this invention to a cell (in culture or in vivo) and/or for administering a cell to a patient.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, a disorder or condition associated with dysfunctional PXR activity such as, for example, cancer, a bowel disorder, and an adverse drug reaction.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

### 4. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a disorder or condition associated with dysfunctional PXR activity such as, for example, cancer, a bowel disorder, or an adverse drug reaction, prior to the administering step. In some aspects of the disclosed methods, the subject has been identified with a need for treatment prior to the administering step. In one aspect, a subject can be treated prophylactically with a disclosed compound or composition, as discussed elsewhere herein.

### a. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

### b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### K. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### 1. Chemistry Experimentals

### a. Preparation of PROTACs

### i. 1-(2,5-dimethoxyphenyl)-N-(2-(2,6-dioxopiperidin-3-yl)-1-oxoisoindolin-4-yl)-5-methyl-1H-1,2,3-triazole-4-carboxamide (M1)

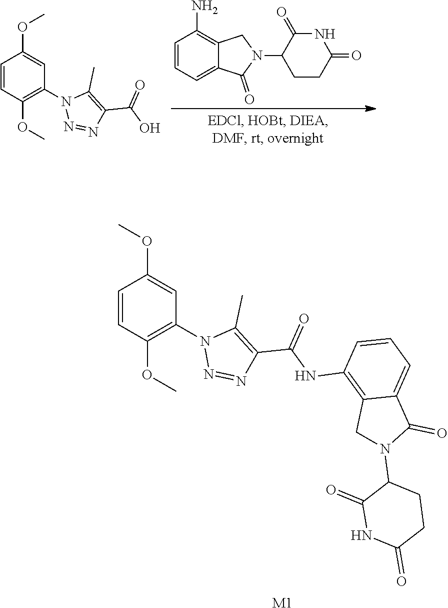

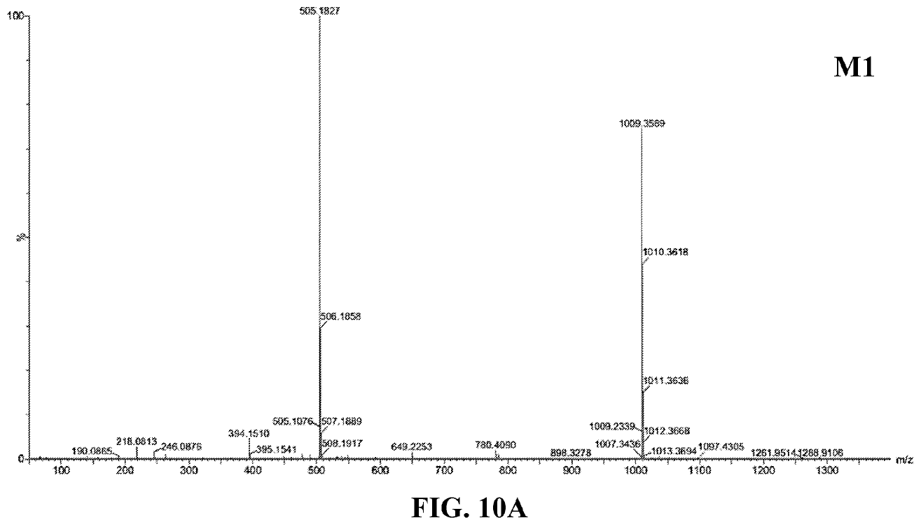

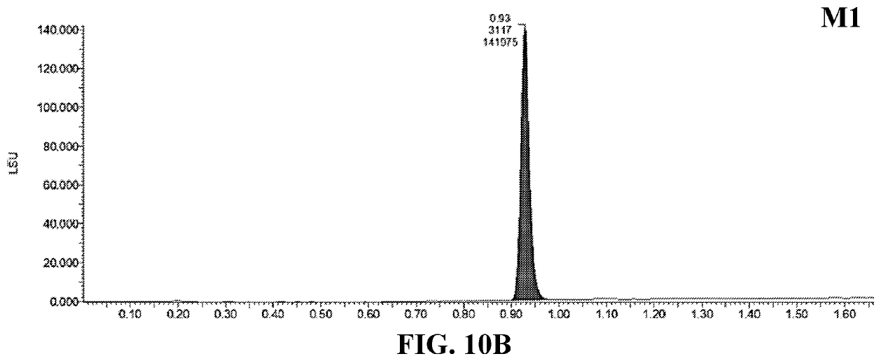

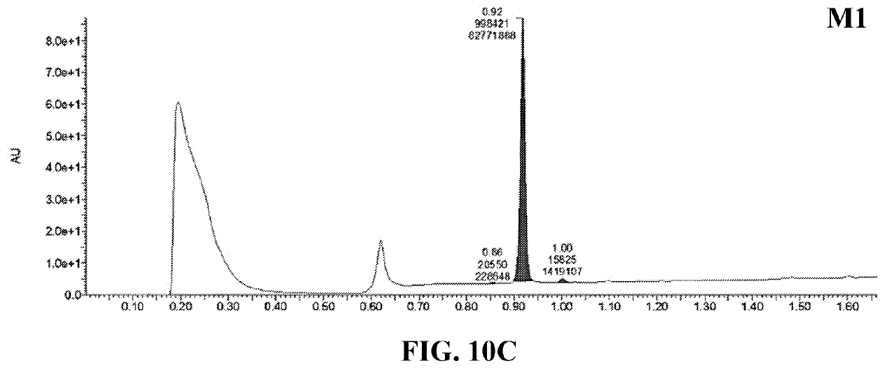

To a solution of 1-(2,5-dimethoxyphenyl)-5-methyl-1H-1,2,3-triazole-4-carboxylic acid (0.263 g, 1 mmol) in DMF 5 mL at rt was added 3-(4-amino-1-oxoisoindolin-2-yl)piperidine-2,6-dione (0.259 g, 1.000 mmol), HOBt, 80% (0.203 g, 1.200 mmol) and EDCI (0.288 g, 1.500 mmol), followed by N-ethyl-N-isopropylpropan-2-amine (0.331 ml, 2.000 mmol). The suspension was stirred at room temperature for overnight. The reaction mixture was then diluted with water (20 mL) and extracted with EtOAc (25 mL×2). The combined EtOAc layers were washed with water, dried with anhydrous Na₂SO₄ and concentrated. The residue was purified by silica gel chromatography (0% to 100% EA in hexane) to give product as a white solid (315.8 mg, 63% yield). ¹H NMR (500 MHz, DMSO-d₆) δ 11.00 (s, 1H), 10.63 (s, 1H), 7.77 (d, J=7.8 Hz, 1H), 7.62 (d, J=7.4 Hz, 1H), 7.56 (t, J=7.6 Hz, 1H), 7.29 (d, J=9.2 Hz, 1H), 7.23 (dd, J=9.1, 3.1 Hz, 1H), 7.13 (d, J=3.0 Hz, 1H), 5.15 (dd, J=13.3, 5.2 Hz, 1H), 4.49 (q, J=17.4 Hz, 2H), 3.78 (s, 3H), 3.76 (s, 3H), 2.91 (ddd, J=18.0, 13.6, 5.4 Hz, 1H), 2.65-2.55 (m, 1H), 2.43 (dd, J=13.2, 4.4 Hz, 1H), 2.38 (s, 3H), 2.10-1.93 (m, 1H). ¹³C NMR (126 MHz, DMSO-d₆) δ 172.28, 170.40, 167.18, 158.73, 152.46, 147.10, 138.63, 136.49, 135.42, 132.40, 132.18, 127.96, 127.02, 123.05, 119.49, 116.75, 113.46, 113.28, 55.72, 55.26, 50.93, 46.07, 30.61, 21.87, 8.24. ESI-TOF HRMS: m/z 505.1827 (C25H₂₄N₆O₆+H⁺ requires 505.1830). Representative corresponding mass spectra and HPLC traces are shown in FIG. 10A-C.

### ii. 2-(2,6-dioxopiperidin-3-yl)-1,3-dioxoisoindolin-4-yl 1-(2,5-dimethoxyphenyl)-5-methyl-1H-1,2,3-triazole-4-carboxylate (M2)

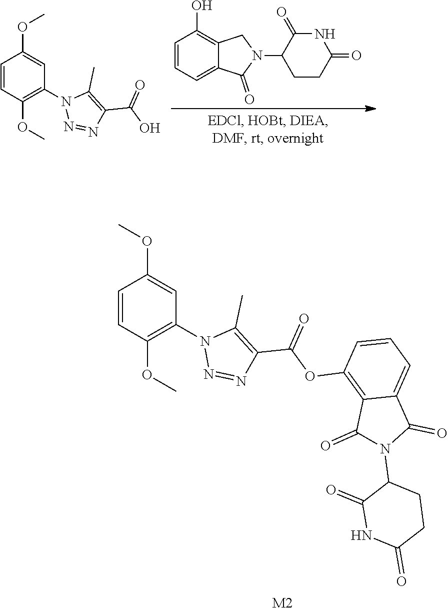

To a solution of 1-(2,5-dimethoxyphenyl)-5-methyl-1H-1,2,3-triazole-4-carboxylic acid (0.263 g, 1 mmol) in DMF 5 mL was added 2-(2,6-dioxopiperidin-3-yl)-4-hydroxyisoindoline-1,3-dione (0.274 g, 1.000 mmol), HOBt, 80% (0.203 g, 1.200 mmol) and EDCI (0.288 g, 1.500 mmol), followed by N-ethyl-N-isopropylpropan-2-amine (0.331 ml, 2.000 mmol). The suspension was stirred at room temperature for overnight. The reaction mixture was then diluted with water (20 mL) and extracted with EtOAc (25 mL×2). The combined EtOAc layers were washed with water, dried with anhydrous Na₂SO₄ and concentrated. The residue was purified by silica gel chromatography (0% to 100% EA in hexane) to give product as a white solid (316.2 mg, 61% yield). ¹H NMR (400 MHz, Chloroform-d) δ 8.20 (s, 1H), 7.88-7.79 (m, 2H), 7.61 (dd, J=6.9, 2.1 Hz, 1H), 7.09 (dd, J=9.1, 3.0 Hz, 1H), 7.03 (d, J=9.1 Hz, 1H), 6.97 (d, J=3.0 Hz, 1H), 5.04-4.79 (m, 1H), 3.81 (s, 3H), 3.78 (s, 3H), 2.89-2.63 (m, 3H), 2.50 (s, 3H), 2.18-2.08 (m, 1H). ¹³C NMR (101 MHz, CDCl₃) δ 170.90, 167.74, 166.37, 164.70, 158.93, 153.72, 147.93, 146.48, 142.73, 136.28, 134.39, 133.28, 129.30, 124.04, 122.84, 121.68, 117.81, 113.72, 113.42, 56.40, 56.03, 49.35, 31.31, 22.51, 9.66. ESI-TOF HRMS: m/z 520.1464 (C₂₅H₂₁N₅O₈+H⁺ requires 520.1463).

### iii. 1-(2,5-dimethoxyphenyl)-N-(2-(2,6-dioxopiperidin-3-yl)-1,3-dioxoisoindolin-4-yl)-5-methyl-1H-1,2,3-triazole-4-carboxamide (M3)

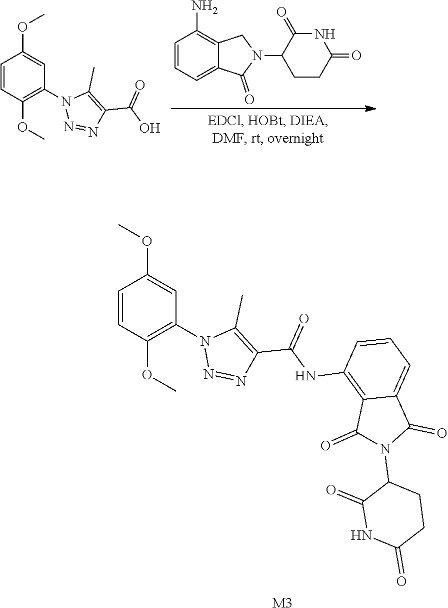

To a solution of 4-amino-2-(2,6-dioxopiperidin-3-yl)isoindoline-1,3-dione (125 mg, 0.456 mmol) in DMF (5 mL) was added 1-(2,5-dimethoxyphenyl)-5-methyl-1H-1,2,3-triazole-4-carboxylic acid (100 mg, 0.380 mmol) at 0° C. Propylphosphonic anhydride (1450 mg, 2.279 mmol) and pyridine (1 mL) were added to the reaction mixture. Then the mixture was stirred at 80° C. for 3 hours. The reaction mixture was diluted with water (50 mL) and extracted with EtOAc (50 mL×2). The EtOAc layer was washed with water, dried with anhydrous Na₂SO₄ and concentrated. The residue was purified by silica gel chromatography (0% to 100% EA in hexane) to give product as white solid, 137.8 mg, 70% yield. ¹H NMR (500 MHz, DMSO-d₆) δ 11.18 (s, 1H), 11.06 (s, 1H), 8.84 (d, J=8.4 Hz, 1H), 7.92 (t, J=7.9 Hz, 1H), 7.67 (d, J=7.3 Hz, 1H), 7.30 (d, J=9.2 Hz, 1H), 7.24 (dd, J=9.2, 3.1 Hz, 1H), 7.18 (d, J=3.0 Hz, 1H), 5.20 (dd, J=13.0, 5.4 Hz, 1H), 3.78 (s, 3H), 3.76 (s, 3H), 2.91 (ddd, J=17.5, 14.0, 5.5 Hz, 1H), 2.68-2.56 (m, 2H), 2.43 (s, 3H), 2.16-2.04 (m, 1H). ¹³C NMR (126 MHz, DMSO-d₆) δ 172.15, 169.17, 167.84, 166.09, 158.87, 152.48, 147.04, 139.43, 135.97, 135.87, 135.84, 130.80, 123.91, 122.81, 117.70, 117.00, 115.59, 113.39, 113.36, 55.77, 55.28, 48.40, 30.35, 21.37, 8.24. ESI-TOF HRMS: m/z 519.1619 (C₂₅H₂₂N₆O₇+H⁺ requires 519.1623).

### iv. N-(3-(tert-butyl)-5-((2-(2,6-dioxopiperidin-3-yl)-1,3-dioxoisoindolin-4-yl)oxy)phenyl)-1-(2,5-dimethoxyphenyl)-5-methyl-1H-1,2,3-triazole-4-carboxamide (M4)

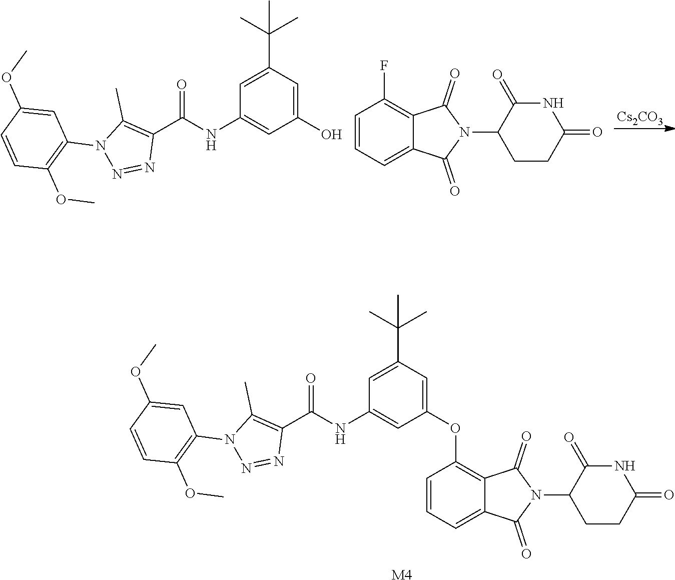

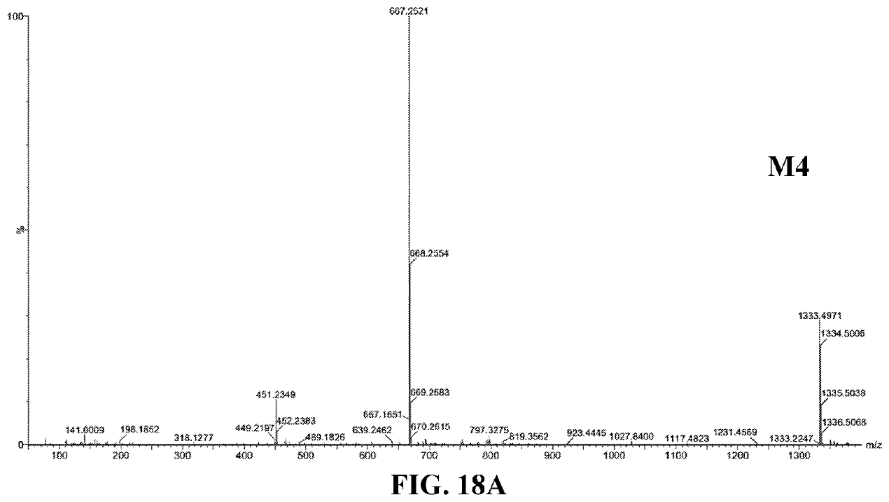

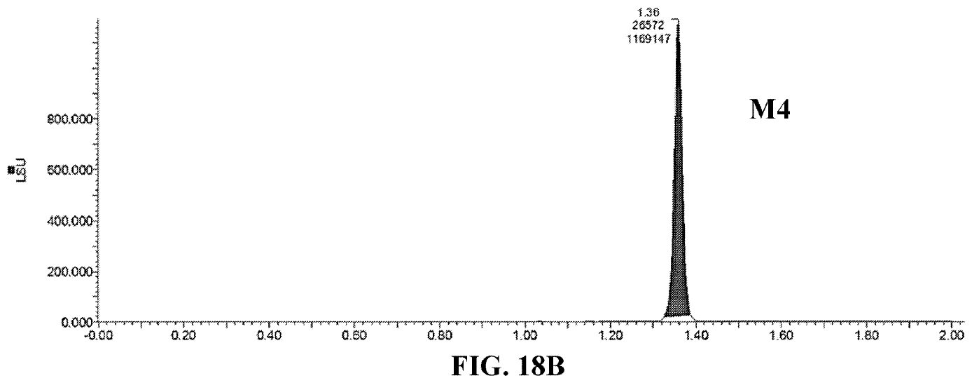

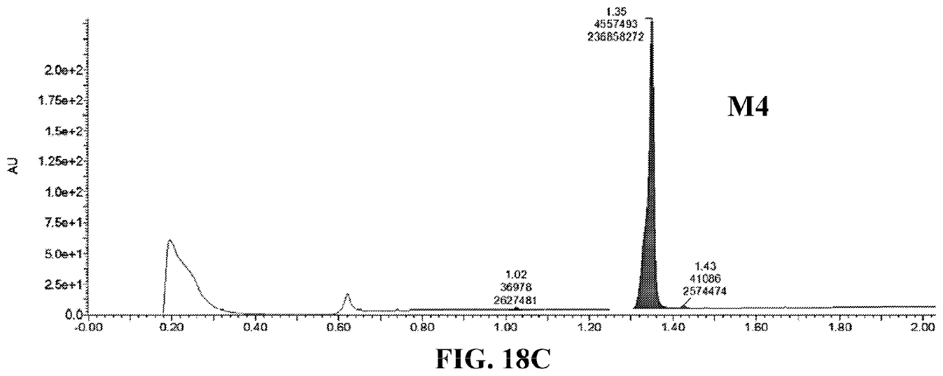

To a solution of N-(3-(tert-butyl)-5-hydroxyphenyl)-1-(2,5-dimethoxyphenyl)-5-methyl-1H-1,2,3-triazole-4-carboxamide (50 mg, 0.122 mmol) in DMF 5 mL was added Cs₂CO₃ (79 mg, 0.244 mmol) and 2-(2,6-dioxopiperidin-3-yl)-4-fluoroisoindoline-1,3-dione (41.0 mg, 0.149 mmol). The suspension was stirred at 80° C. for overnight. The reaction mixture was then diluted with water (50 mL) and extracted with EtOAc (50 mL×2). The combined EtOAc layers were washed with water, dried with anhydrous Na₂SO₄ and concentrated. The residue was purified by silica gel chromatography (0% to 100% EA in hexane) to give product (66.5 mg, 82% yield). ¹H NMR (500 MHz, DMSO-d₆) δ 11.14 (s, 1H), 10.62 (s, 1H), 7.87 (t, J=1.8 Hz, 1H), 7.82 (dd, J=8.5, 7.3 Hz, 1H), 7.66 (t, J=2.1 Hz, 1H), 7.64 (d, J=7.2 Hz, 1H), 7.28 (d, J=9.2 Hz, 1H), 7.22 (td, J=5.9, 2.8 Hz, 2H), 7.14 (d, J=3.0 Hz, 1H), 6.97 (t, J=2.0 Hz, 1H), 5.16 (dd, J=12.8, 5.4 Hz, 1H), 3.77 (s, 3H), 3.74 (s, 3H), 2.91 (ddd, J=16.7, 13.7, 5.4 Hz, 1H), 2.68-2.52 (m, 2H), 2.36 (s, 3H), 2.12-2.03 (m, 1H), 1.30 (s, 9H). ¹³C NMR (126 MHz, DMSO-d₆) δ 172.19, 169.27, 166.01, 164.36, 159.07, 153.77, 153.44, 153.10, 152.47, 147.07, 139.47, 138.63, 136.80, 136.58, 132.91, 123.04, 122.72, 117.67, 117.09, 116.84, 113.49, 113.38, 113.27, 111.55, 107.91, 55.72, 55.27, 48.30, 34.25, 30.35, 21.37, 8.23. ESI-TOF HRMS: m/z 667.2521 (C₃₅H₃₄N₆O₈+H⁺ requires 667.2511). Representative corresponding mass spectra and HPLC traces are shown in FIG. 18A-C.

### v. N-(3-(tert-butyl)-5-((2-(2,6-dioxopiperidin-3-yl)-1,3-dioxoisoindolin-4-yl)amino)phenyl)-1-(2,5-dimethoxyphenyl)-5-methyl-1H-1,2,3-triazole-4-carboxamide (M5)

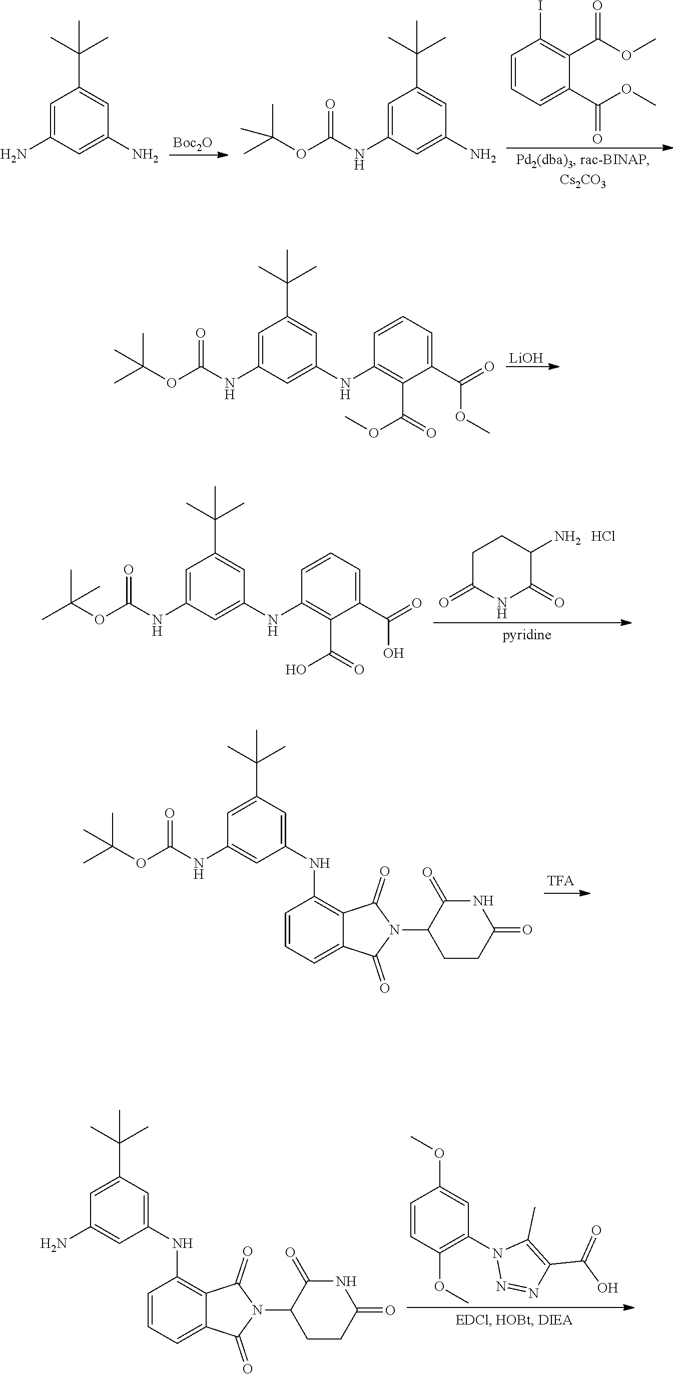

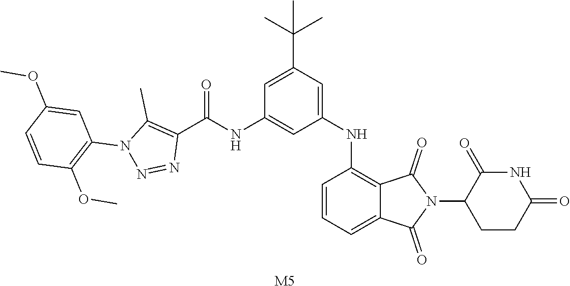 [SMILES: COc1ccc(OC)c(-n2nnc(C(=O)Nc3cc(Nc4cccc5c4C(=O)N(C4CCC(=O)NC4=O)C5=O)cc(C(C)(C)C)c3)c2C)c1 ; "M5"]

To a solution of 5-(tert-butyl)benzene-1,3-diamine (5 g, 30.4 mmol) in DCM (30 mL) on ice was added a solution of di-tert-butyl dicarbonate (11.07 g, 15.22 mmol) 30% in dioxane dropwise. The mixture was stirred at room temperature for overnight. Then the solvent was evaporated, and the residue was purified by column chromatography using silica gel as stationary phase (elution system—EA/Hexane) to give the desired product as a solid (3.67 g, 91%). ¹H NMR (500 MHz, DMSO-d₆) δ 8.04 (s, 1H), 5.77 (t, J=1.6 Hz, 2H), 5.38 (t, J=1.9 Hz, 1H), 4.02 (s, 2H), 0.61 (s, 9H), 0.33 (s, 9H). ¹³C NMR (126 MHz, DMSO) δ 153.21, 151.78, 148.87, 140.08, 106.04, 104.42, 102.15, 78.84, 34.61, 31.64, 28.66.

A mixture of dimethyl 3-iodophthalate (2.35 g, 7.34 mmol), tert-butyl (3-amino-5-(tert-butyl)phenyl)carbamate (2.135 g, 8.08 mmol), Pd₂(dba)₃ (0.336 g, 0.367 mmol), rac-BINAP (0.229 g, 0.367 mmol), and cesium carbonate (3.59 g, 11.01 mmol), in 50 mL toluene was heated to reflux under nitrogen for 24 hours. The reaction mixture was cooled, diluted with CH₂Cl₂ (100 mL), and then filtered through Celite, and the filter was washed with additional CH₂Cl₂ (200 mL). The filtrate was evaporated, and the residue was chromatographed using a hexanes-ethyl acetate gradient, eluting 2.42 g of the product in 72% yield, gray-white solid. ¹H NMR (500 MHz, Chloroform-d) δ 7.96 (s, 1H), 7.45-7.35 (m, 1H), 7.34-7.27 (m, 1H), 7.13 (s, 1H), 7.08 (dd, J=7.4, 1.3 Hz, 1H), 6.98 (q, J=1.9 Hz, 1H), 6.84 (q, J=1.9 Hz, 1H), 6.45 (s, 1H), 3.88 (s, 3H), 3.86 (s, 3H), 1.50 (s, 9H), 1.28 (s, 9H). ¹³C NMR (126 MHz, CDCl₃) δ 168.89, 168.50, 153.53, 152.66, 144.99, 141.22, 139.13, 134.01, 131.71, 119.23, 118.65, 115.91, 113.33, 110.87, 108.60, 80.50, 52.55, 52.50, 34.86, 31.22, 28.36.

Lithium hydroxide (0.629 g, 26.3 mmol) was added to a suspension solution of dimethyl 3-((3-((tert-butoxycarbonyl)amino)-5-(tert-butyl)phenyl)amino)phthalate (2.4 g, 5.26 mmol) in acetone/H₂O=20/10 mL, then the mixture was stirred at 60° C. for overnight. the reaction mixture was added EA 200 mL, adjust ph-4 by 0.5 N HCl and washed by water. the organic phase was concentrated under the reduced pressure. The residue was used for next step without purified. Yellow solid, 1.99 g, 88%. ¹H NMR (500 MHz, DMSO-d₆) δ 9.17 (s, 1H), 7.96 (s, 1H), 7.42-7.28 (m, 2H), 7.15 (dd, J=6.6, 1.9 Hz, 1H), 7.13-7.09 (m, 2H), 6.75 (t, J=1.8 Hz, 1H), 1.45 (d, J=1.9 Hz, 9H), 1.22 (s, 9H). 13C NMR (126 MHz, DMSO-d₆) δ 168.45, 168.13, 152.13, 151.60, 142.10, 141.45, 139.42, 133.34, 130.03, 120.47, 119.43, 119.28, 110.06, 108.54, 105.32, 78.23, 33.85, 30.44, 27.53.

To the residue was added pyridine (20 mL), 3-((3-((tert-butoxycarbonyl)amino)-5-(tert-butyl)phenyl)amino)phthalic acid (1.99 g, 4.64 mmol), and 3-aminopiperidine-2,6-dione hydrochloride (0.841 g, 5.11 mmol). the resulting mixture was stirred at 118° C. for overnight. Then the reaction mixture was concentrated under the reduced pressure, the residue was added EA 100 mL washed by water. the organic phase was dried over anhydrous MgSO₄ and concentrated under the reduced pressure. The residue was purified by column chromatography to provide the title compound as a yellow solid (1.22 g, 50%). ¹H NMR (500 MHz, DMSO-d₆) δ 11.13 (s, 1H), 9.31 (s, 1H), 8.36 (s, 1H), 7.62 (dd, J=8.6, 7.1 Hz, 1H), 7.43 (d, J=8.6 Hz, 1H), 7.34-7.28 (m, 2H), 7.23 (d, J=7.1 Hz, 1H), 6.94 (t, J=1.9 Hz, 1H), 5.12 (dd, J=12.8, 5.4 Hz, 1H), 2.91 (ddd, J=17.0, 13.8, 5.3 Hz, 1H), 2.65-2.52 (m, 2H), 2.06 (ddq, J=13.2, 5.8, 3.4, 2.6 Hz, 1H), 1.46 (s, 9H), 1.25 (s, 9H). ¹³C NMR (126 MHz, DMSO-d₆) δ 172.22, 169.41, 167.71, 166.46, 152.16, 151.93, 142.36, 139.53, 138.63, 135.56, 131.85, 118.88, 112.63, 112.43, 111.21, 110.56, 107.97, 78.44, 48.09, 33.95, 30.40, 27.53, 24.18, 21.52.

A stirred mixture containing tert-butyl (3-(tert-butyl)-5-((2-(2,6-dioxopiperidin-3-yl)-1,3-dioxoisoindolin-4-yl)amino)phenyl)carbamate (1.22 g, 2.344 mmol) in 20 ml dry DCM, 10 mL of TFA was added and stirred at room temperature for 20 hours. Then the mixture was concentrated, and the residue was diluted with ethyl acetate (100 mL) and water (100 mL). The organic layer was washed with water (2×100 mL) and brine (1×50 mL). The organic layer was collected, dried over anhydrous MgSO₄ and concentrated under reduced pressure. The residue was purified by column chromatography to provide the title compound as a yellow solid, 823.9 mg, 84%. ¹H NMR (500 MHz, DMSO-d₆) δ 11.13 (s, 1H), 8.14 (s, 1H), 7.61 (dd, J=8.6, 7.1 Hz, 1H), 7.42 (d, J=8.6 Hz, 1H), 7.19 (d, J=7.0 Hz, 1H), 6.46 (t, J=1.8 Hz, 1H), 6.42 (t, J=1.8 Hz, 1H), 6.37 (t, J=2.0 Hz, 1H), 5.15-5.08 (m, 3H), 2.90 (ddd, J=16.9, 13.7, 5.3 Hz, 1H), 2.65-2.53 (m, 2H), 2.10-2.03 (m, 1H), 1.22 (s, 9H). ¹³C NMR (126 MHz, DMSO-d₆) δ 172.21, 169.43, 167.96, 166.49, 152.06, 148.78, 142.82, 138.65, 135.49, 131.74, 118.76, 112.12, 110.54, 106.93, 106.47, 103.82, 48.06, 33.65, 30.48, 30.37, 21.51.

To a solution of 1-(2,5-dimethoxyphenyl)-5-methyl-1H-1,2,3-triazole-4-carboxylic acid (62.6 mg, 0.238 mmol) in DMSO 3 mL was added 4-((3-amino-5-(tert-butyl)phenyl)amino)-2-(2,6-dioxopiperidin-3-yl)isoindoline-1,3-dione (100 mg, 0.238 mmol), HOBt, 80% (48.2 mg, 0.285 mmol) and EDCI (68.4 mg, 0.357 mmol), followed by N-ethyl-N-isopropylpropan-2-amine (61.5 mg, 0.476 mmol) was added. The reaction was stirred at room temperature for overnight. The reaction was diluted with ethyl acetate (50 mL) and water (50 mL). The organic layer was washed with water (2×50 mL) and brine (1×50 mL). The organic layer was collected, dried over anhydrous MgSO₄ and concentrated under reduced pressure. The residue was purified by column chromatography to provide the title compound as a yellow solid (118.9 mg, 75%). ¹H NMR (500 MHz, DMSO-d₆) δ 11.14 (s, 1H), 10.51 (s, 1H), 8.45 (s, 1H), 7.81 (t, J=1.9 Hz, 1H), 7.73 (t, J=1.7 Hz, 1H), 7.66 (dd, J=8.6, 7.1 Hz, 1H), 7.55 (d, J=8.5 Hz, 1H), 7.29 (d, J=9.2 Hz, 1H), 7.26 (d, J=7.0 Hz, 1H), 7.22 (dd, J=9.2, 3.1 Hz, 1H), 7.15 (d, J=3.0 Hz, 1H), 7.09 (t, J=1.9 Hz, 1H), 5.13 (dd, J=12.9, 5.4 Hz, 1H), 3.78 (s, 3H), 3.75 (s, 3H), 2.91 (ddd, J=17.5, 14.0, 5.4 Hz, 1H), 2.66-2.55 (m, 2H), 2.38 (s, 3H), 2.12-2.03 (m, 1H), 1.31 (s, 9H). ¹³C NMR (126 MHz, DMSO-d₆) δ 172.23, 169.42, 167.74, 166.47, 158.96, 152.47, 151.91, 147.09, 142.23, 138.57, 138.55, 138.47, 136.94, 135.61, 131.85, 123.09, 118.97, 116.81, 113.86, 113.40, 113.28, 112.79, 112.74, 111.32, 109.78, 55.72, 55.28, 48.10, 34.07, 30.43, 30.37, 21.52, 8.24. ESI-TOF HRMS: m/z 666.2681 (C₃₅H₃₅N₇O₇+H⁺ requires 666.2671).

### vi. N-(3-(tert-butyl)-5-(3-(2-(2,6-dioxopiperidin-3-yl)-1,3-dioxoisoindolin-4-yl)ureido)phenyl)-1-(2,5-dimethoxyphenyl)-5-methyl-1H-1,2,3-triazole-4-carboxamide (M6)

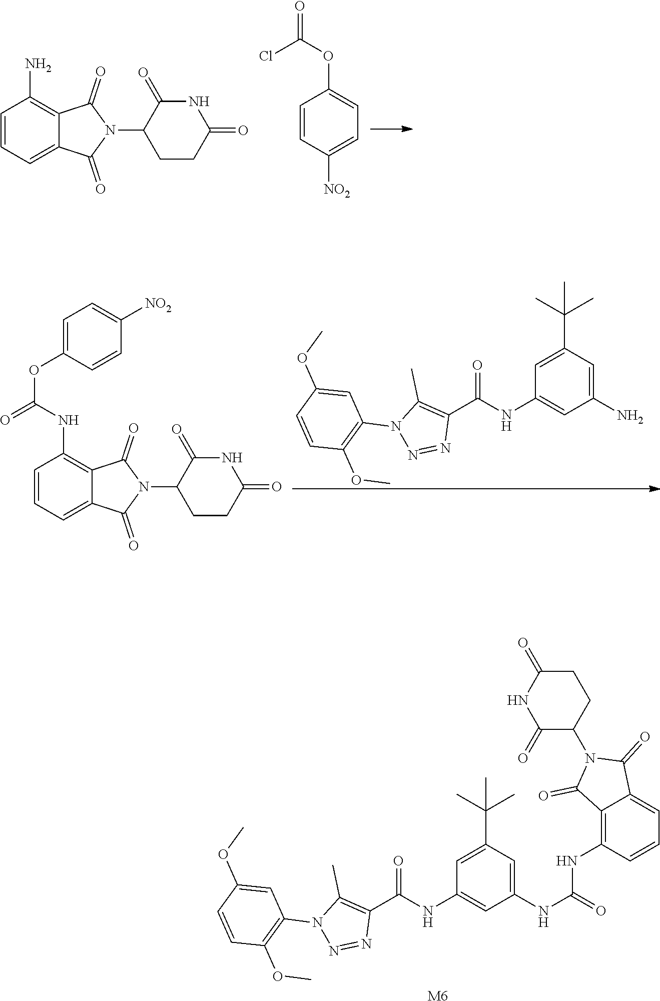 [SMILES: Nc1cccc2c1C(=O)N(C1CCC(=O)NC1=O)C2=O ; O=C(Cl)Oc1ccc([N+](=O)[O-])cc1 ; O=C1CCC(N2C(=O)c3cccc(NC(=O)Oc4ccc([N+](=O)[O-])cc4)c3C2=O)C(=O)N1 ; COc1ccc(OC)c(-n2nnc(C(=O)Nc3cc(N)cc(C(C)(C)C)c3)c2C)c1 ; COc1ccc(OC)c(-n2nnc(C(=O)Nc3cc(NC(=O)Nc4cccc5c4C(=O)N(C4CCC(=O)NC4=O)C5=O)cc(C(C)(C)C)c3)c2C)c1 ; "M6"]

To a solution of 4-amino-2-(2,6-dioxopiperidin-3-yl)isoindoline-1,3-dione (624 mg, 2.284 mmol) in dry THF (60 ml) was added 4-nitrophenyl carbonochloridate (690 mg, 3.43 mmol). The mixture was stirred at reflux for 4 hours. The mixture was concentrated to about 15 mL, and filtered to give 4-nitrophenyl (2-(2,6-dioxopiperidin-3-yl)-1,3-dioxoisoindolin-4-yl)carbamate as a solid (443 mg, 44% yield).

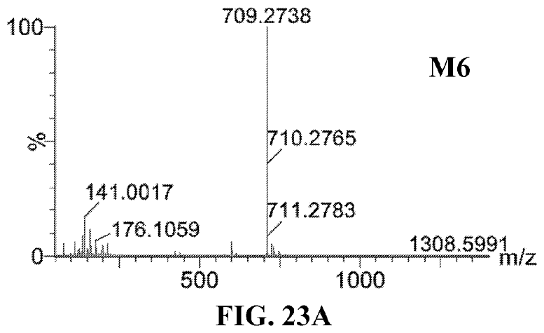

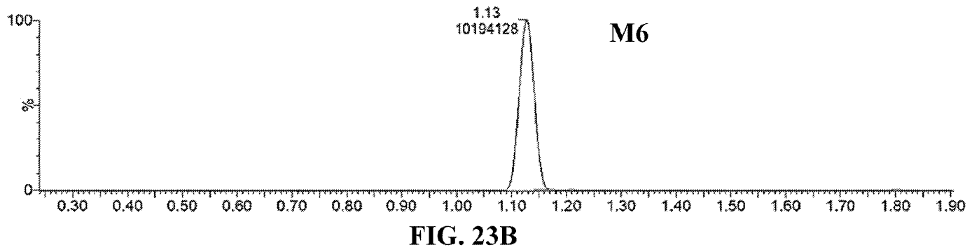

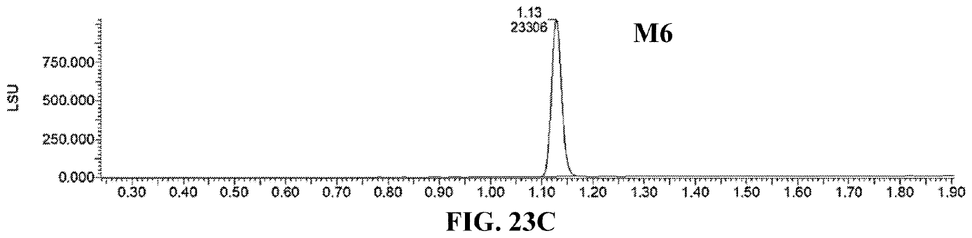

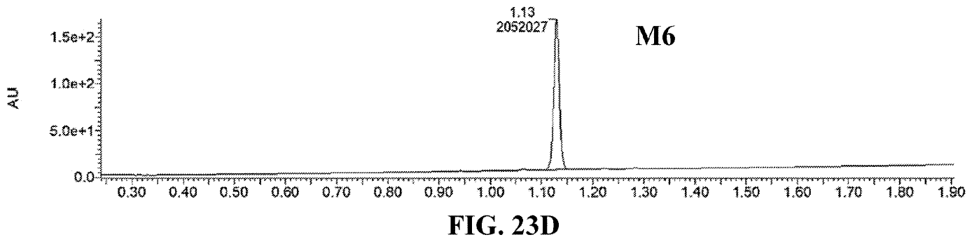

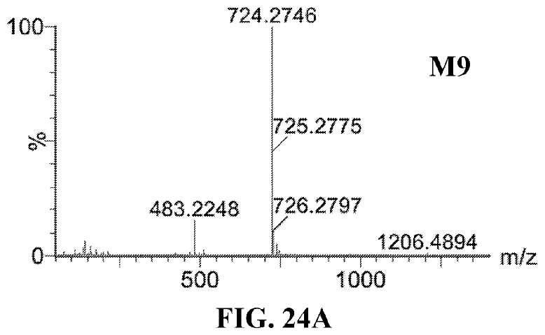

4-Nitrophenyl (2-(2,6-dioxopiperidin-3-yl)-1,3-dioxoisoindolin-4-yl)carbamate (160 mg, 0.366 mmol) added to the reaction mixture of N-(3-amino-5-(tert-butyl)phenyl)-1-(2,5-dimethoxyphenyl)-5-methyl-1H-1,2,3-triazole-4-carboxamide (150 mg, 0.366 mmol) and triethylamine (0.4 mL, 3.0 mmol) and stirred for 18 h. The reaction evaporated in vacuo. The residue was purified by column to get a yellow solid, 103.2 mg, yield 40%. ¹H NMR (500 MHz, DMSO-d₆) δ 11.17 (s, 1H), 10.37 (s, 1H), 10.12 (s, 1H), 9.12 (s, 1H), 8.69 (d, J=8.6 Hz, 1H), 8.05 (q, J=2.0 Hz, 1H), 7.79 (dd, J=8.6, 7.2 Hz, 1H), 7.53-7.47 (m, 2H), 7.42 (t, J=1.8 Hz, 1H), 7.29 (d, J=9.2 Hz, 1H), 7.23 (dd, J=9.2, 3.1 Hz, 1H), 7.16 (d, J=3.1 Hz, 1H), 5.15 (dd, J=12.8, 5.5 Hz, 1H), 3.78 (s, 3H), 3.76 (s, 3H), 2.91 (ddd, J=17.0, 13.8, 5.4 Hz, 1H), 2.66-2.51 (m, 4H), 2.40 (s, 3H), 2.15-2.09 (m, 1H), 1.30 (s, 9H). ¹³C NMR (126 MHz, DMSO-d₆) δ 172.21, 169.34, 167.62, 166.23, 158.83, 152.48, 151.19, 150.98, 147.11, 138.40, 138.36, 138.10, 137.80, 137.04, 135.41, 130.83, 123.86, 123.13, 116.81, 115.79, 113.80, 113.40, 113.28, 111.65, 110.53, 107.54, 55.73, 55.28, 54.31, 48.26, 34.01, 30.49, 8.20. ESI-TOF HRMS: m/z 709.2738 (C36H₃₆N₈O₈+H⁺ requires 709.2734). Representative corresponding mass spectra and HPLC traces are shown in FIG. 23A-D.

### vii. 3-(tert-butyl)-5-(1-(2,5-dimethoxyphenyl)-5-methyl-1H-1,2,3-triazole-4-carboxamido)phenyl (2-(2,6-dioxopiperidin-3-yl)-1,3-dioxoisoindolin-4-yl)glycinate (M7)

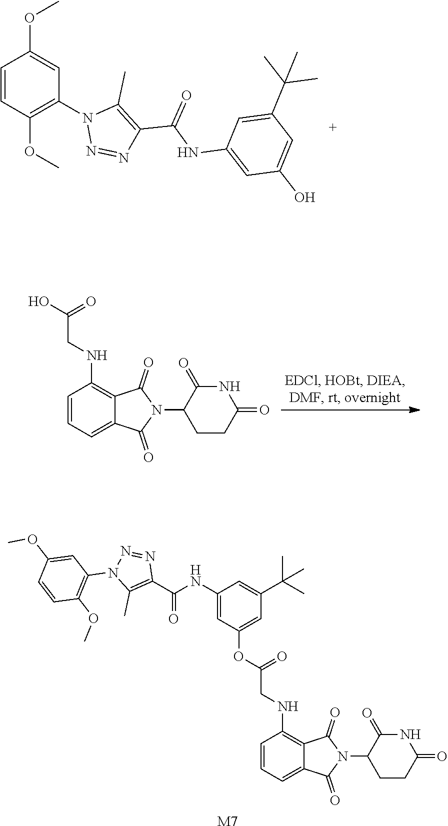

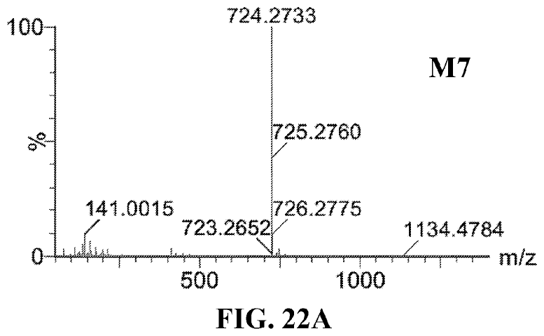

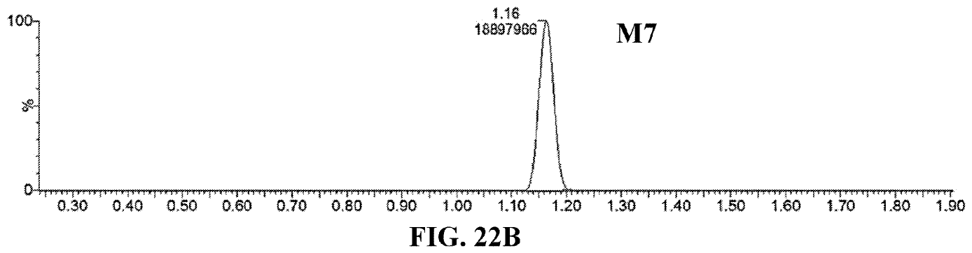

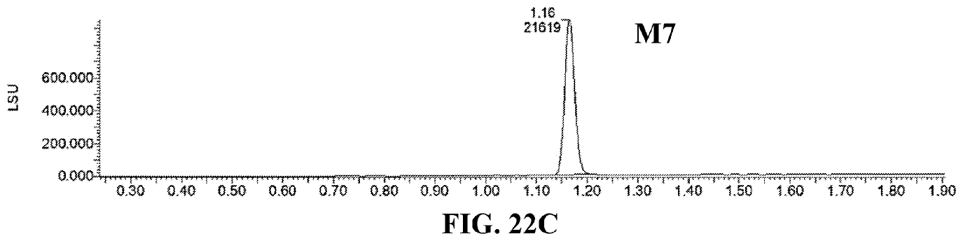

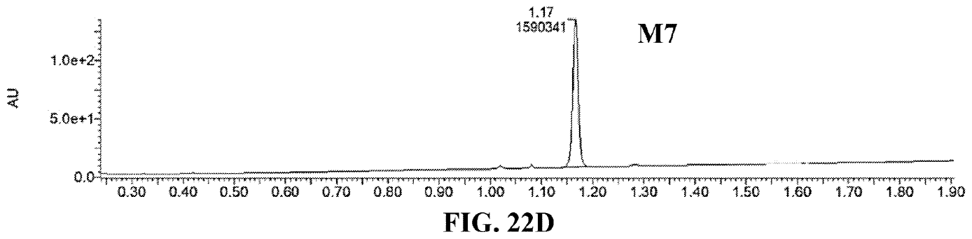

To a solution of N-(3-(tert-butyl)-5-hydroxyphenyl)-1-(2,5-dimethoxyphenyl)-5-methyl-1H-1,2,3-triazole-4-carboxamide (149 mg, 0.362 mmol) in DMF 20 mL was added (2-(2,6-dioxopiperidin-3-yl)-1,3-dioxoisoindolin-4-yl)glycine (100 mg, 0.302 mmol), HOBt, 80% (61.2 mg, 0.362 mmol) and EDCI (87 mg, 0.453 mmol), followed by N-ethyl-N-isopropylpropan-2-amine (78 mg, 0.604 mmol). The suspension stirred at room temperature for overnight. The reaction mixture was diluted with water (50 mL) and extracted with EtOAc (50 mL×2). The EtOAc layer was washed with water, dried with anhydrous Na2SO4, and concentrated with a Rotavapor. The residue was purified by silica gel chromatography (0% to 100% EA in hexane) to give product as solid (152.0 mg, 70% yield). ¹H NMR (500 MHz, DMSO-d₆) δ 11.11 (s, 1H), 10.59 (s, 1H), 7.76 (dt, J=12.3, 1.9 Hz, 2H), 7.64 (dd, J=8.6, 7.1 Hz, 1H), 7.29 (d, J=9.2 Hz, 1H), 7.22 (dd, J=9.1, 3.0 Hz, 2H), 7.15 (d, J=3.1 Hz, 1H), 7.12 (d, J=7.1 Hz, 1H), 7.05 (t, J=6.4 Hz, 1H), 6.91 (t, J=1.9 Hz, 1H), 5.09 (dd, J=12.9, 5.3 Hz, 1H), 4.54 (d, J=6.4 Hz, 2H), 3.78 (s, 3H), 3.75 (s, 3H), 2.89 (ddd, J=17.2, 14.0, 5.4 Hz, 1H), 2.64-2.51 (m, 2H), 2.38 (s, 3H), 2.07-2.02 (m, 1H), 1.28 (s, 9H). ¹³C NMR (126 MHz, DMSO-d₆) δ 172.21, 169.46, 168.76, 168.12, 166.64, 159.01, 152.47, 151.95, 149.55, 147.09, 145.29, 138.68, 138.58, 136.81, 135.58, 131.49, 123.06, 117.30, 116.85, 114.50, 113.39, 113.28, 113.13, 110.81, 110.15, 109.30, 55.73, 55.28, 48.01, 43.42, 34.10, 30.35, 30.09, 21.51, 8.21. ESI-TOF HRMS: m/z 724.2733 (C₃₇H₃₇N₇O₉+H⁺ requires 724.2731). Representative corresponding mass spectra and HPLC traces are shown in FIG. 22A-D.

### viii. N-(3-(tert-butyl)-5-(2-((2-(2,6-dioxopiperidin-3-yl)-1,3-dioxoisoindolin-4-yl)amino)acetamido)phenyl)-1-(2,5-dimethoxyphenyl)-5-methyl-1H-1,2,3-triazole-4-carboxamide (M8)

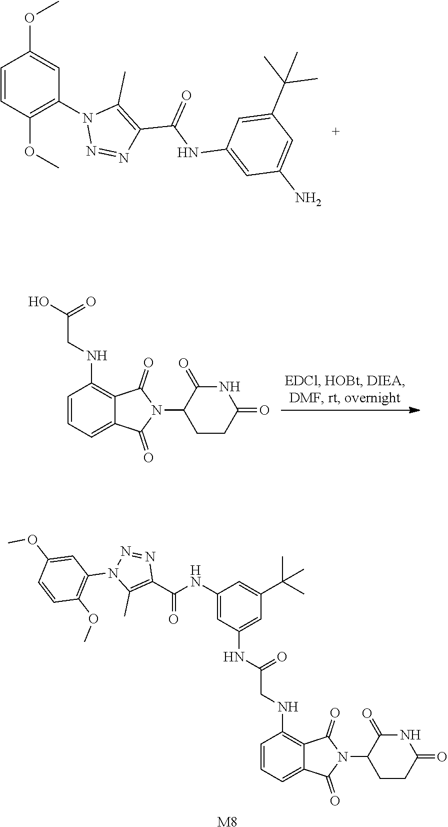

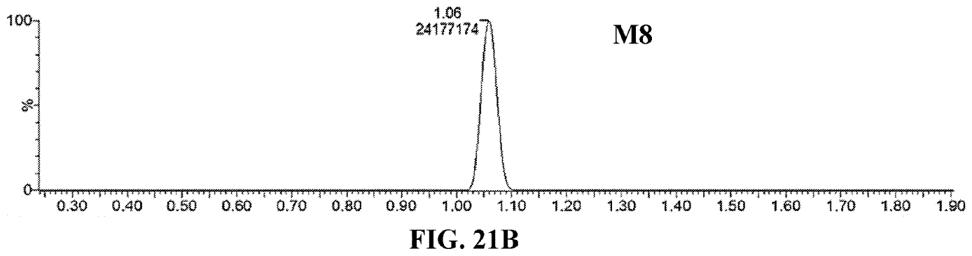

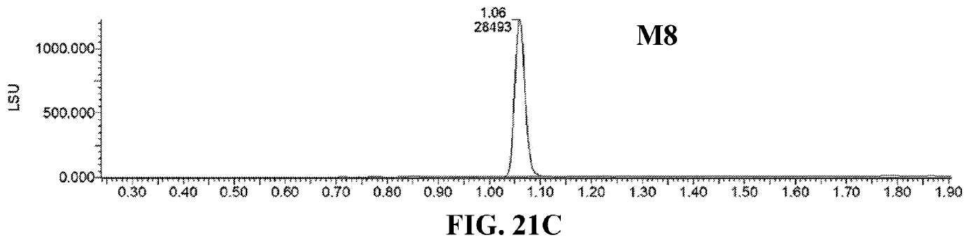

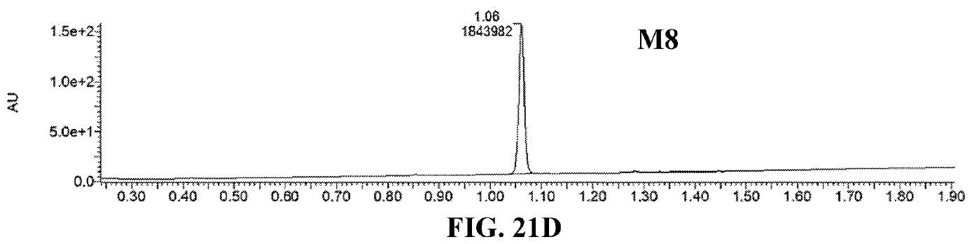

To a solution of N-(3-amino-5-(tert-butyl)phenyl)-1-(2,5-dimethoxyphenyl)-5-methyl-1H-1,2,3-triazole-4-carboxamide (148 mg, 0.362 mmol) in DMF 20 mL was added (2-(2,6-dioxopiperidin-3-yl)-1,3-dioxoisoindolin-4-yl)glycine (100 mg, 0.302 mmol), HOBt, 80% (61.2 mg, 0.362 mmol) and EDCI (87 mg, 0.453 mmol), followed by N-ethyl-N-isopropylpropan-2-amine (78 mg, 0.604 mmol). The suspension stirred at room temperature for overnight. The reaction mixture was diluted with water (50 mL) and extracted with EtOAc (50 mL×2). The EtOAc layer was washed with water, dried with anhydrous Na₂SO₄ and concentrated. The residue was purified by silica gel chromatography (0% to 100% EA in hexane) to give product as a yellow solid (142.0 mg, 65% yield). ¹H NMR (500 MHz, DMSO-d₆) δ 11.12 (s, 1H), 10.38 (s, 1H), 10.20 (s, 1H), 8.14 (s, 1H), 7.62 (dd, J=8.5, 7.1 Hz, 1H), 7.54 (t, J=1.8 Hz, 1H), 7.50 (t, J=1.8 Hz, 1H), 7.28 (d, J=9.2 Hz, 1H), 7.22 (dd, J=9.2, 3.1 Hz, 1H), 7.15 (d, J=3.0 Hz, 1H), 7.09 (d, J=7.0 Hz, 1H), 7.04 (t, J=5.9 Hz, 1H), 6.99 (d, J=8.6 Hz, 1H), 5.09 (dd, J=12.9, 5.4 Hz, 1H), 4.19 (d, J=5.9 Hz, 2H), 3.78 (s, 3H), 3.75 (s, 3H), 2.96-2.85 (m, 1H), 2.65-2.55 (m, 2H), 2.38 (s, 3H), 2.08-2.02 (m, 1H), 1.28 (s, 9H). ¹³C NMR (126 MHz, DMSO-d₆) δ 172.23, 169.47, 168.17, 166.82, 166.73, 158.82, 152.47, 150.91, 147.10, 145.45, 138.38, 137.98, 137.01, 135.65, 131.48, 123.11, 117.06, 116.81, 113.39, 113.27, 112.46, 111.25, 110.43, 109.18, 108.27, 55.72, 55.27, 47.99, 45.03, 33.99, 30.46, 30.09, 21.56, 8.20. ESI-TOF HRMS: m/z 723.2899 (C₃₇H₃₈N₈O₈+H⁺ requires 723.2891). Representative corresponding mass spectra and HPLC traces are shown in FIG. 21A-D.

### ix. N-(3-(tert-butyl)-5-(2-((2-(2,6-dioxopiperidin-3-yl)-1,3-dioxoisoindolin-4-yl)amino)-2-oxoethoxy)phenyl)-1-(2,5-dimethoxyphenyl)-5-methyl-1H-1,2,3-triazole-4-carboxamide (M9)

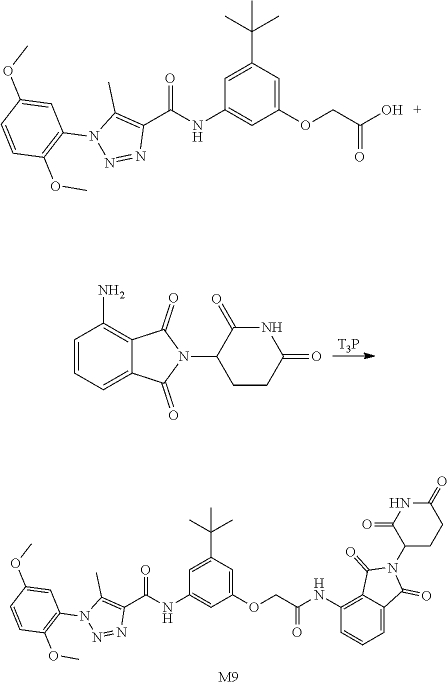

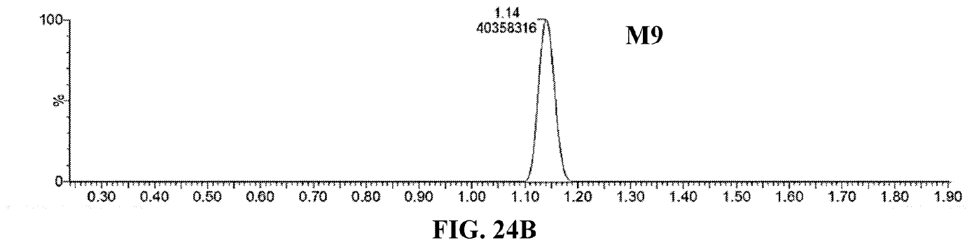

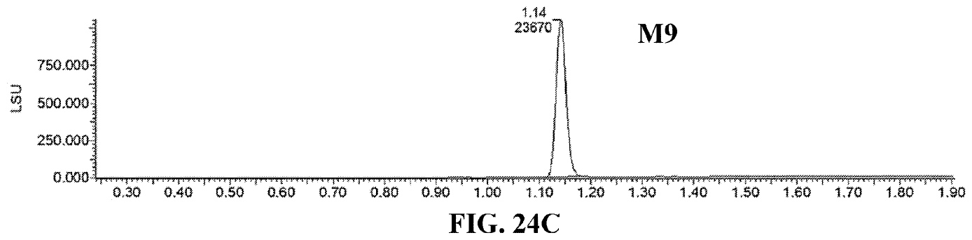

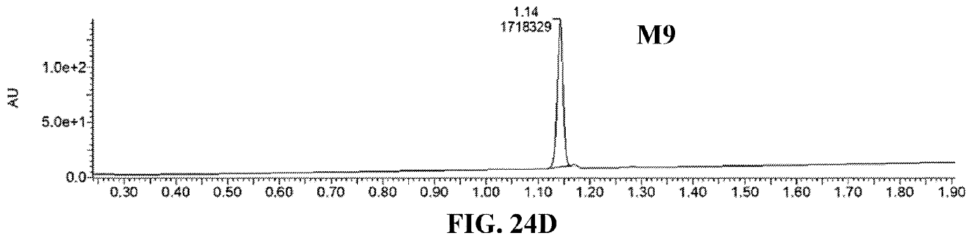

In a solution of 4-amino-2-(2,6-dioxopiperidin-3-yl)isoindoline-1,3-dione (70.0 mg, 0.256 mmol) and 2-(3-(tert-butyl)-5-(1-(2,5-dimethoxyphenyl)-5-methyl-1H-1,2,3-triazole-4-carboxamido)phenoxy)acetic acid (100 mg, 0.213 mmol) in DMF (5 mL) was added T3P (815 mg, 1.281 mmol) and pyridine (1 mL) at 0° C. The mixture was stirred at 80° C. for 3 hours. After cooling down, the reaction mixture was added EA (50 mL) and washed by water. The organic phase was concentrated under the reduced pressure. The residue was purified by biotage column (water/acetonitrile from 100/0 to 0/100) to get a white solid, 103.1 mg, 67% yield. ¹H NMR (400 MHz, DMSO-d₆) δ 11.05 (s, 1H), 10.50 (s, 1H), 10.30 (s, 1H), 8.71 (dd, J=8.5, 0.8 Hz, 1H), 7.89 (dd, J=8.5, 7.3 Hz, 1H), 7.65 (dd, J=7.4, 0.7 Hz, 1H), 7.61 (dt, J=11.8, 1.9 Hz, 2H), 7.29 (d, J=9.2 Hz, 1H), 7.22 (dd, J=9.2, 3.0 Hz, 1H), 7.13 (d, J=3.0 Hz, 1H), 6.96 (dd, J=2.4, 1.6 Hz, 1H), 5.16 (dd, J=12.8, 5.5 Hz, 1H), 4.82 (s, 2H), 3.79 (s, 3H), 3.76 (s, 3H), 2.91 (ddd, J=17.9, 14.4, 5.4 Hz, 1H), 2.66-2.56 (m, 2H), 2.39 (s, 3H), 2.10 (dd, J=9.1, 4.1 Hz, 1H), 1.30 (s, 9H). ¹³C NMR (101 MHz, DMSO-d₆) δ 172.44, 169.52, 168.20, 167.66, 166.56, 159.44, 157.02, 153.14, 152.72, 147.74, 139.42, 138.95, 137.50, 136.43, 135.85, 131.33, 124.67, 123.82, 118.46, 117.42, 116.48, 114.01(2C), 111.39, 108.18, 104.24, 67.49, 56.38, 55.89, 49.06, 34.62, 30.93, 30.87, 21.88, 8.71. ESI-TOF HRMS: m/z 724.2746 (C₃₇H₃₇N₇O₉+H⁺ requires 724.2731). Representative corresponding mass spectra and HPLC traces are shown in FIG. 24A-D.

### x. N-(3-(tert-butyl)-5-(2-((2-(2,6-dioxopiperidin-3-yl)-1,3-dioxoisoindolin-4-yl)amino)ethoxy)phenyl)-1-(2,5-dimethoxyphenyl)-5-methyl-1H-1,2,3-triazole-4-carboxamide (M10)

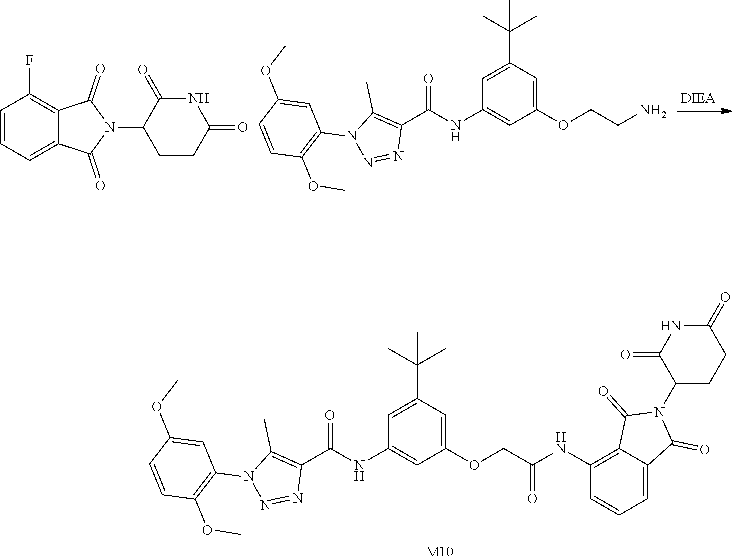

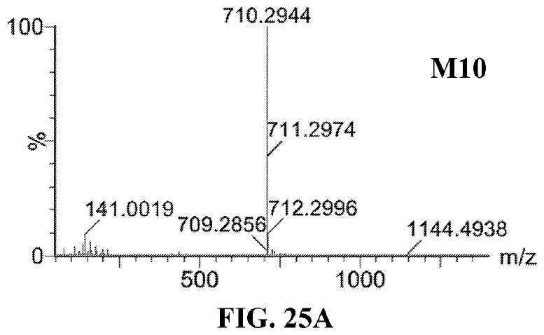

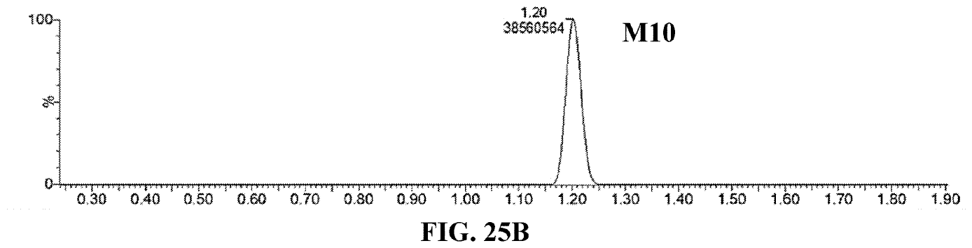

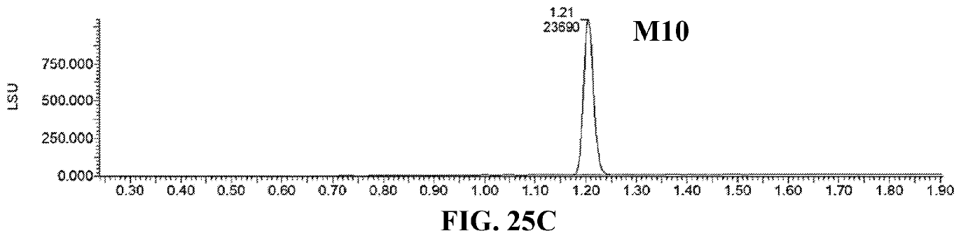

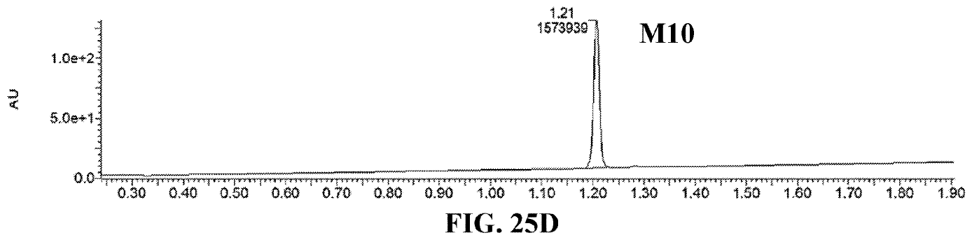

To a solution of 2-(2,6-dioxopiperidin-3-yl)-4-fluoroisoindoline-1,3-dione (60.9 mg, 0.220 mmol) and N-(3-(2-aminoethoxy)-5-(tert-butyl)phenyl)-1-(2,5-dimethoxyphenyl)-5-methyl-1H-1,2,3-triazole-4-carboxamide (50 mg, 0.110 mmol) in DMSO (2 mL) was added N-ethyl-N-isopropylpropan-2-amine (28.5 mg, 0.220 mmol). After stirring at 90° C. for 24 hours, the mixture was concentrated and purified by silica gel flash column chromatography (water/acetonitrile from 100/0 to 0/100) to give N-(3-(tert-butyl)-5-(2-((2-(2,6-dioxopiperidin-3-yl)-1,3-dioxoisoindolin-4-yl)amino)ethoxy)phenyl)-1-(2,5-dimethoxyphenyl)-5-methyl-1H-1,2,3-triazole-4-carboxamide as a solid, 0.6 mg, 64%. H NMR (500 MHz, DMSO-d₆) δ 11.09 (s, 1H), 10.34 (s, 1H), 7.62 (dd, J=8.5, 7.0 Hz, 1H), 7.54 (t, J=1.7 Hz, 1H), 7.49 (t, J=2.1 Hz, 1H), 7.31-7.25 (m, 2H), 7.22 (dd, J=9.1, 3.1 Hz, 1H), 7.14 (d, J=3.0 Hz, 1H), 7.07 (d, J=7.0 Hz, 1H), 6.79 (t, J=6.1 Hz, 1H), 6.68 (t, J=2.0 Hz, 1H), 5.06 (dd, J=12.9, 5.3 Hz, 1H), 4.17 (t, J=5.5 Hz, 2H), 3.78 (s, 3H), 3.77-3.71 (m, 5H), 2.88 (ddd, J=17.3, 14.0, 5.4 Hz, 1H), 2.62-2.51 (m, 2H), 2.38 (s, 3H), 2.06-1.97 (m, 1H), 1.26 (s, 9H). ¹³C NMR (126 MHz, DMSO-d₆) δ 172.19, 169.45, 168.29, 166.66, 158.84, 157.59, 152.47, 151.83, 147.09, 145.72, 138.79, 138.40, 136.95, 135.65, 131.55, 123.09, 116.94, 116.81, 113.38, 113.27, 110.24, 109.62, 108.83, 106.80, 102.98, 65.70, 55.72, 55.27, 47.95, 40.75, 34.03, 30.44, 30.09, 21.52, 8.22. ESI-TOF HRMS: m/z 710.2944 (C₃₇H₃₉N₇O₈+H⁺ requires 710.2938). Representative corresponding mass spectra and HPLC traces are shown in FIG. 25A-D.

### xi. N-(3-(tert-butyl)-5-((2-((2-(2,6-dioxopiperidin-3-yl)-1,3-dioxoisoindolin-4-yl)amino)ethyl)amino)phenyl)-1-(2,5-dimethoxyphenyl)-5-methyl-1H-1,2,3-triazole-4-carboxamide (M11)

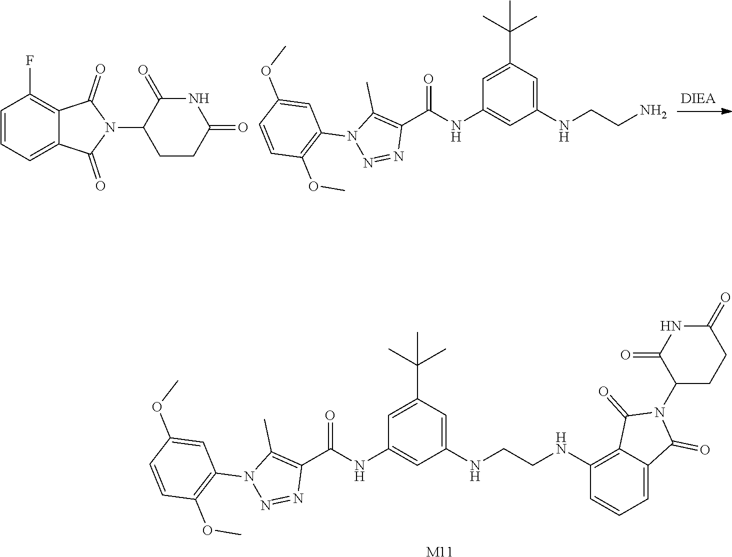

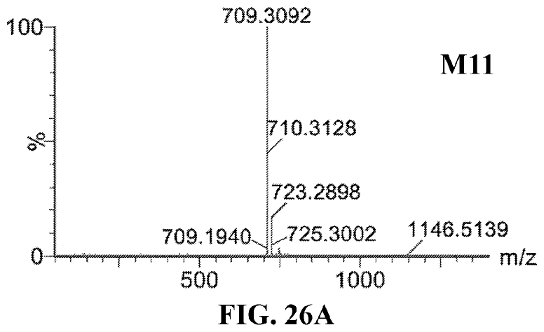

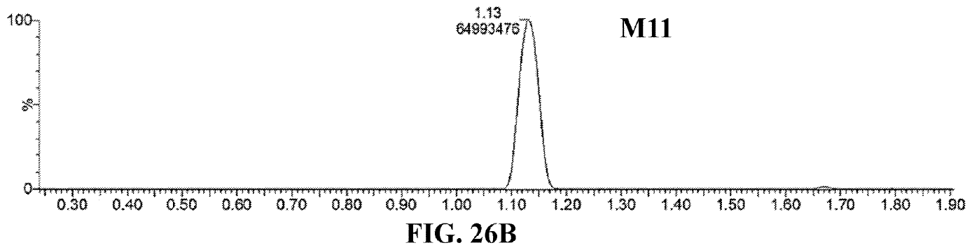

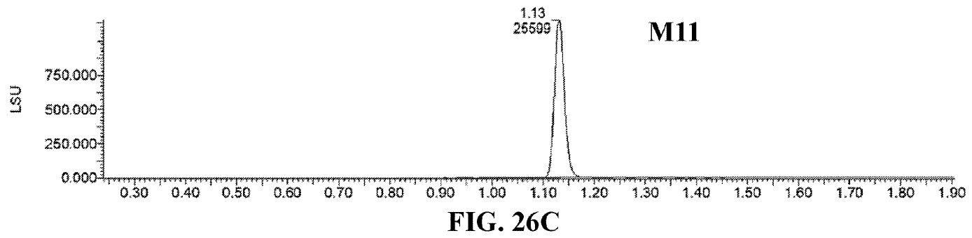

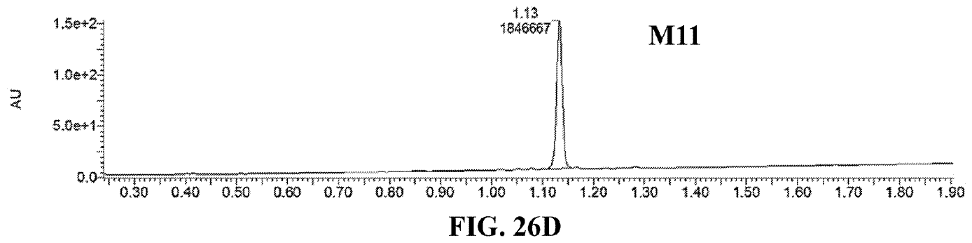

To a solution of 2-(2,6-dioxopiperidin-3-yl)-4-fluoroisoindoline-1,3-dione (61.0 mg, 0.221 mmol) and N-(3-((2-aminoethyl)amino)-5-(tert-butyl)phenyl)-1-(2,5-dimethoxyphenyl)-5-methyl-1H-1,2,3-triazole-4-carboxamide (50 mg, 0.110 mmol) in DMSO (2 mL) was added N-ethyl-N-isopropylpropan-2-amine (28.6 mg, 0.221 mmol). After stirring at 90° C. for 24 h, the mixture was concentrated and purified by silica gel flash column chromatography to give N-(3-(tert-butyl)-5-((2-((2-(2,6-dioxopiperidin-3-yl)-1,3-dioxoisoindolin-4-yl)amino)ethyl)amino)phenyl)-1-(2,5-dimethoxyphenyl)-5-methyl-1H-1,2,3-triazole-4-carboxamide as a solid, 44.9 mg, 58%. ¹H NMR (500 MHz, DMSO-d₆) δ 11.09 (s, 1H), 10.01 (s, 1H), 7.59 (dd, J=8.6, 7.1 Hz, 1H), 7.29 (s, 1H), 7.22 (dd, J=9.2, 3.1 Hz, 1H), 7.19 (d, J=8.7 Hz, 1H), 7.14 (d, J=3.0 Hz, 1H), 7.12 (d, J=1.9 Hz, 2H), 7.03 (d, J=7.0 Hz, 1H), 6.80 (t, J=6.1 Hz, 1H), 6.38 (t, J=1.8 Hz, 1H), 5.05 (dd, J=12.8, 5.4 Hz, 1H), 3.78 (s, 3H), 3.75 (s, 3H), 3.55 (q, J=6.3 Hz, 2H), 3.26 (q, J=6.3 Hz, 2H), 2.88 (ddd, J=17.0, 13.9, 5.4 Hz, 1H), 2.64-2.50 (m, 2H), 2.38 (s, 3H), 2.06-1.99 (m, 1H), 1.24 (s, 9H). ¹³C NMR (126 MHz, DMSO-d₆) δ 172.20, 169.48, 168.20, 166.69, 158.56, 152.47, 150.93, 148.02, 147.10, 145.71, 138.45, 138.13, 137.16, 135.64, 131.65, 123.15, 116.78, 116.67, 113.39, 113.28, 109.93, 108.67, 105.39, 105.01, 100.51, 55.72, 55.27, 47.92, 41.73, 40.60, 33.79, 30.57, 30.09, 21.56, 8.20. ESI-TOF HRMS: m/z 709.3092 (C₃₇H₄₀N₈O₇+H⁺ requires 709.3089). Representative corresponding mass spectra and HPLC traces are shown in FIG. 26A-D.

### xii. N-(5-(tert-butyl)-2-(4-((2-(2,6-dioxopiperidin-3-yl)-1,3-dioxoisoindolin-4-yl)oxy)butoxy)phenyl)-1-(2,5-dimethoxyphenyl)-5-methyl-1H-1,2,3-triazole-4-carboxamide (M12)

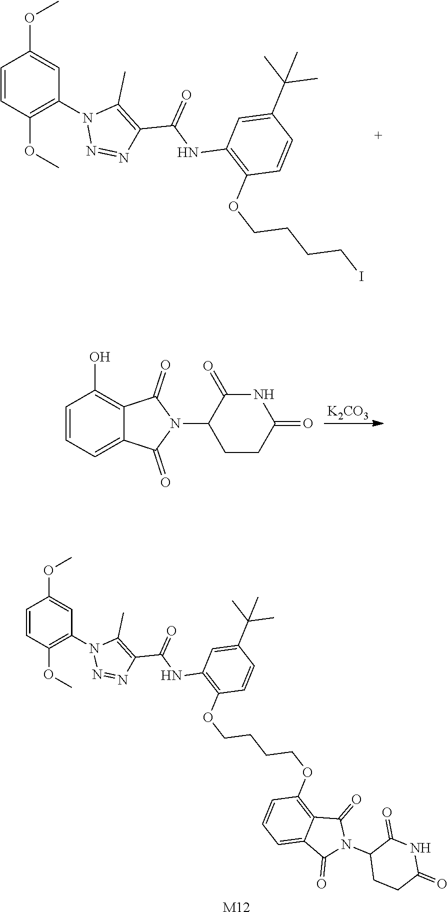

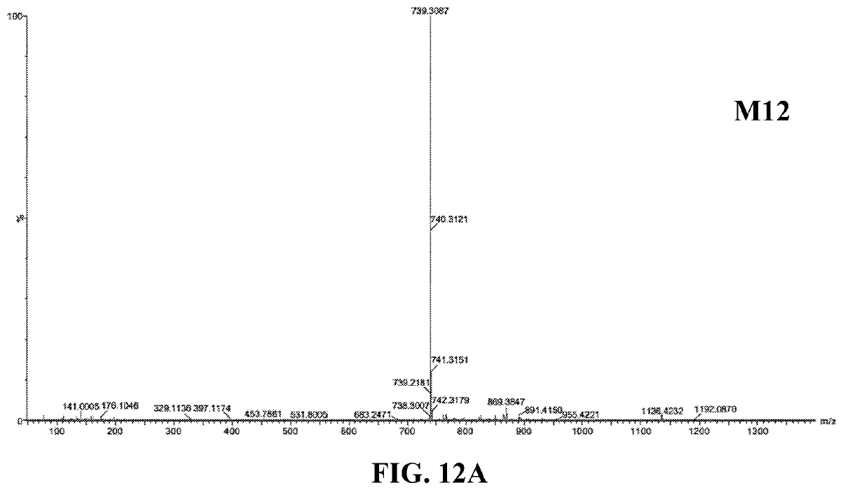

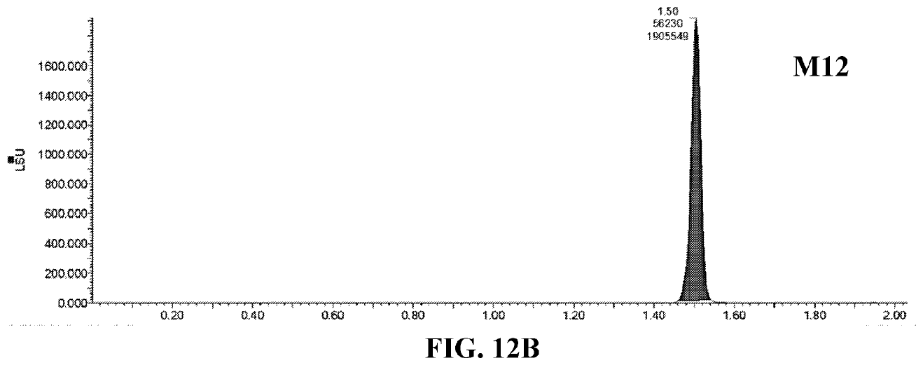

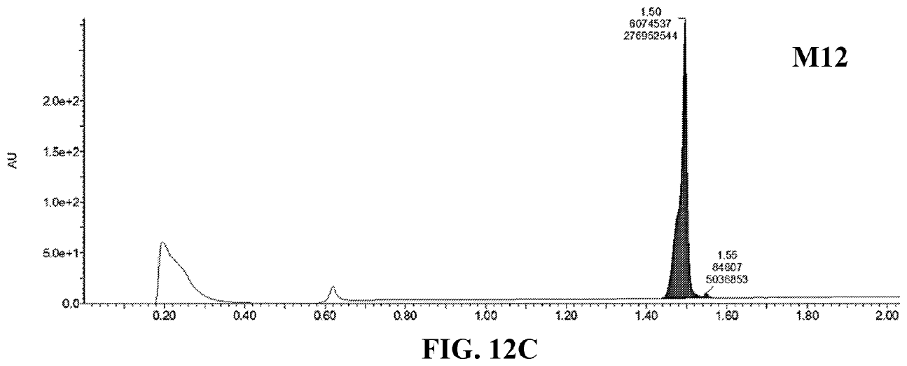

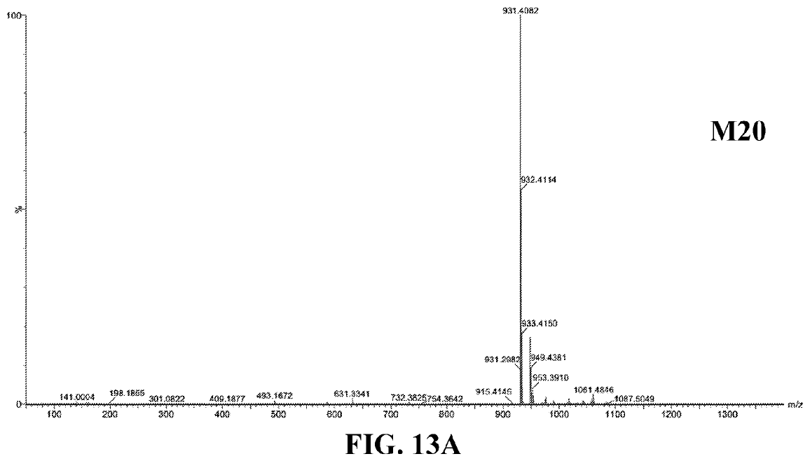

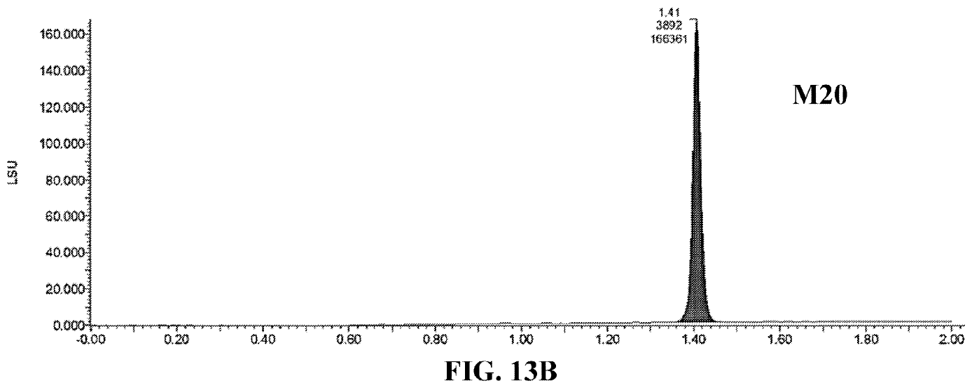

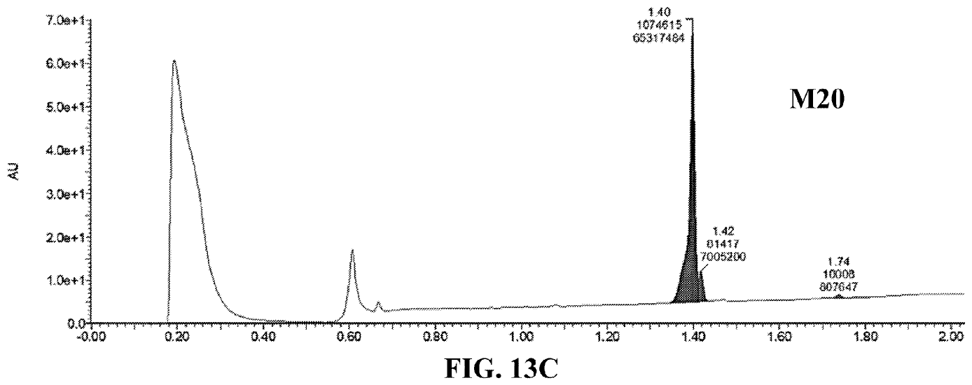

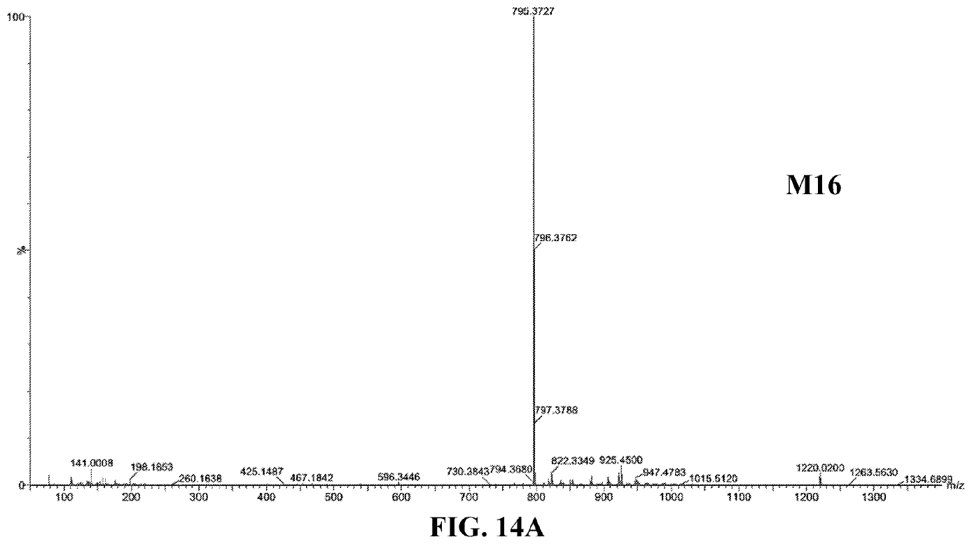

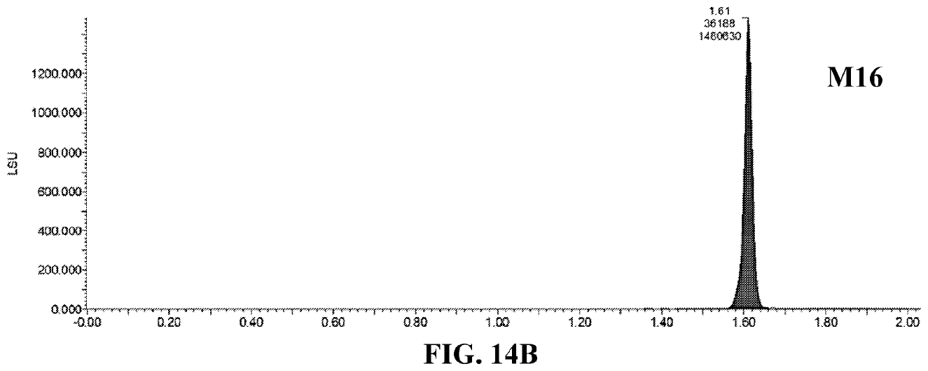

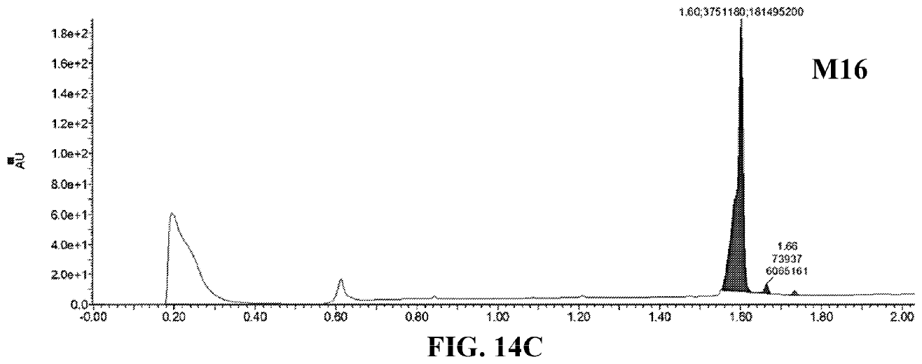

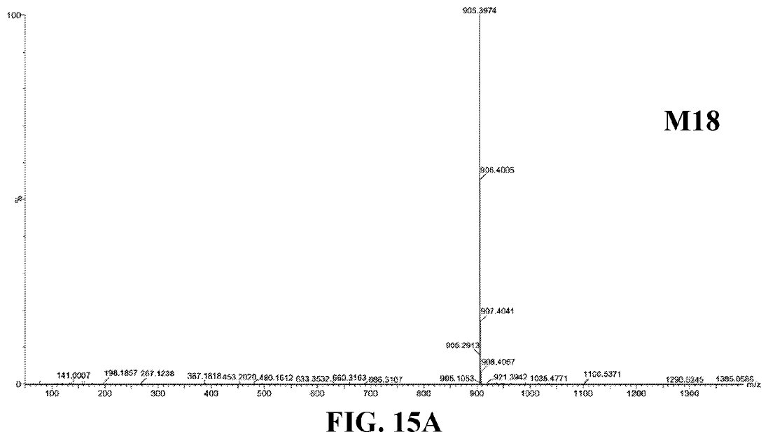

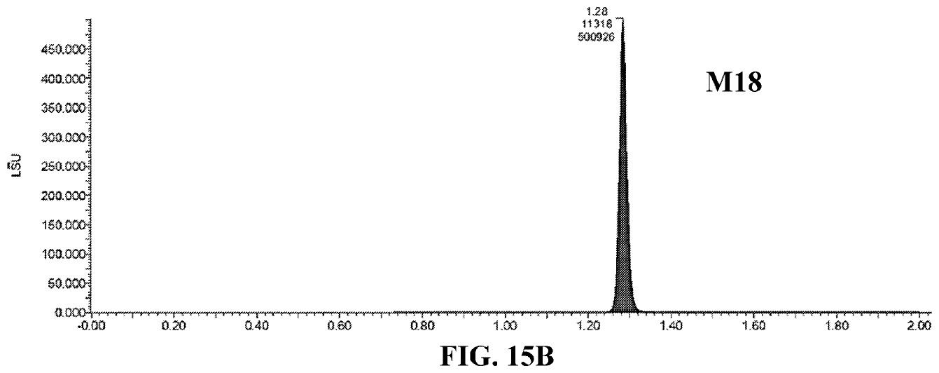

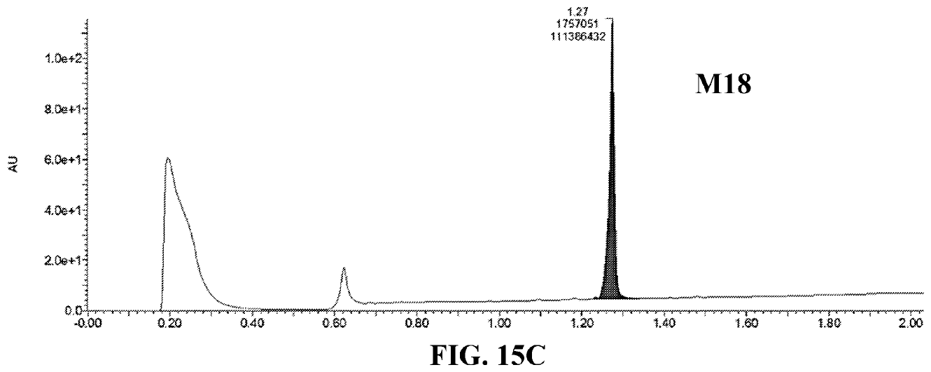

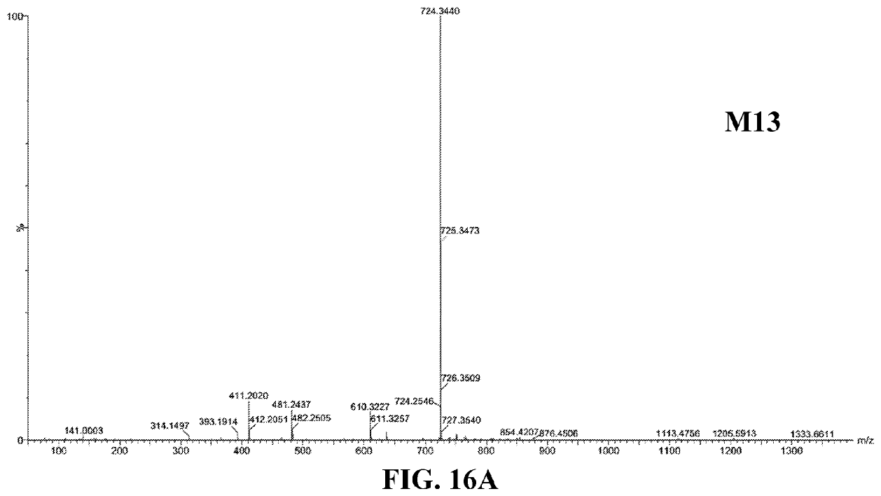

To a solution of 2-(2,6-dioxopiperidin-3-yl)-4-hydroxyisoindoline-1,3-dione (0.060 g, 0.220 mmol) in DMF 15 mL at rt was added K₂CO₃ (0.030 g, 0.220 mmol), and N-(5-(tert-butyl)-2-(4-iodobutoxy)phenyl)-1-(2,5-dimethoxyphenyl)-5-methyl-1H-1,2,3-triazole-4-carboxamide (0.118 g, 0.2 mmol). The suspension was stirred at room temperature for overnight. The reaction mixture was diluted with water (20 mL) and extracted with EtOAc (25 mL×2). The combined EtOAc layers were washed with water, dried with anhydrous Na₂SO₄ and concentrated. The residue was purified by reverse silica gel chromatography (0% to 100% acetonitrile in water) to give product as solid (117.5 mg, 79% yield). ¹H NMR (400 MHz, Chloroform-d) δ 9.78 (s, 1H), 8.63 (d, J=2.4 Hz, 1H), 8.07 (s, 1H), 7.59 (dd, J=8.5, 7.3 Hz, 1H), 7.39 (d, J=7.2 Hz, 1H), 7.26 (d, J=8.5 Hz, 1H), 7.13-7.00 (m, 3H), 6.94 (d, J=2.9 Hz, 1H), 6.87 (d, J=8.5 Hz, 1H), 4.94 (dd, J=12.3, 5.3 Hz, 1H), 4.34 (t, J=5.8 Hz, 2H), 4.19 (t, J=5.7 Hz, 2H), 3.82 (s, 3H), 3.76 (s, 3H), 2.91-2.64 (m, 3H), 2.54 (s, 3H), 2.28-2.03 (m, 5H), 1.35 (s, 9H). ¹³C NMR (101 MHz, CDCl₃) δ 170.89, 168.01, 167.09, 165.69, 159.26, 156.57, 153.69, 148.01, 145.44, 144.07, 139.15, 138.47, 136.55, 133.69, 127.39, 124.42, 120.17, 119.18, 117.35, 117.10, 117.02, 115.68, 113.88, 113.28, 110.60, 69.05, 68.21, 56.31, 56.01, 49.06, 34.46, 31.56, 31.38, 25.95, 25.83, 22.64, 9.28. ESI-TOF HRMS: m/z 739.3087 (C₃₉H₄₂N₆O₉+H⁺ requires 739.3086). Representative corresponding mass spectra and HPLC traces are shown in FIG. 12A-C.

### xiii. N-(5-(tert-butyl)-2-(4-((2-(2,6-dioxopiperidin-3-yl)-1-oxoisoindolin-4-yl)amino)butoxy)phenyl)-1-(2,5-dimethoxyphenyl)-5-methyl-1H-1,2,3-triazole-4-carboxamide (M13)

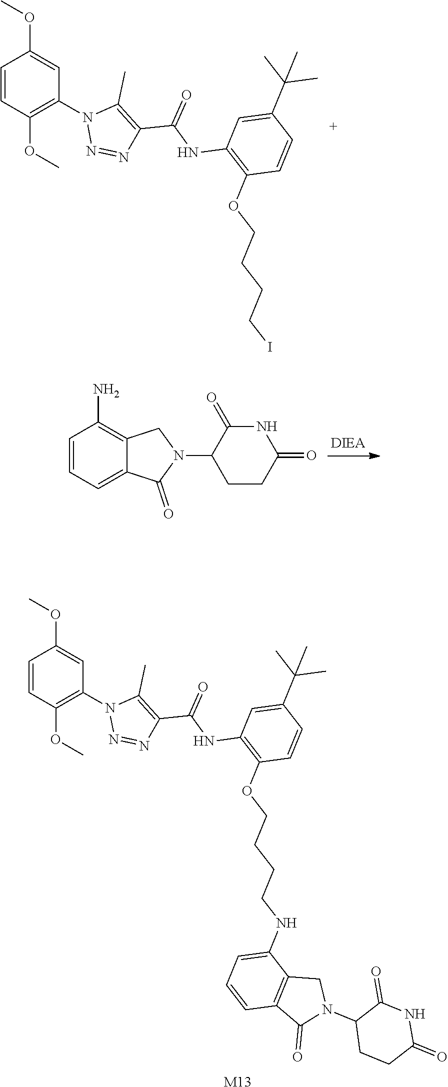 [SMILES: COc1ccc(OC)c(-n2nnc(C(=O)Nc3cc(C(C)(C)C)ccc3OCCCCI)c2C)c1 ; Nc1cccc2c1CN(C1CCC(=O)NC1=O)C2=O ; COc1ccc(OC)c(-n2nnc(C(=O)Nc3cc(C(C)(C)C)ccc3OCCCCNc3cccc4c3CN(C3CCC(=O)NC3=O)C4=O)c2C)c1 ; "M13"]

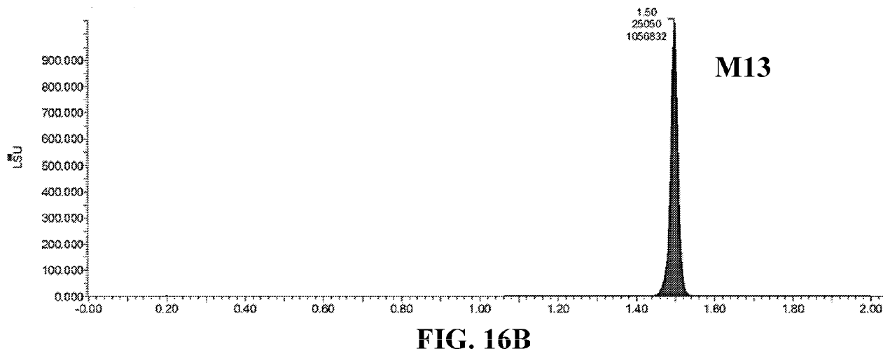

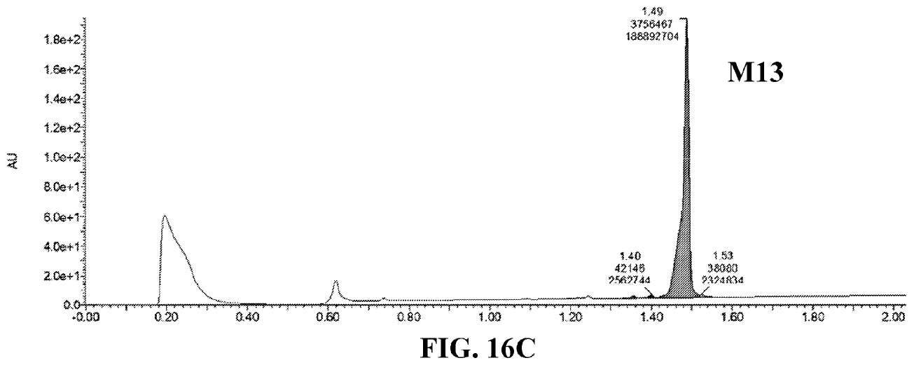

To a solution of N-(5-(tert-butyl)-2-(4-iodobutoxy)phenyl)-1-(2,5-dimethoxyphenyl)-5-methyl-1H-1,2,3-triazole-4-carboxamide (100 mg, 0.169 mmol) in NMP 5 mL was added N-ethyl-N-isopropylpropan-2-amine (88 μl, 0.506 mmol) and 3-(4-amino-1-oxoisoindolin-2-yl)piperidine-2,6-dione (48.1 mg, 0.186 mmol). The suspension was stirred at 110° C. for overnight. The reaction mixture was then diluted with water (50 mL) and extracted with EtOAc (50 mL×2). The combined EtOAc layers were washed with water, dried with anhydrous Na₂SO₄ and concentrated. The residue was purified by column chromatography (0% to 100% acetonitrile in water) to give product as a white solid (84.3 mg, 69% yield). ¹H NMR (400 MHz, DMSO-d₆) δ 10.88 (s, 1H), 9.63 (s, 1H), 8.45 (d, J=2.4 Hz, 1H), 7.29 (d, J=9.2 Hz, 1H), 7.25-7.20 (m, 2H), 7.14-7.08 (m, 2H), 7.04 (d, J=8.6 Hz, 1H), 6.91 (dd, J=7.5, 0.8 Hz, 1H), 6.77 (dd, J=8.1, 0.8 Hz, 1H), 5.52 (t, J=5.6 Hz, 1H), 5.06 (dd, J=13.2, 5.1 Hz, 1H), 4.27-4.10 (m, 4H), 3.78 (s, 3H), 3.75 (s, 3H), 3.30-3.12 (m, 4H), 2.89 (ddd, J=17.3, 13.5, 5.4 Hz, 1H), 2.62-2.56 (m, 1H), 2.42 (s, 3H), 2.26 (qd, J=13.3, 4.5 Hz, 1H), 2.04-1.80 (m, 4H), 1.30 (s, 9H). ¹³C NMR (101 MHz, DMSO-d₆) δ 172.66, 171.05, 168.82, 158.47, 153.19, 147.73, 145.47, 143.69, 143.10, 138.87, 137.35, 132.06, 129.10, 126.83, 126.52, 123.75, 120.47, 117.54, 116.52, 114.09, 114.01, 111.88, 111.70, 110.01, 68.48, 56.43, 55.92, 51.58, 45.76, 42.46, 34.01, 31.31, 30.58, 26.49, 25.11, 22.79, 8.70. ESI-TOF HRMS: m/z 724.3440 (C₃₉H₄₅N₇O₇+H⁺ requires 724.3453). Representative corresponding mass spectra and HPLC traces are shown in FIG. 16A-C.

### xiv. N-(3-(tert-butyl)-5-(4-((2-(2,6-dioxopiperidin-3-yl)-1-oxoisoindolin-4-yl)amino)butoxy)phenyl)-1-(2,5-dimethoxyphenyl)-5-methyl-1H-1,2,3-triazole-4-carboxamide (M14)

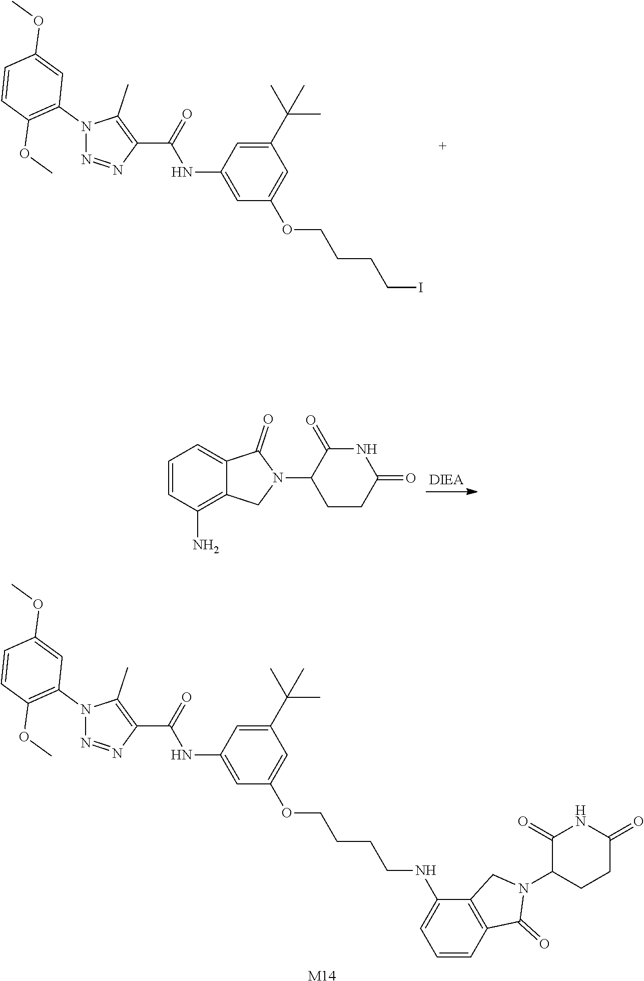 [SMILES: COc1ccc(OC)c(-n2nnc(C(=O)Nc3cc(OCCCCI)cc(C(C)(C)C)c3)c2C)c1 ; Nc1cccc2c1CN(C1CCC(=O)NC1=O)C2=O ; COc1ccc(OC)c(-n2nnc(C(=O)Nc3cc(OCCCCNc4cccc5c4CN(C4CCC(=O)NC4=O)C5=O)cc(C(C)(C)C)c3)c2C)c1 ; "M14"]

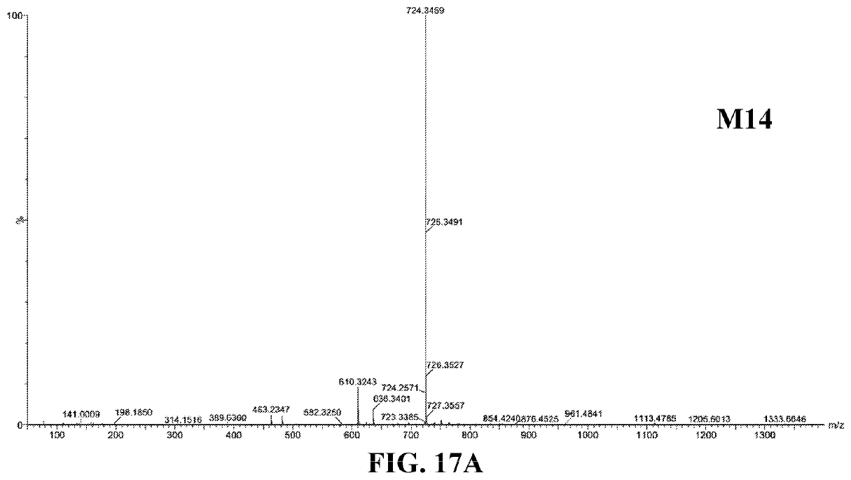

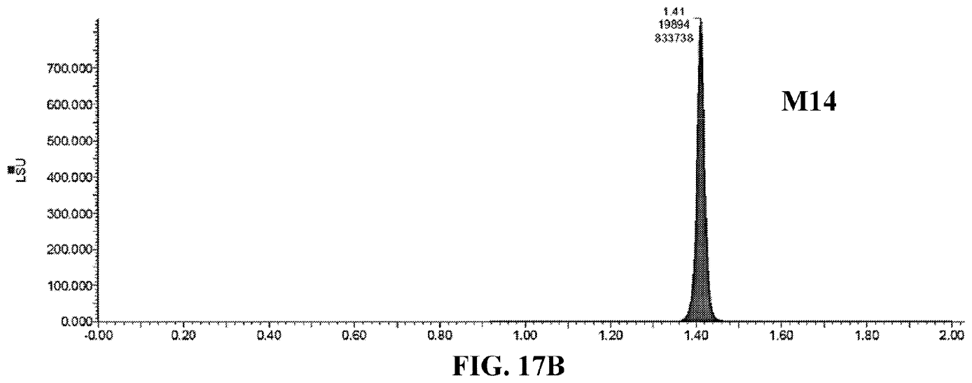

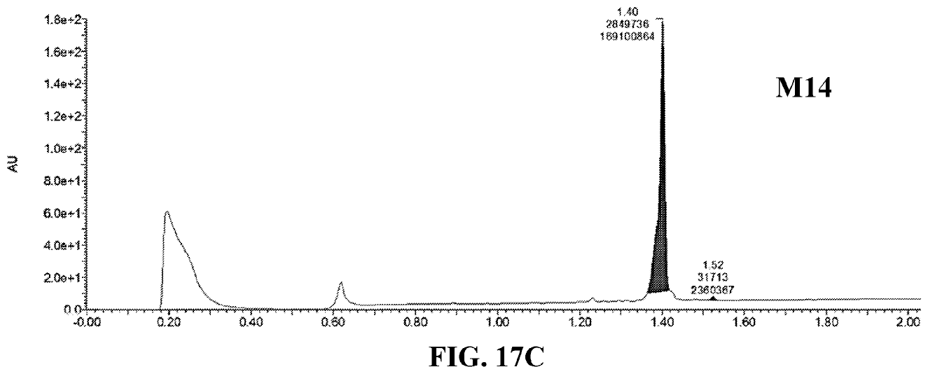

To a solution of N-(3-(tert-butyl)-5-(4-iodobutoxy)phenyl)-1-(2,5-dimethoxyphenyl)-5-methyl-1H-1,2,3-triazole-4-carboxamide (100 mg, 0.169 mmol) in NMP 5 mL was added N-ethyl-N-isopropylpropan-2-amine (88 μl, 0.506 mmol) and 3-(4-amino-1-oxoisoindolin-2-yl)piperidine-2,6-dione (48.1 mg, 0.186 mmol). The suspension was stirred at 110° C. for overnight. The reaction mixture was diluted with water (50 mL) and extracted with EtOAc (50 mL×2). The combined EtOAc layers were washed with water, dried with anhydrous Na₂SO₄ and concentrated. The residue was purified by column chromatography (0% to 100% acetonitrile in water) to give product as a solid (86.2 mg, 71% yield). ¹H NMR (400 MHz, DMSO-d₆) δ 10.90 (s, 1H), 10.19 (s, 1H), 7.50 (d, J=1.8 Hz, 1H), 7.46 (t, J=2.1 Hz, 1H), 7.33-7.26 (m, 2H), 7.22 (dd, J=9.1, 3.1 Hz, 1H), 7.13 (d, J=3.1 Hz, 1H), 6.94 (dd, J=7.4, 0.8 Hz, 1H), 6.83-6.77 (m, 1H), 6.66 (dd, J=2.3, 1.6 Hz, 1H), 5.53 (t, J=5.5 Hz, 1H), 5.09 (dd, J=13.1, 5.2 Hz, 1H), 4.20 (q, J=17.1 Hz, 2H), 4.03 (t, J=6.2 Hz, 2H), 3.79 (s, 3H), 3.76 (s, 3H), 3.25-3.18 (m, 2H), 2.91 (ddd, J=17.9, 13.4, 5.3 Hz, 1H), 2.66-2.57 (m, 1H), 2.39 (s, 3H), 2.34-2.25 (m, 1H), 2.09-1.97 (m, 1H), 1.90-1.72 (m, 4H), 1.28 (s, 9H). ¹³C NMR (101 MHz, DMSO-d₆) δ 172.69, 171.08, 168.83, 159.42, 158.60, 153.19, 152.36, 147.80, 143.76, 139.31, 138.89, 137.62, 132.10, 129.14, 126.56, 123.88, 119.44, 117.46, 114.07, 111.88, 110.03, 109.94, 107.53, 103.61, 67.25, 56.43, 55.94, 51.59, 45.76, 42.50, 34.56, 31.06, 30.58, 26.43, 25.29, 22.82, 8.75. ESI-TOF HRMS: m/z 724.3459 (C₃₉H₄₅N₇O₇+H⁺ requires 724.3453). Representative corresponding mass spectra and HPLC traces are shown in FIG. 17A-C.

### xv. N-(3-(tert-butyl)-5-(4-(2-((2-(2,6-dioxopiperidin-3-yl)-1,3-dioxoisoindolin-4-yl)oxy)acetamido)butanamido)phenyl)-1-(2,5-dimethoxyphenyl)-5-methyl-1H-1,2,3-triazole-4-carboxamide (M15)

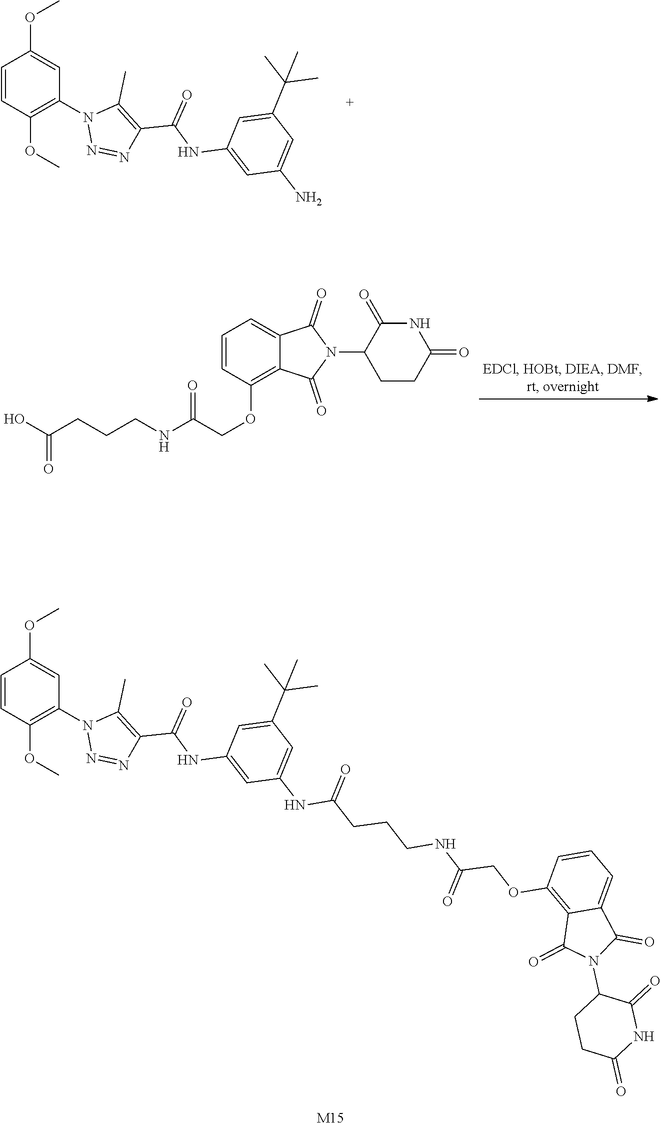 [SMILES: COc1ccc(OC)c(-n2nnc(C(=O)Nc3cc(N)cc(C(C)(C)C)c3)c2C)c1 ; O=C(O)CCCNC(=O)COc1cccc2c1C(=O)N(C1CCC(=O)NC1=O)C2=O ; COc1ccc(OC)c(-n2nnc(C(=O)Nc3cc(NC(=O)CCCNC(=O)COc4cccc5c4C(=O)N(C4CCC(=O)NC4=O)C5=O)cc(C(C)(C)C)c3)c2C)c1 ; "M15"]

A solution of 4-(2-((2-(2,6-dioxopiperidin-3-yl)-1,3-dioxoisoindolin-4-yl)oxy)acetamido)butanoic acid (25 mg, 0.060 mmol) in DMSO 2 mL was added N-(3-amino-5-(tert-butyl)phenyl)-1-(2,5-dimethoxyphenyl)-5-methyl-1H-1,2,3-triazole-4-carboxamide (24.53 mg, 0.060 mmol), HOBt, 80% (12.14 mg, 0.072 mmol) and EDCI (17.22 mg, 0.090 mmol), followed by N-ethyl-N-isopropylpropan-2-amine (7.74 mg, 0.060 mmol). The suspension was stirred at room temperature for overnight. Then the reaction mixture was diluted with water (50 mL) and extracted with EtOAc (50 mL×2). The combined EtOAc layers were washed with water, dried with anhydrous Na₂SO₄ and concentrated. The residue was purified by column chromatography (0% to 100% acetonitrile in water) to give product as solid (32.8 mg, 68% yield). ¹H NMR (400 MHz, Chloroform-d) δ 9.16 (s, 1H), 9.11 (s, 1H), 8.27 (s, 1H), 7.78 (t, J=6.1 Hz, 1H), 7.73-7.65 (m, 3H), 7.48 (dd, J=7.4, 0.6 Hz, 1H), 7.33 (t, J=1.8 Hz, 1H), 7.22-7.17 (m, 1H), 7.08 (dd, J=9.1, 3.0 Hz, 1H), 7.02 (d, J=9.2 Hz, 1H), 6.95 (d, J=3.0 Hz, 1H), 4.99 (dd, J=12.4, 5.4 Hz, 1H), 4.68 (s, 2H), 3.81 (s, 3H), 3.75 (s, 3H), 3.56-3.41 (m, J=7.4 Hz, 2H), 2.92-2.64 (m, 3H), 2.49 (s, 3H), 2.38 (t, J=7.1 Hz, 2H), 2.20-2.13 (m, 1H), 2.03-1.95 (m, 2H), 1.31 (s, 9H). ¹³C NMR (101 MHz, CDCl₃) δ 171.03, 170.75, 168.97, 167.44, 166.62, 166.55, 159.45, 154.82, 153.69, 153.19, 148.01, 139.62, 138.68, 137.81, 137.79, 137.18, 133.36, 124.29, 120.47, 118.29, 117.62, 117.55, 113.80, 113.32, 113.07, 112.59, 108.21, 68.81, 56.34, 56.02, 49.44, 38.44, 34.99, 34.49, 31.40, 31.25, 25.08, 22.49, 9.24. ESI-TOF HRMS: m/z 809.3257 (C₄₁H₄₄N₈O₁₀+H⁺ requires 809.3253).

### xvi. N-(3-(tert-butyl)-5-((8-((2-(2,6-dioxopiperidin-3-yl)-1,3-dioxoisoindolin-4-yl)oxy)octyl)oxy)phenyl)-1-(2,5-dimethoxyphenyl)-5-methyl-1H-1,2,3-triazole-4-carboxamide (M16)

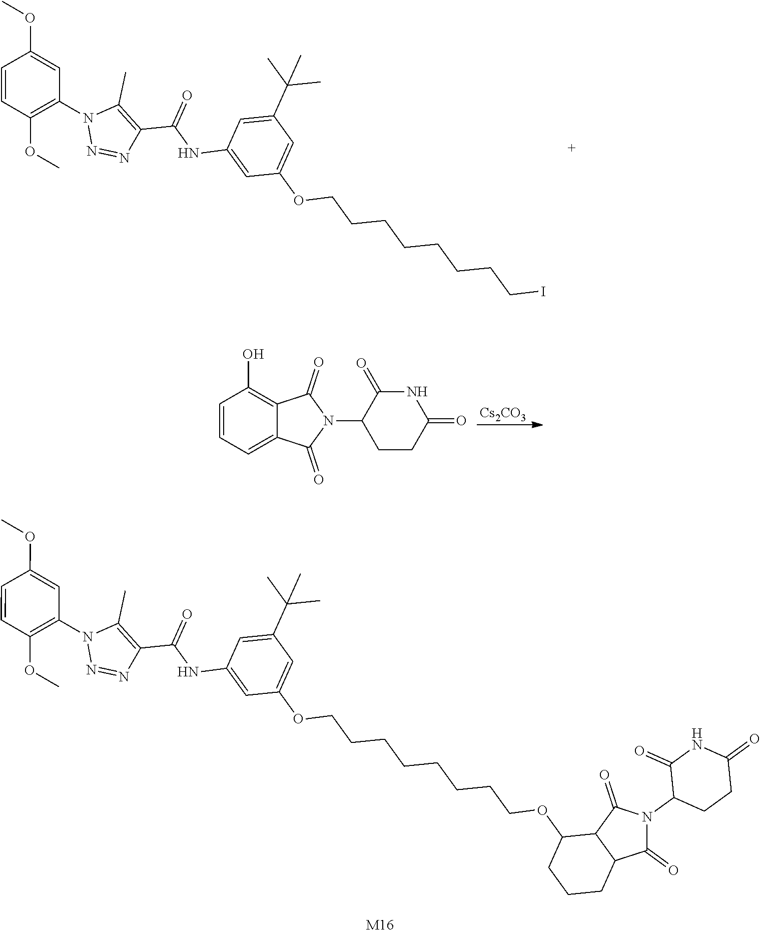

To a solution of N-(3-(tert-butyl)-5-((8-iodooctyl)oxy)phenyl)-1-(2,5-dimethoxyphenyl)-5-methyl-1H-1,2,3-triazole-4-carboxamide (84.20 mg, 0.130 mmol) in DMF 5 mL was added 2-(2,6-dioxopiperidin-3-yl)-4-hydroxyisoindoline-1,3-dione (35.6 mg, 0.130 mmol) and Cs₂CO₃ (50.8 mg, 0.156 mmol). The reaction mixture was stirred at room temperature for overnight. The reaction mixture was diluted with water (50 mL) and extracted with EtOAc (50 mL×2). The combined EtOAc layers were washed with water, dried with anhydrous Na₂SO₄ and concentrated. The residue was purified by column chromatography (0% to 100% acetonitrile in water) to give product as solid (57.5 mg, 56% yield). ¹H NMR (400 MHz, Chloroform-d) δ 9.08 (s, 1H), 8.21 (s, 1H), 7.65 (dd, J=8.5, 7.3 Hz, 1H), 7.43 (d, J=7.3 Hz, 1H), 7.38 (d, J=2.1 Hz, 1H), 7.21 (d, J=8.5 Hz, 1H), 7.14 (t, J=1.8 Hz, 1H), 7.09 (dd, J=9.1, 3.0 Hz, 1H), 7.03 (d, J=9.1 Hz, 1H), 6.95 (d, J=3.0 Hz, 1H), 6.73 (t, J=2.0 Hz, 1H), 4.95 (dd, J=12.1, 5.3 Hz, 1H), 4.18 (t, J=6.6 Hz, 2H), 3.99 (t, J=6.5 Hz, 2H), 3.82 (s, 3H), 3.76 (s, 3H), 2.92-2.65 (m, 3H), 2.51 (s, 3H), 2.11 (dq, J=10.7, 4.0, 3.2 Hz, 1H), 1.89 (p, J=6.8 Hz, 2H), 1.79 (p, J=6.6 Hz, 2H), 1.59-1.37 (m, 8H), 1.32 (s, 9H). ¹³C NMR (101 MHz, CDCl₃) δ 170.96, 168.09, 167.10, 165.66, 159.51, 159.36, 156.75, 153.70, 153.41, 148.02, 139.40, 138.54, 137.96, 136.46, 133.82, 124.32, 118.95, 117.50, 117.12, 115.65, 113.83, 113.34, 109.25, 109.03, 102.42, 67.95, 56.34, 56.02, 49.08, 34.89, 31.40, 31.27, 29.32, 29.27, 29.23, 29.13, 28.84, 25.97, 25.71, 22.63, 9.25. ESI-TOF HRMS: m/z 795.3727 (C₄₃H₅₀N₆O₉+H⁺ requires 795.3712). Representative corresponding mass spectra and HPLC traces are shown in FIG. 16A-C.

### xvii. N-(3-(tert-butyl)-5-(14-((2-(2,6-dioxopiperidin-3-yl)-1,3-dioxoisoindolin-4-yl)amino)-3,6,9,12-tetraoxatetradecanamido)phenyl)-1-(2,5-dimethoxyphenyl)-5-methyl-1H-1,2,3-triazole-4-carboxamide (M17)

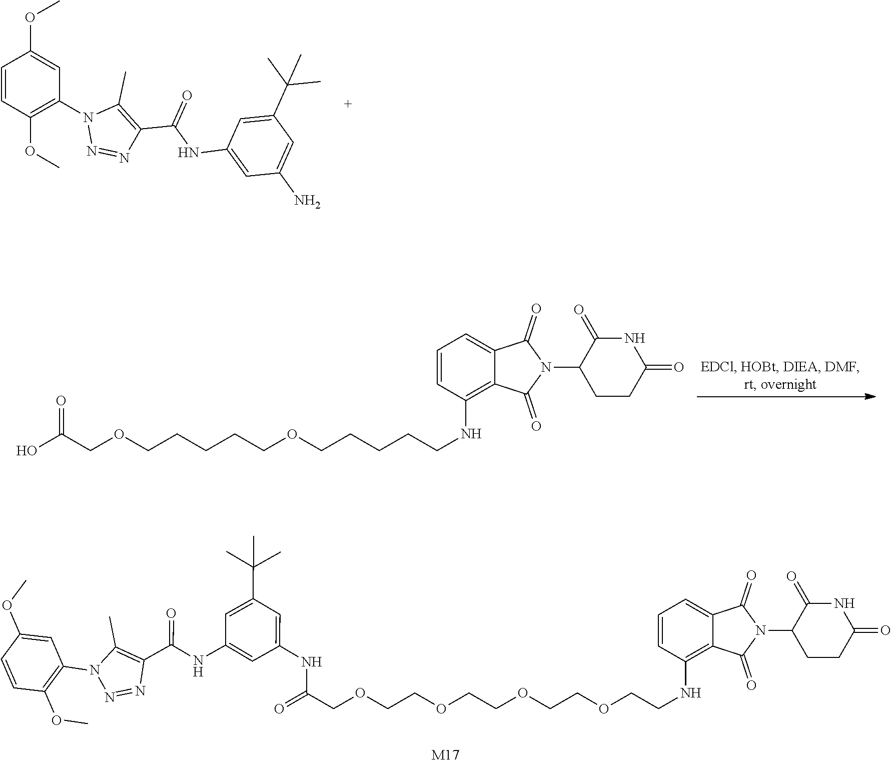 [SMILES: COc1ccc(OC)c(-n2nnc(C(=O)Nc3cc(N)cc(C(C)(C)C)c3)c2C)c1 ; O=C(O)COCCCCCOCCCCCNc1cccc2c1C(=O)N(C1CCC(=O)NC1=O)C2=O ; COc1ccc(OC)c(-n2nnc(C(=O)Nc3cc(NC(=O)COCCOCCOCCOCCNc4cccc5c4C(=O)N(C4CCC(=O)NC4=O)C5=O)cc(C(C)(C)C)c3)c2C)c1 ; "M17"]

To a solution of 14-((2-(2,6-dioxopiperidin-3-yl)-1,3-dioxoisoindolin-4-yl)amino)-3,6,9,12-tetraoxatetradecan-1-oic acid (25 mg, 0.049 mmol) in DMSO 2 mL was added N-(3-amino-5-(tert-butyl)phenyl)-1-(2,5-dimethoxyphenyl)-5-methyl-1H-1,2,3-triazole-4-carboxamide (20.17 mg, 0.049 mmol), HOBt, 80% (9.98 mg, 0.059 mmol) and EDCI (14.17 mg, 0.074 mmol), followed by N-ethyl-N-isopropylpropan-2-amine (6.37 mg, 0.049 mmol). The suspension was stirred at room temperature for overnight. The reaction mixture was then diluted with water (50 mL) and extracted with EtOAc (50 mL×2). The combined EtOAc layers were washed with water, dried with anhydrous Na₂SO₄ and concentrated. The residue was purified by silica gel chromatography (0% to 100% acetonitrile in water) to give product as yellow solid, 31.2 mg, 70% yield. ¹H NMR (400 MHz, Chloroform-d) δ 9.14 (s, 1H), 8.78 (s, 1H), 8.42 (s, 1H), 7.83 (t, J=1.9 Hz, 1H), 7.53 (dt, J=5.0, 1.8 Hz, 2H), 7.45 (dd, J=8.5, 7.1 Hz, 1H), 7.11-7.00 (m, 3H), 6.95 (d, J=2.9 Hz, 1H), 6.89-6.85 (m, 1H), 6.45 (s, 1H), 4.91 (dd, J=11.8, 5.4 Hz, 1H), 4.10 (s, 2H), 3.82 (s, 3H), 3.78-3.69 (m, 11H), 3.68-3.58 (m, 6H), 3.44-3.36 (m, 2H), 2.92-2.70 (m, 3H), 2.50 (s, 3H), 2.13-2.08 (m, 1H), 1.33 (s, 9H). ¹³C NMR (101 MHz, CDCl₃) δ 171.22, 169.25, 168.51, 168.31, 167.61, 159.42, 153.70, 153.12, 148.00, 146.82, 139.46, 138.03, 137.89, 137.73, 136.01, 132.49, 124.33, 117.50, 116.77, 113.81, 113.39, 113.34, 113.17, 111.57, 110.26, 108.93, 71.26, 70.69, 70.65, 70.63, 70.60, 70.58, 70.15, 69.43, 56.34, 56.03, 48.87, 42.36, 35.01, 31.41, 31.29, 22.79, 9.24.

ESI-TOF HRMS: m/z 899.3939 (C₄₅H₅₄N₈O₁₂+H⁺ requires 899.3934).

### xviii. N-(3-(tert-butyl)-5-(3-(1-(4-(2-((2-(2,6-dioxopiperidin-3-yl)-1,3-dioxoisoindolin-4-yl)oxy)acetamido)butyl)-1H-1,2,3-triazol-4-yl)propoxy)phenyl)-1-(2,5-dimethoxyphenyl)-5-methyl-1H-1,2,3-triazole-4-carboxamide (M18)

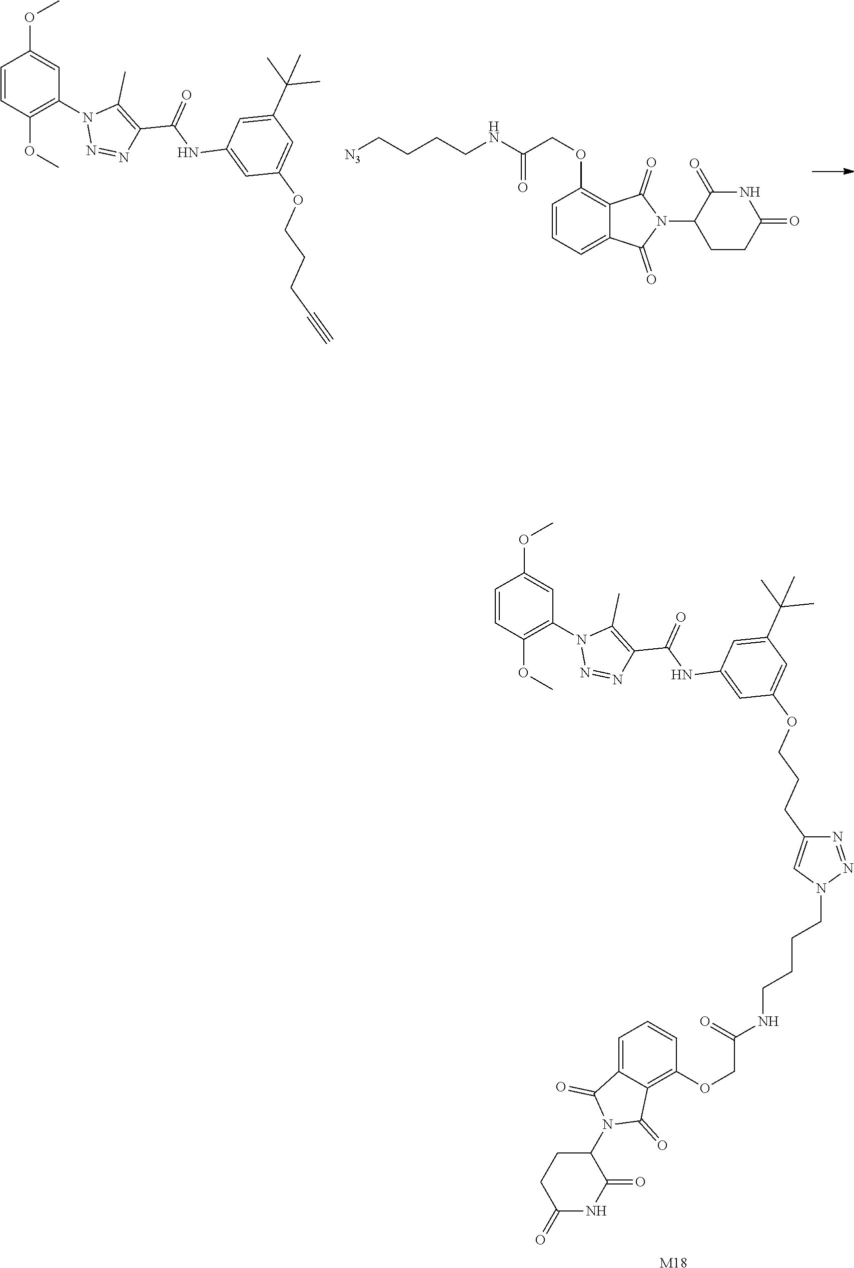 [SMILES: C#CCCCOc1cc(NC(=O)c2nnn(-c3cc(OC)ccc3OC)c2C)cc(C(C)(C)C)c1 ; [N-]=[N+]=NCCCCNC(=O)COc1cccc2c1C(=O)N(C1CCC(=O)NC1=O)C2=O ; COc1ccc(OC)c(-n2nnc(C(=O)Nc3cc(OCCCc4cn(CCCCNC(=O)COc5cccc6c5C(=O)N(C5CCC(=O)NC5=O)C6=O)nn4)cc(C(C)(C)C)c3)c2C)c1 ; "M18"]

N-(3-(tert-butyl)-5-(pent-4-yn-1-yloxy)phenyl)-1-(2,5-dimethoxyphenyl)-5-methyl-1H-1,2,3-triazole-4-carboxamide (55.8 mg, 0.117 mmol), copper sulfate pentahydrate (29.2 mg, 0.117 mmol), sodium ascorbate (232 mg, 1.170 mmol) and N-(4-azidobutyl)-2-((2-(2,6-dioxopiperidin-3-yl)-1,3-dioxoisoindolin-4-yl)amino)acetamide (50 mg, 0.117 mmol) were dissolved in 5 ml DMF. The reaction was stirred at room temperature under N2 protect for 12 hours. Then the solution filter and concentrated. The residue was purified to give product as a solid, 77.3 mg, 73%. ¹H NMR (400 MHz, Chloroform-d) δ 9.11 (s, 1H), 9.04 (s, 1H), 7.69 (dd, J=8.4, 7.4 Hz, 1H), 7.63 (t, J=5.8 Hz, 1H), 7.50 (d, J=7.3 Hz, 1H), 7.40-7.31 (m, 2H), 7.19-7.12 (m, 2H), 7.07 (dd, J=9.1, 3.0 Hz, 1H), 7.01 (d, J=9.1 Hz, 1H), 6.94 (d, J=3.0 Hz, 1H), 6.72 (t, J=2.0 Hz, 1H), 5.01 (dd, J=12.1, 5.6 Hz, 1H), 4.61 (d, J=1.4 Hz, 2H), 4.34 (t, J=7.1 Hz, 2H), 4.02 (t, J=6.1 Hz, 2H), 3.80 (s, 3H), 3.74 (s, 3H), 3.38 (dq, J=14.4, 6.8 Hz, 2H), 2.95-2.70 (m, 5H), 2.49 (s, 3H), 2.20-2.07 (m, 3H), 2.00-1.90 (m, 2H), 1.60 (p, J=6.9 Hz, 2H), 1.30 (s, 9H). ¹³C NMR (101 MHz, CDCl₃) δ 171.43, 168.43, 167.09, 166.62, 166.26, 159.39, 159.27, 154.59, 153.67, 153.47, 148.00, 147.31, 139.45, 138.55, 137.92, 137.06, 133.49, 124.27, 121.10, 120.01, 118.30, 117.51, 117.48, 113.85, 113.33, 109.49, 108.86, 102.69, 68.38, 66.83, 56.33, 56.02, 49.70, 49.37, 38.42, 34.89, 31.41, 31.26, 28.93, 27.64, 26.33, 22.63, 22.14, 9.24. ESI-TOF HRMS: m/z 905.3941 (C₄₆H₅₂N₁₀O₁₀+H⁺ requires 905.3974). Representative corresponding mass spectra and HPLC traces are shown in FIG. 18A-C.

### xix. N-(5-(tert-butyl)-2-((8-((2-(2,6-dioxopiperidin-3-yl)-1,3-dioxoisoindolin-4-yl)oxy)octyl)oxy)phenyl)-1-(2,5-dimethoxyphenyl)-5-methyl-1H-1,2,3-triazole-4-carboxamide (M19)

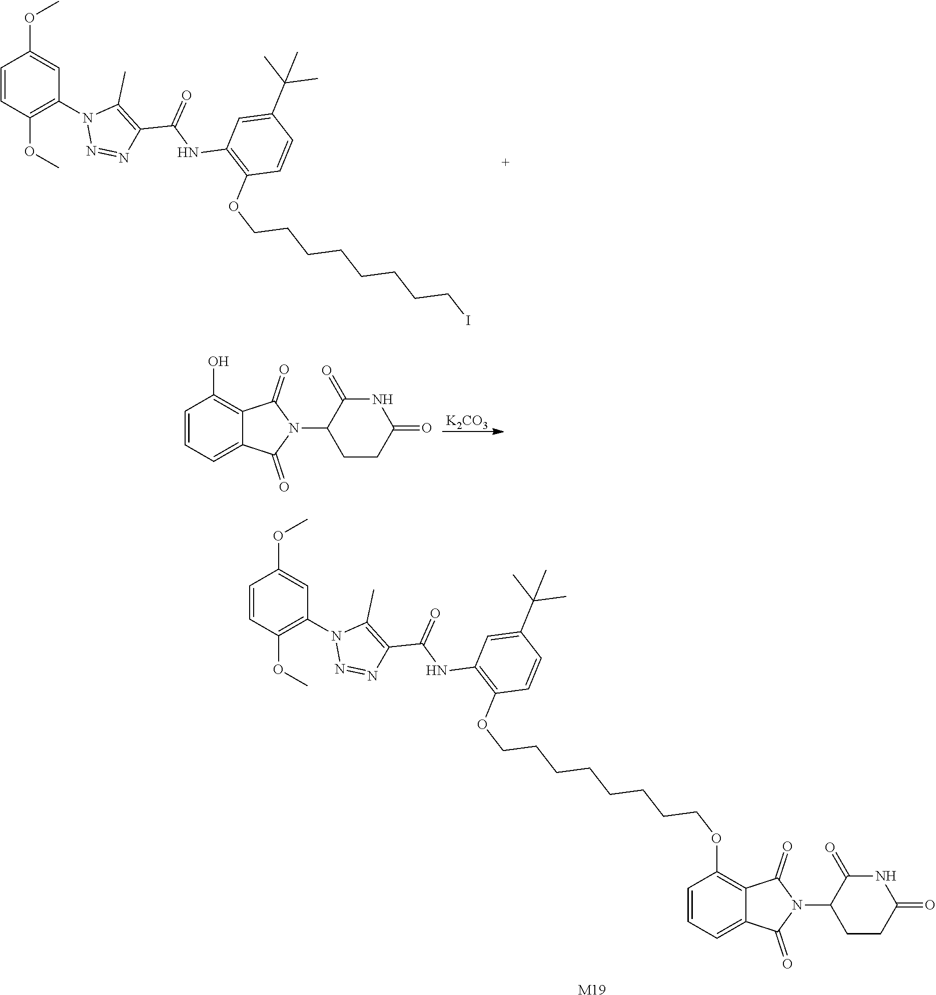

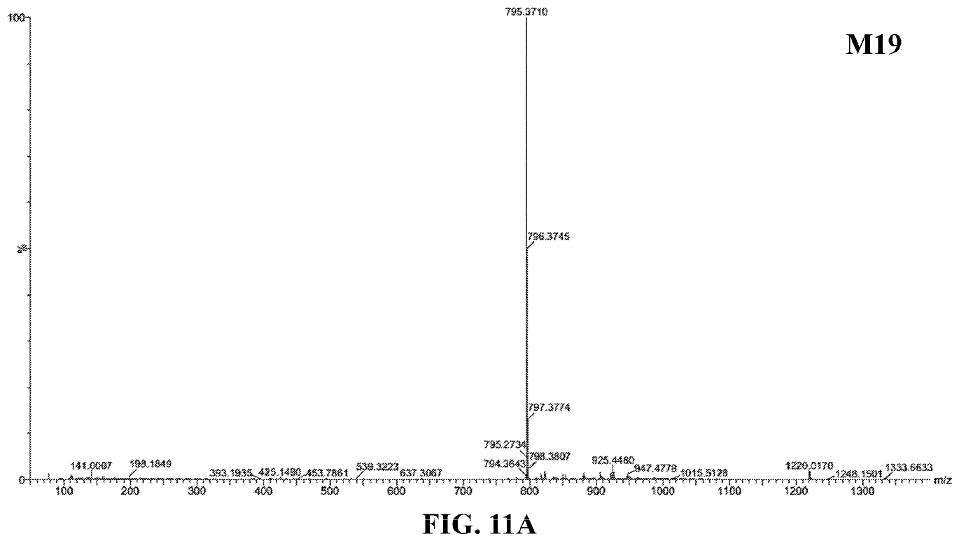

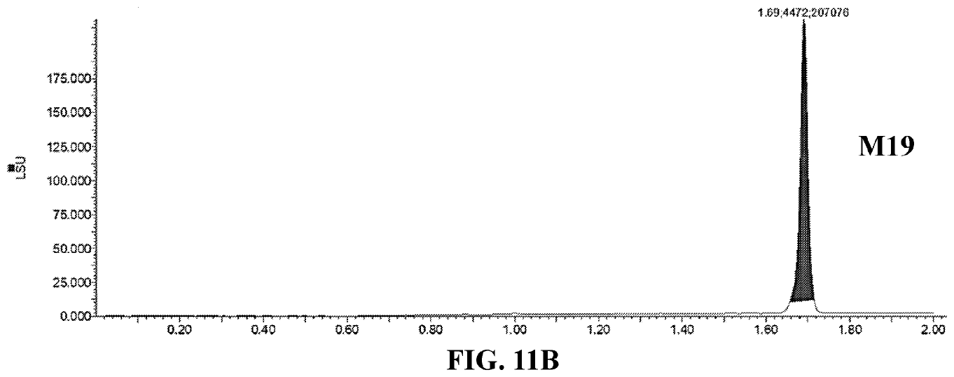

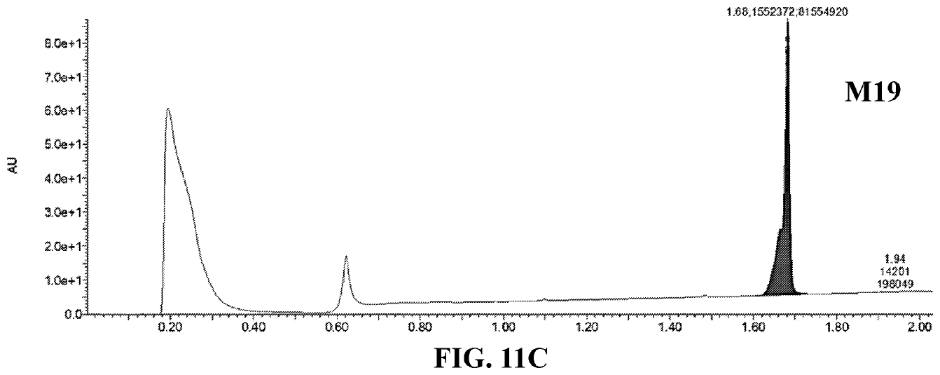

To a solution of 2-(2,6-dioxopiperidin-3-yl)-4-hydroxyisoindoline-1,3-dione (0.060 g, 0.220 mmol) in DMF 15 mL was added 2-(2,6-dioxopiperidin-3-yl)-4-hydroxyisoindoline-1,3-dione (0.060 g, 0.220 mmol), and N-(5-(tert-butyl)-2-((8-iodooctyl)oxy)phenyl)-1-(2,5-dimethoxyphenyl)-5-methyl-1H-1,2,3-triazole-4-carboxamide (0.130 g, 0.2 mmol). The suspension was stirred at room temperature for overnight. The reaction mixture was diluted with water (20 mL) and extracted with EtOAc (25 mL×2). The combined EtOAc layers were washed with water, dried with anhydrous Na₂SO₄ and concentrated. The residue was purified by reverse silica gel chromatography (0% to 100% acetonitrile in water) to give product as a solid (108.3 mg, 68% yield). ¹H NMR (400 MHz, DMSO-d₆) δ 11.09 (s, 1H), 9.65 (s, 1H), 8.45 (d, J=2.4 Hz, 1H), 7.73 (dd, J=8.5, 7.2 Hz, 1H), 7.41 (dd, J=7.9, 5.1 Hz, 2H), 7.26 (d, J=9.2 Hz, 1H), 7.19 (dd, J=9.1, 3.1 Hz, 1H), 7.13 (d, J=3.0 Hz, 1H), 7.09 (dd, J=8.6, 2.4 Hz, 1H), 7.02 (d, J=8.7 Hz, 1H), 5.06 (dd, J=12.8, 5.4 Hz, 1H), 4.16 (t, J=6.5 Hz, 2H), 4.10 (t, J=6.2 Hz, 2H), 3.75 (s, 3H), 3.74 (s, 3H), 2.87 (ddd, J=18.2, 13.9, 5.6 Hz, 1H), 2.62-2.53 (m, 2H), 2.40 (s, 3H), 2.03-1.95 (m, 1H), 1.77 (dp, J=26.6, 6.6 Hz, 4H), 1.60-1.50 (m, 2H), 1.50-1.43 (m, 2H), 1.43-1.34 (m, 4H), 1.29 (s, 9H). ¹³C NMR (101 MHz, CDCl₃) δ 170.80, 167.97, 167.12, 165.65, 159.41, 156.75, 153.64, 148.01, 145.68, 143.84, 139.11, 138.47, 136.41, 133.75, 127.54, 124.48, 120.10, 118.99, 117.27, 117.07, 117.02, 115.54, 113.85, 113.28, 110.60, 69.46, 68.75, 56.31, 55.98, 49.07, 34.45, 31.58, 31.40, 29.24, 29.18, 29.07, 28.80, 25.87, 25.58, 22.63, 9.29. ESI-TOF HRMS: m/z 795.3710 (C₄₃H₅₀N₆O₉+H⁺ requires 795.3712). Representative corresponding mass spectra and HPLC traces are shown in FIG. 11A-C.

### xx. N-(5-(tert-butyl)-2-((17-((2-(2,6-dioxopiperidin-3-yl)-1,3-dioxoisoindolin-4-yl)oxy)-3,6,9,12,15-pentaoxaheptadecyl)oxy)phenyl)-1-(2,5-dimethoxyphenyl)-5-methyl-1H-1,2,3-triazole-4-carboxamide (M20)

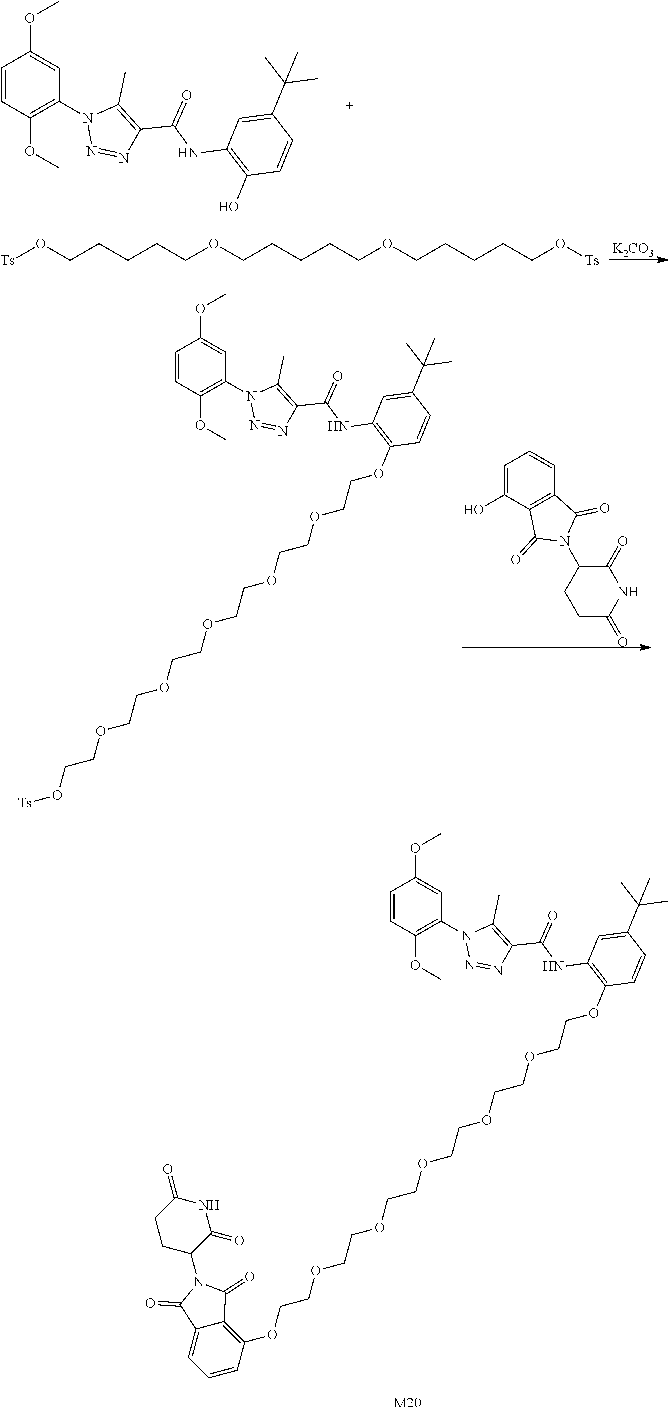

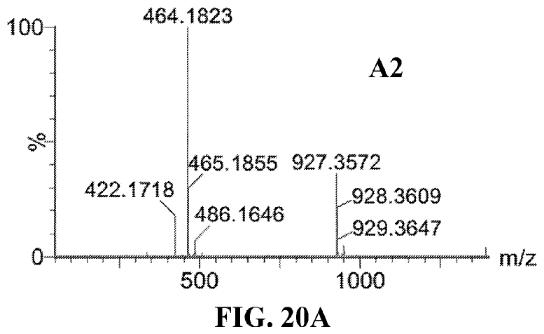

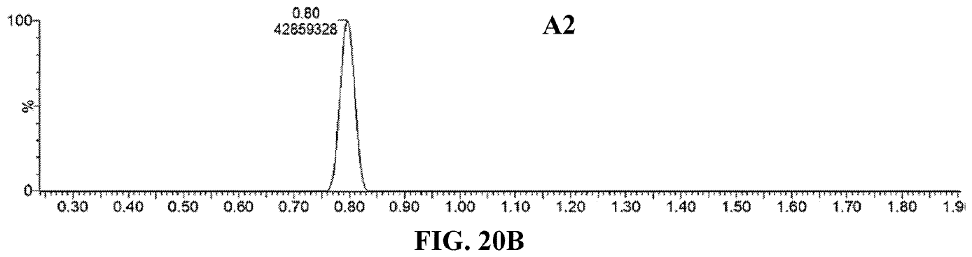

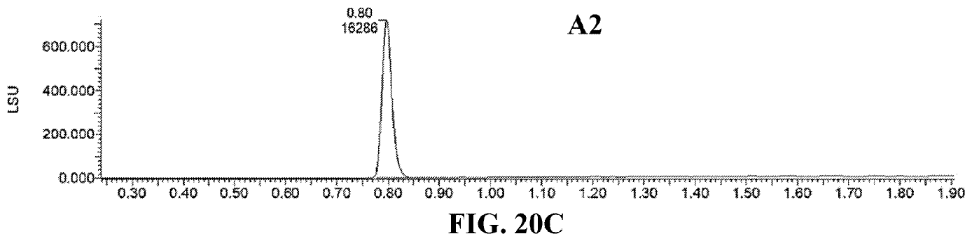

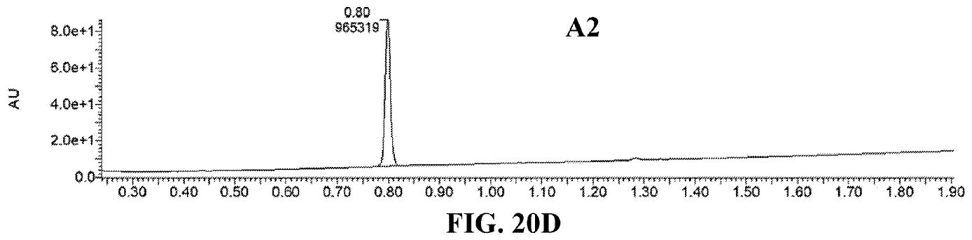

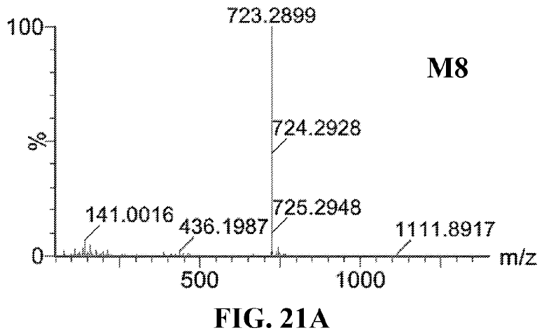

To a solution of N-(5-(tert-butyl)-2-hydroxyphenyl)-1-(2,5-dimethoxyphenyl)-5-methyl-1H-1,2,3-triazole-4-carboxamide (0.205 g, 0.5 mmol) in DMF 5 mL was added K₂CO₃ (0.207 g, 1.500 mmol) and 3,6,9,12,15-pentaoxaheptadecane-1,17-diyl bis(4-methylbenzenesulfonate) (0.295 g, 0.500 mmol). The suspension was stirred at 60° C. for overnight. Then the reaction mixture was cool down to room temperature and added 2-(2,6-dioxopiperidin-3-yl)-4-hydroxyisoindoline-1,3-dione (0.206 g, 0.750 mmol). The reaction mixture was stirred at room temperature for 6 hours. The reaction mixture was diluted with water (20 mL) and extracted with EtOAc (25 mL×2). The EtOAc layer was washed with water, dried with anhydrous Na₂SO₄ and concentrated. The residue was purified by reverse silica gel chromatography (0% to 100% acetonitrile in water) to give product as a solid (312.0 mg, 67% yield). ¹H NMR (400 MHz,) δ 9.75 (s, 1H), 8.62 (d, J=2.4 Hz, 1H), 8.16 (d, J=13.5 Hz, 1H), 7.65 (dd, J=8.5, 7.3 Hz, 1H), 7.44 (dd, J=7.3, 0.7 Hz, 1H), 7.26 (dd, J=8.5, 0.7 Hz, 1H), 7.11-7.04 (m, 2H), 7.02 (d, J=9.1 Hz, 1H), 6.95 (d, J=2.9 Hz, 1H), 6.87 (d, J=8.6 Hz, 1H), 4.96-4.88 (m, 1H), 4.33 (dd, J=5.7, 4.0 Hz, 2H), 4.23 (dd, J=5.6, 4.3 Hz, 2H), 3.97-3.88 (m, 4H), 3.84-3.80 (m, 5H), 3.74 (d, J=8.3 Hz, 5H), 3.71-3.67 (m, 2H), 3.65-3.59 (m, 10H), 2.92-2.65 (m, 3H), 2.52 (s, 3H), 2.14-2.07 (m, 1H), 1.35 (s, 9H). ¹³C NMR (101 MHz, CDCl₃) δ 170.88, 168.00, 166.99, 165.54, 159.42, 156.48, 153.68, 148.04, 145.51, 144.38, 139.13, 138.38, 136.46, 133.74, 127.67, 124.49, 120.23, 119.59, 117.36, 117.31, 117.28, 116.11, 113.85, 113.31, 111.23, 71.26, 71.14, 70.70, 70.61, 70.54, 70.50, 69.74, 69.37, 69.31, 68.95, 56.33, 56.01, 49.10, 34.48, 31.56, 31.39, 22.62, 9.28. ESI-TOF HRMS: m/z 931.4082 (C₄₇H₅₈N₆O₁₄+H⁺ requires 931.4084). Representative corresponding mass spectra and HPLC traces are shown in FIG. 20A-C.

### xxi. N-(2-(4-(3-(4-amino-1-oxoisoindolin-2-yl)-2,6-dioxopiperidin-1-yl)butoxy)-5-(tert-butyl)phenyl)-1-(2,5-dimethoxyphenyl)-5-methyl-1H-1,2,3-triazole-4-carboxamide (N1)

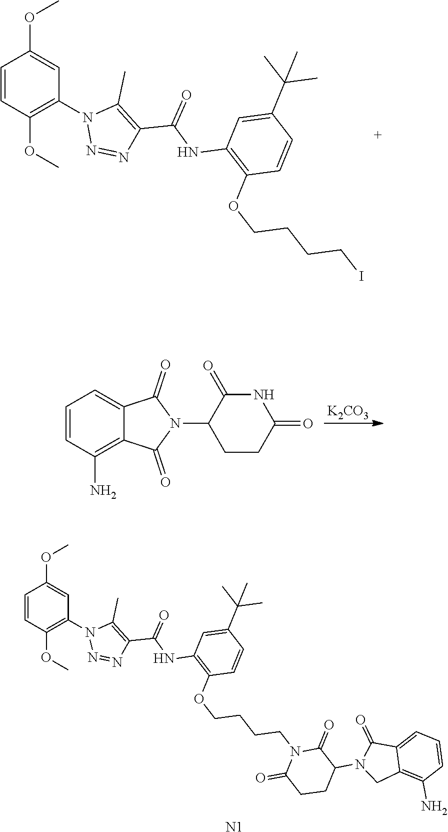

To a solution of N-(5-(tert-butyl)-2-(4-iodobutoxy)phenyl)-1-(2,5-dimethoxyphenyl)-5-methyl-1H-1,2,3-triazole-4-carboxamide (0.118 g, 0.2 mmol) in DMF 5 mL was added K₂CO₃ (0.041 g, 0.300 mmol) and 3-(4-amino-1-oxoisoindolin-2-yl)piperidine-2,6-dione (0.078 g, 0.300 mmol). The suspension was stirred at room temperature for overnight. The reaction mixture was diluted with water (25 mL). and extracted with EtOAc (25 mL×2). The combined EtOAc layers were washed by NaHCO₃ saturated solution, water, and brine. The organic phase dried by MgSO₄, filtered, and evaporated in vacuum. The residue was purified to providing N-(5-(tert-butyl)-2-(4-((2-(2,6-dioxopiperidin-3-yl)-1-oxoisoindolin-4-yl)amino)butoxy)phenyl)-1-(2,5-dimethoxyphenyl)-5-methyl-TH-1,2,3-triazole-4-carboxamide (79.8 mg, 55% yield) as a white solid. ¹H NMR (400 MHz, DMSO-d₆) δ 9.62 (s, 1H), 8.45 (d, J=2.3 Hz, 1H), 7.28 (d, J=9.2 Hz, 1H), 7.24-7.15 (m, 3H), 7.10 (dd, J=8.6, 2.4 Hz, 1H), 7.03 (d, J=8.6 Hz, 1H), 6.92 (d, J=7.3 Hz, 1H), 6.80 (d, J=7.9 Hz, 1H), 5.39 (s, 2H), 5.18 (dd, J=13.3, 5.1 Hz, 1H), 4.23-3.97 (m, 4H), 3.77 (s, 3H), 3.75 (s, 3H), 3.00 (ddd, J=18.2, 13.5, 5.4 Hz, 1H), 2.74 (dt, J=14.7, 2.7 Hz, 1H), 2.41 (s, 3H), 2.28 (qd, J=13.2, 4.3 Hz, 1H), 2.03 (q, J=4.4 Hz, 1H), 1.85-1.61 (m, 4H), 1.28 (s, 9H). ¹³C NMR (101 MHz, DMSO-d₆) δ 171.68, 170.69, 168.84, 158.45, 153.06, 147.61, 145.35, 143.59, 143.06, 138.88, 137.29, 132.20, 128.80, 126.81, 125.56, 123.59, 120.55, 117.49, 116.51, 116.43, 113.89(2C), 111.75, 110.45, 68.13, 56.30, 55.82, 52.06, 45.52, 34.02, 31.43, 31.29, 26.15, 24.11, 22.03, 8.73. ESI-TOF HRMS: m/z 724.3469 (C₃₉H₄₅N₇O₇+H⁺ requires 724.3453).

### xxii. N-(2-(2-(2-(2-(2-(3-(4-amino-1-oxoisoindolin-2-yl)-2,6-dioxopiperidin-1-yl)ethoxy)ethoxy)ethoxy)ethoxy)-5-(tert-butyl)phenyl)-1-(2,5-dimethoxyphenyl)-5-methyl-1H-1,2,3-triazole-4-carboxamide (N2)

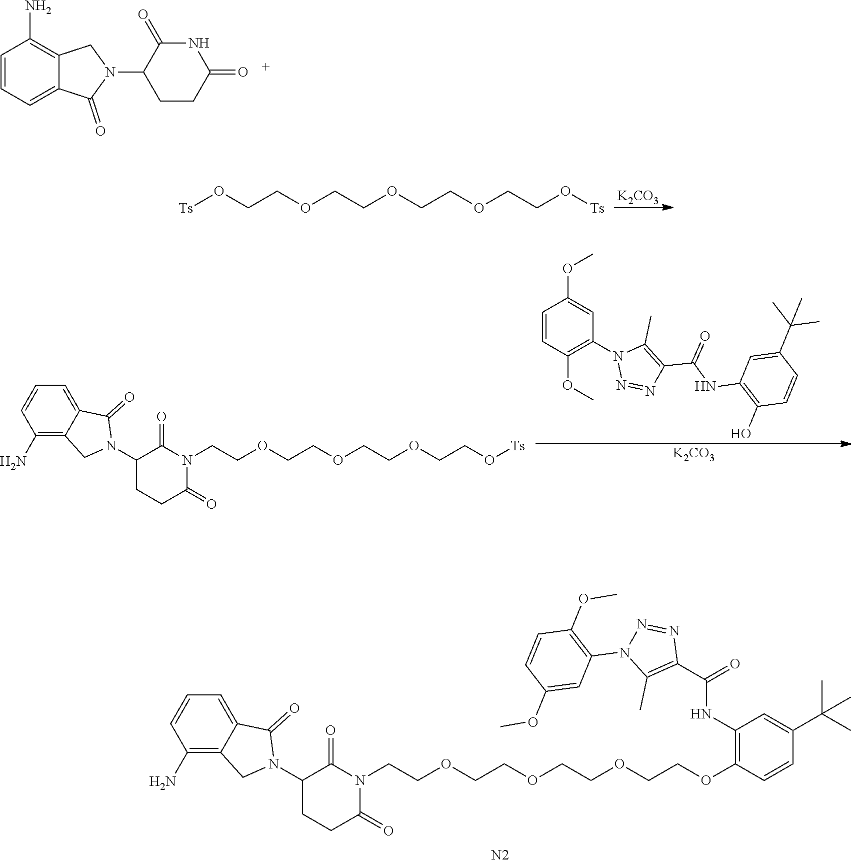

To a solution of 3-(4-amino-1-oxoisoindolin-2-yl)piperidine-2,6-dione (0.052 g, 0.2 mmol) in DMF 5 mL was added K₂CO₃ (0.033 g, 0.240 mmol) and ((oxybis(ethane-2,1-diyl))bis(oxy))bis(ethane-2,1-diyl) bis(4-methylbenzenesulfonate) (0.101 g, 0.200 mmol). The suspension was stirred at room temperature for overnight. Then N-(5-(tert-butyl)-2-hydroxyphenyl)-1-(2,5-dimethoxyphenyl)-5-methyl-1H-1,2,3-triazole-4-carboxamide (0.103 g, 0.25 mmol) and K₂CO₃ (0.041 g, 0.300 mmol) was added to the reaction mixture. The suspension was stirred at 80° C. for overnight. The reaction mixture was diluted with water and extracted with EtOAc (25 mL×2). The combined EtOAc layers were washed by NaHCO₃ saturated solution, water and brine. The organic phase was dried by MgSO₄, filtered and evaporated in vacuum. The residue was purified to providing N-(2-(2-(2-(2-(2-(3-(4-amino-1-oxoisoindolin-2-yl)-2,6-dioxopiperidin-1-yl)ethoxy)ethoxy)ethoxy)ethoxy)-5-(tert-butyl)phenyl)-1-(2,5-dimethoxyphenyl)-5-methyl-1H-1,2,3-triazole-4-carboxamide (65.3 mg, 39% yield) as a light-brown solid. ¹H NMR (500 MHz, DMSO-d₆) δ 9.63 (s, 1H), 8.44 (d, J=2.4 Hz, 1H), 7.28 (d, J=9.2 Hz, 1H), 7.23-7.16 (m, 2H), 7.15 (d, J=3.1 Hz, 1H), 7.09 (dd, J=8.6, 2.4 Hz, 1H), 7.04 (d, J=8.6 Hz, 1H), 6.91 (dd, J=7.3, 0.9 Hz, 1H), 6.79 (dd, J=7.9, 0.9 Hz, 1H), 5.40 (s, 2H), 5.16 (dd, J=13.4, 5.1 Hz, 1H), 4.25-4.17 (m, 3H), 4.05 (d, J=16.8 Hz, 1H), 3.86-3.71 (m, 10H), 3.67-3.64 (m, 2H), 3.55-3.51 (m, 2H), 3.47-3.43 (m, 4H), 3.39 (t, J=6.6 Hz, 2H), 2.98 (ddd, J=17.4, 13.6, 5.4 Hz, 1H), 2.74 (ddd, J=17.3, 4.5, 2.5 Hz, 1H), 2.40 (s, 3H), 2.30-2.21 (m, 1H), 2.02 (ddq, J=10.2, 5.3, 2.6 Hz, 1H), 1.28 (s, 9H). ¹³C NMR (126 MHz, DMSO-d₆) δ 172.12, 171.08, 169.35, 159.00, 153.54, 148.12, 145.84, 144.11, 143.82, 139.39, 137.73, 132.68, 129.32, 127.51, 126.07, 124.07, 121.07, 117.94, 117.06, 116.92, 114.44, 114.38, 112.78, 110.93, 70.61, 70.29, 70.11, 69.90, 69.42, 69.25, 67.21, 56.81, 56.34, 52.48, 45.93, 38.98, 34.55, 31.86, 31.80, 22.49, 9.24. ESI-TOF HRMS: m/z 828.3948 (C₄₃H₅₃N₇O₁₀+H⁺ requires 828.3927).

### xxiii. N-(2-((8-(3-(4-amino-1-oxoisoindolin-2-yl)-2,6-dioxopiperidin-1-yl)octyl)oxy)-5-(tert-butyl)phenyl)-1-(2,5-dimethoxyphenyl)-5-methyl-1H-1,2,3-triazole-4-carboxamide (N₃)

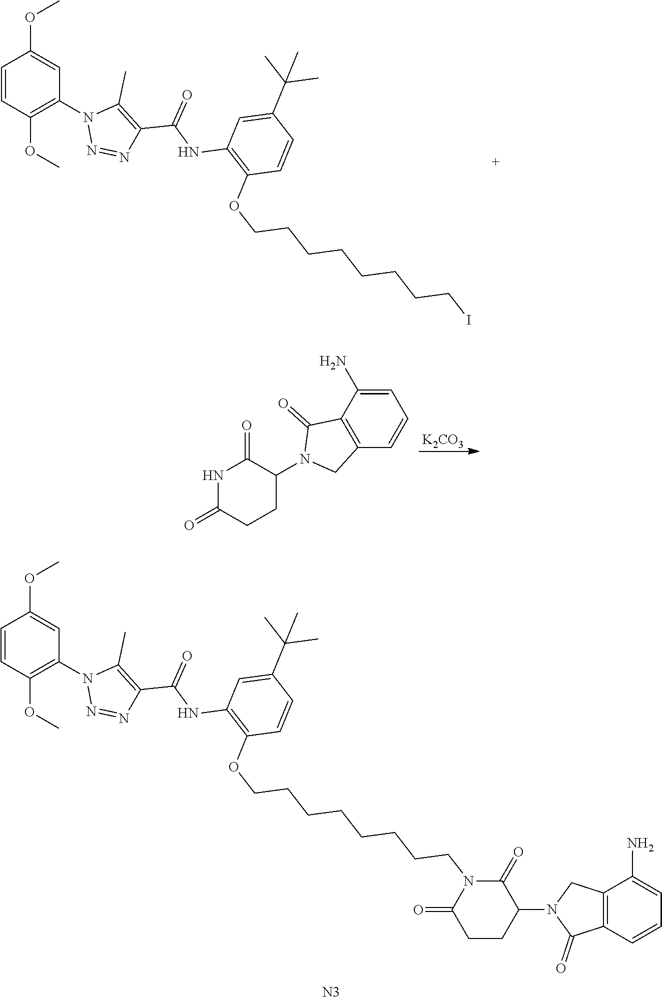

To a solution of 3-(7-amino-1-oxoisoindolin-2-yl)piperidine-2,6-dione (0.078 g, 0.300 mmol) in DMF 3 mL was added K₂CO₃ (0.028 g, 0.200 mmol) and N-(5-(tert-butyl)-2-((8-iodooctyl)oxy)phenyl)-1-(2,5-dimethoxyphenyl)-5-methyl-1H-1,2,3-triazole-4-carboxamide (0.130 g, 0.2 mmol). The suspension was stirred at room temperature for overnight. The solvent was diluted with water (25 mL). and extracted with EtOAc (25 mL×2). The combined EtOAc layers were washed by NaHCO₃ saturated solution, water and brine. The organic phase was dried by MgSO₄, filtered, evaporated in vacuum. The residue was purified to providing N-(2-((8-(3-(4-amino-1-oxoisoindolin-2-yl)-2,6-dioxopiperidin-1-yl)octyl)oxy)-5-(tert-butyl)phenyl)-1-(2,5-dimethoxyphenyl)-5-methyl-1H-1,2,3-triazole-4-carboxamide (75.3 mg, 48% yield) as a white solid. ¹H NMR (400 MHz, DMSO-d₆) δ 9.65 (s, 1H), 8.45 (d, J=2.4 Hz, 1H), 7.27 (d, J=9.2 Hz, 1H), 7.25-7.14 (m, 2H), 7.14 (d, J=3.0 Hz, 1H), 7.09 (dd, J=8.6, 2.4 Hz, 1H), 7.01 (d, J=8.7 Hz, 1H), 6.91 (dd, J=7.4, 0.9 Hz, 1H), 6.80 (dd, J=7.9, 0.9 Hz, 1H), 5.40 (s, 2H), 5.14 (dd, J=13.4, 5.1 Hz, 1H), 4.22-4.05 (m, 4H), 3.77 (s, 3H), 3.74 (s, 3H), 3.67-3.50 (m, 2H), 2.98 (ddd, J=17.4, 13.5, 5.4 Hz, 1H), 2.73 (ddd, J=17.2, 4.5, 2.5 Hz, 1H), 2.40 (s, 3H), 2.32-2.18 (m, 1H), 2.01 (ddd, J=12.7, 6.4, 3.9 Hz, 1H), 1.83-1.73 (m, 2H), 1.51 (p, J=7.2 Hz, 2H), 1.28 (s, 17H). ¹³C NMR (101 MHz, DMSO-d₆) δ 171.58, 170.49, 168.87, 158.41, 153.04, 147.61, 145.36, 143.59, 142.92, 138.85, 137.25, 132.21, 128.80, 126.74, 125.58, 123.58, 120.47, 117.45, 116.41, 116.29, 113.91, 113.88, 111.50, 110.43, 68.38, 56.30, 55.82, 52.05, 45.52, 34.01, 31.41, 31.30, 30.65, 28.69, 28.61(2C), 27.39, 26.26, 25.31, 22.05, 8.71. ESI-TOF HRMS: m/z 780.4079 (C₄₃H₅₃N₇O₇+H⁺ requires 780.4079).

### xxiv. N-(3-(4-(3-(4-amino-1-oxoisoindolin-2-yl)-2,6-dioxopiperidin-1-yl)butoxy)-5-(tert-butyl)phenyl)-1-(2,5-dimethoxyphenyl)-5-methyl-1H-1,2,3-triazole-4-carboxamide (N4)

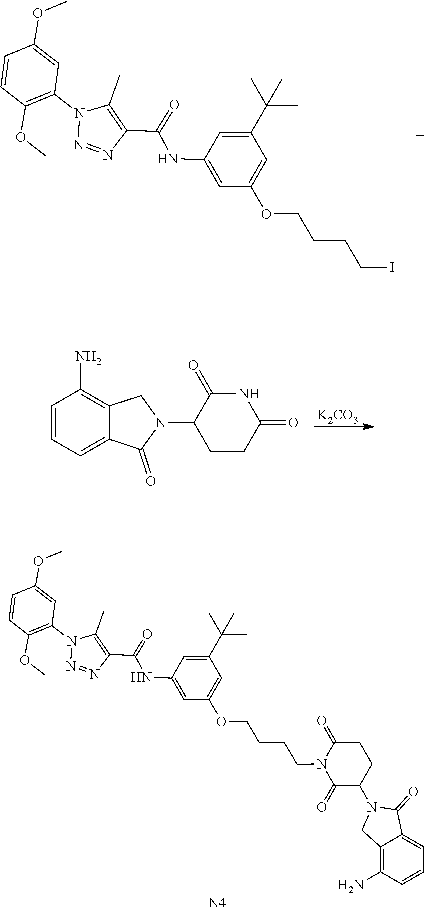

To a solution of N-(3-(tert-butyl)-5-(4-iodobutoxy)phenyl)-1-(2,5-dimethoxyphenyl)-5-methyl-1H-1,2,3-triazole-4-carboxamide (0.118 g, 0.2 mmol) in DMF 5 mL was added K₂CO₃ (0.055 g, 0.400 mmol) and 3-(4-amino-1-oxoisoindolin-2-yl)piperidine-2,6-dione (0.052 g, 0.200 mmol). Then stirred at room temperature for overnight. The reaction mixture was then diluted with water (20 mL) and extracted with EtOAc (25 mL×2). The EtOAc layer was washed with water, dried with anhydrous Na₂SO₄ and concentrated. The residue was purified by column chromatography (0% to 100% acetonitrile in water) to give product as a solid (77.1 mg, 53% yield). ¹H NMR (400 MHz, DMSO-d₆) δ 10.31 (s, 1H), 7.51 (t, J=1.7 Hz, 1H), 7.44 (t, J=2.0 Hz, 1H), 7.28 (d, J=9.2 Hz, 1H), 7.24-7.17 (m, 2H), 7.14 (d, J=3.0 Hz, 1H), 6.92 (dd, J=7.4, 0.9 Hz, 1H), 6.80 (dd, J=7.9, 1.0 Hz, 1H), 6.67-6.63 (m, 1H), 5.41 (s, 2H), 5.20 (dd, J=13.4, 5.1 Hz, 1H), 4.26-4.07 (m, 2H), 3.95 (t, J=6.0 Hz, 2H), 3.78 (s, 3H), 3.75 (s, 3H), 3.75-3.67 (m, OH), 3.03 (ddd, J=17.9, 13.6, 5.3 Hz, 1H), 2.83-2.74 (m, 1H), 2.38 (s, 3H), 2.35-2.24 (m, 1H), 2.11-2.02 (m, 1H), 1.76-1.57 (m, 4H), 1.26 (s, 9H). ¹³C NMR (101 MHz, CDCl₃) δ 171.16, 171.04, 169.99, 169.71, 159.38, 159.32, 153.71, 153.51, 148.02, 139.41, 138.49, 137.94, 132.49, 129.50, 124.31, 118.62, 117.51, 115.09, 113.83, 113.34, 109.42, 108.91, 102.67, 67.43, 56.34, 56.02, 52.53, 45.02, 40.32, 34.91, 32.19, 31.27, 26.84, 24.84, 22.84, 9.25. ESI-TOF HRMS: m/z 724.3456 (C₃₉H₄₅N₇O₇+H⁺ requires 724.3453).

### xxv. N-(3-((8-(3-(4-amino-1,3-dioxoisoindolin-2-yl)-2,6-dioxopiperidin-1-yl)octyl)oxy)-5-(tert-butyl)phenyl)-1-(2,5-dimethoxyphenyl)-5-methyl-1H-1,2,3-triazole-4-carboxamide (N5)

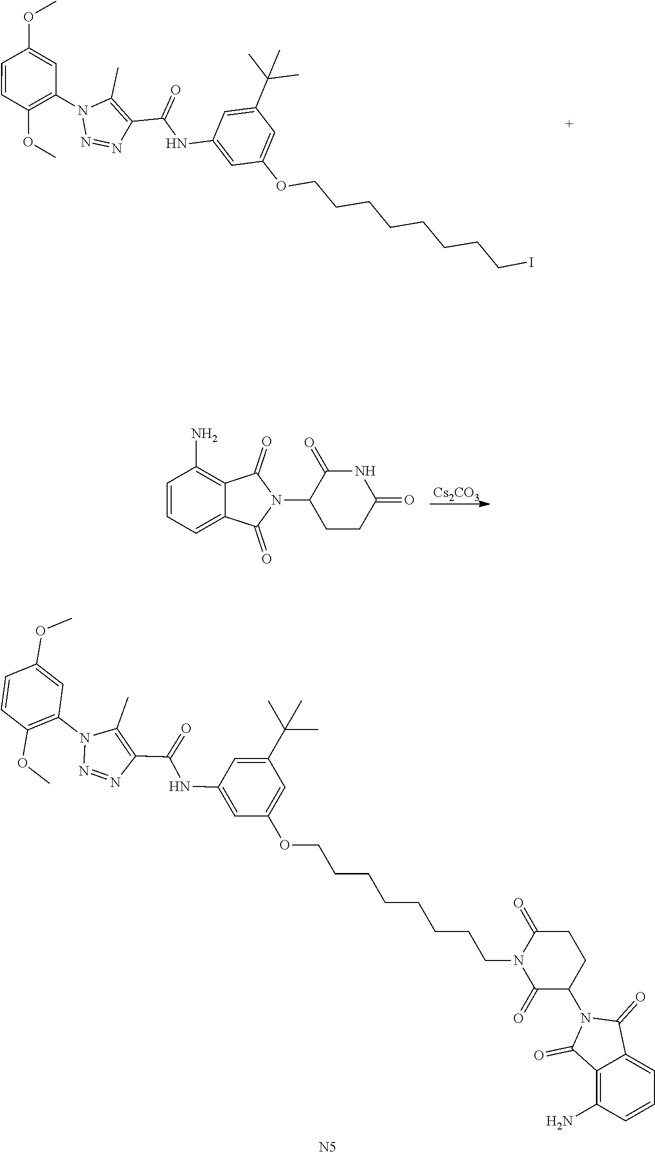

To a solution of N-(3-(tert-butyl)-5-((8-iodooctyl)oxy)phenyl)-1-(2,5-dimethoxyphenyl)-5-methyl-1H-1,2,3-triazole-4-carboxamide (84.20 mg, 0.130 mmol) in DMF 5 mL was added 3-(4-amino-1-oxoisoindolin-2-yl)piperidine-2,6-dione (33.7 mg, 0.130 mmol) and Cs₂CO₃ (50.8 mg, 0.156 mmol). Then stirred at room temperature for overnight. The reaction mixture was then diluted with water (50 mL) and extracted with EtOAc (50 mL×2). The combined EtOAc layers were washed with water and brine. The organic phase was dried with anhydrous Na₂SO₄ and concentrated. The residue was purified by silica gel chromatography (0% to 100% acetonitrile in water) to give product as a solid (77.2 mg, 75% yield). ¹H NMR (400 MHz, DMSO-d₆) δ 10.29 (s, 1H), 7.51 (t, J=1.7 Hz, 1H), 7.49-7.42 (m, 2H), 7.28 (d, J=9.2 Hz, 1H), 7.22 (dd, J=9.1, 3.1 Hz, 1H), 7.14 (d, J=3.0 Hz, 1H), 7.05-6.97 (m, 2H), 6.64 (t, J=2.0 Hz, 1H), 6.52 (s, 2H), 5.13 (dd, J=12.9, 5.4 Hz, 1H), 3.94 (t, J=6.5 Hz, 2H), 3.78 (s, 3H), 3.75 (s, 3H), 3.64 (td, J=7.2, 2.9 Hz, 2H), 2.97 (ddd, J=17.0, 14.0, 5.3 Hz, 1H), 2.79-2.69 (m, 1H), 2.59-2.51 (m, 1H), 2.38 (s, 3H), 2.06-1.98 (m, 1H), 1.72 (p, J=6.5 Hz, 2H), 1.43 (d, J=7.5 Hz, 4H), 1.37-1.29 (m, 6H), 1.26 (s, 9H). ¹³C NMR (101 MHz, DMSO-d₆) δ 171.56, 169.55, 168.53, 167.32, 159.39, 158.56, 153.06, 152.25, 147.67, 146.72, 139.31, 138.93, 137.58, 135.44, 131.97, 123.70, 121.69, 117.38, 113.97, 113.86, 110.96, 109.75, 108.50, 107.49, 103.29, 67.27, 56.30, 55.85, 49.07, 34.56, 31.16, 31.05, 30.65, 28.73, 28.68, 28.64, 27.32, 26.16, 25.47, 21.44, 8.79. ESI-TOF HRMS: m/z 794.3881 (C₄₃H₅₁N₇O₈+H⁺ requires 794.3872).

### xxvi. N-(3-((8-(3-(4-amino-1-oxoisoindolin-2-yl)-2,6-dioxopiperidin-1-yl)octyl)oxy)-5-(tert-butyl)phenyl)-1-(2,5-dimethoxyphenyl)-5-methyl-1H-1,2,3-triazole-4-carboxamide (N6)

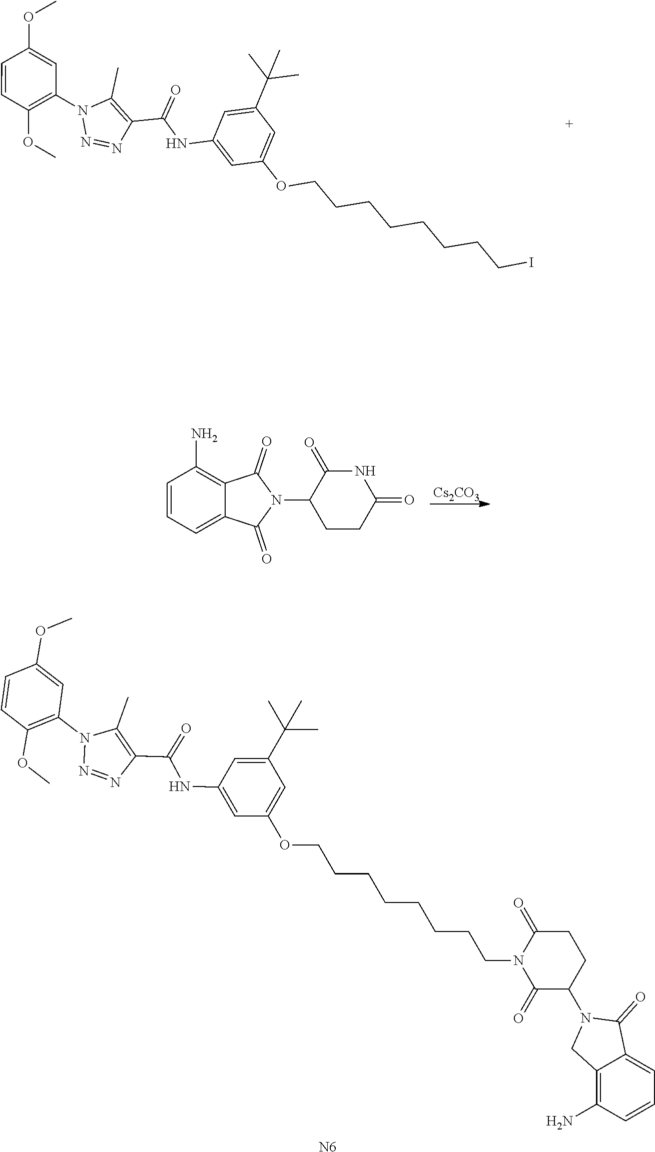

To a solution of N-(3-(tert-butyl)-5-((8-iodooctyl)oxy)phenyl)-1-(2,5-dimethoxyphenyl)-5-methyl-1H-1,2,3-triazole-4-carboxamide (84.20 mg, 0.130 mmol) in DMF 5 mL was added 4-amino-2-(2,6-dioxopiperidin-3-yl)isoindoline-1,3-dione (35.5 mg, 0.130 mmol) and Cs₂CO₃ (50.8 mg, 0.156 mmol). Then stirred at room temperature for overnight. The reaction mixture was then diluted with water (50 mL) and extracted with EtOAc (50 mL×2). The combined EtOAc layers were washed with water, dried with anhydrous Na₂SO₄ and concentrated. The residue was purified by silica gel chromatography (0% to 100% acetonitrile in water) to give product as a solid (55.9 mg, 55% yield). ¹H NMR (400 MHz, Chloroform-d) δ 9.07 (s, 1H), 7.38 (t, J=2.1 Hz, 1H), 7.36-7.33 (m, 1H), 7.29 (t, J=7.6 Hz, 1H), 7.14 (t, J=1.7 Hz, 1H), 7.09 (dd, J=9.1, 3.0 Hz, 1H), 7.03 (d, J=9.1 Hz, 1H), 6.95 (d, J=2.9 Hz, 1H), 6.90 (d, J=7.6 Hz, 1H), 6.73 (dd, J=2.3, 1.7 Hz, 1H), 5.19 (dd, J=13.4, 5.1 Hz, 1H), 4.39-4.15 (m, 2H), 3.99 (t, J=6.5 Hz, 2H), 3.82 (s, 3H), 3.79-3.74 (m, 5H), 2.97 (ddd, J=17.7, 4.8, 2.5 Hz, 1H), 2.84 (ddd, J=17.9, 13.3, 5.4 Hz, 1H), 2.51 (s, 3H), 2.28 (qd, J=13.2, 4.7 Hz, 1H), 2.16-2.12 (m, 1H), 1.77 (p, J=6.7 Hz, 2H), 1.57-1.42 (m, 4H), 1.39-1.28 (m, 15H). ¹³C NMR (101 MHz, CDCl₃) δ 170.99, 169.90, 169.74, 159.51, 159.35, 153.71, 153.43, 148.03, 140.45, 139.39, 138.50, 137.97, 132.49, 129.50, 126.71, 124.32, 118.49, 117.50, 114.96, 113.82, 113.34, 109.24, 109.06, 102.46, 67.97, 56.34, 56.02, 52.51, 44.98, 40.64, 34.89, 32.21, 31.28, 29.31, 29.24, 29.12, 27.95, 26.84, 25.98, 22.91, 9.26. ESI-TOF HRMS: m/z 780.4079 (C₄₃H₅₃N₇O₇+H⁺ requires 780.4079).

### xxvii. N-(3-((17-(3-(4-amino-1,3-dioxoisoindolin-2-yl)-2,6-dioxopiperidin-1-yl)-3,6,9,12,15-pentaoxaheptadecyl)oxy)-5-(tert-butyl)phenyl)-1-(2,5-dimethoxyphenyl)-5-methyl-1H-1,2,3-triazole-4-carboxamide (N7)

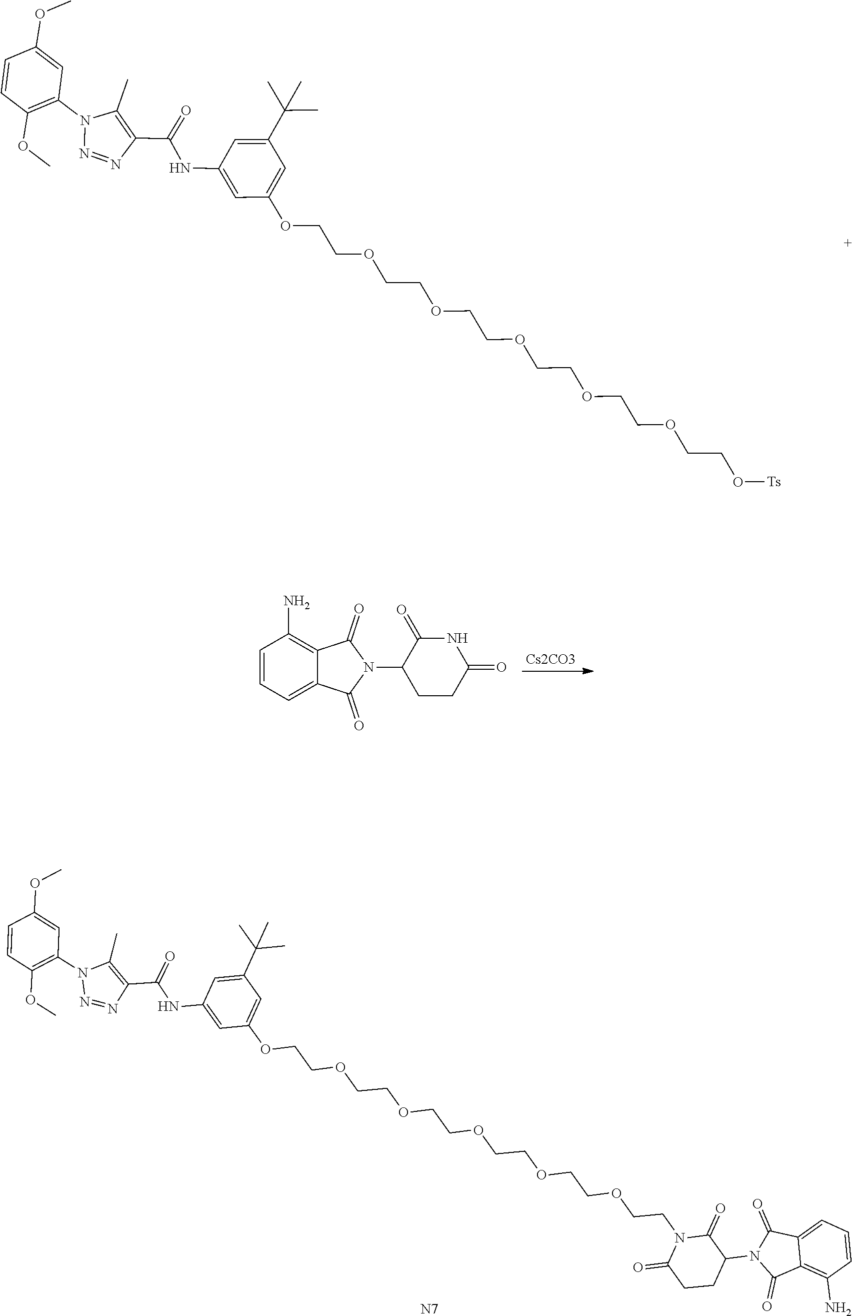

To a solution of 17-(3-(tert-butyl)-5-(1-(2,5-dimethoxyphenyl)-5-methyl-1H-1,2,3-triazole-4-carboxamido)phenoxy)-3,6,9,12,15-pentaoxaheptadecyl 4-methylbenzenesulfonate (60 mg, 0.072 mmol) in DMSO 5 mL was added 4-amino-2-(2,6-dioxopiperidin-3-yl)isoindoline-1,3-dione (39.6 mg, 0.145 mmol) and Cs₂CO₃ (28.3 mg, 0.087 mmol). Then the reaction was stirred at room temperature for overnight. The reaction mixture was then diluted with water (50 mL) and extracted with EtOAc (50 mL×2). The combined EtOAc layers were washed with water, dried with anhydrous Na₂SO₄ and concentrated. The residue was purified by silica gel chromatography (0% to 100% acetonitrile in water) to give product as a solid (45.9 mg, 68% yield). ¹H NMR (400 MHz, CDCl₃) δ 9.07 (s, 1H), 7.42-7.35 (m, 2H), 7.15 (t, J=1.7 Hz, 1H), 7.14-7.11 (m, 1H), 7.09 (dd, J=9.1, 3.0 Hz, 1H), 7.03 (d, J=9.1 Hz, 1H), 6.95 (d, J=2.9 Hz, 1H), 6.86 (dd, J=8.4, 0.7 Hz, 1H), 6.76 (dd, J=2.3, 1.6 Hz, 1H), 5.31 (s, 2H), 5.02-4.84 (m, 1H), 4.16 (dd, J=5.8, 3.9 Hz, 2H), 4.03 (tq, J=13.3, 6.4, 6.0 Hz, 2H), 3.86 (dd, J=5.7, 4.0 Hz, 2H), 3.82 (s, 3H), 3.76 (s, 3H), 3.75-3.71 (m, 2H), 3.70-3.67 (m, 2H), 3.67-3.58 (m, 14H), 3.00-2.88 (m, 1H), 2.80-2.71 (m, 2H), 2.51 (s, 3H), 2.13-2.02 (m, 1H), 1.31 (s, 9H). ¹³C NMR (101 MHz, CDCl₃) δ 171.04, 169.06, 168.85, 167.64, 159.34, 159.13, 153.70, 153.47, 148.03, 145.75, 139.42, 138.51, 137.95, 135.49, 132.39, 124.32, 121.42, 117.51, 113.82, 113.34, 113.01, 110.81, 109.55, 109.20, 102.55, 70.79, 70.63, 70.60, 70.57, 70.55, 70.07, 69.77, 67.48, 67.44, 56.34, 56.02, 49.73, 39.33, 34.91, 32.02, 31.26, 22.10, 9.26. ESI-TOF HRMS: m/z 930.4241 (C₄₃H₅₃N₇O₇+H⁺ requires 930.4244).

### xxviii. 4-(3-amino-5-(tert-butyl)phenoxy)-2-(2,6-dioxopiperidin-3-yl)isoindoline-1,3-dione (A1)

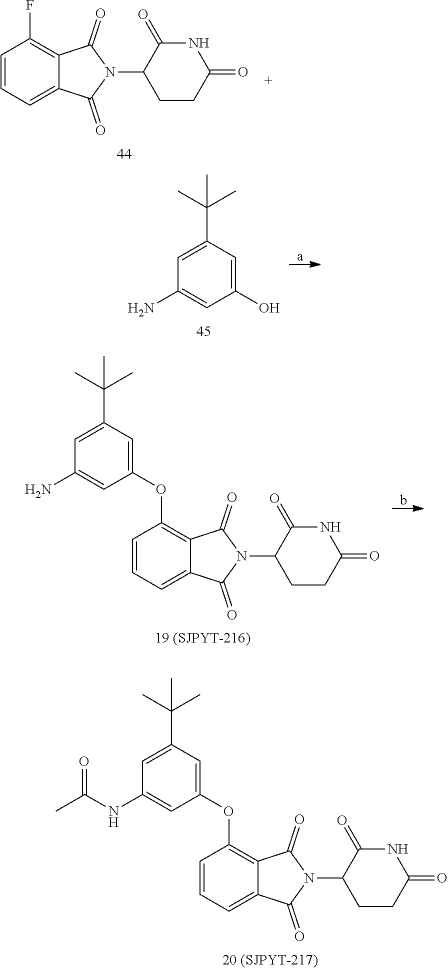 [SMILES: O=C1CCC(N2C(=O)c3cccc(F)c3C2=O)C(=O)N1 ; CC(C)(C)c1cc(N)cc(O)c1 ; CC(C)(C)c1cc(N)cc(Oc2cccc3c2C(=O)N(C2CCC(=O)NC2=O)C3=O)c1 ; CC(=O)Nc1cc(Oc2cccc3c2C(=O)N(C2CCC(=O)NC2=O)C3=O)cc(C(C)(C)C)c1]

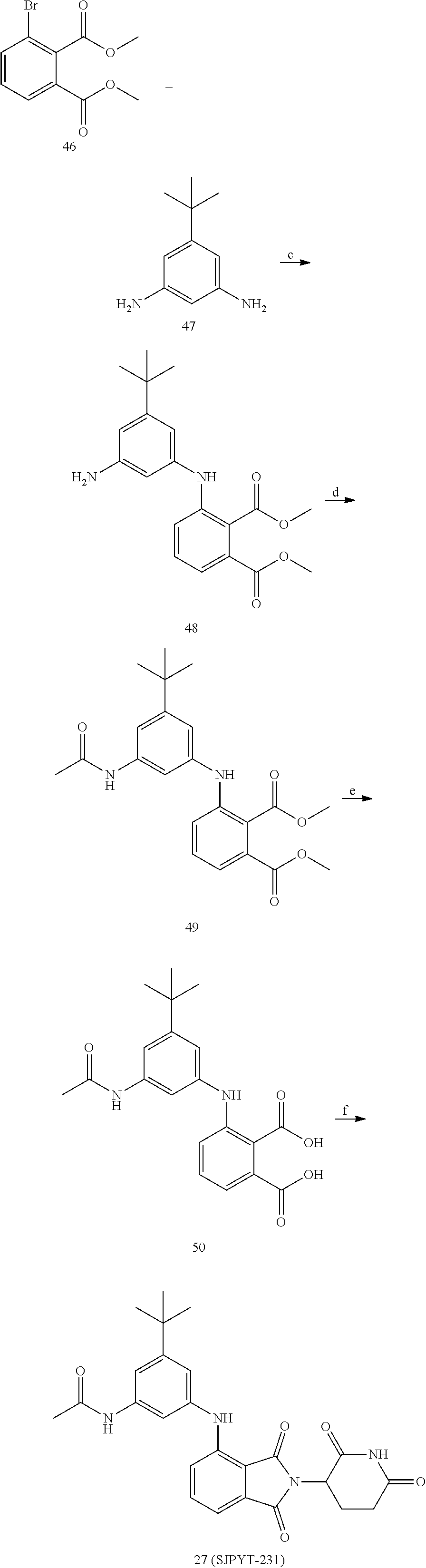

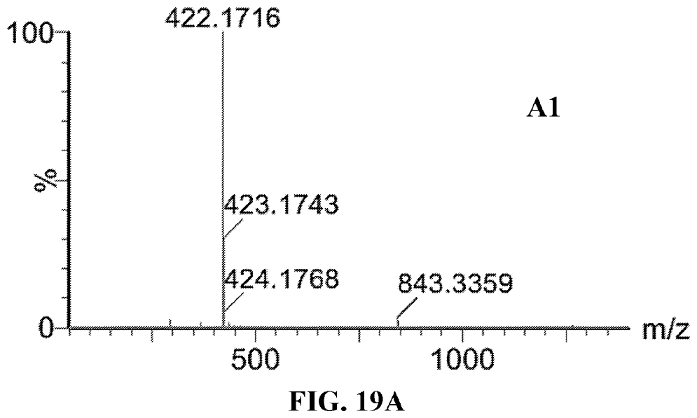

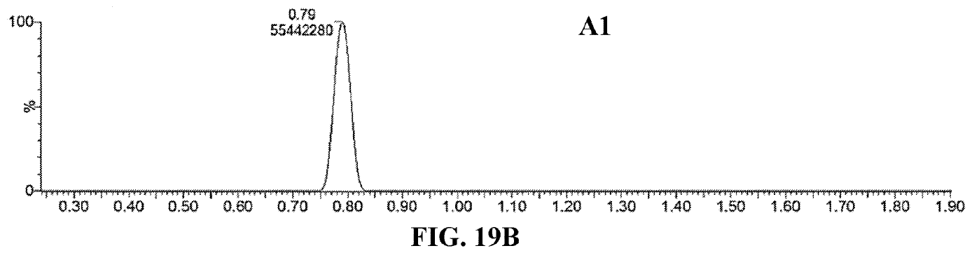

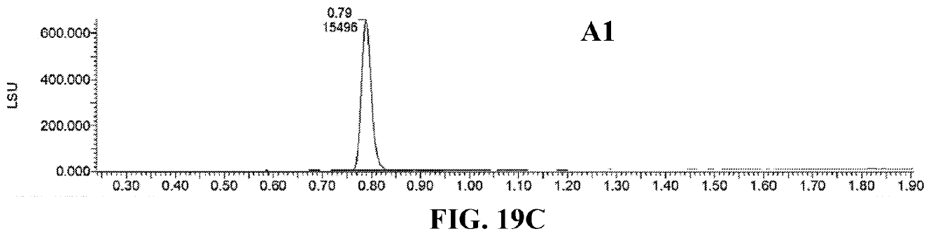

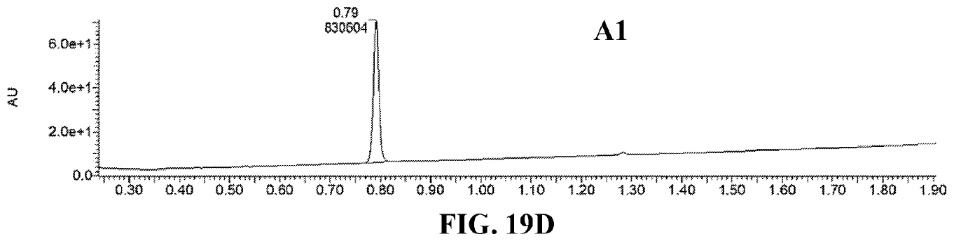

Cs₂CO₃ (2.359 g, 7.24 mmol) and 2-(2,6-dioxopiperidin-3-yl)-4-fluoroisoindoline-1,3-dione (1 g, 3.62 mmol) were added to a solution of 3-amino-5-(tert-butyl)phenol (1.196 g, 7.24 mmol) in DMSO (20 mL). The reaction mixture was stirred at 70° C. overnight. The reaction mixture was diluted with water (50 mL) and extracted with EtOAc (50 mL×2). The combined EtOAc layers were washed with water (50 mL), dried with anhydrous Na₂SO₄ and concentrated. The residue was purified by flash column chromatography (0% to 100% EtOAc in hexane) to give product as a light-yellow solid, 1.33 g, 87% yield, 100% purity. ¹H NMR (500 MHz, DMSO-d₆) δ 11.13 (s, 1H), 7.78 (dd, J=8.5, 7.3 Hz, 1H), 7.57 (d, J=7.2 Hz, 1H), 7.14 (d, J=8.5 Hz, 1H), 6.51 (t, J=1.8 Hz, 1H), 6.32 (t, J=1.9 Hz, 1H), 6.12 (t, J=2.1 Hz, 1H), 5.27 (s, 2H), 5.14 (dd, J=12.8, 5.4 Hz, 1H), 2.90 (ddd, J=16.8, 13.7, 5.4 Hz, 1H), 2.68-2.52 (m, 2H), 2.12-2.03 (m, 1H), 1.22 (s, 9H). ¹³C NMR (126 MHz, DMSO-d₆) δ 173.27, 170.38, 167.12, 165.51, 155.50, 155.15, 154.22, 150.79, 137.46, 133.83, 123.41, 118.17, 117.58, 108.47, 104.78, 102.58, 49.33, 34.88, 31.48, 31.42, 22.44. ESI-TOF HRMS: m/z 422.1716 (C₂₃H₂₃N₃O₅+H⁺ requires 422.1710). Representative corresponding mass spectra and HPLC traces are shown in FIG. 19A-D.

### xxix. N-(3-(tert-butyl)-5-((2-(2,6-dioxopiperidin-3-yl)-1,3-dioxoisoindolin-4-yl)amino)phenyl)acetamide (A2)

Acetic anhydride (242 mg, 2.373 mmol) was added to a solution of 4-(3-amino-5-(tert-butyl)phenoxy)-2-(2,6-dioxopiperidin-3-yl)isoindoline-1,3-dione (100 mg, 0.237 mmol) in DCM (2 mL). The reaction mixture was stirred at room temperature overnight, then filtered and the solid washed by cold DCM (2 mL). The solid was dried to obtain an off-white solid, 89.3 mg, 81% yield, 100% purity. ¹H NMR (500 MHz, DMSO-d₆) δ 11.13 (s, 1H), 7.78 (dd, J=8.5, 7.3 Hz, 1H), 7.57 (d, J=7.2 Hz, 1H), 7.14 (d, J=8.5 Hz, 1H), 6.51 (t, J=1.8 Hz, 1H), 6.32 (t, J=1.9 Hz, 1H), 6.12 (t, J=2.1 Hz, 1H), 5.27 (s, 2H), 5.14 (dd, J=12.8, 5.4 Hz, 1H), 2.90 (ddd, J=16.8, 13.7, 5.4 Hz, 1H), 2.68-2.52 (m, 2H), 2.12-2.03 (m, 1H), 1.22 (s, 9H). ¹³C NMR (126 MHz, DMSO-d₆) δ 173.27, 170.38, 167.12, 165.51, 155.50, 155.15, 154.22, 150.79, 137.46, 133.83, 123.41, 118.17, 117.58, 108.47, 104.78, 102.58, 49.33, 34.88, 31.48, 31.42, 22.44. ESI-TOF HRMS: m/z 464.1823 (C₂₅H₂₅N₃O₆+H⁺ requires 464.1816). Representative corresponding mass spectra and HPLC traces are shown in FIG. 20A-D.

### xxx. N-(3-(tert-butyl)-5-((2-(2,6-dioxopiperidin-3-yl)-1,3-dioxoisoindolin-4-yl)amino) phenyl)acetamide (A3)

Oxybis(2,1-phenylene))bis(diphenylphosphane) (0.197 g, 0.366 mmol), palladium diacetate (0.041 g, 0.183 mmol), Cs₂CO₃ (3.58 g, 10.99 mmol) and 5-(tert-butyl)benzene-1,3-diamine (2.406 g, 14.65 mmol) were added to a solution of dimethyl 3-bromophthalate (2 g, 7.32 mmol) in DMF (20 mL). The resulting mixture was heated to 100° C. with stirring under nitrogen overnight. The mixture was allowed to cool to room temperature and diluted with EtOAc (300 mL) and washed by water (150 mL×2), 1N HCl solution (150 mL) and brine (150 mL). The organic phase was dried by MgSO₄, filtered, and the filtrate was evaporated in vacuo. The residue was added Ac20 (10 mL). After being stirred at room temperature for 3 hours, the mixture was concentrated and diluted with Na₂CO₃ saturated solution (100 mL) and stirred at room temperature for 10 minutes. The mixture was extracted by EtOAc (100 mL). The organic phase was washed by water, dried by MgSO₄ and phase filtered. The filtrate was evaporated in vacuo. The residue was purified by flash column chromatography to give dimethyl 3-((3-acetamido-5-(tert-butyl)phenyl)amino)phthalate as a yellow solid, 1.4374 g, 49% yield. ¹H NMR (400 MHz, DMSO-d₆) δ 9.72 (s, 1H), 7.46-7.36 (m, 2H), 7.32 (s, 1H), 7.23 (dd, J=7.0, 1.5 Hz, 1H), 7.15 (t, J=1.8 Hz, 1H), 6.81 (t, J=1.9 Hz, 1H), 3.82 (s, 3H), 3.78 (s, 3H), 2.00 (s, 3H), 1.25 (s, 9H). ¹³C NMR (126 MHz, DMSO-d₆) δ 167.57, 166.84, 166.47, 151.53, 142.12, 141.53, 139.17, 131.12, 130.52, 120.80, 120.43, 119.80, 111.12, 109.23, 106.38, 51.96, 51.88, 33.84, 30.41, 23.44.

Lithium hydroxide (0.432 g, 18.04 mmol) was added to a solution of dimethyl 3-((3-acetamido-5-(tert-butyl)phenyl)amino)phthalate (1.4374 g, 3.61 mmol) in H₂O (10 mL) and acetone (20 mL). The resulting mixture was stirred at room temperature overnight. The reaction mixture was concentrated, 1N HCl (10 mL) was added, and extracted by EtOAc (100 mL). The organic phase was washed by water and concentrated under the reduced pressure. The residue was used without purification for the next step. 729 mg, 55% yield. ¹H NMR (400 MHz, DMSO-d₆) δ 12.89 (s, 2H), 9.71 (s, 1H), 7.92 (s, 1H), 7.38-7.35 (m, 2H), 7.31 (s, 1H), 7.16 (dd, J=5.9, 2.7 Hz, 1H), 7.14 (t, J=1.8 Hz, 1H), 6.82 (t, J=1.9 Hz, 1H), 2.00 (s, 3H), 1.25 (s, 9H). ¹³C NMR (126 MHz, DMSO-d₆) δ 168.36, 167.99, 167.56, 151.59, 141.80, 141.73, 139.19, 133.10, 129.97, 121.24, 120.00, 119.61, 110.70, 108.90, 105.71, 33.84, 30.43, 23.44.

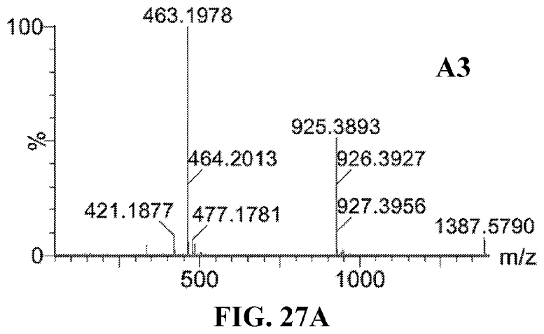

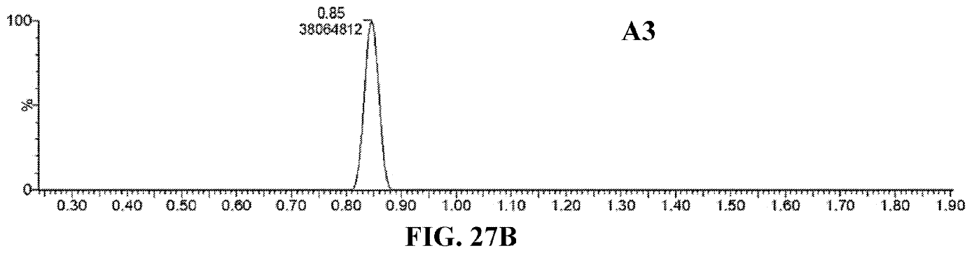

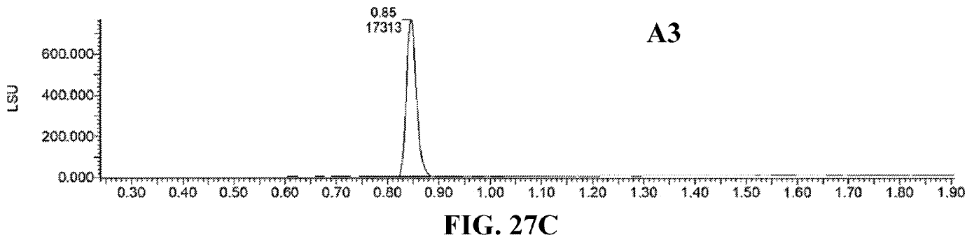

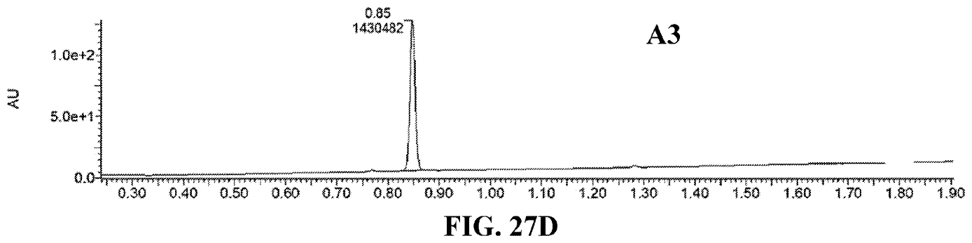

To a stirred solution of 3-((3-acetamido-5-(tert-butyl)phenyl)amino)phthalic acid (670 mg, 1.809 mmol), in pyridine (20 mL) 3-aminopiperidine-2,6-dione hydrochloride (298 mg, 1.809 mmol) was added. The resulting mixture was stirred at 118° C. overnight. Then the reaction mixture was cooled down and concentrated. The residue was diluted with water (50 mL) and extracted by EtOAc (50 mL). The organic phase was washed by water (50 mL), brine (50 mL) and concentrated. The residue was purified by flash column chromatography to give N-(3-(tert-butyl)-5-((2-(2,6-dioxopiperidin-3-yl)-1,3-dioxoisoindolin-4-yl)amino)phenyl)acetamide as a yellow solid, 386.6 mg, 46% yield, 100% purity. ¹H NMR (500 MHz, DMSO-d₆) δ 11.13 (s, 1H), 9.95 (s, 1H), 8.40 (s, 1H), 7.63 (dd, J=8.6, 7.1 Hz, 1H), 7.59 (t, J=1.9 Hz, 1H), 7.45 (d, J=8.5 Hz, 1H), 7.27 (t, J=1.8 Hz, 1H), 7.24 (d, J=7.0 Hz, 1H), 7.01 (t, J=1.8 Hz, 1H), 5.12 (dd, J=12.8, 5.4 Hz, 1H), 2.91 (ddd, J=16.6, 13.6, 5.2 Hz, 1H), 2.65-2.52 (m, 2H), 2.12-2.04 (m, 1H), 2.03 (s, 3H), 1.27 (s, 9H). ¹³C NMR (126 MHz, DMSO-d₆) δ 172.21, 169.40, 167.76, 167.66, 166.44, 151.92, 142.23, 139.24, 138.67, 135.54, 131.88, 118.88, 113.23, 112.71, 111.35, 111.08, 108.64, 48.08, 33.93, 30.38, 30.08, 23.45, 21.50. ESI-TOF HRMS: m/z 463.1978 (C₂₅H₂₆N₄O₅+H⁺ requires 463.1976). Representative corresponding mass spectra and HPLC traces are shown in FIG. 27A-D.

### 2. Biology Experimentals

### a. Cell Culture, Plasmids, and Cell Line Generation

HepG2/C3A cells were obtained from the American Type Culture Collection (ATCC, cat. #CRL-10741) and maintained in Eagle's Minimum Essential Medium (ATCC) with 10% FBS (HyClone). 293T/17 cells were obtained from ATCC (cat. #CRL-11268) and maintained in Dulbecco's Modified Eagle Medium (DMEM, ATCC) with 10% FBS. SNU—C4 cells were obtained from the Korean Cell Line Bank (KCLB, cat. #0000C4) and maintained in RPMI-1640 medium (ATCC) with 10% FBS. Cells were incubated in a humidified atmosphere at 37° C. with 5% CO₂ and routinely verified to be mycoplasma free by using the MycoProbe Mycoplasma Detection Kit (R&D Systems). Cell counts were obtained with a Countess II Automated Cell Counter using trypan blue staining. The “assay medium” used for relevant experiments below was phenol red-free DMEM supplemented with 5% charcoal/dextran-treated FBS (HyClone). Sf9 cells (Thermo Fisher Scientific) for baculoviral protein expression were propagated in Sf-900 III SFM with shaking at 27° C. without CO₂ or humidity.

Complete information for all plasmids is included in Table 1 below. SNU—C4 cells stably overexpressing GSPT1 or GSPT1^{G575N} were generated by lentiviral transduction and subsequent selection. Lentiviruses for pCDH-GSPT1 or pCDH-GSPT1^{G575N} were generated in 293T cells using psPAX2 and pMD2.G packaging vectors. The packaging vectors were gifts from Dr. Didier Trono (Addgene plasmid #12259 and 12260). SNU—C4 cells were transduced with lentiviral particles and selected with 5 μg/mL puromycin for 2 weeks.

TABLE 1

| Construct Name | Vector Backbone | Cloning | Notes |
|---|---|---|---|
| pcDNA3-HiBiT- | pcDNA3 (Thermo | Traditional PCR and | Mammalian |
| FKBPF36V-2xHA- | Fisher Scientific) | ligation | expression of PXR |
| PXR | | | fused to HiBiT tag, |
| | | | FKBPF36V, and 2x |
| | | | HA tags |
| pcDNA3-HA-CRBN | pcDNA3 (Thermo | Traditional PCR and | Mammalian |
| | Fisher Scientific) | ligation | expression of CRBN |
| | | | with N-terminal HA |
| | | | tag |
| pCDH-GSPT1 | pCDH-CMV-MCS- | Traditional PCR and | Lentiviral-based |
| | EF1α-GreenPuro | ligation | expression of |
| | (System | | GSPT1 (UniProt |
| | Biosciences) | | P15170-1) with N- |
| | | | terminal HA tag |
| pCDH- | pCDH-CMV-MCS- | Traditional PCR and | Lentiviral-based |
| GSPT1G575N | EF1α-GreenPuro | ligation | expression of |
| | (System | | GSPT1 (UniProt |
| | Biosciences) | | P15170-1) G575N |
| | | | mutant with N- |
| | | | terminal HA tag |
| | | | (G575 numbering is |
| | | | based on length of |
| | | | full-length protein |
| | | | (UniProt P15170-3) |
| pMD2.G | pMD2.G (Addgene | Addgene plasmid # | Lentiviral packaging |
| | plasmid # 12259) | 12259 | plasmid |
| psPAX2 | psPAX2 (Addgene | Addgene plasmid # | Lentiviral packaging |
| | plasmid # 12260) | 12260 | plasmid |
| pFastBacDual- | pFastBac Dual | GenScript gene | Baculoviral |
| 6xHis-CRBN/DDB1 | (Thermo Fisher | synthesis | production of His- |
| | Scientific) | | CRBN/DDB1 |
| | | | protein; contains 2 |
| | | | ORFs (His-CRBN |
| | | | and untagged |
| | | | DDB1) |

SNU—C4 3xFLAG-PXR KI cells were generated using CRISPR-Cas9 technology. Briefly, 400,000 SNU—C4 cells were transiently transfected with 100 pmol of chemically modified sgRNA (Synthego), 35 pmol SpCas9 protein (St. Jude Protein Production Facility), 3 μg of ssODN donor (Integrated DNA Technologies), and 500 ng of pMaxGFP (Lonza) via nucleofection (Lonza 4D-Nucleofector™ X-unit) using solution P3 and program EN158 in small cuvettes according to the manufacturer's recommended protocol. Cells were single cell sorted by FACS to enrich for GFP+(transfected) cells, clonally selected, and verified for the desired targeted modification via targeted deep sequencing as previously described. Briefly, cell pellets of approximately 10,000 cells were lysed and used to generate gene-specific amplicons with partial Illumina adapters in PCR #1. Amplicons were indexed in PCR #2 and pooled with targeted amplicons from other loci to create sequence diversity. Additionally, 10% PhiX Sequencing Control V3 (Illumina) was added to the pooled amplicon library prior to running the sample on a MiSeq Sequencer System (Illumina) to generate paired 2×250 bp reads. Samples were demultiplexed using the index sequences, fastq files were generated, and NGS analysis was performed using CRIS.py. Clones were identified for each modification and assessed in relevant assays. Editing construct sequences and relevant primers are listed in Table 2 below.

TABLE 2

| Oligonucleotide | Oligonucleotide Sequence |
|---|---|
| Name | (5′ to 3′) |
| SS124.NR112.g12 | UUUCUUUGGGUCUCACCUCC |
| sgRNA spacer | (SEQ ID NO: 1) |
| SS124.hNR112. | AGACTCTGTGTCCTCACAGTGTACAAAGTC |
| g12.3XFlag.anti. | AGCATGGTTCCAGCTTTCTTTGGGTCTCAC |
| SSODN | CTTGTCATCGTCATCCTTGTAATCGATGTC |
| | ATGATCTTTATAATCACCGTCATGGTCTTT |
| | GTAGTCCTCCAGGTTTGCTTCTGGGCCTCT |
| | TGGACTAGAAATGAGAAAACCACAGAGAAT |
| | GAAAAGA (SEQ ID NO: 2) |
| SS124.F | AGGTCAGCTCCCGAGTTCACAGGCC |
| | (SEQ ID NO: 3) |
| SS124.R | CAGTGGGTCAGTGGTGGCACGTGGG |
| | (SEQ ID NO: 4) |

### b. HIBIT ASSAYS

HepG2 cells (5×10⁶ cells/dish) were plated in tissue culture-treated 10 cm dishes in 10 mL EMEM with 10% FBS. The next day, cells were transfected with 5 μg pcDNA3-HiBiT-FKBP^{F36}V-2xHA-PXR and either 5 μg pcDNA3 vector or 5 μg pcDNA3-HA-CRBN using Lipofectamine 3000 (Thermo Fisher Scientific). After 24 h, cells were trypsinized, resuspended in assay media, and plated in white tissue culture-treated 384-well plates (1.5×10⁴ cells/well in 25 μL medium). An Echo 655T Acoustic Liquid Handler was used to dispense 125 nL/well of DMSO or stock compounds, resulting in 0.5% final DMSO concentration. The plates were incubated for 24 h, and the Nano-Glo HiBiT Lytic Detection System (Promega) and an EnVision microplate reader were used to measure HiBiT signal. Results are reported as fold change relative to DMSO control cells.

### c. Western Blotting

For experiments with HepG2 cells, cells were plated in tissue culture-treated six-well plates (7.5×10⁵ cells/well in 2 mL EMEM with 10% FBS). The next day, cells were transfected with 1.5 μg pcDNA3-HiBiT-FKBP^{F36}V-2xHA-PXR and either 1.5 μg pcDNA3 vector or 1.5 μg pcDNA3-HA-CRBN using Lipofectamine 3000. After 24 h, the media was removed, cells were washed with DPBS, and assay media containing 0.5% DMSO or compound was added. After 24 h, cells were washed with DPBS, trypsinized, pelleted by centrifugation, and lysed in 50 μL radioimmunoprecipitation assay (RIPA) buffer [50 mM Tris (pH 8.0), 150 mM NaCl, 1% NP-40, 0.5% sodium deoxycholate, 0.1% SDS] supplemented with Halt Protease Inhibitor Cocktail (Thermo Fisher Scientific). Protein in the lysate was quantified with the Pierce BCA Protein Assay Kit (Thermo Fisher Scientific), and 40 μg was diluted with NuPAGE LDS Sample Buffer (Thermo Fisher Scientific), heated at 95° C. for 5 min, and loaded into NuPAGE 4-12% Bis-Tris gels (Thermo Fisher Scientific). Separated proteins were transferred to nitrocellulose membranes using the iBlot 2 Dry Blotting System (Thermo Fisher Scientific). Membranes were blocked with TBST [50 mM Tris (pH 7.4), 150 mM NaCl, 0.1% Tween 20] containing 5% milk for 1 h at room temperature. Antibodies against HA (Sigma-Aldrich, cat. #H6908, 1:1,000 dilution) and 0-Actin (Sigma-Aldrich, cat. #A5441, 1:1,000 dilution) were bound overnight at 4° C. in TBST containing 5% milk. Membranes were washed with TBST three times for 10 min each, and IRDye 800CW Goat anti-Mouse IgG Secondary Antibody (LI-COR, cat. #926-32210, 1:10,000 dilution) and IRDye 680LT Goat anti-Rabbit IgG Secondary Antibody (LI-COR, cat. #926-68021, 1:10,000 dilution) were added in TBST containing 5% milk for 1 h at room temperature. Membranes were washed as above and imaged with an Odyssey CLx imaging system (LI-COR). Bands were quantified with Image Studio Lite Software (LI-COR).

For experiments with SNU—C4 3xFLAG-PXR KI cells, cells were plated in tissue culture-treated six-well plates (1×10⁶ cells/well in 2 mL assay media). The following day, DMSO or compounds were added to result in 0.5% DMSO and the indicated compound concentrations, and cells were incubated for the indicated time points. Cells were lysed and subjected to western blotting as above using antibodies against FLAG (Sigma-Aldrich, cat. #F3165, 1:2,000 dilution), GSPT1 (Abcam, cat. #ab126090, 1:4,000 dilution), and R-Actin (Cell Signaling Technology, cat. #4967, 1:2,000 dilution).

### d. TMT-MS

SNU—C4 3xFLAG-PXR KI cells were plated in tissue culture-treated 10 cm dishes (6×10⁶ cells/dish in 6 mL assay media). The next day, 0.5% DMSO or 5 μM SJPYT-195 was added to triplicate dishes. After 12 h, the media were removed, cells were washed twice with ice cold PBS, 500 μL of 50 mM HEPES (pH 8.0) and 8 M urea was added to each dish, cells were scraped and transferred to 1.5 mL tubes, and the tubes were frozen on dry ice and stored at −80° C. Processing for TMT-MS was conducted according to a previously optimized protocol. To profile the whole proteome, the cell lysates (approximately 100 μg of protein per sample) were proteolyzed with Lys-C(Wako) at an enzyme-to-substrate ratio of 1:100 (w/w) for 3 h at room temperature. Samples were diluted with 50 mM HEPES (pH 8.5) to a final 2 M urea concentration. Disulfide bonds were reduced with DTT (5 mM and 45 min incubation at room temperature) and alkylated with iodoacetamide (20 mM and 45 min incubation at room temperature in the dark. Proteins were further digested with trypsin (Promega) at an enzyme-to-substrate ratio of 1:100 (w/w) for 3 h at room temperature. The digestion was stopped by adding FA to 0.5%, and samples were centrifuged at 21,000×g at 4° C. to remove insoluble material, desalted on tC18 SepPak solid-phase extraction cartridges (Waters Corporation), dried by SpeedVac, and stored at −80° C.

Dried peptides were resuspended in 50 μL 100 mM HEPES (pH 8.5) and labeled with TMTpro 16plex Label Reagents [Thermo Fisher Scientific, 10 μg/μL in 100% ACN, 1:2 (w/w) ratio, room temperature for 1 h]. Reactions were quenched by addition of hydroxylamine to 0.3% (v/v), and labeled peptides were combined equally, acidified with FA (final 1%, (v/v)), desalted on tC18 SepPak solid-phase extraction cartridges, and dried by SpeedVac. High pH reversed-phase fractionation was performed on a Xbridge 4.6 mm×250 mm column (Waters Corporation) with an Agilent 1200 HPLC system. Peptides were reconstituted in Buffer A [10 mM ammonium formate (pH 8)], and fractionated using a 180 mn gradient of 15%-50% Buffer B [90% ACN, 10 mM ammonium formate (pH8)] collected into 90 concatenated fractions. Fractions were dried in SpeedVac and resuspended in 5 μL 5% FA/1% trifluoroacetic acid immediately prior to mass spectrometry and quantitation analysis.

A QE-HF (Thermo Fisher Scientific) mass spectrometer connected in-line to a Dionex Ultimate 3000 ultra-high performance liquid chromatography (UHPLC) system was used for LC-MS/MS analysis. Peptides were separated on a CoAnn 75 μm×15 cm 1.9 μm C18 column (at 50° C.) using a 60 min gradient of 12%-56% Buffer B [67% ACN/2.5% DMSO/0.1% FA] at an optimal ˜0.250 mL/min flow rate. The mass spectrometer was operated in “high-high” data-dependent mode with a survey scan in Orbitrap (60,000 resolution, scan range 450-1600 m/z, 1×10⁶ AGC target, −50 ms maximal ion time), followed by 20 data-dependent MS2 scans in Orbitrap (60,000 resolution, scan range starting from 120 m/z, 1×10⁵ AGC target, ˜105 ms maximal ion time, 32 HCD normalized collision energy, 1.0 m z isolation window, and 15 s dynamic exclusion, charge state screening enabled to reject precursor charge states that were unassigned, +1, or >+4).

MS raw files were converted to mzXML files, and database searches were performed against Uniprot HUMAN protein database using our in-house developed JUMP pipeline. Search parameters included precursor ions mass tolerance 20 ppm and fragment ions 0.02 Da, fully tryptic with maximal 2 missed cleavages, and maximal 3 modification sites per peptide. TMT16 modification of Lys residues and of N termini (+304.20715 Da) and carbamidomethylation of Cys residues (+57.02146 Da) were set as static modifications while Met oxidation (+15.99492 Da) was set as dynamic modifications. Peptide-spectrum match (PSM) filtering was performed based on precursor ion mass accuracy, and then grouped by peptide length, tryptic ends, modifications, mis-cleavage sites, and precursor ion charge state as parameters to reduce false discovery rate (FDR) below 1% for peptides, based on the target-decoy strategy. TMT-based quantification and statistical analysis were performed based on our previous method. TMT quantification data quality was evaluated by comparing positive control protein intensities with western blot results and by utilizing statistical methods (PCA or unsupervised clustering) to determine outliers or sample mislabeling. Protein fold change and p values of different comparisons were calculated based on protein intensities. Differential expression analysis was performed based on our previous method, by applying different combinations of cutoff threshold [i.e., FDR (Benjamini-Hochberg corrected p)<0.05] based on the positive protein list (proteins known to be dysregulated in the comparisons).

### e. Cytotoxicity Assays

An Echo 655T Acoustic Liquid Handler was used to dispense 125 nL/well of DMSO or stock compounds into white tissue culture-treated 384-well plates. The indicated cells (2.5×10³/well in 40 μL RPMI with 10% FBS) were plated in the 384-well plates, resulting in 0.5% DMSO and the indicated concentrations of chemicals. After 72 h compound treatment, the CellTiter-Glo Luminescent Cell Viability Assay (Promega) and an EnVision microplate reader were used to assess cytotoxicity. Wells containing DMSO alone or 10 μM staurosporine served as negative and positive controls, respectively. The percent cell viability for each well was calculated using equation 1:

$$\% Cell Viability = 100 \% \times \frac{( {Signal}_{Compound} - {Signal}_{Staurosporine} )}{( {Signal}_{DMSO} - {Signal}_{Staurosporine} )}$$

### f. Time-Resolved Fluorescence Resonance Energy Transfer CRBN Competitive Binding Assay

The pFastBacDual-6xHis-CRBN/DDB1 plasmid was cloned by GenScript and used for baculoviral expression of His-CRBN and untagged DDB1. Protein was expressed and purified essentially as described previously. The bacmid was produced in MAX Efficiency DH10Bac Competent Cells (Thermo Fisher Scientific) according to the manufacturer's instructions. To generate the P1 virus, 1×10⁶ Sf9 cells were plated in each well of a six-well plate in 2 mL Sf-900 III SFM and transfected with 7 μg bacmid using Cellfectin II reagent (Thermo Fisher Scientific). After 6 h at 27° C., the media was replaced with 2 mL fresh Sf-900 III SFM. After three days, the supernatant was collected, centrifuged at 500×g for 5 min to remove cells and debris, transferred to new tubes, and stored at 4° C. To generate P2 virus, 2 mL P1 virus was added to 1×10⁸ Sf9 cells in 50 mL Sf-900 III SFM and shaken at 27° C. for three days. The supernatant was collected and clarified as above. For protein expression, 5 mL P2 virus was added to each liter of Sf cells (2×10⁶ cells/mL) and shaken at 27° C. for three days. Cells containing expressed protein were pelleted by centrifugation at 500×g for 5 min, the supernatant was removed, and pellets were stored at −80° C. until processing. Cells were resuspended in lysis buffer [50 mM Tris (pH 7.5), 500 mM NaCl, 1 mM TCEP, 10 mM imidazole, 10% glycerol] containing SIGMAFAST Protease Inhibitor Cocktail Tablets, EDTA-Free (Sigma-Aldrich) and 125 U/mL Benzonase Nuclease (Sigma-Aldrich). The suspension was incubated on ice for 1 h, gently sonicated, and centrifuged for 1 h at 40,000×g at 4° C. The supernatant was applied to Ni-NTA Superflow resin (QIAGEN) equilibrated with lysis buffer. The resin was washed with 10 column volumes (CV) of lysis buffer, and proteins were eluted with 10 CV lysis buffer containing 500 mM imidazole. The solution was diluted to 200 mM NaCl using lysis buffer without NaCl and applied to a HiTrap ANX FF (High Sub) column (Cytiva) equilibrated with ANX binding buffer [50 mM Tris (pH 7.5), 200 mM NaCl, 3 mM TCEP]. The column was washed with 10 CV ANX binding buffer followed by 10 CV ANX wash buffer [50 mM Bis-Tris (pH 6.0), 200 mM NaCl, 3 mM TCEP]. A 20 CV linear gradient was then immediately applied from ANX binding buffer to ANX binding buffer containing 1 M NaCl using an AKTA avant chromatography system (Cytiva). The purest fractions were applied to a HiLoad 26/600 Superdex 200 pg size exclusion column equilibrated with 10 mM HEPES (pH 7.0), 240 mM NaCl, and 3 mM TCEP. Pure protein was concentrated to 20-30 mg/mL in Pierce PES Protein Concentrators (Thermo Fisher Scientific), flash frozen in liquid nitrogen, and stored at −80° C.

The time-resolved fluorescence resonance energy transfer (TR-FRET) CRBN competitive binding assay was performed as previously described {33070611}. The assay buffer composition was 50 mM Tris (pH 7.5), 0.1% Triton X-100, 0.01% bovine serum albumin, and 1 mM DTT. BODIPY FL thalidomide (15 μL/well, 5.3 nM in assay buffer) was dispensed into 384-well low-volume black assay plates. An Echo 655T Acoustic Liquid Handler then dispensed 20 nL/well of compound stocks or DMSO. Lastly, 5 μL/well of 8 nM Tb-anti-His and 8 nM His-CRBN/DDB1 in assay buffer was added. The final concentrations of the assay components (in a 20-μL final assay volume per well) were: 4 nM BODIPY FL thalidomide, 2 nM Tb-anti-His, 2 nM His-CRBN/DDB1, and 0.2% DMSO. The BODIPY FL thalidomide stock contributed 0.1% DMSO, and the test compounds contributed 0.1% DMSO. DMSO alone and 3 μM unlabeled thalidomide were included in each plate to serve as negative and positive controls, respectively. The plates were shaken at 900 rpm (80×g) on an IKA MTS 2/4 digital microtiter shaker (IKA Works) for 1 minute then centrifuged at 1,000 rpm (201×g) for 30 seconds in an Eppendorf 5810 centrifuge equipped with an A-4-62 swing-bucket rotor. The plates were then protected from light exposure and incubated for 60 minutes at room temperature (RT). After incubation, the TR-FRET signal from each well was collected with a PHERAstar FS Microplate Reader (BMG Labtech). The percent inhibition for each well was calculated using equation 2:

$$CRBN Binding ( \% Inhibition ) = 100 \% - 100 \% \times \frac{( {Signal}_{Chemical} - {Signal}_{Thalidomide} )}{( {Signal}_{DMSO} - {Signal}_{Thalidomide} )}$$

### 3. Development of a High-Throughput Assay for Degradation of Overexpressed Protein

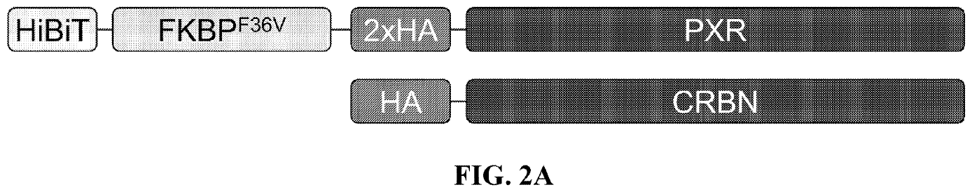

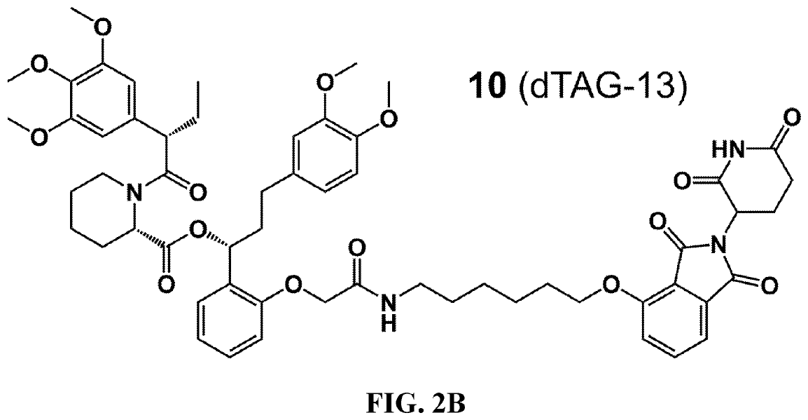

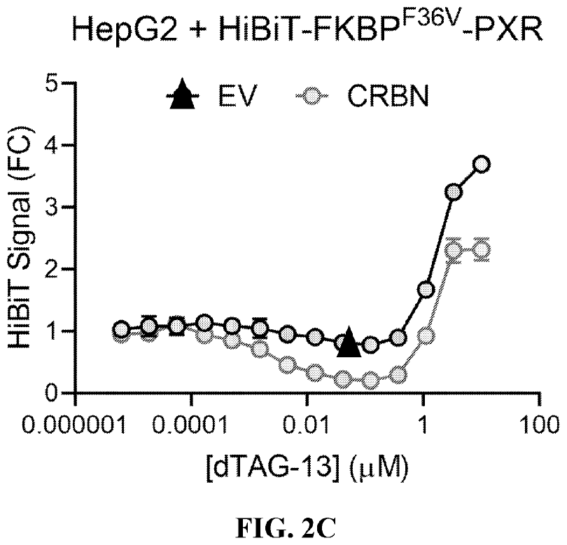

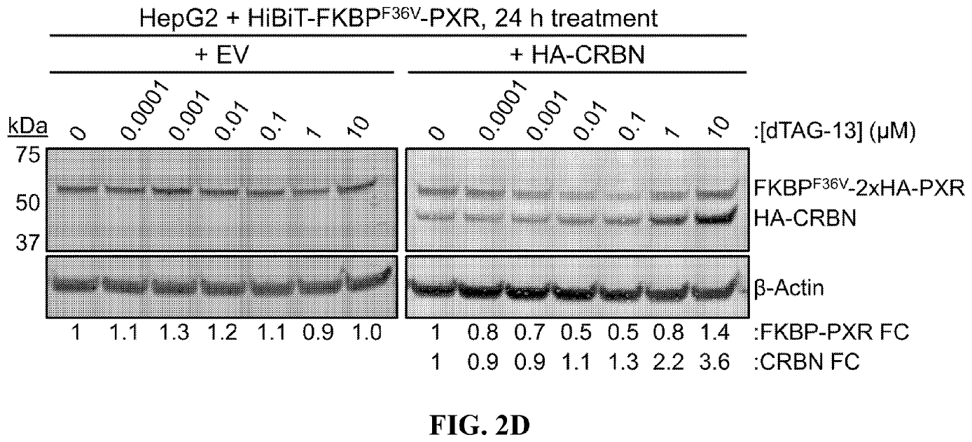

To establish a method for discovery of PXR degraders in the absence of a known PXR-degrading control, the degradation tag (dTAG) system was used. With this method, a protein of interest (PXR) is fused to a cytosolic prolyl isomerase, FKBP12, with Phe36 mutated to Val (FKBP12^{F36V}) (FIG. 2A). A PROTAC molecule, dTAG-13 (FIG. 2B), is then used to induce degradation of the fusion protein through recruitment of the CUL4-RBX1-DDB1-CRBN (CRL4^{CRBN}) E3 ubiquitin ligase, without affecting the cellular FKBP12 protein. An HA tag was added for western blot detection, and a HiBiT tag was fused to the N-terminus to allow high-throughput plate-based quantification of HiBiT-FKBP12^{F36V}-PXR protein levels (FIG. 2A). When overexpressed in HepG2 cells, only a slight reduction in HiBiT signal was observed with dTAG-13 (FIG. 2C). However, when CRBN was co-overexpressed, dTAG-13 potently reduced HiBiT signal (FIG. 2C). This was also observed when the HA tag was detected by western blot (FIG. 2D). The HiBiT detection was more sensitive to protein level changes than western blot, and, importantly, overexpression of CRBN was required to achieve dTAG-13-mediated degradation of the overexpressed HiBiT-FKBP12^{F36V}-PXR protein. A hook effect was observed as expected, and the potency of dTAG-13 in this assay was comparable to previously reported results.

### 4. Discovery of a Putative PXR Degrader

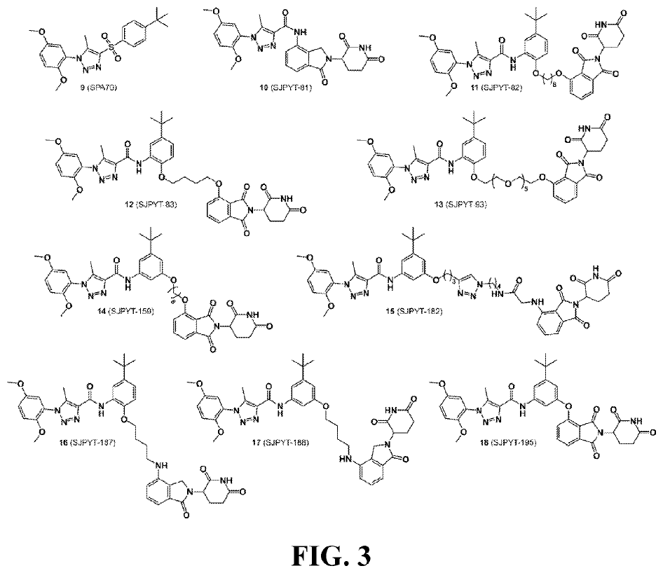

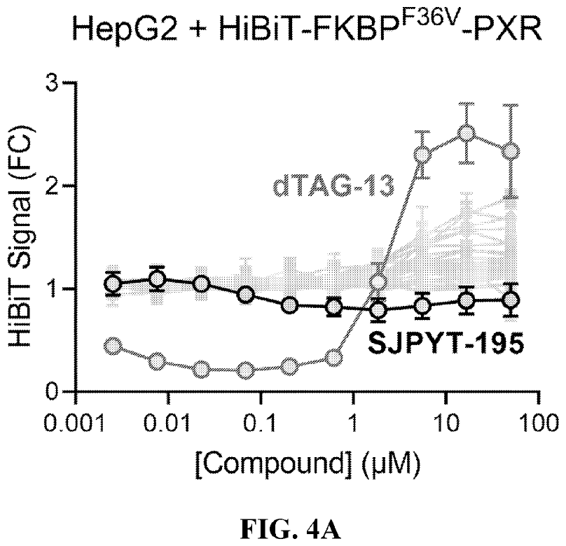

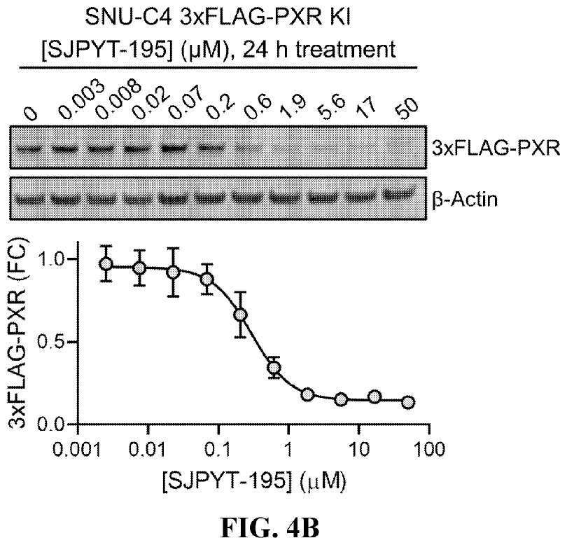

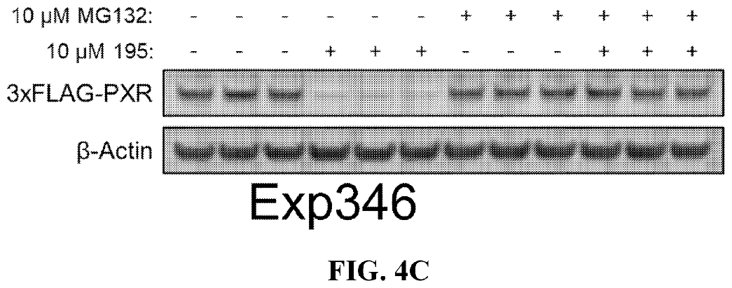

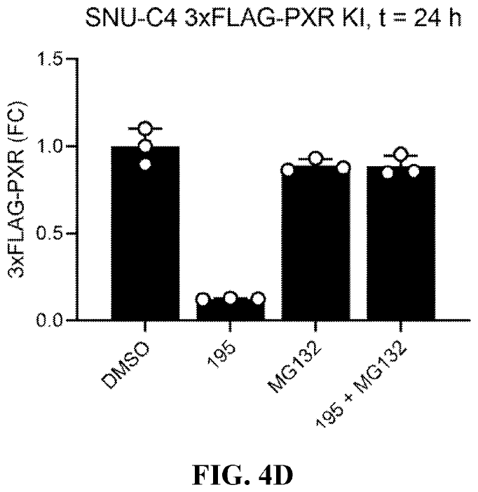

Because the HiBiT-based high-throughput assay was successfully validated, compounds that reduce PXR protein level were screened. Nine compounds that linked PXR ligands to CRBN ligands (FIG. 3) were synthesized and screened against HiBiT-FKBP12^{F36V}-PXR, using dTAG-13 as an assay control. Most of the compounds either had no effect or increased HiBiT signal, but one compound, SJPYT-195, reduced HiBiT signal (FIG. 4A). To assess SJPYT-195 activity against endogenous PXR, CRISPR-Cas9 was used to knock in a 3xFLAG tag to the PXR N-terminus in the colorectal SNU—C4 cell line (henceforth referred to as SNU—C4 3xFLAG-PXR KI cells). SJPYT-195 potently and efficaciously reduced endogenous PXR protein in this system, with a half maximal degradation concentration (DC₅₀) of 310±130 nM and maximum degradation efficacy (DNax) of 85% ±1% (FIG. 4B). The loss of PXR protein was dependent on the proteasome, evidenced by rescue of PXR levels by the proteasome inhibitor MG132 (FIG. 4C and FIG. 4D).

### 5. SJPYT-195 Impacts Pxr in Manners Inconsistent with Direct Degradation

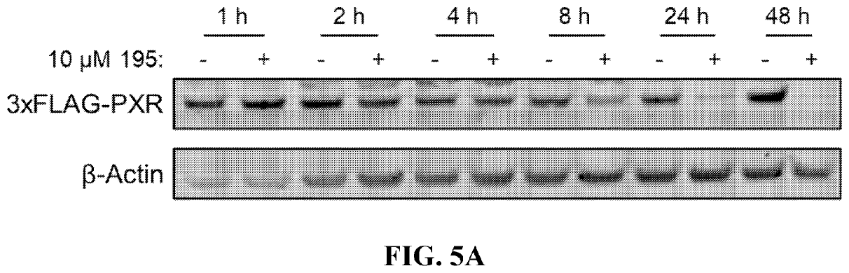

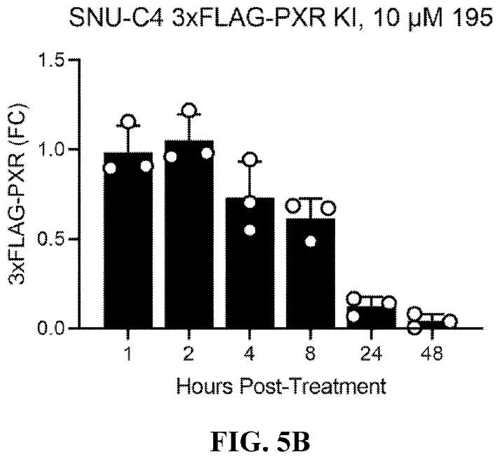

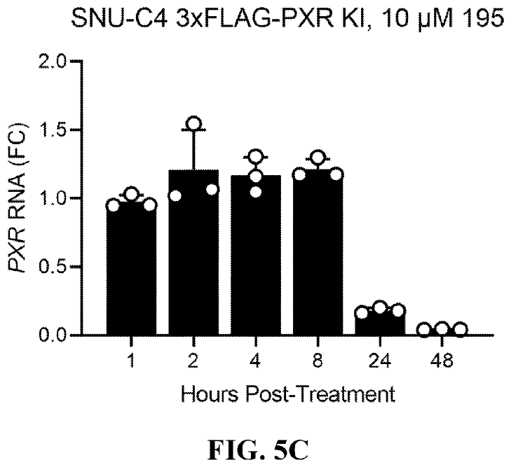

Without wishing to be bound by theory, the results suggested that SJPYT-195 is indeed a proteasome-dependent PXR degrader. To ascertain this mechanism, time-course analysis of PXR protein and mRNA levels was performed. The kinetics of PXR protein decrease in response to SJPYT-195 were quite slow (FIG. 5A and FIG. 5B), contrary to the expectation that a direct protein degrader generally results in rapid protein loss. Furthermore, SJPYT-195 drastically reduced PXR RNA level (FIG. 5C). This effect was unexpected, as modulation of PXR protein is not known to significantly alter transcription of the PXR gene. These results suggest that SJPYT-195 may indirectly reduce PXR protein through, for example, a transcriptional or translational mechanism. The observation that PXR protein decrease occurs at earlier time points than RNA decrease indicates that the effect is at least partially independent of transcription defects.

### 6. Whole-Cell Assessment of SJPYT-195 Protein-Degrading Activity

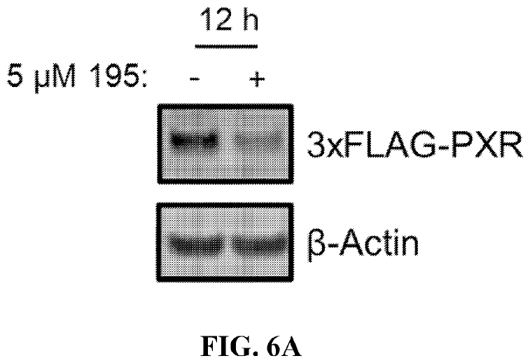

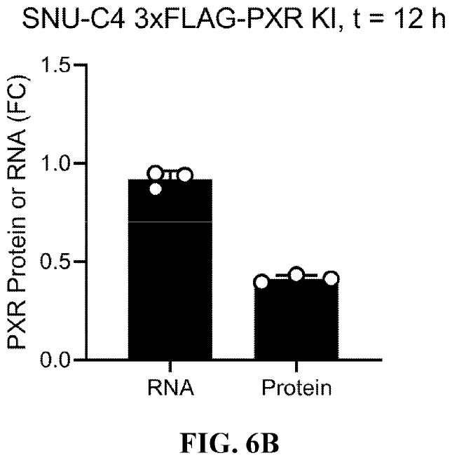

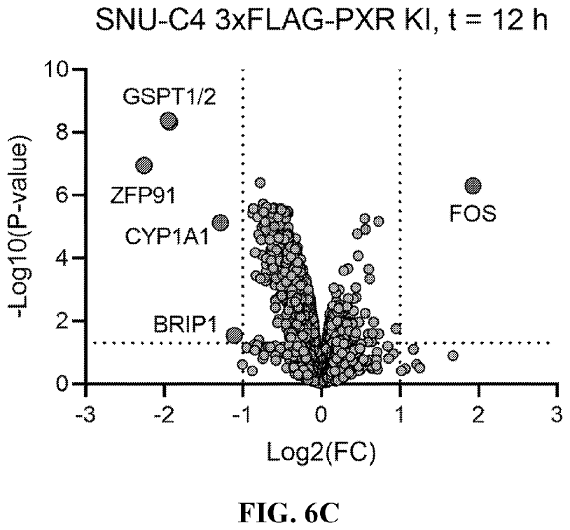

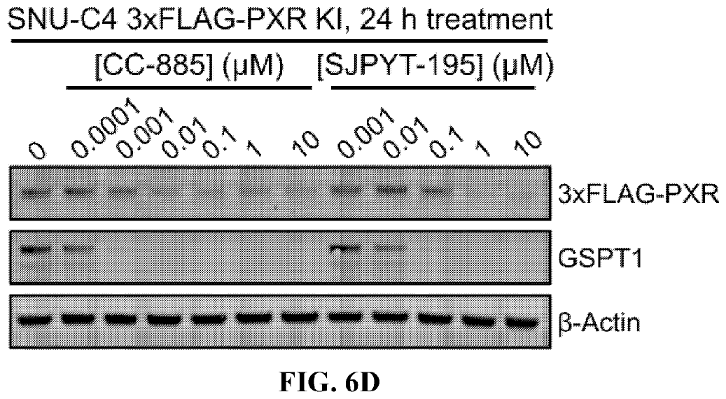

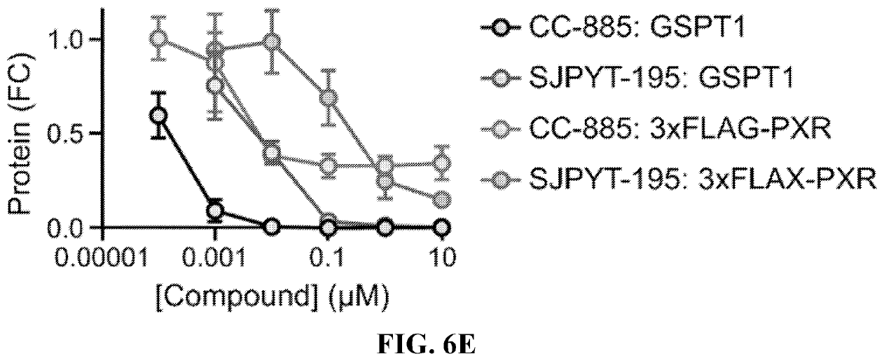

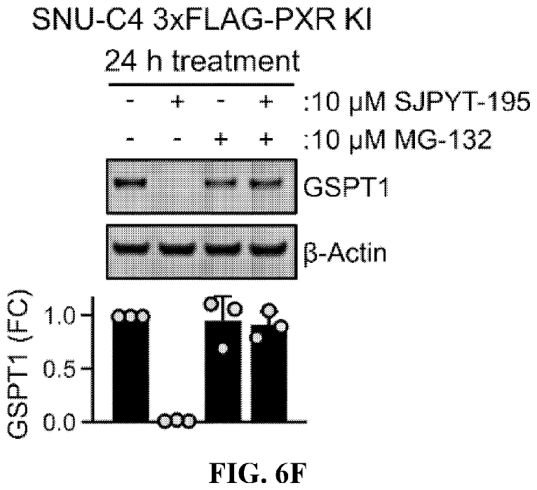

The kinetics of PXR protein loss in response to SJPYT-195 treatment suggested that PXR may not be a direct target of SJPYT-195. To determine cellular SJPYT-195 targets, tandem mass tag mass spectrometry (TMT-MS) was performed to quantify protein changes in SNU—C4 3xFLAG-PXR KI cells at the whole-proteome scale. First, the treatment time point that markedly decreased PXR protein without affecting RNA was identified (FIG. 6A and FIG. 6B). At 12 h, 5 μM SJPYT-195 reduced PXR protein ˜50% with negligible reduction of PXR RNA. Next, TMT-MS for cells treated for 12 h was performed with either 5 μM SJPYT-195 or DMSO control (FIG. 6C). The analysis identified five down-regulated proteins (GSPT1, GSPT2, ZFP91, CYP1A1, and BRIP1) and one upregulated protein (FOS). SJPYT-195 was remarkably selective, only down-regulating five proteins at the relatively high treatment concentration (5 μM) and duration (12 h). However, additional targets existing cannot be ruled out. The dataset had high coverage, detecting 9,692 proteins (11,685 isoforms), representing the majority of expressed human proteome in a specific cell type. Proteins at extremely low abundance, such as PXR, were not observed in the data.

### 7. SJPYT-195 is a GSPT1 Degrader

In the proteomics analysis, GSPT1 was identified as one of the most significantly down-regulated proteins in response to SJPYT-195. G1 to S Phase Transition Protein 1 Homolog (GSPT1) is a translation termination factor that participates in nascent protein release from ribosomes. Loss of GSPT1 results in translation defects that could lead to decrease of unstable or lowly abundant proteins, such as PXR. Using western blot, it was validated that SJPYT-195 does indeed deplete GSPT1 at concentrations much lower than those required to reduce PXR (FIG. 6D and FIG. 6E). The activity of SJPYT-195 on GSPT1 was ˜100-fold lower than that of the previously reported GSPT1 degrader CC-885, which is the most potent GSPT1 degrader known to date. MG-132 rescued GSPT1 in the presence of SJPYT-195, indicating that GSPT1 loss was proteasome-dependent (FIG. 6F). In addition to degrading GSPT1, CC-885 also reduced PXR protein, suggesting that PXR reduction is a secondary effect of GSPT1 degradation (FIG. 6D and FIG. 6E). Interestingly, though, SJPYT-195 reduced PXR to a greater extent than CC-885. Therefore, SJPYT-195 may impact PXR protein level by more than one mechanism.

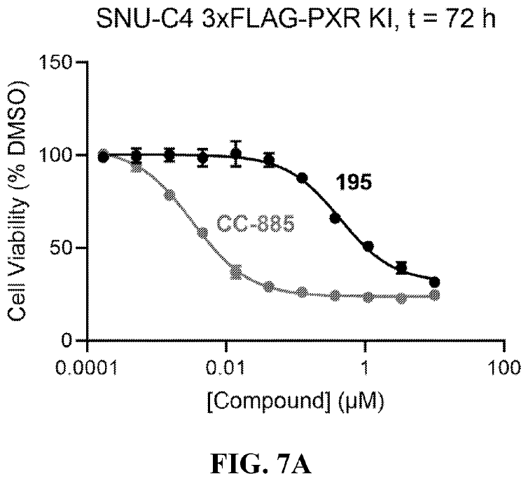

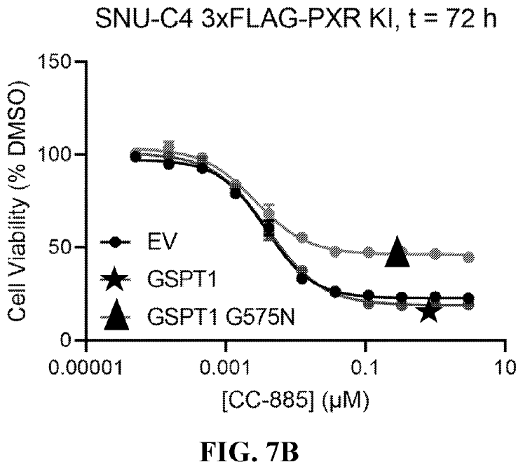

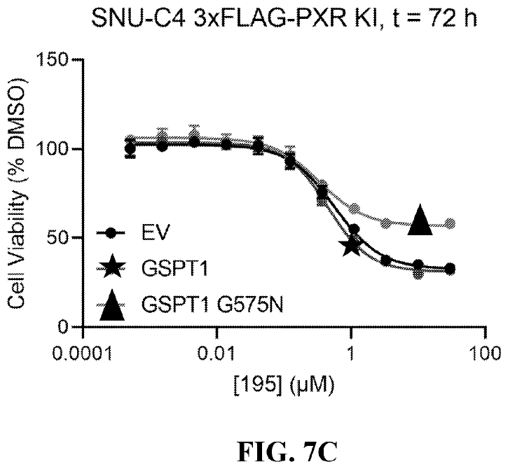

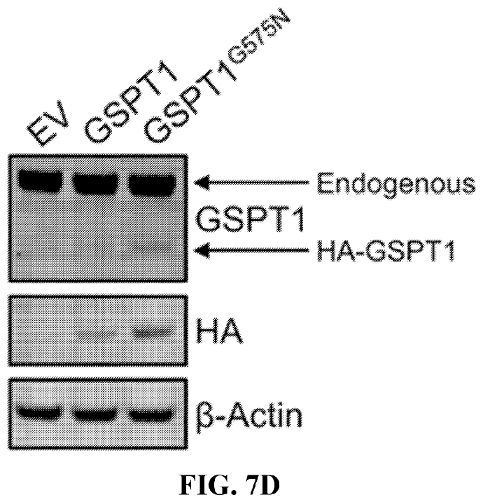

GSPT1 degraders are actively studied as potential anticancer agents, and CC-885 is a highly potent killer of various cell models. The cytotoxicity of CC-885 and SJPYT-195 was tested in the SNU—C4 3xFLAG-PXR KI cells and found half maximal cytotoxic concentration (CC₅₀) values of 3.3±0.3 nM and 440±80 nM, respectively (FIG. 7A). The 133-fold difference in CC₅₀ values correlated well with the observed differences in potencies for GSPT1 degradation in FIG. 6D-E. To assess the role of GSPT1 degradation in mediating the cytotoxicity of SJPYT-195, SNU—C4 3xFLAG-PXR KI cell lines were generated stably expressing either empty vector (EV), HA-tagged wild-type GSPT1, or HA-tagged GSPT1^{G575N}, a mutant that was previously shown to be resistant to molecular glue-mediated degradation. Overexpression of GSPT1^{G575N} rendered the cells partially resistant to both CC-885 and SJPYT-195, suggesting that compound cytotoxicity is mediated through GSPT1 degradation (FIG. 7B and FIG. 7C). Full rescue could not be achieved because the plasmid-derived GSPT1 was expressed at a significantly lower level than the endogenous GSPT1 (FIG. 7D). By western blot, the overexpressed GSPT1 was well-separated from endogenous GSPT1 because we cloned the short isoform that lacks residues 1-138 of the long isoform. In SNU—C4 cells, the long isoform appears to be the dominantly expressed protein and therefore migrates more slowly in the gel than the overexpressed short isoform.

### 8. Structure-Activity Relationship (SAR)

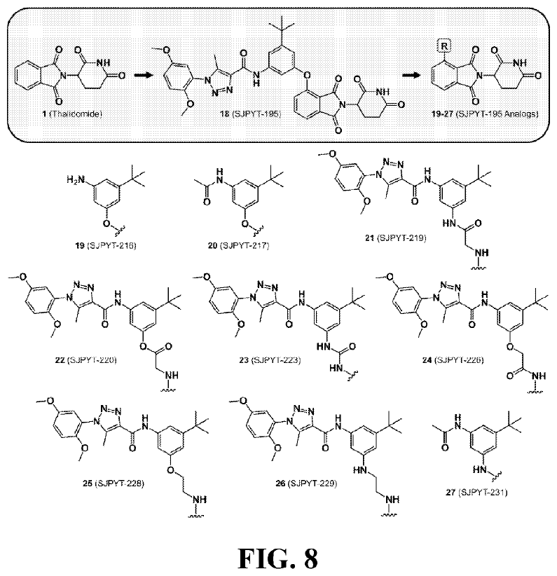

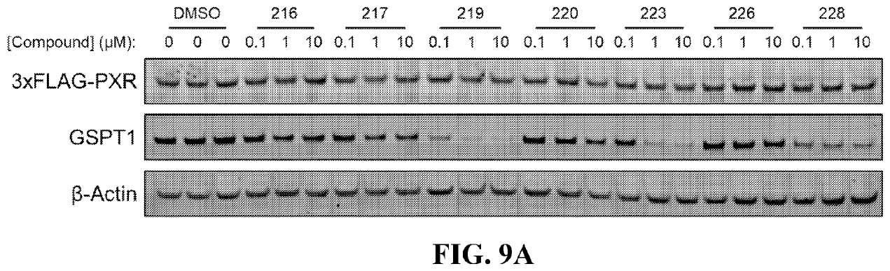

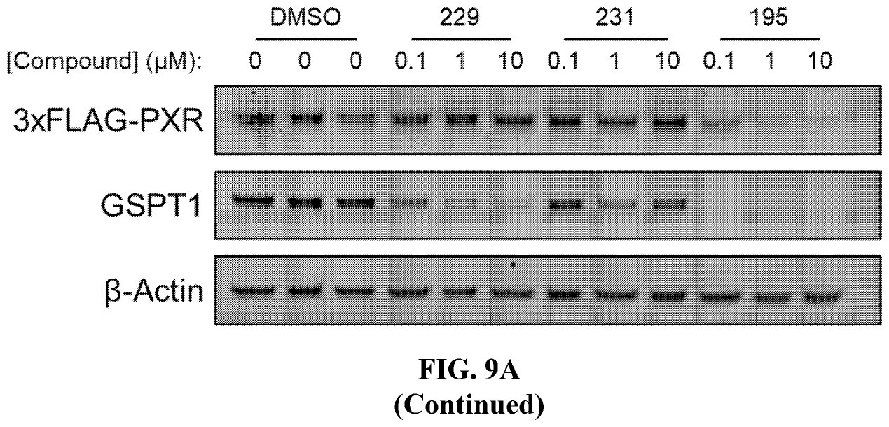

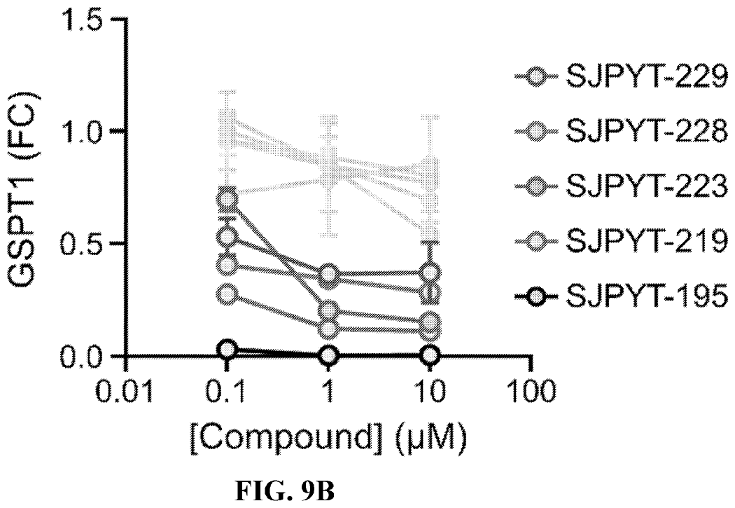

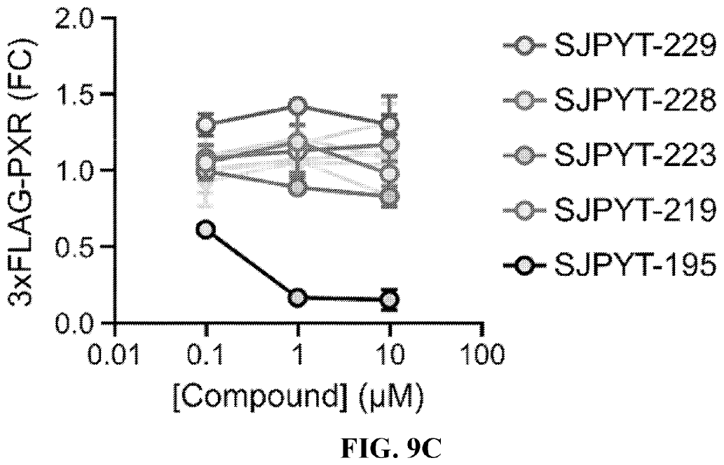

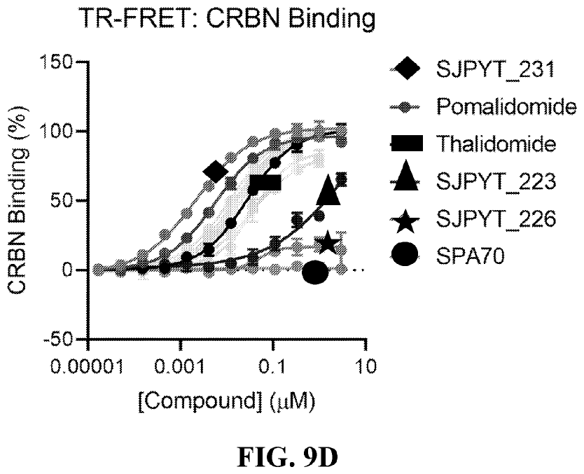

To evaluate the relationship between SJPYT-195 structure and GSPT1 degradation, nine analogs with varying linker lengths and compositions and truncations of the SPA70 component were synthesized (FIG. 8). Western blot analysis showed that all analogs had substantially reduced activity for both PXR and GSPT1 (FIG. 9A-FIG. 9C and Table 3). However, most compounds showed similar binding affinity to CRBN itself (FIG. 9D), suggesting that either recruitment or ubiquitination of GSPT1 is impaired in the inactive analogs. In fact, SJPYT-231 had the strongest CRBN binding of all tested compounds but did not affect either PXR or GSPT1 levels.

The results seem to suggest that short linkers and bulky substituents are favored for GSPT1 degradation by this particular class of chemicals. Importantly, CRBN binding did not correlate with GSPT1 degradation. For example, by truncating a part of the PXR ligand, SJPYT-216, SJPYT-217, and SJPYT-231 still maintained strong CRBN binding, but lost the degradation activity of GSPT1. This indicates that the integrity of the PXR ligand is crucial to the degradation of GSPT1, possibly by influencing the molecular glue-induced CRBN-GSPT1 interface. On the other hand, although SJPYT-223 had greatly reduced CRBN binding, it still exhibited strong GSPT1 degradation activity, indicating that the short 3 atom linker was beneficial to GSPT1 degradation activity (compared to the longer linkers of SJPYT-220 and −226). For SJPYT-226 (4 atom linker), amino acetylation on the CRBN ligand (SJPYT-226) also greatly reduced CRBN binding. The other 4 atom linker compounds (SJPYT-219, SJPYT-220, SJPYT-228, and SJPYT-229) all retained strong binding of CRBN, with SJPYT-219, SJPYT-228, and SJPYT-229 showing strong GSPT1 degradation. SJPYT-220 only weakly degraded GSPT1, possibly due to an unstable ester bond of the phenolic hydroxyl group. With the full PXR ligand and only a 1 atom linker, SJPYT-195 was the most potent inducer of GSPT1 degradation in the group of chemicals.

TABLE 3

| | CRBN Binding | GSPT1 Loss at |
|---|---|---|
| Compound | EC50 ± SD (nM)^{a} | 10 μM (% ± SD)^{c} |
| M4 | 31 ± 1.7 | 100 ± 0.1 |
| A1 | 14 ± 1.0 | 23 ± 2.8 |
| A2 | 11 ± 0.6 | 31 ± 9.8 |
| M8 | 15 ± 1.1 | 89 ± 3.1 |
| M7 | 17 ± 1.7 | 46 ± 1.5 |
| M6 | 410 ± 37 | 85 ± 2.2 |
| M9 | >333^{b} | 20 ± 4.8 |
| M10 | 23 ± 1.4 | 72 ± 2.3 |
| M11 | 24 ± 1.9 | 63 ± 13 |
| A3 | 3.2 ± 0.2 | 15 ± 21 |
| SPA70 | >3000^{b} | Not Tested |
| Thalidomide | 36 ± 1.2 | Not Tested |
| Pomalidomide | 9.0 ± 0.6 | Not Tested |

^{a}CRBN binding half maximal effective concentrations (EC₅₀) are derived from FIG. 9D.
^{b}The EC₅₀ for SJPYT-226 and SPA70 could not be derived because the fitted curves did not reach 50%. For SJPYT-226, assay interference was observed at concentrations greater than 333 nM, and these points were excluded from the analysis. SPA70 was inactive.
^{c}The percentage of GSPT1 protein reduction at 10 μM compound was calculated from the western blots in

### FIG. 9A.

### 9. Discussion

Because PXR is a master transcriptional regulator of drug metabolism and efflux programs, inhibitors of this pathway could be valuable clinical tools to mitigate adverse effects such as drug-induced liver injury and drug-drug interactions. Previously, the discovery of SPA70 as a potent and selective dual inverse agonist and antagonist of PXR was reported. Further derivatization of SPA70 yielded only antagonists and agonists, suggesting that PXR inverse agonism is difficult to achieve. This challenge is echoed in the abundance of small molecules that activate PXR and the aforementioned lack of inhibitors.³² This persistent problem makes targeted protein degradation an enticing concept for decreasing PXR activity in cells. The discovery that PXR mutation converts an inverse agonist to an agonist further highlights the value of a PXR degrader in reducing the level and activity of PXR, as a degrader might be less likely to be affected by mutations of the target protein.

In an effort to obtain PXR-degrading small molecules, an assay to detect degradation of overexpressed PXR was first optimized (FIG. 2). Importantly, it was found that co-overexpression of CRBN was required to observe reduction of overexpressed FKBP^{F36V}-PXR by the control compound dTAG-13. This finding may accelerate degrader discovery efforts because transiently or stably overexpressed protein can be used rather than generating CRISPR/Cas9-mediated tag knock-ins for protein detection. After validating the assay, a panel of candidate PXR degraders was made by linking a SPA70 derivative to a CRBN ligand. One molecule, SJPYT-195, markedly decreased PXR protein in a dose- and time-dependent manner. However, upon further investigation, GSPT1 was found to be a primary degraded target of SJPYT-195. Precedents for this “off-target” effect exist in the literature, such as GSPT1 degraders discovered from molecules designed to degrade kinases or MDM2. Furthermore, previous derivatization of thalidomide resulted in the potent GSPT1 degraders CC-885 and CC-90009. CC-885 and SJPYT-195 both substantially decreased PXR protein level, indicating that PXR loss is a secondary effect of GSPT1 degradation. Interestingly, though, SJPYT-195 decreases PXR to a greater extent than CC-885 (FIG. 6D and FIG. 6E), suggesting that SJPYT-195 may have GSPT1-independent effects on PXR. The additional mechanisms could be direct or indirect and are subjects for future study. For example, SJPYT-195 may indeed directly bind PXR, inducing degradation through CRL4C^{RBN}. Another possibility is that a separate target, such as ZFP91 that was identified through TMT-MS (FIG. 6C), is mediating the observed additional decrease in PXR protein. ZFP91 has been shown to control various gene networks by ubiquitinating key transcription factors. Like GSPT1, ZFP91 is a previously reported target of thalidomide-based molecules.

SJPYT-195 represents a new subclass of GSPT1-degrading small molecules. GSPT1 is a translation termination factor that participates in nascent protein release from ribosomes, and is therefore a vital component to cell health. The small molecule CC-885 was previously identified as a GSPT1 degrader and potent cytotoxic agent in acute myeloid leukemia (AML) cell lines, suggesting that GSPT1 is a potential anti-cancer target. RNA sequencing analysis in pediatric tumor samples has also shown that GSPT1 is overexpressed in hematopoietic malignancies like AML. These observations have led to interest in developing GSPTT-degrading small molecules. SJPYT-195 shows promise as a lead compound for further modification and study, and it has been shown that a chemical truncation of this molecule produces a higher affinity CRBN binder than thalidomide or pomalidomide (SJPYT-231, FIG. 9D). Thus, the study has identified chemical mailer for future use as 1) GSPT1 degraders or 2) high potency CRBN ligands in PROTAC synthesis.

### 10. Biological Evaluation of Exemplary Compounds

A list of exemplary compounds is shown in Table 4 and their accompanying data is shown in Table 5.

TABLE 4

| Compound | Structure |
|---|---|
| T0901317 | 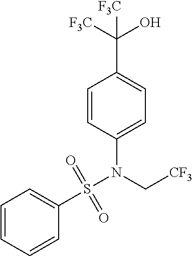 [SMILES: O=S(=O)(c1ccccc1)N(CC(F)(F)F)c1ccc(C(O)(C(F)(F)F)C(F)(F)F)cc1] |
| Rifampicin | 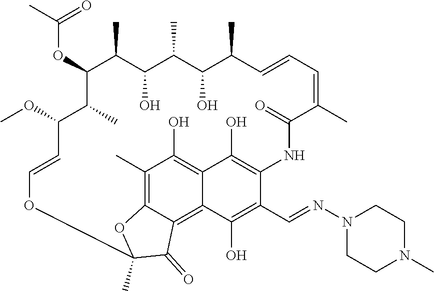 |
| SPA70 | 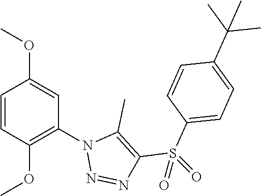 [SMILES: COc1ccc(OC)c(-n2nnc(S(=O)(=O)c3ccc(C(C)(C)C)cc3)c2C)c1] |
| M1 | 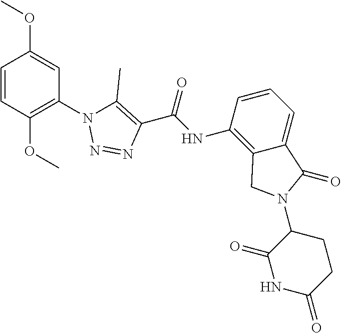 [SMILES: COc1ccc(OC)c(-n2nnc(C(=O)Nc3cccc4c3CN(C3CCC(=O)NC3=O)C4=O)c2C)c1] |
| M2 | 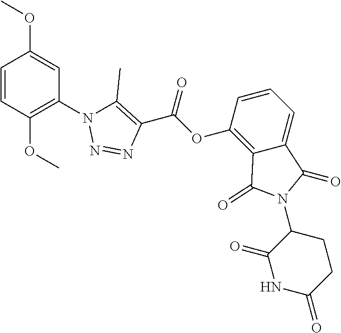 [SMILES: COc1ccc(OC)c(-n2nnc(C(=O)Oc3cccc4c3C(=O)N(C3CCC(=O)NC3=O)C4=O)c2C)c1] |
| M3 | 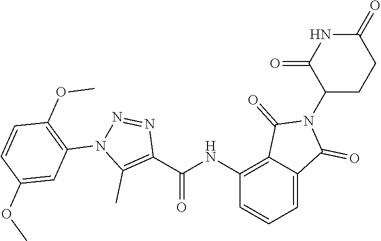 [SMILES: COc1ccc(OC)c(-n2nnc(C(=O)Nc3cccc4c3C(=O)N(C3CCC(=O)NC3=O)C4=O)c2C)c1] |
| M4 | 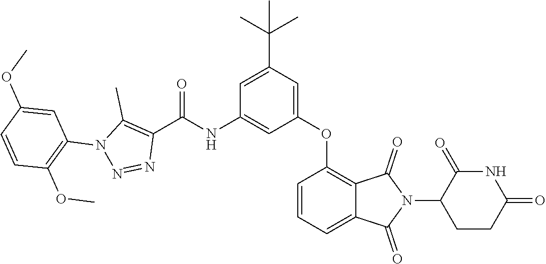 [SMILES: COc1ccc(OC)c(-n2nnc(C(=O)Nc3cc(Oc4cccc5c4C(=O)N(C4CCC(=O)NC4=O)C5=O)cc(C(C)(C)C)c3)c2C)c1] |
| M5 | 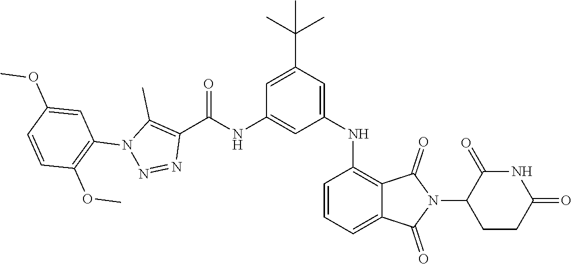 |
| M6 | 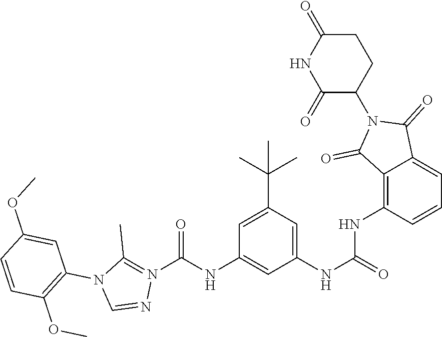 |
| M7 | 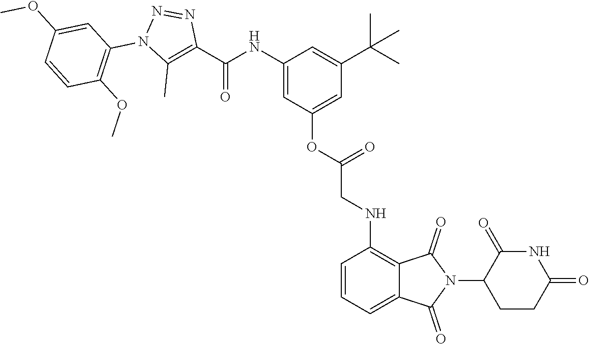 [SMILES: COc1ccc(OC)c(-n2nnc(C(=O)Nc3cc(OC(=O)CNc4cccc5c4C(=O)N(C4CCC(=O)NC4=O)C5=O)cc(C(C)(C)C)c3)c2C)c1] |
| M8 | 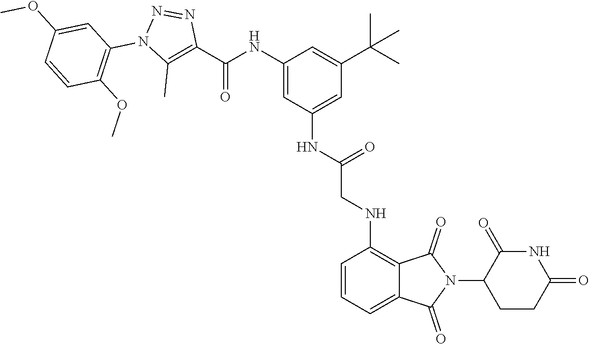 [SMILES: COc1ccc(OC)c(-n2nnc(C(=O)Nc3cc(NC(=O)CNc4cccc5c4C(=O)N(C4CCC(=O)NC4=O)C5=O)cc(C(C)(C)C)c3)c2C)c1] |
| M9 | 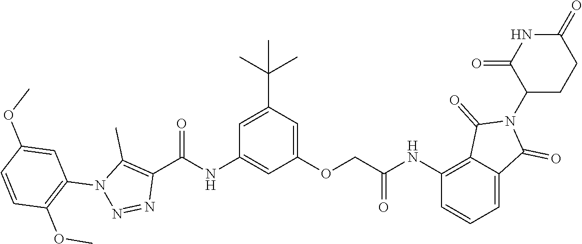 [SMILES: COc1ccc(OC)c(-n2nnc(C(=O)Nc3cc(OCC(=O)Nc4cccc5c4C(=O)N(C4CCC(=O)NC4=O)C5=O)cc(C(C)(C)C)c3)c2C)c1] |
| M10 | 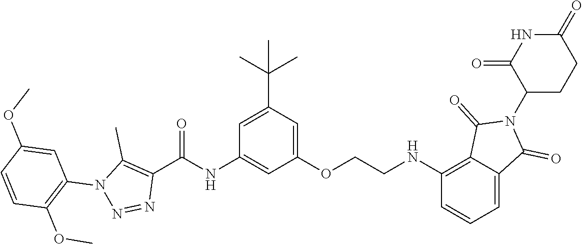 [SMILES: COc1ccc(OC)c(-n2nnc(C(=O)Nc3cc(OCCNc4cccc5c4C(=O)N(C4CCC(=O)NC4=O)C5=O)cc(C(C)(C)C)c3)c2C)c1] |
| M11 | 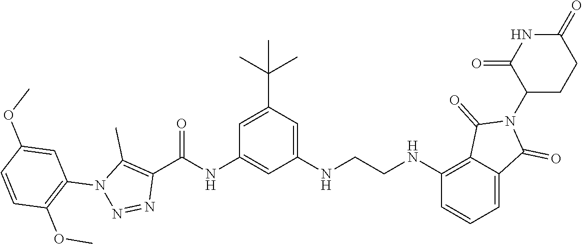 [SMILES: COc1ccc(OC)c(-n2nnc(C(=O)Nc3cc(NCCNc4cccc5c4C(=O)N(C4CCC(=O)NC4=O)C5=O)cc(C(C)(C)C)c3)c2C)c1] |
| M12 | 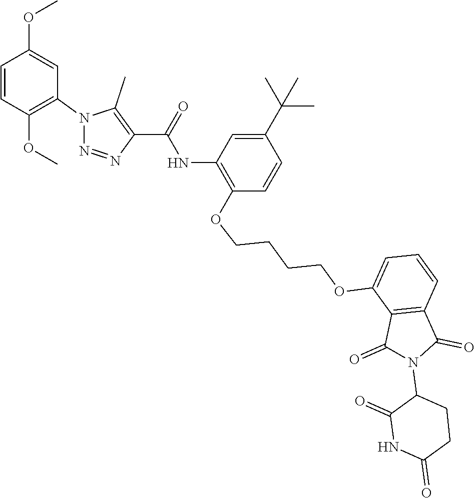 [SMILES: COc1ccc(OC)c(-n2nnc(C(=O)Nc3cc(C(C)(C)C)ccc3OCCCCOc3cccc4c3C(=O)N(C3CCC(=O)NC3=O)C4=O)c2C)c1] |
| M13 | 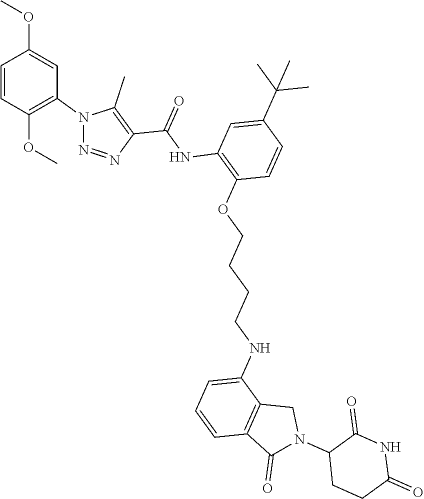 [SMILES: COc1ccc(OC)c(-n2nnc(C(=O)Nc3cc(C(C)(C)C)ccc3OCCCCNc3cccc4c3CN(C3CCC(=O)NC3=O)C4=O)c2C)c1] |
| M14 | 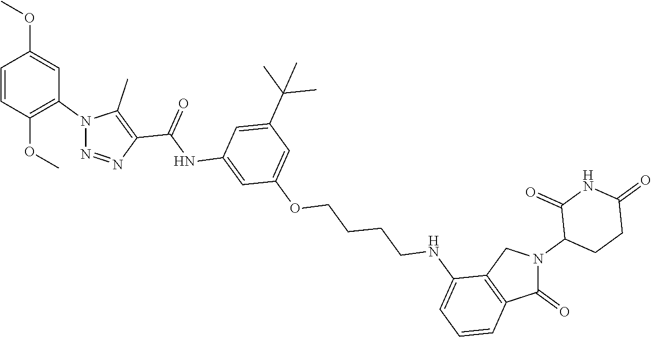 [SMILES: COc1ccc(OC)c(-n2nnc(C(=O)Nc3cc(OCCCCNc4cccc5c4CN(C4CCC(=O)NC4=O)C5=O)cc(C(C)(C)C)c3)c2C)c1] |
| M15 | 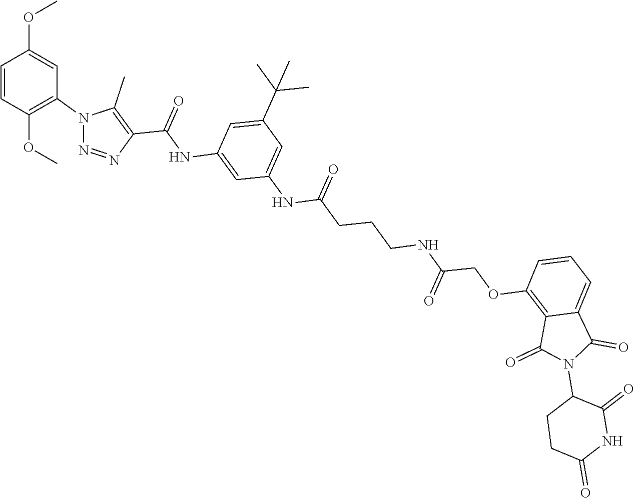 [SMILES: COc1ccc(OC)c(-n2nnc(C(=O)Nc3cc(NC(=O)CCCNC(=O)COc4cccc5c4C(=O)N(C4CCC(=O)NC4=O)C5=O)cc(C(C)(C)C)c3)c2C)c1] |
| M16 | 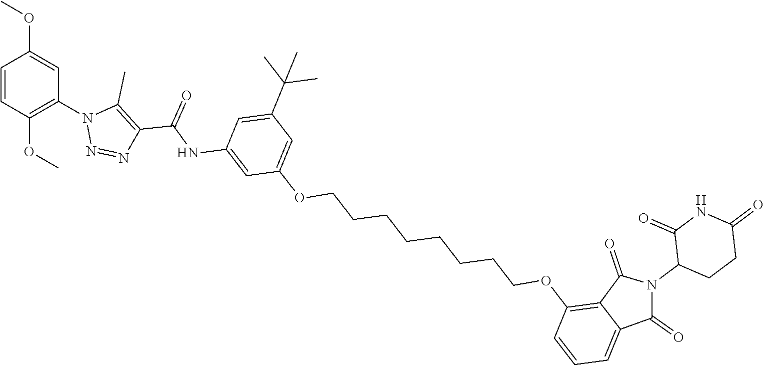 [SMILES: COc1ccc(OC)c(-n2nnc(C(=O)Nc3cc(OCCCCCCCCOc4cccc5c4C(=O)N(C4CCC(=O)NC4=O)C5=O)cc(C(C)(C)C)c3)c2C)c1] |
| M17 | 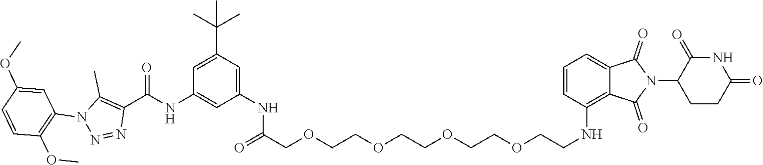 [SMILES: COc1ccc(OC)c(-n2nnc(C(=O)Nc3cc(NC(=O)COCCOCCOCCOCCNc4cccc5c4C(=O)N(C4CCC(=O)NC4=O)C5=O)cc(C(C)(C)C)c3)c2C)c1] |
| M18 | 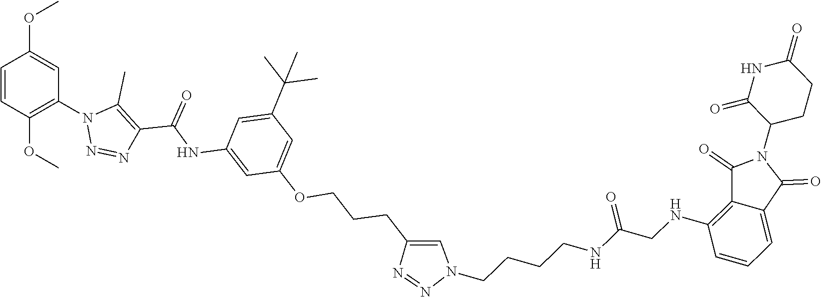 [SMILES: COc1ccc(OC)c(-n2nnc(C(=O)Nc3cc(OCCCc4cn(CCCCNC(=O)CNc5cccc6c5C(=O)N(C5CCC(=O)NC5=O)C6=O)nn4)cc(C(C)(C)C)c3)c2C)c1] |
| M19 | 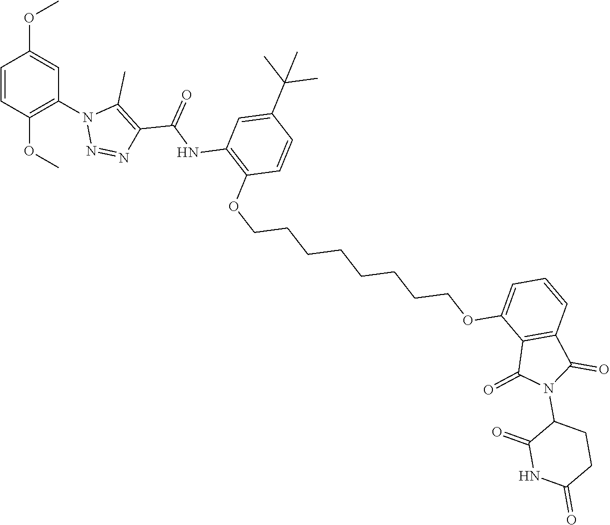 [SMILES: COc1ccc(OC)c(-n2nnc(C(=O)Nc3cc(C(C)(C)C)ccc3OCCCCCCCCOc3cccc4c3C(=O)N(C3CCC(=O)NC3=O)C4=O)c2C)c1] |
| M20 | 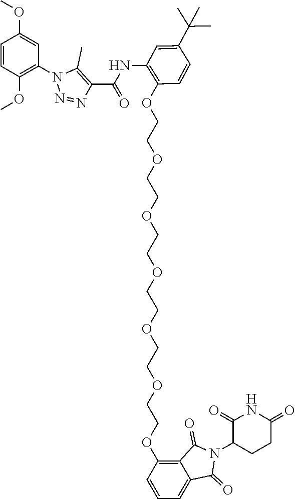 [SMILES: COc1ccc(OC)c(-n2nnc(C(=O)Nc3cc(C(C)(C)C)ccc3OCCOCCOCCOCCOCCOCCOc3cccc4c3C(=O)N(C3CCC(=O)NC3=O)C4=O)c2C)c1] |
| N1 | 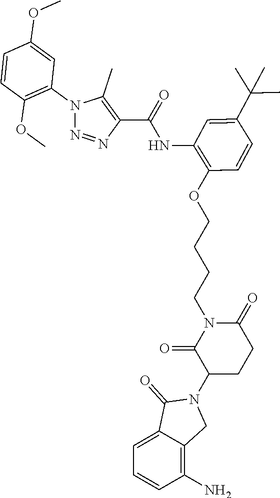 [SMILES: COc1ccc(OC)c(-n2nnc(C(=O)Nc3cc(C(C)(C)C)ccc3OCCCCN3C(=O)CCC(N4Cc5c(N)cccc5C4=O)C3=O)c2C)c1] |
| N2 | 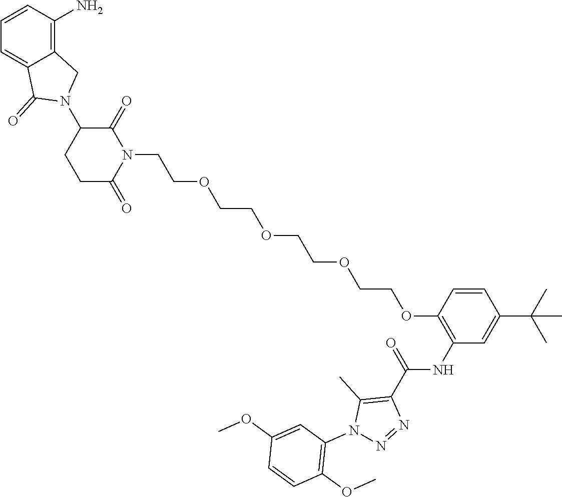 [SMILES: COc1ccc(OC)c(-n2nnc(C(=O)Nc3cc(C(C)(C)C)ccc3OCCOCCOCCOCCN3C(=O)CCC(N4Cc5c(N)cccc5C4=O)C3=O)c2C)c1] |
| N3 | 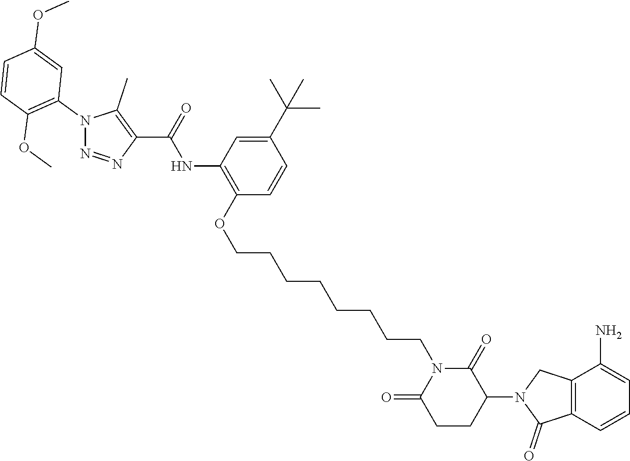 [SMILES: COc1ccc(OC)c(-n2nnc(C(=O)Nc3cc(C(C)(C)C)ccc3OCCCCCCCCN3C(=O)CCC(N4Cc5c(N)cccc5C4=O)C3=O)c2C)c1] |
| N4 | 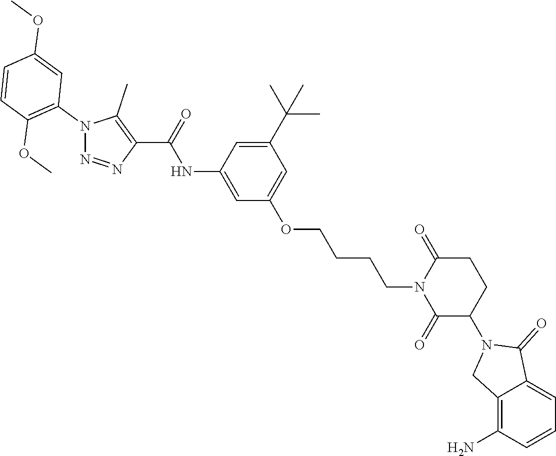 [SMILES: COc1ccc(OC)c(-n2nnc(C(=O)Nc3cc(OCCCCN4C(=O)CCC(N5Cc6c(N)cccc6C5=O)C4=O)cc(C(C)(C)C)c3)c2C)c1] |
| N5 | 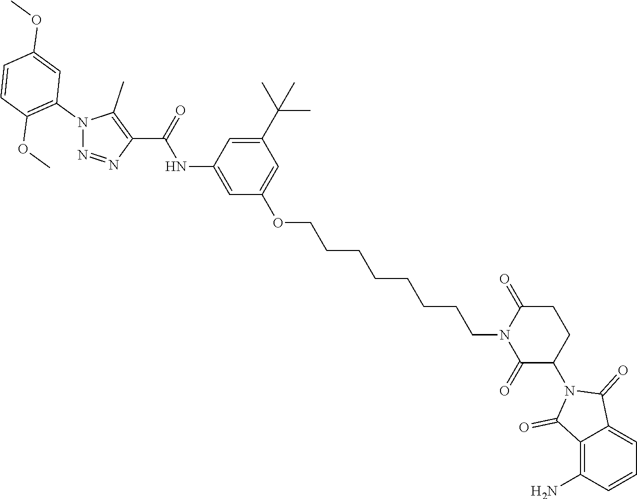 [SMILES: COc1ccc(OC)c(-n2nnc(C(=O)Nc3cc(OCCCCCCCCN4C(=O)CCC(N5C(=O)c6cccc(N)c6C5=O)C4=O)cc(C(C)(C)C)c3)c2C)c1] |
| N6 | 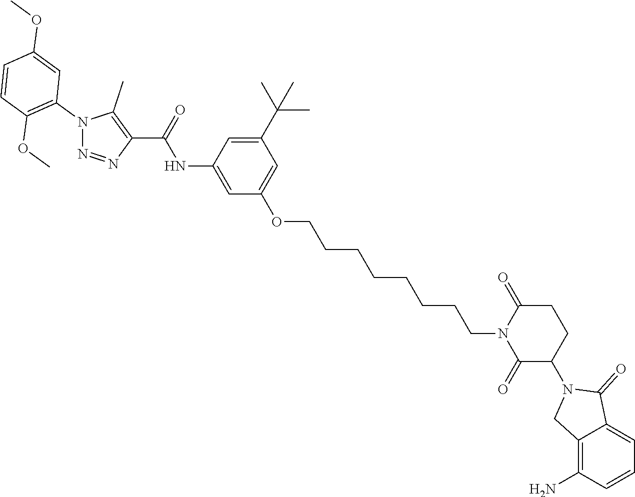 [SMILES: COc1ccc(OC)c(-n2nnc(C(=O)Nc3cc(OCCCCCCCCN4C(=O)CCC(N5Cc6c(N)cccc6C5=O)C4=O)cc(C(C)(C)C)c3)c2C)c1] |
| N7 | 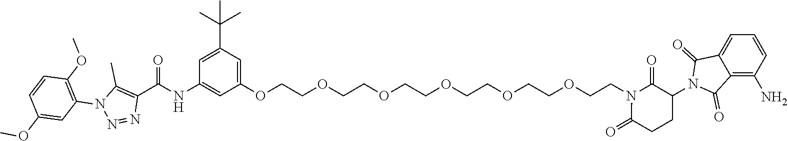 [SMILES: COc1ccc(OC)c(-n2nnc(C(=O)Nc3cc(OCCOCCOCCOCCOCCOCCN4C(=O)CCC(N5C(=O)c6cccc(N)c6C5=O)C4=O)cc(C(C)(C)C)c3)c2C)c1] |
| A1 | 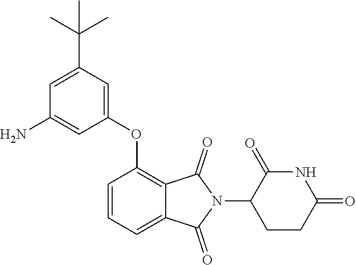 [SMILES: CC(C)(C)c1cc(N)cc(Oc2cccc3c2C(=O)N(C2CCC(=O)NC2=O)C3=O)c1] |
| A2 | 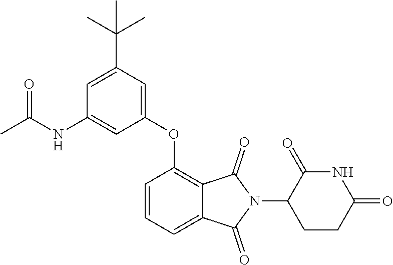 [SMILES: CC(=O)Nc1cc(Oc2cccc3c2C(=O)N(C2CCC(=O)NC2=O)C3=O)cc(C(C)(C)C)c1] |
| A3 | 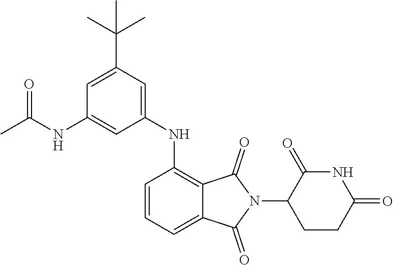 [SMILES: CC(=O)Nc1cc(Nc2cccc3c2C(=O)N(C2CCC(=O)NC2=O)C3=O)cc(C(C)(C)C)c1] |

TABLE 5

| | | | Inverse | |
|---|---|---|---|---|
| | Binding^{a} | Agonist^{b} | agonist^{c} | Antagonist^{d} |
| | (IC₅₀/μM) | (EC₅₀/μM) | (IC₅₀/μM) | (IC₅₀/μM) |
| | (% inhibition | (% activation | (% inhibition | (% inhibition |
| Compound | at 10 μM) | at 10 μM) | at 10 μM) | at 10 μM) |
| T0901317 | 0.026 ± 0.0034 | NT^{e} | NT | NT |
| | (100 ± 1)% | | | |
| Rifampicin | NT | 1.17 ± 0.095 | NA | NT |
| | | (100 ± 2)% | | |
| SPA70 | 0.21 ± 0.037 | NA^{f} | 0.022 ± 0.0035 | 0.23 ± 0.024 |
| | (87 ± 3)% | | (100 ± 2)% | (100 ± 1)% |
| M1 | NA | NA | NA | NA |
| M2 | 2.69 ± 1.33 | NA | NA | NA |
| | (46 ± 2)% | | | |
| M3 | NA | NA | NA | NA |
| M4 | NA | NA | 0.18 ± 0.055 | 0.94 ± 0.20 |
| | | | (133 ± 11)% | (96 ± 3)% |
| M5 | 0.083 ± 0.0041 | NA | 0.0078 ± 0.002 | 0.052 ± 0.0066 |
| | (69 ± 3)% | | 7 (141 ± 9)% | (107 ± 5)% |
| M6 | NA | NA | NA | NA |
| M7 | NA | NA | 0.20 ± 0.038 | 1.38 ± 0.47 |
| | | | (103 ± 11)% | (71 ± 4)% |
| M8 | NA | NA | 13.2 ± 1.28 | NA |
| | | | (19 ± 2)% | |
| M9 | NA | NA | NA | NA |
| M10 | NA | NA | 0.042 ± 0.0085 | 0.23 ± 0.023 |
| | | | (128 ± 10)% | (102 ± 1)% |
| M11 | 0.67 ± 0.014 | NA | 0.0079 ± 0.000 | 0.026 ± 0.0008 |
| | (61 ± 5)% | | 63 (135 ± 14)% | 4 (109 ± 2)% |
| M12 | 0.032 ± 0.002 | 0.27 ± 0.078 | NA | 0.67 ± 0.26 |
| | (56 ± 2)% | (33 ± 1)% | | (28 ± 6)% |
| M13 | NA | NA | NA | 41 ± 5.3 |
| | | | | (40 ± 5)% |
| M14 | 0.13 ± 0.051(22 ± 4)% | NA | 0.43 ± 0.14 | 1.86 ± 0.53 |
| | | | (94 ± 6)% | (82 ± 6)% |
| M15 | 2.19 ± 1.35 | NA | NA | NA |
| | (30 ± 1)% | | | |
| M16 | NA | NA | NA | 2.02 ± 0.47 |
| | | | | (57 ± 2)% |
| M17 | NA | NA | 1.98 ± 0.25 | 0.3790.19 |
| | | | (84 ± 7)% | (70 ± 2)% |
| M18 | NA | NA | 10.35 ± 1.06 | NA |
| | | | (42 ± 6)% | |
| M19 | NA | NA | NA | 2.52 ± 1.38 |
| | | | | (30 ± 5)% |
| M20 | 4.9 ± 0.8 (64 ± 7)% | NA | NA | 6.33 ± 0.74 |
| | | | | (58 ± 3)% |
| N1 | 1.27 ± 0.48 | 0.75 ± 0.11 | NA | NA |
| | (34 ± 0.5)% | (55 ± 5)% | | |
| N2 | 7.23 ± 4.88 | NA | NA | 5.0 ± 1.16 |
| | (27 ± 7)% | | | (32 ± 9)% |
| N3 | NA | NA | NA | NA |
| N4 | 0.087 ± 0.022 | NA | 0.044 ± 0.0080 | 0.17 ± 0.0045 |
| | (37 ± 7)% | | (97 ± 7)% | (103 ± 5)% |
| N5 | NA | 0.860.24 | NA | NA |
| | | (81 ± 5)% | | |
| N6 | NA | NA | NA | 6.51 ± 0.71 |
| | | | | (11 ± 2)% |
| N7 | 0.43 ± 0.027 | NA | 1.12 ± 0.3 | 0.52 ± 0.095 |
| | (48 ± 3)% | | (36 ± 13)% | (83 ± 12)% |

^{a}T0901317 at 10 μM as 100% inhibition and DMSO (0.3%) as 0% inhibition for the hPXR binding assay;
^{b}Rifampicin at 10 μM as 100% activation and DMSO (0.3%) as 0% activation for the cell-based hPXR agonistic assay;
^{c}SPA70 at 10 μM as 100% inhibition and DMSO (0.3%) as 0% inhibition for the cell-based hPXR inverse agonistic assay;
^{d}SPA70 at 10 μM with rifampicin (5 μM) as 100% inhibition and rifampicin alone (5 μM) as 0% inhibition for the cell-based hPXR antagonistic assay;
^{e}NT: not tested;
^{f}NA: no IC₅₀ value could be experimentally determined within the concentration range tested (highest concentration tested at 30 μM).

### 11. REFERENCES

1. Ito, T.; Ando, H.; Suzuki, T.; Ogura, T.; Hotta, K.; Imamura, Y.; Yamaguchi, Y.; Handa, H., Identification of a primary target of thalidomide teratogenicity. Science 2010, 327 (5971), 1345-50.
2. Kronke, J.; Udeshi, N. D.; Narla, A.; Grauman, P.; Hurst, S. N.; McConkey, M.; Svinkina, T.; Heckl, D.; Comer, E.; Li, X.; Ciarlo, C.; Hartman, E.; Munshi, N.; Schenone, M.; Schreiber, S. L.; Carr, S. A.; Ebert, B. L., Lenalidomide causes selective degradation of IKZF1 and IKZF3 in multiple myeloma cells. Science 2014, 343 (6168), 301-5.
3. Lu, G.; Middleton, R. E.; Sun, H.; Naniong, M.; Ott, C. J.; Mitsiades, C. S.; Wong, K. K.; Bradner, J. E.; Kaelin, W. G., Jr., The myeloma drug lenalidomide promotes the cereblon-dependent destruction of Ikaros proteins. Science 2014, 343 (6168), 305-9.
4. Winter, G. E.; Buckley, D. L.; Paulk, J.; Roberts, J. M.; Souza, A.; Dhe-Paganon, S.; Bradner, J. E., DRUG DEVELOPMENT. Phthalimide conjugation as a strategy for in vivo target protein degradation. Science 2015, 348 (6241), 1376-81.
5. Chamberlain, P. P.; Hamann, L. G., Development of targeted protein degradation therapeutics. Nat Chem Biol 2019, 15 (10), 937-944.
6. Melchert, M.; List, A., The thalidomide saga. Int J Biochem Cell Biol 2007, 39 (7-8), 1489-99.
7. Matyskiela, M. E.; Lu, G.; Ito, T.; Pagarigan, B.; Lu, C. C.; Miller, K.; Fang, W.; Wang, N. Y.; Nguyen, D.; Houston, J.; Carmel, G.; Tran, T.; Riley, M.; Nosaka, L.; Lander, G. C.; Gaidarova, S.; Xu, S.; Ruchelman, A. L.; Handa, H.; Carmichael, J.; Daniel, T. O.; Cathers, B. E.; Lopez-Girona, A.; Chamberlain, P. P., A novel cereblon modulator recruits GSPT1 to the CRL4(CRBN) ubiquitin ligase. Nature 2016, 535 (7611), 252-7.
8. Surka, C.; Jin, L.; Mbong, N.; Lu, C. C.; Jang, I. S.; Rychak, E.; Mendy, D.; Clayton, T.; Tindall, E.; Hsu, C.; Fontanillo, C.; Tran, E.; Contreras, A.; Ng, S. W. K.; Matyskiela, M.; Wang, K.; Chamberlain, P.; Cathers, B.; Carmichael, J.; Hansen, J.; Wang, J. C. Y.; Minden, M. D.; Fan, J.; Pierce, D. W.; Pourdehnad, M.; Rolfe, M.; Lopez-Girona, A.; Dick, J. E.; Lu, G., CC-90009, a novel cereblon E3 ligase modulator, targets acute myeloid leukemia blasts and leukemia stem cells. Blood 2021, 137 (5), 661-677.
9. Malta-Vacas, J.; Ferreira, P.; Monteiro, C.; Brito, M., Differential expression of GSPT1 GGCn alleles in cancer. Cancer Genet Cytogenet 2009, 195 (2), 132-42.
10. Nishiguchi, G.; Keramatnia, F.; Min, J.; Chang, Y.; Jonchere, B.; Das, S.; Actis, M.; Price, J.; Chepyala, D.; Young, B.; McGowan, K.; Slavish, P. J.; Mayasundari, A.; Jarusiewicz, J. A.; Yang, L.; Li, Y.; Fu, X.; Garrett, S. H.; Papizan, J. B.; Kodali, K.; Peng, J.; Pruett Miller, S. M.; Roussel, M. F.; Mullighan, C.; Fischer, M.; Rankovic, Z., Identification of Potent, Selective, and Orally Bioavailable Small-Molecule GSPT1/2 Degraders from a Focused Library of Cereblon Modulators. J Med Chem 2021, 64 (11), 7296-7311.
11. Ishoey, M.; Chorn, S.; Singh, N.; Jaeger, M. G.; Brand, M.; Paulk, J.; Bauer, S.; Erb, M. A.; Parapatics, K.; Muller, A. C.; Bennett, K. L.; Ecker, G. F.; Bradner, J. E.; Winter, G. E., Translation Termination Factor GSPT1 Is a Phenotypically Relevant Off-Target of Heterobifunctional Phthalimide Degraders. ACS Chem Biol 2018, 13 (3), 553-560.
12. Yang, J.; Li, Y.; Aguilar, A.; Liu, Z.; Yang, C. Y.; Wang, S., Simple Structural Modifications Converting a Bona fide MDM2 PROTAC Degrader into a Molecular Glue Molecule: A Cautionary Tale in the Design of PROTAC Degraders. J Med Chem 2019, 62 (21), 9471-9487.
13. Blumberg, B.; Sabbagh, W., Jr.; Juguilon, H.; Bolado, J., Jr.; van Meter, C. M.; Ong, E. S.; Evans, R. M., SXR, a novel steroid and xenobiotic-sensing nuclear receptor. Genes Dev 1998, 12 (20), 3195-205.
14. Bertilsson, G.; Heidrich, J.; Svensson, K.; Asman, M.; Jendeberg, L.; Sydow-Backman, M.; Ohlsson, R.; Postlind, H.; Blomquist, P.; Berkenstam, A., Identification of a human nuclear receptor defines a new signaling pathway for CYP3A induction. Proc Natl Acad Sci USA 1998, 95 (21), 12208-13.
15. Kliewer, S. A.; Moore, J. T.; Wade, L.; Staudinger, J. L.; Watson, M. A.; Jones, S. A.; McKee, D. D.; Oliver, B. B.; Willson, T. M.; Zetterstrom, R. H.; Perlmann, T.; Lehmann, J. M., An orphan nuclear receptor activated by pregnanes defines a novel steroid signaling pathway. Cell 1998, 92 (1), 73-82.
16. Xie, W.; Barwick, J. L.; Downes, M.; Blumberg, B.; Simon, C. M.; Nelson, M. C.; Neuschwander-Tetri, B. A.; Brunt, E. M.; Guzelian, P. S.; Evans, R. M., Humanized xenobiotic response in mice expressing nuclear receptor SXR. Nature 2000, 406 (6794), 435-9.
17. Lehmann, J. M.; McKee, D. D.; Watson, M. A.; Willson, T. M.; Moore, J. T.; Kliewer, S. A., The human orphan nuclear receptor PXR is activated by compounds that regulate CYP3A4 gene expression and cause drug interactions. J Clin Invest 1998, 102 (5), 1016-23.
18. Burk, O.; Koch, I.; Raucy, J.; Hustert, E.; Eichelbaum, M.; Brockmoller, J.; Zanger, U. M.; Wojnowski, L., The induction of cytochrome P450 3A⁵ (CYP3A5) in the human liver and intestine is mediated by the xenobiotic sensors pregnane X receptor (PXR) and constitutively activated receptor (CAR). J Biol Chem 2004, 279 (37), 38379-85.
19. Goodwin, B.; Moore, L. B.; Stoltz, C. M.; McKee, D. D.; Kliewer, S. A., Regulation of the human CYP2B6 gene by the nuclear pregnane X receptor. Mol Pharmacol 2001, 60 (3), 427-31.
20. Chen, C.; Staudinger, J. L.; Klaassen, C. D., Nuclear receptor, pregname X receptor, is required for induction of UDP-glucuronosyltranferases in mouse liver by pregnenolone-16 alpha-carbonitrile. Drug Metab Dispos 2003, 31 (7), 908-15.
21. Synold, T. W.; Dussault, I.; Forman, B. M., The orphan nuclear receptor SXR coordinately regulates drug metabolism and efflux. Nat Med 2001, 7 (5), 584-90.
22. Geick, A.; Eichelbaum, M.; Burk, O., Nuclear receptor response elements mediate induction of intestinal MDR1 by rifampin. J Biol Chem 2001, 276 (18), 14581-7.
23. Lin, W.; Wang, Y. M.; Chai, S. C.; Lv, L.; Zheng, J.; Wu, J.; Zhang, Q.; Wang, Y. D.; Griffin, P. R.; Chen, T., SPA70 is a potent antagonist of human pregnane X receptor. Nat Commun 2017, 8 (1), 741.
24. Lin, W.; Goktug, A. N.; Wu, J.; Currier, D. G.; Chen, T., High-Throughput Screening Identifies 1,4,5-Substituted 1,2,3-Triazole Analogs as Potent and Specific Antagonists of Pregnane X Receptor. Assay Drug Dev Technol 2017, 15 (8), 383-394.
25. Li, Y.; Lin, W.; Wright, W. C.; Chai, S. C.; Wu, J.; Chen, T., Building a Chemical Toolbox for Human Pregnane X Receptor Research: Discovery of Agonists, Inverse Agonists, and Antagonists Among Analogs Based on the Unique Chemical Scaffold of SPA70. J Med Chem 2021, 64 (3), 1733-1761.
26. Huber, A. D.; Wright, W. C.; Lin, W.; Majumder, K.; Low, J. A.; Wu, J.; Buchman, C. D.; Pintel, D. J.; Chen, T., Mutation of a single amino acid of pregnane X receptor switches an antagonist to agonist by altering AF-2 helix positioning. Cell Mol Life Sci 2021, 78 (1), 317-335.
27. Nabet, B.; Roberts, J. M.; Buckley, D. L.; Paulk, J.; Dastjerdi, S.; Yang, A.; Leggett, A. L.; Erb, M. A.; Lawlor, M. A.; Souza, A.; Scott, T. G.; Vittori, S.; Perry, J. A.; Qi, J.; Winter, G. E.; Wong, K. K.; Gray, N. S.; Bradner, J. E., The dTAG system for immediate and target-specific protein degradation. Nat Chem Biol 2018, 14 (5), 431-441.
28. Dixon, A. S.; Schwinn, M. K.; Hall, M. P.; Zimmerman, K.; Otto, P.; Lubben, T. H.; Butler, B. L.; Binkowski, B. F.; Machleidt, T.; Kirkland, T. A.; Wood, M. G.; Eggers, C. T.; Encell, L. P.; Wood, K. V., NanoLuc Complementation Reporter Optimized for Accurate Measurement of Protein Interactions in Cells. ACS Chem Biol 2016, 11 (2), 400-8.
29. Cheng, Z.; Saito, K.; Pisarev, A. V.; Wada, M.; Pisareva, V. P.; Pestova, T. V.; Gajda, M.; Round, A.; Kong, C.; Lim, M.; Nakamura, Y.; Svergun, D. I.; Ito, K.; Song, H., Structural insights into eRF3 and stop codon recognition by eRF1. Genes Dev 2009, 23 (9), 1106-18.
30. Preis, A.; Heuer, A.; Barrio-Garcia, C.; Hauser, A.; Eyler, D. E.; Berninghausen, O.; Green, R.; Becker, T.; Beckmann, R., Cryoelectron microscopic structures of eukaryotic translation termination complexes containing eRF1-eRF3 or eRF1-ABCE1. Cell Rep 2014, 8 (1), 59-65.
31. Zhouravleva, G.; Frolova, L.; Le Goff, X.; Le Guellec, R.; Inge-Vechtomov, S.; Kisselev, L.; Philippe, M., Termination of translation in eukaryotes is governed by two interacting polypeptide chain release factors, eRF1 and eRF3. EMBO J 1995, 14 (16), 4065-72.
32. Chai, S. C.; Wright, W. C.; Chen, T., Strategies for developing pregnane X receptor antagonists: Implications from metabolism to cancer. Med Res Rev 2020, 40 (3), 1061-1083.
33. Chen; Wang, Y.; Lu, R.; Jiang, X.; Chen, X.; Meng, N.; Chen, M.; Xie, S.; Yan, G. R., E3 ligase ZFP91 inhibits Hepatocellular Carcinoma Metabolism Reprogramming by regulating PKM splicing. Theranostics 2020, 10 (19), 8558-8572.
34. Jin, X.; Jin, H. R.; Jung, H. S.; Lee, S. J.; Lee, J. H.; Lee, J. J., An atypical E3 ligase zinc finger protein 91 stabilizes and activates NF-kappaB-inducing kinase via Lys63-linked ubiquitination. J Biol Chem 2010, 285 (40), 30539-47.
35. Tang, D. E.; Dai, Y.; Xu, Y.; Lin, L. W.; Liu, D. Z.; Hong, X. P.; Ou, M. L.; Jiang, H. W.; Xu, S. H., The ubiquitinase ZFP91 promotes tumor cell survival and confers chemoresistance through FOXA1 destabilization. Carcinogenesis 2020, 41 (1), 56-66.
36. Xu, S. H.; Zhu, S.; Wang, Y.; Huang, J. Z.; Chen, M.; Wu, Q. X.; He, Y. T.; Chen; Yan, G. R., ECD promotes gastric cancer metastasis by blocking E3 ligase ZFP91-mediated hnRNP F ubiquitination and degradation. Cell Death Dis 2018, 9 (5), 479.
37. Wang, A.; Ding, L.; Wu, Z.; Ding, R.; Teng, X. L.; Wang, F.; Hu, Z.; Chen, L.; Yu, X.; Zou, Q., ZFP91 is required for the maintenance of regulatory T cell homeostasis and function. J Exp Med 2021, 218 (2).
38. Donovan, K. A.; Ferguson, F. M.; Bushman, J. W.; Eleuteri, N. A.; Bhunia, D.; Ryu, S.; Tan, L.; Shi, K.; Yue, H.; Liu, X.; Dobrovolsky, D.; Jiang, B.; Wang, J.; Hao, M.; You, I.; Teng, M.; Liang, Y.; Hatcher, J.; Li, Z.; Manz, T. D.; Groendyke, B.; Hu, W.; Nam, Y.; Sengupta, S.; Cho, H.; Shin, I.; Agius, M. P.; Ghobrial, I. M.; Ma, M. W.; Che, J.; Buhrlage, S. J.; Sim, T.; Gray, N. S.; Fischer, E. S., Mapping the Degradable Kinome Provides a Resource for Expedited Degrader Development. Cell 2020, 183 (6), 1714-1731 e10.
39. Sentmanat, M. F.; Peters, S. T.; Florian, C. P.; Connelly, J. P.; Pruett-Miller, S. M., A Survey of Validation Strategies for CRISPR-Cas9 Editing. Sci Rep 2018, 8 (1), 888.
40. Connelly, J. P.; Pruett-Miller, S. M., CRIS.py: A Versatile and High-throughput Analysis Program for CRISPR-based Genome Editing. Sci Rep 2019, 9 (1), 4194.
41. High, A. A.; Tan, H.; Pagala, V. R.; Niu, M.; Cho, J. H.; Wang, X.; Bai, B.; Peng, J., Deep Proteome Profiling by Isobaric Labeling, Extensive Liquid Chromatography, Mass Spectrometry, and Software-assisted Quantification. J Vis Exp 2017, (129).
42. Wang, X.; Li, Y.; Wu, Z.; Wang, H.; Tan, H.; Peng, J., JUMP: a tag-based database search tool for peptide identification with high sensitivity and accuracy. Mol Cell Proteomics 2014, 13 (12), 3663-73.
43. Peng, J.; Elias, J. E.; Thoreen, C. C.; Licklider, L. J.; Gygi, S. P., Evaluation of multidimensional chromatography coupled with tandem mass spectrometry (LC/LC-MS/MS) for large-scale protein analysis: the yeast proteome. J Proteome Res 2003, 2 (1), 43-50.
44. Niu, M.; Cho, J. H.; Kodali, K.; Pagala, V.; High, A. A.; Wang, H.; Wu, Z.; Li, Y.; Bi, W.; Zhang, H.; Wang, X.; Zou, W.; Peng, J., Extensive Peptide Fractionation and y1 Ion-Based Interference Detection Method for Enabling Accurate Quantification by Isobaric Labeling and Mass Spectrometry. Anal Chem 2017, 89 (5), 2956-2963.
45. Bai, B.; Wang, X.; Li, Y.; Chen, P. C.; Yu, K.; Dey, K. K.; Yarbro, J. M.; Han, X.; Lutz, B. M.; Rao, S.; Jiao, Y.; Sifford, J. M.; Han, J.; Wang, M.; Tan, H.; Shaw, T. I.; Cho, J. H.; Zhou, S.; Wang, H.; Niu, M.; Mancieri, A.; Messler, K. A.; Sun, X.; Wu, Z.; Pagala, V.; High, A. A.; Bi, W.; Zhang, H.; Chi, H.; Haroutunian, V.; Zhang, B.; Beach, T. G.; Yu, G.; Peng, J., Deep Multilayer Brain Proteomics Identifies Molecular Networks in Alzheimer's Disease Progression. Neuron 2020, 105 (6), 975-991 e7.
46. Lin, W.; Li, Y.; Min, J.; Liu, J.; Yang, L.; Lee, R. E.; Chen, T., Development of BODIPY FL Thalidomide As a High-Affinity Fluorescent Probe for Cereblon in a Time-Resolved Fluorescence Resonance Energy Transfer Assay. Bioconjug Chem 2020, 31 (11), 2564-2575.
It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 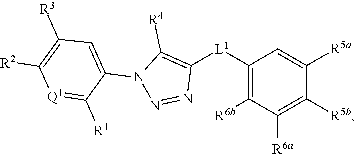
wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—;
wherein R¹⁰ is selected from hydrogen and C1-C4 alkyl;
wherein R¹¹, when present, is selected from hydrogen and C1-C4 alkyl;
wherein Q¹ is selected from N and CH;
wherein R¹ is selected from hydrogen, halogen, —OH, C1-C6 alkyl, C1-C6 alkoxy, C1-C6 haloalkyl, C1-C6 hydroxyalkyl, and C1-C6 haloalkoxy;
wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), —CO₂(C1-C6 alkyl), and R⁷;
wherein R³ is selected from hydrogen, halogen, —OH, C1-C6 alkyl, C1-C6 alkoxy, C1-C6 haloalkyl, C1-C6 haloalkoxy, and C1-C6 hydroxyalkyl;
wherein R⁴ is selected from hydrogen and C1-C4 alkyl;
wherein each of R^{5a} and R^{5b} is independently selected from hydrogen and C1-C8 alkyl;
wherein each of R^{6a} and R^{6b} is independently selected from hydrogen, C1-C8 alkyl, and R⁷;
wherein R⁷ is a structure represented by a formula selected from: 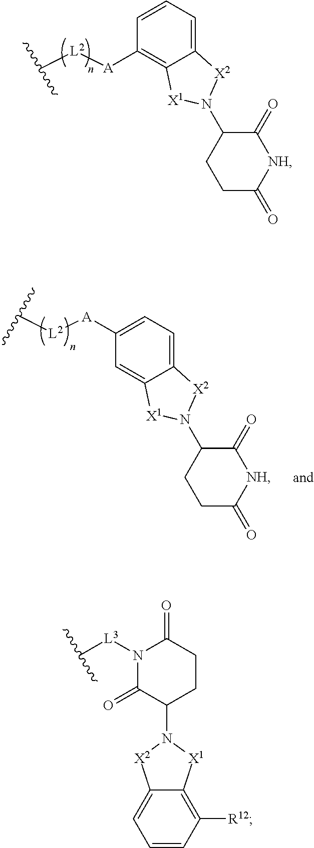
wherein n is selected from 0 and 1;
wherein A is selected from —O—, —NH—, —CH₂—, and —C≡C—;
wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-;
wherein q, when present, is selected from 1, 2, 3, 4, 5, 6, 7, and 8;
wherein Ar¹ is a structure selected from: 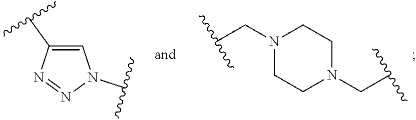
wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-;
wherein r, when present, is selected from 1, 2, 3, 4, 5, 6, 7, and 8;
wherein each of X¹ and X² is independently selected from —CH₂— and —C(O)—, provided that at least one of X¹ and X² is —C(O)—; and
wherein R¹² is selected from hydrogen, —OH, and —NH₂,
provided that one and only one of R², R^{6a}, and R^{6b} is R⁷, or a pharmaceutically acceptable salt thereof.

2. (canceled)

3. The compound of claim 1, wherein L¹ is selected from —C(O)NR¹⁰— and —SO₂NR¹⁰—.

4. (canceled)

5. The compound of claim 1, wherein Q¹ is CH.

6. The compound of claim 1, wherein R¹ is C1-C4 alkoxy.

7. (canceled)

8. The compound of claim 1, wherein R² is hydrogen.

9. The compound of claim 1, wherein R³ is C1-C4 alkoxy.

10-12. (canceled)

13. The compound of claim 1, wherein R⁴ is methyl.

14. The compound of claim 1, wherein R^{5a} is C1-C8 alkyl.

15-16. (canceled)

17. The compound of claim 1, wherein R^{5b} is C1-C8 alkyl.

18-21. (canceled)

22. The compound of claim 1, wherein one of R^{6a} and R^{6b} is a structure represented by a formula:

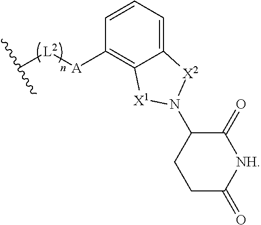

23-27. (canceled)

28. The compound of claim 1, wherein one of R^{6a} and R^{6b} is a structure represented by a formula:

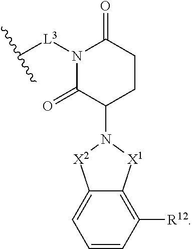

29-31. (canceled)

32. The compound of claim 1, wherein the compound has a structure represented by a formula selected from: 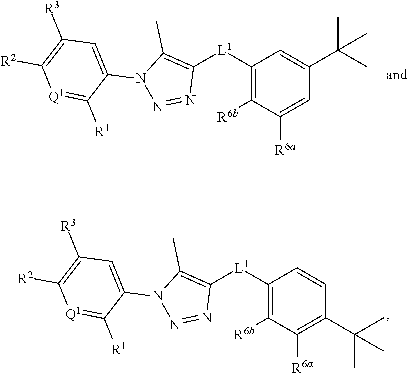
or a pharmaceutically acceptable salt thereof.

33-38. (canceled)

39. The compound of claim 1, wherein the compound has a structure represented by a formula: 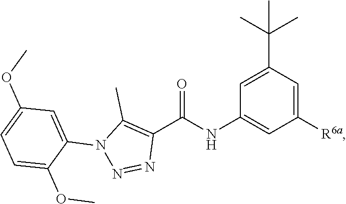
or a pharmaceutically acceptable salt thereof.

40. (canceled)

41. The compound of claim 1, wherein the compound is selected from: 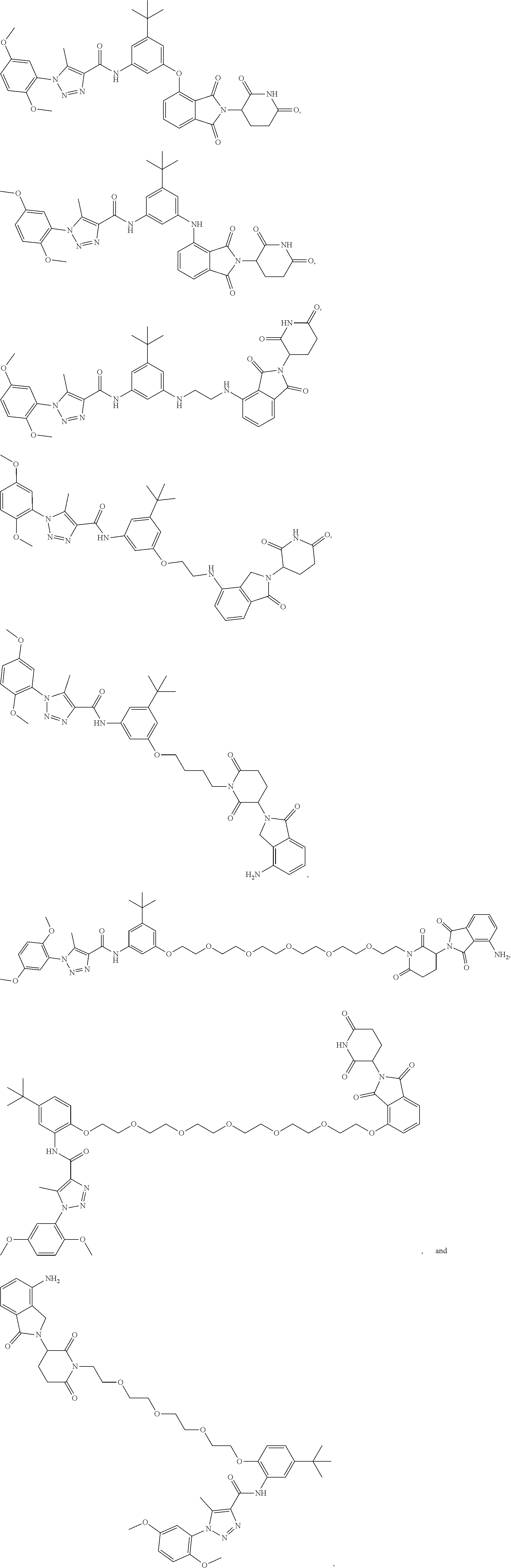
or a pharmaceutically acceptable salt thereof.

42. The compound of claim 1, wherein the compound has a structure represented by a formula: 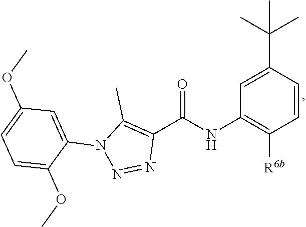
or a pharmaceutically acceptable salt thereof.

43. The compound of claim 0, wherein the compound is: 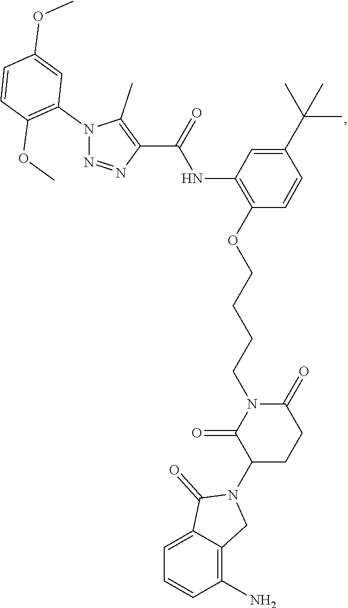 [SMILES: COc1ccc(OC)c(-n2nnc(C(=O)Nc3cc(C(C)(C)C)ccc3OCCCCN3C(=O)CCC(N4Cc5c(N)cccc5C4=O)C3=O)c2C)c1]
or a pharmaceutically acceptable salt thereof.

44. A pharmaceutical composition comprising an effective amount of the compound of claim 1 or a pharmaceutically acceptable salt thereof, and a pharmaceutically acceptable carrier.

45-60. (canceled)

61. A method for treating a disorder of uncontrolled cellular proliferation in a subject in need thereof, the method comprising administering to the subject an effective amount of the compound of claim 1 or a pharmaceutically acceptable salt thereof.

62-76. (canceled)

77. A method for decreasing an adverse drug reaction in a subject in need thereof, the method comprising administering to the subject an effective amount of a compound having a structure represented by a formula: 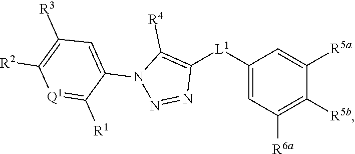
wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—;
wherein R¹⁰ is selected from hydrogen and C1-C4 alkyl;
wherein R¹¹, when present, is selected from hydrogen and C1-C4 alkyl;
wherein Q¹ is selected from N and CH;
wherein R¹ is selected from hydrogen, halogen, —OH, C1-C6 alkyl, C1-C6 alkoxy, C1-C6 hydroxyalkyl, and C1-C6 haloalkoxy;
wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl);
wherein R³ is selected from hydrogen, halogen, —OH, C1-C6 alkyl, C1-C6 alkoxy, and C1-C6 hydroxyalkyl;
wherein R⁴ is selected from hydrogen and C1-C4 alkyl;
wherein each of R^{5a} and R^{5b} is independently selected from hydrogen and C1-C8 alkyl;
wherein R^{6a} is R⁷;
wherein R⁷ is a structure represented by a formula selected from: 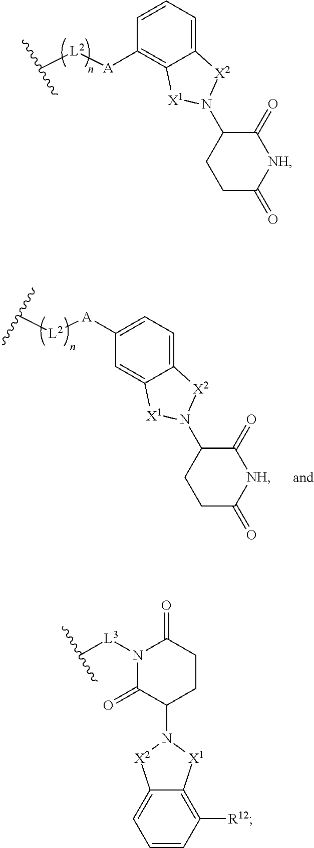
wherein n is selected from 0 and 1;
wherein A is selected from —O—, —NH—, —CH₂—, and —C≡C—;
wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-;
wherein q, when present, is selected from 1, 2, 3, 4, 5, 6, 7, and 8;
wherein Ar¹ is a structure selected from: 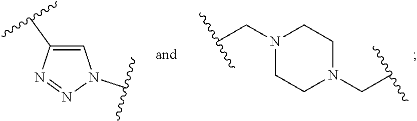
wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-;
wherein r, when present, is selected from 1, 2, 3, 4, 5, 6, 7, and 8;
wherein each of X¹ and X² is independently selected from —CH₂— and —C(O)—, provided that at least one of X¹ and X² is —C(O)—; and
wherein R¹² is selected from hydrogen, —OH, and —NH₂, or a pharmaceutically acceptable salt thereof.

78-125. (canceled)

126. A method for treating a bowel disorder in a subject in need thereof, the method comprising administering to the subject an effective amount of a compound having a structure represented by a formula: 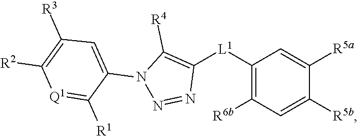
wherein L¹ is selected from —SO₂—, —CO₂—, —OC(O)—, —C(O)NR¹⁰—, —NR¹⁰C(O)—, —SO₂NR¹⁰—, —NR¹⁰SO₂—, and —N(R¹⁰)C(O)NR¹¹—;
wherein R¹⁰ is selected from hydrogen and C1-C4 alkyl;
wherein R¹¹, when present, is selected from hydrogen and C1-C4 alkyl;
wherein Q¹ is selected from N and CH;
wherein R¹ is selected from hydrogen, halogen, —OH, C1-C6 alkyl, C1-C6 alkoxy, C1-C6 hydroxyalkyl, and C1-C6 haloalkoxy;
wherein R² is selected from hydrogen, halogen, —OH, —CN, C1-C6 alkyl, C1-C6 alkoxy, C1-C6 haloalkyl, C1-C4 haloalkoxy, —CO₂H, —(C═O)H, —C(O)(C1-C6 alkyl), and —CO₂(C1-C6 alkyl);
wherein R³ is selected from hydrogen, halogen, —OH, C1-C6 alkyl, C1-C6 alkoxy, and C1-C6 hydroxyalkyl;
wherein R⁴ is selected from hydrogen and C1-C4 alkyl;
wherein each of R^{5a} and R^{5b} is independently selected from hydrogen and C1-C8 alkyl;
wherein R^{6b} is R⁷;
wherein R⁷ is a structure represented by a formula selected from: 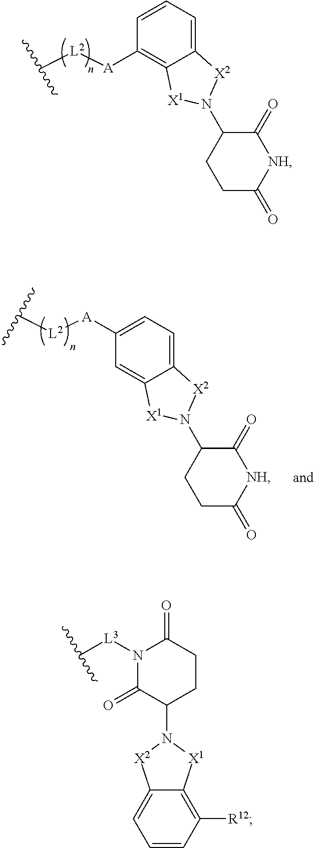
wherein n is selected from 0 and 1;
wherein A is selected from —O—, —NH—, —CH₂—, and —C≡C—;
wherein L² is selected from —O(C1-C8 alkyl)-, —NH(C1-C8 alkyl)-, —OC(O)—, —NHC(O)—, —OC(O)(C1-C8 alkyl)-, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)_{q}—, —(OCH₂CH₂)_{q}—, and —O(C1-C8 alkyl)Ar¹(C1-C8 alkyl)NHC(O)(C1-C4 alkyl)-;
wherein q, when present, is selected from 1, 2, 3, 4, 5, 6, 7, and 8;
wherein Ar¹ is a structure selected from: 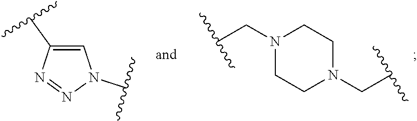
wherein L³ is selected from —O(C1-C8 alkyl)-, —NH(C1-C8 alkyl)-, —OC(O)—, —NHC(O)—, —NHC(O)(C1-C8 alkyl)-, —O(C1-C8 alkyl)C(O)—, —NH(C1-C8 alkyl)C(O)—, —NHC(O)(C1-C8 alkyl)NHC(O)(C1-C8 alkyl)-, —NHC(O)(C1-C4 alkyl)(OCH₂CH₂)ᵣ—, —(OCH₂CH₂)ᵣ—, and —O(C1-C8 alkyl)Ar¹(C1-C8 alkyl)NHC(O)(C1-C4 alkyl)-;
wherein r, when present, is selected from 1, 2, 3, 4, 5, 6, 7, and 8;
wherein each of X¹ and X² is independently selected from —CH₂— and —C(O)—, provided that at least one of X¹ and X² is —C(O)—; and
wherein R¹² is selected from hydrogen, —OH, and —NH₂, or a pharmaceutically acceptable salt thereof.

127-148. (canceled)
